(19) United States
(12) Patent Application Publication
(10) Pub. No.: US 2024/0392003 A1
(43) Pub. Date: Nov. 28, 2024

(54) MULTISPECIFIC ANTIBODIES HAVING SPECIFICITY FOR ROR1 AND CD3

(71) Applicant: Numab Therapeutics AG, Horgen (CH)

(72) Inventors: Bithi CHATTERJEE, Zurich (CH); Daniel SNELL, Thalwil (CH); Alexandre SIMONIN, Rosenau (FR); Tea GUNDE, Zurich (CH); Christian HESS, Zurich (CH); Stefan WARMUTH, Au (CH); Matthias BROCK, Aesch (CH); Julia TIETZ, Schlieren (CH); Maria JOHANSSON, Denges (CH); Fabio Mario SPIGA, Horgen (CH)

(21) Appl. No.: 18/263,753

(22) PCT Filed: Feb. 2, 2022
(86) PCT No.: PCT/EP2022/052425

§ 371 Date: Aug. 1, 2023

(30) Foreign Application Priority Data
Feb. 2, 2021 (EP) 21154786.4
Dec. 23, 2021 (WO) PCT/EP2021/087618

(51) Int. Cl. C07K 16/28; A61P 35/00; C07K 16/18

(52) U.S. Cl. CPC C07K 16/2809; A61P 35/00; C07K 16/18; C07K 16/2803; C07K 2317/31; C07K 2317/569; C07K 2317/622

(57) ABSTRACT

The present invention relates to a multispecific antibody comprising one or two binding domains, which specifically bind to the extracellular domain of ROR1 (ROR1-BDs), and one binding domain, which specifically binds to CD3 (CD3-BD), wherein the multispecific antibody does not comprise an immunoglobulin Fc region. The present invention further relates to nucleic acids encoding said multispecific antibody, vector(s) comprising said nucleic acids, host cell(s) comprising said nucleic acids or said vector(s), and a method of producing said multispecific antibody. Additionally, the present invention relates to pharmaceutical compositions comprising said multispecific antibody and methods of use thereof.

### CROSS-REFERENCE TO RELATED APPLICATIONS

This application is a 371 National Phase of International Patent Application No. PCT/EP2022/052425 filed on Feb. 2, 2022, which claims priority to European Patent Application 21154786.4 filed on Feb. 2, 2021 and International Patent Application No. PCT/EP2021/087618 filed on Dec. 23, 2021, the content of each of which applications is Incorporated herein by reference.

### REFERENCE TO AN ELECTRONIC SEQUENCE LISTING

The Instant application contains a Sequence Listing submitted via EFS-Web, hereby incorporated by reference in its entirety, which was created on Jul. 31, 2023, is named “WR.N27.NP_seglist.txt” and is 212,473 bytes in size and corresponds to the Sequence Listing originally submitted in International Appl. No. PCT/EP2022/052425 filed on Feb. 2, 2022.

### FIELD OF THE INVENTION

The present Invention relates to a multispecific antibody comprising one or two binding domains, which specifically bind to the extracellular domain of ROR1 (ROR1-BDs), and one binding domain, which specifically binds to CD3 (CD3-BD), wherein the multispecific antibody does not comprise an immunoglobulin Fc region. The present Invention further relates to nucleic acids encoding said multispecific antibody, vector(s) comprising said nucleic acids, host cell(s) comprising said nucleic acids or said vector(s), and a method of producing said multispecific antibody. Additionally, the present invention relates to pharmaceutical compositions comprising said multispecific antibody and methods of use thereof.

### BACKGROUND OF THE INVENTION

Tyrosine-protein kinase transmembrane receptor ROR1 is a member of the receptor tyrosine kinase-like orphan receptor (ROR) family. The extracellular domain of the ROR1 receptor is composed of three distinct domains: a membrane-distal Immunoglobulin-Like Domain, an intervening Frizzled Domain and a membrane-proximal Kringle Domain. ROR1 is an onco-fet all antigen required during embryogenesis. Mice with ROR1 homozygous knockouts die perinatally due to respiratory dysfunction (Borcherding at al 2014). The expression of ROR1 in normal adult tissues is limited to adipose tissue, certain cells in the pancreas and lungs, as well as a subset of Intermediate B cells (Baskar et al 2008; Hudecek et all 2010; Bicocca et al., 2012). It is however expressed on a number of hematological and solid cancers as well as on a subset of cancer stem cells. Furthermore, several studies demonstrate that ROR1 μlays an important role in cancer progression and metastasis (see for example Cui et al., 2013). ROR1 is thus an attractive target for tumor-specific therapy.

ROR1 is a cell surface protein that mediates signals through the binding of its ligands, which are believed to be Wnt5a and NKX1-2. Wnt5a has been shown to bind to the Frizzled Domain in the extracellular part of ROR1 and, in transfected cells, has been shown to modulate NF-κB activation and proliferation of normal and lung tumor cell lines. Binding of NKX1-2 to ROR1 has been shown to play a role in the survival of lung cancer cell lines through both kinase-dependent and kinase-independent mechanisms.

Cluster of differentiation 3 (CD3) is a type I transmembrane protein expressed on the surface of certain T cells that is required for T cell activation. It participates in the T cell receptor (TCR) complex and Interacts with other domains of this complex. In mammals, CD3 possesses three distinct chains (γ, δ, and ε), and either a ζ2 (CD247) chain or ζ/η chain. The CD3- and ζ-chains, together with the TCR, form what is known as the T cell receptor complex. Antibodies against CD3, e.g. antibodies against CD3c, have been shown to induce the clustering of CD3 on T cells, causing T cell activation.

Some bispecific antibodies targeting ROR1 and CD3 have been described in the prior art.

For example, WO 2017/127499 (A1) discloses bispecific anti-ROR1xCD3 antibodies, i.e. DuoBody molecules, that specifically bind to either the Ig-like domain, Frizzled domain or the Kringe domain of the extracellular domain of ROR1. The DuoBody® molecules exhibit EC₅₀ values in the range of 0.2 to 17 nM for the T cell-directed killing of ROR1-expressing modified SK-MES-1 cells with a maximum killing typically in the range of 20 to 40%. The improved bispecific anti-ROR1xCD3 DuoBody molecules RORxCD3 D1 and ROR1xCD3 D2 exhibit EC₅₀ values in the range of from 0.2 to 1 nM for the T cell-directed killing of ROR1-expressing cancer cells MAVER-1, JeKo-1, and Z-138, typically with a maximum killing of below 50%.

WO 2017/142928 (A1) discloses anti-ROR1xCD3 bispecific diabodies consisting of two cross-linked and disulfide-stabilized Fv binding domains having specificity for two target antigens, and which are connected to an immunoglobulin Fc region (DART format). Further disclosed are T cell-directed cytotoxcity tests for several of these DART antibodies against different ROR1-expressing cancer cell lines. The EC₅₀ values for killing of these cancer cells typically range from 10 to 100 pM with a maximum killing of from 10 to 40%, with some exceptions. However, only one example is shown where the maximum killing is somewhat higher than 60%. For the best DART antibody DART-D, EC₅₀ values in the range of 1.3 to 56 pM are reported.

WO 2019/008379 (A1) discloses anti-ROR1xCD3 bispecific BITEs, i.e. bispecific antibodies consisting of two scFv fragments connected via a short linker. The best BITE exemplified is Clone F, which exhibits significant cytotoxicity of ROR1-positive B-1643 and B-7 neuroblastoma cells at concentrations of 0.01 micrograms/ml.

WO 2020/237173 (A1) discloses anti-ROR1xCD3 bispecific antibodies having five different bispecific IgG-based configurations. 20 of these IgG-based bispecific antibodies were prepared and analyzed for internalization and their ability to induce T cell-mediated killing of various ROR1-expressing cancer cells. All 20 constructs got internalized albeit at different rates. The 20 constructs exhibit EC₅₀ values for T cell mediated killing of said ROR1-expressing cancer cells typically ranging from a few pM to 50 pM, with a maximum killing of 10 to 50%. Only one example is reported where the maximum killing exceeds 60%.

WO 2014/167022 (A1) and the follow-up patent application WO 2016/055592 (A1) disclose bispecific antibodies comprising one Fab fragment, which specifically binds to human CD3ε and one or two further Fab fragments, which specifically bind to the extracellular domain of human ROR1, wherein said bispecific antibodies exhibit a low rate of internalization in a cell-based assay. Bispecific antibody constructs that are monovalent or bivalent for ROR and that comprise an immunoglobulin Fc region are exemplified and were subjected to internalization experiments. The results of the internalization experiments indicate that the antibody constructs that are bivalent for ROR1 exhibit a faster intemalization rate in primary B-CLL cells than the corresponding antibody constructs that are monovalent for ROR1. T cell directed cytoloxicity tests with these bispecific antibodies against ROR1-expressing RPM18226 MM target cells demonstrate a maximum target cell lysis of 30 to 40% at an antibody concentration of 100 pM. WO 2016/055592 (A1) further discloses T cell directed cytotoxicity tests with the improved antibody Mab2-TCBcv that is bivalent for ROR1, tested against several target cells expressing different levels of ROR1. The data show that Mab2-TCBcv exhibits a maximum target cell lysis of 20 to 50% for Colo-704 and OVCAR-5 ovarian cancer cells expressing medium levels of ROR1, and a maximum target cell lysis of 20% for SKOV-3 ovarian cancer cells expressing low levels of ROR1.

As apparent from the above references, prior art anti-ROR1xCD3 antibodies often exhibit a significantly reduced potency against cancer cells expressing low levels of ROR1. Besides, these prior art antibodies typically exhibit a low percentage of maximum killing—often well below 50%—in cytotoxicity assays against many ROR1-expressing cancer cells, in particular against low-ROR1-expressing cancer cells, which Indicates that their mode of action is not ideal.

Accordingly, there is a clear need for novel and Improved anti-ROR1xCD3 therapeutic antibodies.

Said therapeutic antibodies should have a high potency against high-ROR1-expressing cancer cells as well as against cancer cells that express ROR1 at lower levels. Furthermore, they should exhibit a reasonable percentage of maximum killing, e.g. 40% and more, for these cancer cells. At the same time they should exhibit a tolerable safety profile to keep dose limiting side effects at a low level. Furthermore, it is desirable that said therapeutic antibodies have superior biophysical properties, such as a high stability, in order to facilitate developability and producibility in high yields.

### SUMMARY OF THE INVENTION

It is an object of the present Invention to provide a medicament to improve treatment of cancers that express ROR1. In particular, it is an object of the present invention to provide novel and improved therapeutic antibodies that are also able to efficiently eradicate cancer cells expressing low levels of ROR1. It is a further object of the present Invention to identify anti-ROR1xCD3 antibodies, which have an improved mode of action, i.e. which exhibit a high percentage of maximum killing in cytotoxicity assays against ROR1-expressing cancer cells. Said novel and improved therapeutic antibodies should further exhibit a tolerable safety profile, i.e. should not affect healthy cells or should not cause dose limiting toxicities by systemic activation of T cells through unspecific binding to CD3.

The Inventors have now found that multispecific antibodies comprising one or two binding domains that specifically target ROR1 (ROR1-BD9) and one binding domain that specifically targets CD3 (CD3-BDs), as defined herein, in particular binding domains characterized by particular sequence characteristics, exhibit a surprisingly high potency in killing cancer cells that express ROR1 at high to low levels. Furthermore, it has surprisingly been found that said multispecific antibodies comprising one ROR1-BD, as defined herein, typically exhibit a maximum killing of 60% and higher in T cell directed cytotoxicity assays against cancer cells that express ROR1 at high to low levels.

Accordingly, in a first aspect, the present Invention relates to a multispecific antibody comprising:
a) one or two binding domains, which specifically bind to the extracellular domain of ROR1 (ROR1-BDs); and
b) one binding domain, which specifically binds to CD3 (CD3-BD);
   wherein
   the multispecific antibody does not comprises an immunoglobulin Fc region;
   said ROR1-BDs comprise independently from each other a set of CDR sequences selected from the set consisting of
   the HCDR1 sequence of SEQ ID NO: 1,
   the HCDR2 sequence of SEQ ID NO: 2,
   the HCDR3 sequence of SEQ ID NO: 3,
   the LCDR1 sequence of SEQ ID NO: 4,
   the LCDR2 sequence of SEQ ID NO: 5, and
   the LCDR3 sequence of SEQ ID NO: 6;
   and/or the set consisting of
   the HCDR1 sequence of SEQ ID NO: 13 or 14,
   the HCDR2 sequence of SEQ ID NO: 15,
   the HCDR3 sequence of SEQ ID NO: 16,
   the LCDR1 sequence of SEQ ID NO: 17,
   the LCDR2 sequence of SEQ ID NO: 18, and
   the LCDR3 sequence of SEQ ID NO: 19;
   said CD3-BD comprises the set of CDR sequences consisting of
   the HCDR1 sequence of SEQ ID NO: 30,
   the HCDR2 sequence of SEQ ID NO: 31,
   the HCDR3 sequence of SEQ ID NO: 32,
   the LCDR1 sequence of SEQ ID NO: 33,
   the LCDR2 sequence of SEQ ID NO: 34, and
   the LCDR3 sequence of SEQ ID NO: 35.

In a second aspect, the present invention relates to specific ROR1“binding domains.

In a third aspect, the present invention relates to a nucleic acid or two nucleic acids encoding the multispecific antibody or the specific ROR1“binding domain of the present invention.

In a fourth aspect, the present Invention relates to a vector or two vectors comprising the nucleic acid or the two nucleic acids of the present invention.

In a fifth aspect, the present Invention relates to a host cell or host cells comprising the vector or the two vectors of the present invention.

In a sixth aspect, the present invention relates to a method for producing the multispecific antibody of the present invention, comprising (1) providing the nucleic acid or the two nucleic acids of the present invention, or the vector or the two vectors of the present invention, expressing said nucleic acid or nucleic acids, or said vector or vectors, and collecting said multispecific antibody or said specific binding domain from the expression system, or (ii) providing a host cell or host cells of the present invention, culturing said host cell or said host cells; and collecting said multispecific antibody or said specific binding domain from the cell culture.

In a seventh aspect, the present invention relates to a pharmaceutical composition comprising the multispecific antibody of the present invention and a pharmaceutically acceptable carrier.

In an eighth aspect, the present invention relates to a multispecific antibody of the present invention for use as a medicament.

In a ninth aspect, the present invention relates to a multispecific antibody of the present invention for use in the treatment of a disease, more particularly a human disease selected from cancer, particularly from cancer that expresses ROR1.

In a tenth aspect, the present invention relates to a method for the teatment of a disease, particularly a human disease, more particularly a human disease selected from cancer, comprising the step of administering the multispecific antibody of the present invention to a patient in need thereof.

The aspects, advantageous features and preferred embodiments of the present invention summarized in the following items, respectively alone or in combination, further contribute to solving the object of the inventiorn
1. A multispecific antibody comprising:
   a) one or two binding domains, which specifically bind to the extracellular domain of ROR1 (ROR1-BDs); and
   b) one binding domain, which specifically binds to CD3 (CD3-BD);
   wherein
      said ROR1-BDs comprise independently from each other a set of CDR sequences
      selected from the set consisting of
      the HCDR1 sequence of SEQ ID NO: 1,
      the HCDR2 sequence of SEQ ID NO: 2,
      the HCDR3 sequence of SEQ ID NO: 3,
      the LCDR1 sequence of SEQ ID NO: 4,
      the LCDR2 sequence of SEQ ID NO: 5, and
      the LCDR3 sequence of SEQ ID NO: 6;
      and/or the set consisting of
      the HCDR1 sequence of SEQ ID NO: 13 or 14,
      the HCDR2 sequence of SEQ ID NO: 15,
      the HCDR3 sequence of SEQ ID NO: 16,
      the LCDR1 sequence of SEQ ID NO: 17,
      the LCDR2 sequence of SEQ ID NO: 18, and
      the LCDR3 sequence of SEQ ID NO: 19;
      said CD3-BD comprises the set of CDR sequences consisting of
      the HCDR1 sequence of SEQ ID NO: 30,
      the HCDR2 sequence of SEQ ID NO: 31,
      the HCDR3 sequence of SEQ ID NO: 32,
      the LCDR1 sequence of SEQ ID NO: 33,
      the LCDR2 sequence of SEQ ID NO: 34, and
      the LCDR3 sequence of SEQ ID NO: 35.
2. The multispecific antibody of item 1, wherein the multispecific antibody comprises:
   a) one or two binding domains, which specifically bind to the extracellular domain of ROR1 (ROR1-BDs); and
   b) one binding domain, which specifically binds to CD3 (CD3-BD);
   wherein
      said ROR1-BDs comprise Independently from each other a set of CDR sequences
      selected from the set consisting of
      the HCDR1 sequence of SEQ ID NO: 1,
      the HCDR2 sequence of SEQ ID NO: 2,
      the HCDR3 sequence of SEQ ID NO: 3,
      the LCDR1 sequence of SEQ ID NO: 4,
      the LCDR2 sequence of SEQ ID NO: 5, and
      the LCDR3 sequence of SEQ ID NO: 6;
      or the set consisting of
      the HCDR1 sequence of SEQ ID NO: 13 or 14,
      the HCDR2 sequence of SEQ ID NO: 15,
      the HCDR3 sequence of SEQ ID NO: 16,
      the LCDR1 sequence of SEQ ID NO: 17,
      the LCDR2 sequence of SEQ ID NO: 18, and
      the LCDR3 sequence of SEQ ID NO: 19;
      said CD3-BD comprises the set of CDR sequences consisting of
      the HCDR1 sequence of SEQ ID NO: 30,
      the HCDR2 sequence of SEQ ID NO: 31,
      the HCDR3 sequence of SEQ ID NO: 32,
      the LCDR1 sequence of SEQ ID NO: 33,
      the LCDR2 sequence of SEQ ID NO: 34, and
      the LCDR3 sequence of SEQ ID NO: 35.
3. The multispecific antibody of item 1 or 2, wherein said multispecfic antibody has one or more of the following features 1) to 3):
   1) said multispecific antibody does not comprise an immunoglobulin Fc region;
   2) said multispecific antibody does not comprise CH1 and/or CL regions;
   3) said multispecfic antibody is humanized, in particular said multispecific antibody is humanized and comprises rabbit-derived CDRs.
4. The multispecific antibody of any one of the preceding items, wherein said one or two ROR1-BDs bind to the ig-lke domain of ROR1 and/or do not block the binding of Wnt5a to ROR1.
5. The multispecific antibody of any one of the preceding Items, where in case said multispecific antibody comprises two ROR1-BDs, said two ROR1-BDs comprise two different sets of CDRs, i.e. one ROR1-BD comprises CDRs of SEQ ID NOs: 1 to 6 and the other ROR1-BD comprises CDRs of SEQ ID NOs: 13/14 to 19, or said two ROR1-BDs comprise the same sets of CDRs, i.e. both ROR1-BDs either comprise CDRs of SEQ ID NOs: 1 to 6 or CDRs of SEQ ID NOs: 13/14 to 19, particularly said two ROR1-BDs comprise the same sets of CDRs.
6. The multispecific antibody of any one of the preceding items, wherein the multispecific antibody is trispecific.
7. The multispecific antibody of em 6, wherein said multispecific antibody comprises one binding domain, which specifically binds to human serum albumin (hSA-BD).
8. The multispecific antibody of any one of the preceding items, wherein the multispecific antibody istrispecific and trivalent or trispecific and tetravalent.
9. The multispecific antibody of any one of the preceding items, wherein the format of said multispecific antibody is selected from the group consisting of: a tandem tri-scFv (triplebody); a triabody; an scDb-scFv; a tandem tri-scFv or an scDb-scFv fused to the N- and/or the C-terminus of a heterodimerization domain other than heterodimeric Fc domains; and a MATCH.
10. The multispecific antibody of any one of the preceding items, wherein the format of said mulbspecific antibody is selected from an scDb-scFv, an scMATCH3, a MATCH3 and a MATCH4.
11. The multispecific antibody of any of the preceding items, wherein each of said one or two ROR1-BDs, when being in scFv format:
   a. binds to the extracellular domain of human ROR1 with a monovalent dissociation constant (K_{D}) of 1 pM to 2 nM, particularly with a K_{D} of 1 pM to 1 nM, particularly of 1 to 500 pM, as measured by surface plasmon resonance (SPR);
   b. binds to human ROR1-expressing MDA-MB-231 cells with an EC₅₀ of 5 pM to 10 nM, particularly with an EC₅₀ of 5 pM to 5 nM, particularly with an EC₅₀ of 5 pM to 4 nM, particularly with an EC₅₀ of 5 pM to 3 nM;
   c. has a melting temperature (Tm), determined by differential scanning fluorimetry (DSF), of at least 58° C., particularly of at least 59° C., particularly of at least 60° C., particularly of at least 61° C., in particular wherein said scFvs are formulated in 50 mM phosphate citrate buffer with 150 mM NaCl at pH 6.4;
   d. has a loss in monomer content, after storage for four weeks at 4° C. of less than 3%, particularly less than 2%, particularly less than 1%, when said scFvs are at a starting concentration of 10 mg/ml, and in particular wherein said scFvs are formulated in 50 mM phosphate citrate buffer with 150 mM NaCl at pH 6.4; and/or
   e. has a loss in monomer content, after storage for four weeks at 40° C. of less than 10%, when said scFvs are at a starting concentration of 10 mg/ml, and in particular wherein said scFvs are formulated in 50 mM phosphate citrate buffer with 150 mM NaCl at pH 6.4; and/or
   f. has a loss in protein content, after storage for four weeks at 4° C. or 40° C. of less than 1%, when said scFvs are at a starting concentration of 10 mg/ml, and in particular wherein said scFvs are formulated in 50 mM phosphate citrate buffer with 150 mM NaCl at pH 6.4.
12. The multispecific antibody of any one of the preceding items, wherein said one or two ROR1-BDs comprise VH1a, VHib, VH3 or VH4 domain framework sequences FR1 to FR4; particularly VH3 or VH4 domain framework sequences FR1 to FR4; particularly VH3 domain framework sequences FR1 to FR4.
13. The multispecific antibody of any one of the preceding Items, wherein said one or two ROR1-BDs further have one or both of the following features 1) and 2):
   1) a VL domain comprising framework regions FR1. FR2 and FR3, which are selected from V_{K} subtypes, particularly from the V_{K}1 and V_{K}3 subtypes, particularly are of the V_{K}1 subtype, and a framework FR4, which Is selected from a VA FR4, particularly is a VA FR4 comprising an amino acid sequence having at least 70, 80, 90 percent identity to any of SEQ ID NO: 76 to SEQ ID NO: 83, more particularly a VA FR4 selected from any of SEQ ID NO: 76 to SEQ ID NO: 83, particularly a VA FR4 according to SEQ ID NO: 76 or 83;
   2) a VH domain comprising VH framework regions FR1, FR2, FR3 and FR4, which are selected from a VH framework subtype, particularly from the VH framework subtypes VH1a, VH1b, VH3 and VH4, particularly from the VH framework subtypes VH3 and VH4, particularly are of the VH3 subtype; wherein said VH framework regions FR1, FR2, FR3 and FR4 have the following substitutions (AHo numbering): an arginine (R) at amino acid position 12; a threonine (T) at amino acid position 103 and a glutamine (Q) at amino acid position 144.
14. The multispecific antibody of any one of the preceding items, wherein said one or two ROR1-BDs comprise
   a) a VH sequence being at least 90, 91, 92, 93, 94, 95, 96, 97, 98 or 99 percent identical to any one of the amino acid sequences selected from SEQ ID NOs: 7 and 10; and
   b) a VL sequence being at least 90, 91, 92, 93, 94, 95, 96, 97, 98 or 99 percent identical to any one of the amino acid sequences selected from SEQ ID NOs: 9 and 12;
   or
   a) a VH sequence being at least 90, 91, 92, 93, 94, 95, 96, 97, 98 or 99 percent identical to any one of the amino acid sequences selected from SEQ ID NOs: 20, 23, 26 and 28; and
   b) a VL sequence being at least 90, 91, 92, 93, 94, 95, 96, 97, 98 or 99 percent identical to any one of the amino acid sequences selected from SEQ ID NOs: 22, 25, 27 and 29;
   or
   a) a VH sequence being at least 90, 91, 92, 93, 94, 95, 96, 97, 98 or 99 percent identical to any one of the amino acid sequences selected from SEQ ID NOs: 8 and 11; and
   b) a VL sequence being at least 90, 91, 92, 93, 94, 95, 96, 97, 98 or 99 percent identical to any one of the amino acid sequences selected from SEQ ID NOs: 9 and 12;
   or
   a) a VH sequence being at least 90, 91, 92, 93, 94, 95, 96, 97, 98 or 99 percent identical to any one of the amino acid sequences selected from SEQ ID NOs: 21 and 24; and
   b) a VL sequence being at least 90, 91, 92, 93, 94, 95, 96, 97, 98 or 99 percent identical to any one of the amino acid sequences selected from SEQ ID NOs: 22 and 25.
15. The multispecific antibody of any one of the preceding Items, wherein said one or two ROR1-BDs comprise
   a) a VH sequence being at least 90, 91, 92, 93, 94, 95, 96, 97, 98 or 99 percent identical to any one of the amino acid sequences selected from SEQ ID NOs: 7, 20 and 26; and
   b) a VL sequence being at least 90, 91, 92, 93, 94, 95, 96, 97, 98 or 99 percent identical to any one of the amino acid sequences selected from SEQ ID NOs: 9, 22 and 27;
   or
   1) a VH sequence being at least 90, 91, 92, 93, 94, 95, 96, 97, 98 or 99 percent identical to any one of the amino acid sequences selected from SEQ ID NOs: 8, 11 and 24; and
   2) a VL sequence being at least 90, 91, 92, 93, 94, 95, 96, 97, 98 or 99 percent identical to any one of the amino acid sequences selected from SEQ ID NOs: 9, 12 and 25.
16. The multispecific antibody of any one of the items 1 to14, wherein said one or two ROR1-BDs comprise
   a) a VH sequence selected from SEQ ID NOs: 7 and 10,
   b) a VL sequence selected from SEQ ID NOs: 9 and 12;
   or
   a) a VH sequence selected from SEQ ID NOs: 20, 23, 26 and 28,
   b) a VL sequence selected from SEQ ID NOs: 22, 25, 27 and 29;
   or
   a) a VH sequence selected from SEQ ID NOs: 8, 11 and 14,
   b) a VL sequence selected from SEQ ID NOs: 9, 12 and 25.
17. The multispecific antibody of any one of the preceding items, wherein said one or two ROR1-BDs comprise
   a) a VH sequence of SEQ ID NO: 7 and a VL sequence of SEQ ID NO: 9; or
   b) a VH sequence of SEQ ID NO: 10 and a VL sequence of SEQ ID NO: 12; or
   c) a VH sequence of SEQ ID NO: 20 and a VL sequence of SEQ ID NO: 22; or
   d) a VH sequence of SEQ ID NO: 23 and a VL sequence of SEQ ID NO. 25; or
   e) a VH sequence of SEQ ID NO: 8 and a VL sequence of SEQ ID NO: 9; or
   f) a VH sequence of SEQ ID NO: 11 and a VL sequence of SEQ ID NO: 12; or
   g) a VH sequence of SEQ ID NO: 21 and a VL sequence of SEQ ID NO: 22; or
   h) a VH sequence of SEQ ID NO: 24 and a VL sequence of SEQ ID NO: 25; or
   i) a VH sequence of SEQ ID NO: 26 and a VL sequence of SEQ ID NO: 27; or
   j) a VH sequence of SEQ ID NO: 28 and a VL sequence of SEQ ID NO: 29;
   particulary wherein said one or two ROR1-BDs comprise
   a) a VH sequence of SEQ ID NO: 7 and a VL sequence of SEQ ID NO: 9; or
   b) a VH sequence of SEQ ID NO: 10 and a VL sequence of SEQ ID NO: 12; or
   c) a VH sequence of SEQ ID NO: 23 and a VL sequence of SEQ ID NO: 25; or
   d) a VH sequence of SEQ ID NO: 8 and a VL sequence of SEQ ID NO: 9; or
   e) a VH sequence of SEQ ID NO: 11 and a VL sequence of SEQ ID NO: 12; or
   f) a VH sequence of SEQ ID NO: 24 and a VL sequence of SEQ ID NO: 25.
18. The multispecific antibody of any one of the preceding Items, wherein said CD3-BD is binding to CD3c.
19. The multispecific antibody of any one of the preceding items, wherein said CD3-BD, when being in scFv format:
   a) binds to human CD3ε with a dissociation constant (K_{D}) of less than 50 nM, particularly with a K_{D} of 0.1 to 50 nM, particularly of 0.1 to 30 nM, particularly of 0.1 to 15 nM, particularly of 0.1 to 10 nM, as measured by SPR;
   b) binds to Macaca fascicularis (Cynomolgus) CD3 with a K_{D} of less than 50 nM, particularly with a K_{D} of 0.1 to 50 nM, particularly of 0.1 to 30 nM, particularly of 0.1 to 15 nM, particularly of 0.1 to 10 nM, as measured by SPR; and
   c) has a melting temperature (Tm), determined by differential scanning fluorimetry (DSF), of at least 60° C., particularly of at least 65° C., more particularly of at least 67° C.
20. The multispecific antibody of any one of the preceding items, wherein said CD3-BD comprises VH1a, VH1b, VH3 or VH4 domain framework sequences FR1 to FR4; particularly VH3 or VH4 domain framework sequences FR1 to FR4; particularly VH3 domain framework sequences FR1 to FR4. 21. The multispecific antibody of items 18 to 20, wherein said CD3-BD has one or both of the following features 1) and 2):
   1) a VL domain comprising framework regions FR1. FR2 and FR3, which are selected from V_{K} subtypes, particularly from the V_{K}1 and V_{K}3 subtypes, particularly are of the V_{K}1 subtype, and a framework FR4, which is selected from a VA FR4, particularly is a VA FR4 comprising an amino acid sequence having at least 70, 80, 90 percent identity to any of SEQ ID NO: 76 to SEQ ID NO: 83, more particularly a VA FR4 selected from any of SEQ ID NO: 76 to SEQ ID NO: 83, particularly a VA FR4 according to SEQ ID NO: 76 or 83;
   2) a VH domain comprising VH framework regions FR1, FR2, FR3 and FR4, which are selected from a VH framework subtype, particularly from the VH framework subtypes VH1a, VHib, VH3 and VH4, particularly from the VH framework subtypes VH3 and VH4, particularly are of the VH3 subtype; wherein said VH framework regions FR1, FR2, FR3 and FR4 have the following substitutions (AHo numbering): an arginine (R) at amino acid position 12; a threonine (T) at amino acid position 103 and a glutamine (Q) at amino acid position 144.
22. The multispecific antibody of any one of the preceding items, wherein said CD3-BD comprises
   a) a VH sequence being at least 90, 91, 92, 93, 94, 95, 96, 97, 98 or 99 percent identical to any one of the amino acid sequences selected from SEQ ID NO: 36 or 37; and
   b) a VL sequence being atleast 90, 91, 92, 93, 94, 95, 96, 97, 98 or 99 percent identical to any one of the amino acd sequences selected from SEQ ID NO: 38.
23. The multispecific antibody of item 22, wherein said CD3-BD comprises
   a) a VH domain comprising the amino acid sequence of SEQ ID NO: 36 or 37, and
   b) a VL domain comprising the amino acid sequence of SEQ ID NO: 38.
24. The multispecific antibody of item 22 or 23, wherein said VH sequence comprises a C51 amino acid residue (AHo numbering) and said VL sequence comprises a C141 amino acid residue (AHo numbering).
25. The multispecific antibody of item 7, wherein said hSA-BD comprises
   (i) HCDR1, HCDR2, and HCDR3 sequences of SEQ ID NOs: 39, 40 and 41, respectively, and human VH framework sequences FR1, FR2, FR3 and FR4, which are selected from a VH framework subtype, particularly from the VH framework subtypes VH1a, VH1b, VH3 and VH4, particularly from the VH framework subtypes VH3 and VH4, particularly are of the VH3 subtype; wherein said VH framework regions FR1, FR2, FR3 and FR4 optionally have the following substitutions (AHo numbering): an arginine (R) at amino acid position 12; a threonine (T) at amino acid position 103 and a gktamine (Q) at amino acid position 144; and
(ii) LCDR1, LCDR2, and LCDR3 sequences of SEQ ID NOs: 42, 43 and 44, respectively, and human antibody VL framework sequences FR1, FR2 and FR3, which are selected from V_{K} subtypes, particularly from the V_{K}1 and V_{K}3 subtypes, particularly are of the V_{K}1 subtype, and a human antibody VL framework FR4 sequence, which is a Vλ FR4 sequence, particularly a VA FR4 sequence selected from any of SEQ ID NO: 76 to SEQ ID NO: 83, particularly a VA FR4 sequence according to SEQ ID NO: 76 or 83;
   or
   (i) HCDR1, HCDR2, and HCDR3 sequences of SEQ ID NOs: 49, 50 and 51, respectively, and human VH framework sequences FR1, FR2, FR3 and FR4, which are selected from a VH framework subtype, particularly from the VH framework subtypes VH1a, VH1b, VH3 and VH4, particularly from the VH framework subtypes VH3 and VH4, particularly are of the VH3 subtype; wherein said VH framework regions FR1, FR2, FR3 and FR4 optionally have the following substitutions (AHo numbering): an arginine (R) at amino acid position 12; a threonine (T) at amino acid position 103 and a glutamine (Q) at amino acid position 144; and
   (ii) LCDR1, LCDR2, and LCDR3 sequences of SEQ ID NOs: 52, 53 and 54, respectively, and human antibody VL framework sequences FR1, FR2 and FR3, which are selected from V_{K} subtypes, particularly from the V_{K}1 and V_{K}3 subtypes, particularly are of the V_{K}1 subtype, and a human antibody VL framework FR4 sequence, which is a VA FR4 sequence, particularly a VA FR4 sequence selected from any of SEQ ID NO: 76 to SEQ ID NO: 83, particularly a VA FR4 sequence according to SEQ ID NO: 76 or 83;
   or
   (i) HCDR1, HCDR2, and HCDR3 sequences of SEQ ID NOs: 59, 60 and 61, respectively, and human VH framework sequences FR1, FR2, FR3 and FR4, which are selected from a VH framework subtype, particularly from the VH framework subtypes VHia, VH1b, VH3 and VH4, particularly from the VH framework subtypes VH3 and VH4, particularly are of the VH3 subtype, wherein said VH framework regions FR1, FR2, FR3 and FR4 optionally have the following substitutions (AHo numbering): an arginine (R) at amino acid position 12; a threonine (T) at amino acid position 103 and a glutamine (Q) at amino acid position 144; and
   (ii) LCDR1, LCDR2, and LCDR3 sequences of SEQ ID NOs: 62, 63 and 74, respectively, and human antibody VL framework sequences FR1, FR2 and FR3, which are selected from V_{K} subtypes, particularly from the V_{K}1 and V_{K}3 subtypes, particularly are of the V_{K}1 subtype, and a human antibody VL framework FR4 sequence, which is a VA FR4 sequence, particularly a VA FR4 sequence selected from any of SEQ ID NO: 76 to SEQ ID NO: 83, particularly a VA FR4 sequence according to SEQ ID NO: 76 or 83.
26. The multispecific antibody of items 7 or 25, wherein said hSA-BD comprises
   (i) a VH domain comprising the amino acid sequence of SEQ ID NO: 45 and a VL domain comprising the amino acid sequence of SEQ ID NO: 46; or
   (ii) a VH domain comprising the amino acid sequence of SEQ ID NO: 47 and a VL domain comprising the amino acid sequence of SEQ ID NO: 48; or
   (iii) a VH domain comprising the amino acid sequence of SEQ ID NO: 55 and a VL domain comprising the amino acid sequence of SEQ ID NO: 56; or (iv) a VH domain comprising the amino acid sequence of SEQ ID NO: 57 and a VL domain comprising the amino acid sequence of SEQ ID NO: 58; or
   (v) a VH domain comprising the amino acid sequence of SEQ ID NO: 65 and a VL domain comprising the amino acid sequence of SEQ ID NO: 67; or
   (vi) a VH domain comprising the amino acid sequence of SEQ ID NO: 66 and a VL domain comprising the amino acid sequence of SEQ ID NO: 67; or
   (vii) a VH domain comprising the amino acid sequence of SEQ ID NO: 68 and a VL domain comprising the amino acid sequence of SEQ ID NO: 70; or
   (villi) a VH domain comprising the amino acid sequence of SEQ ID NO: 69 and a VL domain comprising the amino acid sequence of SEQ ID NO: 70.
27. The multispecific antibody of items 7 or 25, wherein said hSA-BD comprises
   (i) a VH domain comprising the amino acid sequence of SEQ ID NO: 47 and a VL domain comprising the amino acid sequence of SEQ ID NO: 48; or
   (ii) a VH domain comprising the amino acid sequence of SEQ ID NO: 57 and a VL domain comprising the amino acid sequence of SEQ ID NO: 58; or
   (iii) a VH domain comprising the amino acid sequence of SEQ ID NO: 68 and a VL domain comprising the amino acid sequence of SEQ ID NO: 70; or
   (iv) a VH domain comprising the amino acid sequence of SEQ ID NO: 69 and a VL domain comprising the amino acid sequence of SEQ ID NO: 70.
28. The multispecific antibody of any one of the preceding items, wherein said binding domains are independently selected from the group consisting of a Fab, a F(ab)₂, an Fv, an scFv, a dsFv, and an scAb, in particular from the group consisting of an Fv, an scFv, and a dsFv.
29. The multispecific antibody of item 28, wherein each of said binding domains is independently selected from
   a) a cognate pair of a VL domain and a VH domain (Fv fragment); or
   b) a cognate pair of a VL domain and a VH domain linked by an oligo- or polypeptide linker (scFv fragment).
30. The multispecific antibody of any one of the items 1 to 29, wherein the multispecific antibody comprises:
   a) one ROR1-BD;
   b) one CD3-BD; and
   c) one hSA-BD.
31. The multispecific antibody of item 30, wherein said multispecific antibody is a single-chain protein, wherein said single-chain protein comprises an amino acid sequence consisting of:
   (i) a first VL domain,
   (ii) a first polypeptide linker,
   (iN) a first VH domain,
   (iv) a second polypeptide linker,
   (v) a second VL domain,
   (vi) a third polypeptide linker, and
   (vii) a second VH domain,
   arranged one after another in the stated order,
   wherein said first VL domain associates with said second VH domain to form a first binding domain, and said second VL domain associates with said first VH domain to form a second binding domain,
   and wherein said single-chain protein further comprises
   (viii) a third binding domain, which is formed by a third VL domain and a third VH domain that are connected via a fourth polypeptide linker, where said third binding domain is fused C-terminaly or N-terminally via a fifth polypeptide linker to said amino acid sequence,
   wherein said three binding domains have the folowing specificities:
   a) one binding domain specificaly binds to human ROR1 (ROR1-BD);
   b) another binding domain specifically binds to CD3 (CD3-BD); and
   c) the remaining binding domain specificaly binds to hSA (hSA-BD).
32. The multispecific antibody of Rem 31, wherein the three binding domains have the following specificities:
   a) one of the first and second binding domains specifically binds to human serum albumin (hSA-BD);
   b) the other one of the first and second binding domain specifically binds to human CD3 (CD3-BD); and
   c) the third binding domain specifically binds to ROR1 (ROR1-BD);
   in particular the three binding domains have the following specificities:
   a) the first binding domain specifically binds to human serum albumin (hSA-BD);
   b) the second binding domain specifically binds to human CD3 (CD3-BD); and
   c) the third binding domain specifically binds to ROR1 (ROR1-BD).
33. The multispecific antibody of item 30, wherein said multispecific antibody is a hetero-dimeric protein comprising a first and a second single-chain protein,
   wherein said first single-chain protein comprises a first amino acid sequence consisting of (from the N- to the C-terminus):
   (is) a first VL domain,
   (iia) a first polypeptide linker, and
   (ilia) a second VL domain, and
   wherein said second single-chain protein comprises a second amino acid sequence consisting of (from the N- to the C-terminus):
   (ib) a first VH domain,
   (iib) a second polypeptide linker, and
   (iib) a second VH domain, and
   wherein said first VL domain associates with either said first or said second VH domain to form a first binding domain, and said second VL domain associates with the other of said VH domains to form a second binding domain,
   and wherein at least one of said first and said second single-chain proteins further comprises
   (iv) a third binding domain, which is formed by a third VL domain and a third VH domain that are connected via a third polypeptide linker, where said third binding domain is fused via a fourth polypeptide linker to said first or said second amino acid sequence,
   wherein said three binding domains have the following specificities:
   a) one binding domain specifically binds to human CD3 (CD3-BD);
   b) another binding domain specifically binds to ROR1 (ROR1-BD); and
   c) the remaining binding domain specifically binds to human serum albumin (hSA-BD).
34. The multispecific antibody of any one of the items 30 to 33, wherein said multispecific antibody:
   a. binds to the extracellular domain of human ROR1 with a monovalent dissociation constant (KD) of less than 5 nM, particularly with a monovalent K_{D} of 1 pM to 5 nM, particularly with a KID of 1 pM to 1 nM, particularly of 1 to 500 pM, as measured by surface plasmon resonance (SPR);
   b. binds to human CD3ε with a (monovalent) K_{D} of less than 20 nM, particularly with a monovalent K_{D} of 0.1 to 20 nM, particularly of 0.1 to 15 nM, particularly of 0.1 to 10 nM, as measured by SPR;
   c. Is cross-reactive with Macaca fascicularis (Cynomolgus) CD3ε, in particular binds to Cynomolgus CD3 with a monovalent K_{D} of 30 nM or less, particularly with a monovalent K_{D} of 0.1 to 30 nM, particularly of 0.1 to 20 nM, as measured by SPR;
   d. binds to human ROR1-expressing MDA-MB-231 cells with an EC₅₀ of 0.01 to 50 nM, particularly with an ECs of 0.01 to 30 nM, particularly with an EC₅₀ of 0.01 to 20 nM, particularly with an EC₅₀ of 0.01 to 10 nM, particularly with an EC₅₀ of 0.01 to 5 nM;
   e. has an EC₅₀ for killing human ROR1-expressing MDA-MB-231 target cells of less than 5 nM, particularly of 1 pM to 5 nM, particularly of 1 pM to 2 nM, particularly of 1 pM to 1 nM, as determined in a T-cell-driven cytotoxicity assay against said target cells;
   f. has a melting temperature (Tm), determined by differential scanning fluorimetry, of at least 55° C., preferably of at least 58° C., more preferably of at least 60° C., in particular wherein said antigen-binding fragment is 50 mM phosphate citrate buffer with 300 mM D(+)-sucrose at pH 6.5;
   g. has a loss in monomer content, after storage for four weeks at 4° C. of less than 1%, e.g. less than 0.5%, less than 0.3%, less than 0.2%, when said scFvs are at a starting concentration of 1 mg/ml, and in particular wherein said scFvs are formulated in 50 mM phosphate citrate buffer with 300 mM D(+)-sucrose at pH 6.5; and/or
   h. has a loss in monomer content, after storage for four weeks at 40° C. of less than 5%, e.g. less than 4%, when said scFvs are at a starting concentration of 1 mg/mi, and in particular wherein said scFvs are formulated in 50 mM phosphate citrate buffer with 300 mM D(+)-sucrose at pH 6.5.
35. The multispecific antibody of any one of the items 30, 31, 33 and 34, wherein said multispecific antibody is selected from an antibody defined by SEQ ID NOs: 87, 88, 89, 90, 91, 92, 93, 94, 107 and 108.
36. The multispecific antibody of any one of the items 1 to 29, wherein the multispecific antibody comprises:
   a) two ROR1-BDs;
   b) one CD3-BD; and
   c) one hSA-BD.
37. The multispecific antibody of Item 36, wherein the two ROR1-BDs bind to the same epitope on the extracellular domain of ROR1 or to different epitopes on the extracellular domain of ROR1, particularly to the same epitope on the extracellular domain of ROR1.
38. The multispecific antibody of items 36 or 37, wherein said multispeciflc antibody is a hetero-dimeric protein comprising a first and a second single-chain protein,
   wherein said first single-chain protein comprises a first amino acid sequence consisting of (from the N- to the C-terminus):
   (ia) a first VL domain,
   (iia) a first polypeptide linker, and
   (iia) a second VL domain, and
   wherein said second single-chain protein comprises a second amino acid sequence consisting of (from the N- to the C-terminus):
   (ib) a first VH domain,
   (iib) a second polypeptide linker, and
   (iiib) a second VH domain, and
   wherein said first VL domain associates with either said first or said second VH domain to form a first binding domain, and said second VL domain associates with the other of said VH domains to form a second binding domain,
   and wherein at least one of said first and said second single-chain proteins further comprises
   (iv) a third binding domain, which is formed by a third VL domain and a third VH domain that are connected via a third polypeptide linker, where said third binding domain is fused via a fourth polypeptide linker to said first or said second amino acid sequence,
   and wherein at least one of said first and said second single-chain proteins further comprises
   (v) a fourth binding domain, which is formed by a fourth VL domain and a fourth VH domain that are connected via a fifth polypeptide linker, where said fourth binding domain is fused via a sixth polypeptide linker to said first or said second amino acid sequence,
   wherein the four binding domains have the following specificities:
   a) one binding domain specifically binds to human CD3 (CD3-BD);
   b) another two binding domains specifically bind to ROR1 (ROR1-BD); and
   c) the remaining binding domain specifically binds to human serum albumin (hSA-BD).
39. The multispecific antibody of item 38, wherein the third binding domain is fused to either the first or the second amino acid sequence, and the fourth binding domain is fused to the other one of the two said amino acid sequences.
40. The multispecific antibody of Item 38 or 39, wherein the four binding domains have the following specificities:
   a) the first binding domain specifically binds to human CD3 (CD3-BD);
   b) the second binding domain specifically binds to human serum albumin (hSA-BD); and
   c) the third and the fourth binding domains specifically bind to ROR1 (ROR1-BD).
41. The multispecific antibody of any one of the Items 36 to 40, wherein said multispecific antibody:
   a. binds to the extracellular domain of human ROR1 with a monovalent dissociation constant (KD) of less than 10 nM, particularly with a monovalent K_{D} of 10 pM to 5 nM, particularly with a K_{D} of 10 pM to 7 nM, particularly of 10 pM to 5 nM, as measured by surface plasmon resonance (SPR);
   b. binds to human CD3ε with a monovalent K_{D} of with a monovalent K_{D} of less than 20 nM, particularly with a monovalent K_{D} of 0.1 to 20 nM, particularly of 0.1 to 15 nM, particularly of 0.1 to 10 nM, as measured by SPR;
   c. Is cross-reactive with Macaca fascicularis (Cynomolgus) CD3ε, in particular binds to Cynomolgus CD3ε with a monovalent K_{D} of 30 nM or less, particularly with a monovalent K_{D} of 0.1 to 30 nM, particularly of 0.1 to 20 nM, as measured by SPR;
   d. binds to human ROR1-expressing MDA-MB-231 with an EC₅₀ of 0.01 to 10 nM, particularly with an EC₅₀ of 0.01 to 10 nM, particularly with an EC₅₀ of 0.01 to 5 nM, particularly with an EC₅₀ of 0.01 to 3 nM, particularly with an EC₅₀ of 0.01 to 2 nM;
   e. has an EC₅₀ for killing human ROR1-expressing MDA-MB-231 target cells of less than 500 pM, particularly of 0.1 pM to 500 pM, particularly of 0.1 pM to 200 pM, particularly of 0.1 to 100 pM, particularly of 0.1 to 50 pM, as determined in a T-cell driven cytotoxicity assay against said target cells;
   f. has an EC₅₀ for killing human ROR1-expressing JEKO-1 target cells of less than 20 pM, particularly of 0.01 pM to 20 pM, particularly of 0.01 pM to 10 pM, particularly of 0.01 to 5 pM, particularly of 0.01 to 3 pM, as determined in a T-cell-driven cytotoxicity assay against said target cells;
   g. has an EC₅₀ for killing human ROR1-expressing SKOV-3 target cells of less than 1000 pM, particularly of 0.1 pM to 1000 pM, particularly of 0.1 pM to 500 pM, particularly of 0.1 to 200 pM, particularly of 0.1 to 100 pM, as determined in a T-cell-driven cytotoxicity assay against said target cells;
   h. has a melting temperature (Tm), determined by differential scanning fluorimetry, of at least 53° C., preferably of at least 55° C., more preferably of at least 58° C., in particular wherein said antigen-binding fragment is formulated in 50 mM phosphate-citrate buffer with 300 mM sucrose at pH 6.5;
   i. has a loss in monomer content, after storage for two weeks at 4° C. of less than 1%, particularly of less than 0.5%, when said multispecific antibody is at a starting concentration of 1 mg/mi, and in particular wherein said multispecific antibody is formulated in 50 mM phosphate citrate buffer with 300 mM D(+)-sucrose at pH 6.5; and/or
   j. has a loss in monomer content, after storage for at least four weeks at 40° C. of less than 5% when said multispecific antibody is at a starting concentration of 1 mg/mi, and in particular wherein said multispecific antibody is formulated in 50 mM phosphate citrate buffer with 300 mM D(+)-sucrose at pH 6.5.
42. The multispecific antibody of any one of the items 36 to 41, wherein said first single-chain protein comprises the amino acid sequence of SEQ ID NO: 95, more particularly consists of the amino acid sequence SEQ ID NO: 95 and said second single-chain protein comprises the amino acid sequence of SEQ ID NO: 96, more particularly consists of the amino acid sequence SEQ ID NO: 96; or said first single-chain protein comprises the amino acid sequence of SEQ ID NO: 97, more particularly consists of the amino acid sequence SEQ ID NO: 97 and said second single-chain protein comprises the amino acid sequence of SEQ ID NO: 98, more particularly consists of the amino acid sequence SEQ ID NO: 98; or said first single-chain protein comprises the amino acid sequence of SEQ ID NO: 99, more particularly consists of the amino acid sequence SEQ ID NO: 99 and said second single-chain protein comprises the amino acid sequence of SEQ ID NO: 100, more particularly consists of the amino acid sequence SEQ ID NO: 100; or said first single-chain protein comprises the amino acid sequence of SEQ ID NO: 101, more particularly consists of the amino acid sequence SEQ ID NO: 101 and said second single-chain protein comprises the amino acid sequence of SEQ ID NO: 102, more particularly consists of the amino acid sequence SEQ ID NO: 102; or said first single-chain protein comprises the amino acid sequence of SEQ ID NO: 103, more particularly consists of the amino acid sequence SEQ ID NO: 103 and said second single-chain protein comprises the amino acid sequence of SEQ ID NO: 104, more particularly consists of the arnino acid sequence SEQ ID NO: 104; or said first single-chain protein comprises the amino acid sequence of SEQ ID NO: 105, more particularly consists of the amino acid sequence SEQ ID NO: 105 and said second single-chain protein comprises the amino acid sequence of SEQ ID NO: 106, more particulaly consists of the amino acid sequence SEQ ID NO: 106; or said first single-chain protein comprises the amino acid sequence of SEQ ID NO: 109, more particularly consists of the amino acid sequence SEQ ID NO: 109 and said second single-chain protein comprises the amino acid sequence of SEQ ID NO: 110, more particularly consists of the amino acid sequence SEQ ID NO: 110; or said first single-chain protein comprises the amino acid sequence of SEQ ID NO: 111, more particularly consists of the amino acid sequence SEQ ID NO: 111 and said second single-chain protein comprises the amino acid sequence of SEQ ID NO: 112, more particularly consists of the amino acid sequence SEQ ID NO: 112; or said first single-chain protein comprises the amino acid sequence of SEQ ID NO: 113, more particularly consists of the amino acid sequence SEQ ID NO: 113 and said second single-chain protein comprises the amino acid sequence of SEQ ID NO: 114, more particularly consists of the amino acid sequence SEQ ID NO: 114; or said first single-chain protein comprises the amino acid sequence of SEQ ID NO: 115, more particularly consists of the amino acid sequence SEQ ID NO: 115 and said second single-chain protein comprises the amino acid sequence of SEQ ID NO: 116, more particularly consists of the amino acid sequence SEQ ID NO: 116.
43. The multispecific antibody of any one of the items 32 and 38 to 41, wherein said hetero-dimeric protein does not comprise a cognate pair of a first and a second proteinaceous interaction domain, other than said first and second VL and VH domains, wherein said first proteinaceous interaction domain is comprised in said first single-chain protein and wherein said second proteinaceous interaction domain is comprised in said second single-chain protein.
44. The multispecific antibody of any one of the items 43, wherein said first single-chain protein and said second single-chain protein hetero-dimerize in a parallel orientation, i.e. said first VL domain associates with said first VH domain and said second VL domain associates with said second VH domain.
45. The multispecific antibody of any one of the items 43, wherein said first single-chain protein and said second single-chain protein hetero-dimerize in an anti-parallel orientation, i.e. said first VL domain associates with said second VH domain and said second VL domain associates with said first VH domain.
46. The multispecific antibody of any one of the items 30 to 45, wherein any binding domains comprised in said single-chain protein or hetero-dimeric protein are immunoglobulin variable domains, arranged in said first and second single-chain protein, and typically connected via linkers.
47. The multispecific antibody of any one of the preceding items, wherein at least one of said antibody variable domains comprises CDR regions derived from a parental rabbit antibody.
48. The multispecific antibody of any one of the preceding items, wherein the ROR1-BD or, in case where two ROR1-BDs are present in the multispecific antibody, at least one of the ROR1-BDs and the CD3-BD are capable of binding to their respective antigens simultaneously, or, in case where two ROR1-BDs are present in the multispecific antibody, both ROR1-BDs and the CD3-BD are capable of binding to their respective antigens simultaneously.
49. A ROR1-BD as defined in any one of items 1, 4 and 11 to 17.
50. A multispecific antibody comprising:
   a) one or two ROR1-BDs as defined in item 49;
   b) at least one binding domain, which specifically binds to atarget different from ROR1.
51. A nucleic acid or two nucleic acids encoding the multispecific antibody of any one of items 1 to 48 and 50 or the ROR1-BD of item 49.
52. A vector or two vectors comprising the nucleic acid or the two nucleic acids of item 51.
53. A host cell or host cells comprising the vector or the two vectors of item 52.
54. A method for producing the multispecific antibody of any one of items 1 to 48 and 50, or the ROR1-BD of item 49, comprising (i) providing the nucleic acid or the two nucleic acids of item 51, or the vector or the two vectors of item 52, expressing said nucleic acid or nucleic acids, or said vector or vectors, and collecting said multispecific antibody or said ROR1-BD from the expression system, or (ii) providing a host ceil or host cells according to item 53, culturing said host cell or said host cells; and collecting said multispecific antibody or said ROR1-BD from the cell culture.
55. A pharmaceutical composition comprising the multispecfic antibody of any one of items 1 to 48 and 50 and a pharmaceutically acceptable carrier.
56. The multispecific antibody of any one of items 1 to 48 and 50 for use as a medicament.
57. The multispecific antibody of any one of items 1 to 48 and 50 for use in the treatment of a disease, particularly a human disease, more particularly a human disease selected from cancer.
58. The multispecific antibody of any one of items 1 to 48 and 50 for use in the treatment of a disease according to Item 57, wherein said disease is a ROR1-expressing cancer.
59. The multispecific antibody of item 58, wherein said ROR1-expressing cancer is selected from chronic lymphocytic leukemia/small lymphocytic lymphoma (CLL/SLL), acute myeloid leukemia (AML), acute lymphoblastic leukemia (ALL), mantle cell lymphoma (MCL), hairy cell leukemia, follicular lymphoma (FL), marginal zone lymphoma (MZL), diffuse large B cell lymphoma (DLBCL), Non-Hodgkin lymphoma (NHL), Richter's syndrome (RS), lung cancer, pancreatic cancer, prostate cancer, colon cancer, bladder cancer, breast cancer, ovarian cancer, glioblastoma, testicular cancer, uterine cancer, adrenal cancer, melanoma, neuroblastoma, sarcoma and renal cancer.
60. A method for the treatment of a disease, particularly a human disease, more particularly a human disease selected from cancer, comprising the step of administering the multispecific antibody of any one of items 1 to 48 to a patient in need thereof.
61. The method of item 60, wherein said disease is a ROR1-expressing cancer.
62. The method of item 61, wherein said ROR1-expressing cancer is selected from chronic lymphocytic leukemia/small lymphocyticlymphoma (CLL/SLL), acute myeloid leukemia (AML), acute lymphoblastic leukemia (ALL), mantle cell lymphoma (MCL), hairy cell leukemia, follicular lymphoma (FL), marginal zone lymphoma (MZL), diffuse large B cell lymphoma (DLBCL), Non-Hodgkin lymphoma (NHL), Richter's syndrome (RS), lung cancer, pancreatic cancer, prostate cancer, colon cancer, bladder cancer, breast cancer, ovarian cancer, glioblastoma, testicular cancer, uterine cancer, adrenal cancer, melanoma, neuroblastoma, sarcoma and renal cancer.

### BRIEF DESCRIPTION OF THE DRAWINGS

FIG. 1 shows representative concentration-response curves of anti-ROR1 scFvs PRO2060 (A, B) and PRO2062 to human ROR1 expressed on MDA-MB-231 cells and to ROR1-negative MCF-7 cells by flow cytometry. PRO2060 and PR02062 demonstrated binding to ROR1-positive MDA-MB-231 cefls (A, C) while no binding to ROR1-negative MCF-7 cells was observed (B, D). Compared to the reference Fab fragment PRO2213, PRO2060 showed an around three times lower EC₅₀ binding and PRO2062 showed an around three times higher EC₅₀ binding. Data points in the figure represent the mean of two technical replicates.

FIG. 2 shows the concentration-response curves of optimized anti-ROR1 scFvs PRO2271 (A, B) as well as PRO2291 and PRO2292 (C, D) to human ROR1 expressed on MDA-MB-231 cells and to ROR1-negative MCF-7 cells by flow cytometry. All scFvs demonstrated binding to ROR1-positive MDA-MB-231 cells (A, C) while no binding was found to ROR1-negative MCF-7 cells (B, D). Compared to PRO2062, optimized PRO2271 showed an around two times lower EC₅₀ binding. Variants of PR02060 and PRO2062 Including VL—V_{H} interdomain disulfide bond for stabilization, PRO2291 and PRO2292, demonstrated similar EC₅₀ binding when compared to PR02060 and PRO2062. Data points in the figure represent the mean of two technical replicates.

FIG. 3 shows a schematic representation of the trispecific scMATCH3 format.

FIG. 4 shows the concentration-response curves of anti-ROR1xhSAxCD3 scMATCH3 molecules PRO2286, PRO2287 (A, B) and PRO2257 (C, D) to human ROR1 expressed on MDA-MB-231 cells and to ROR1-negative MCF-7 cells by flow cytometry. AN three scMATCH3 molecules demonstrated binding to ROR1-positive MDA-MB-231 cells (A, C) while no binding was found to ROR1-negative MCF-7 cells (B, D). PRO2213 was included as reference anti-ROR1 antibody. Data points in the figure represent the mean of two technical replicates.

FIG. 5 shows the concentration-response curves of anti-ROR1xhSAxCD3 scMATCH3 molecules PR02507, PR02508, PR02509, PRO2510 and PRO2668 to human ROR1 expressed on MDA-MB-231 cells and to ROR1-negative MCF-7 cells by flow cytometry. All scMATCH3 molecules demonstrated binding to ROR1-positive MDA-MB-231 cells (A, B, C) while no binding was found to ROR1-negative MCF-7 cells (D). Optimized variant of PR02510, PRO2668, demonstrated binding to MDA-MB-231 (E) cells while no binding was found to ROR1-negative MCF-7 cells (F). PRO2213 was included as reference anti-ROR1 antibody. Data points in the figure represent the mean of two technical replicates.

FIG. 6 shows the ROR1 expression and binding site quantification of MDA-MB-231 and MCF7 cells. (A) ROR1 expression levels were determined by ROR1 antibody staining of the tumor cells indicated, followed by flow cytometry. The blue histograms indicate ROR1 expression, while the control is indicated in red. (B) ROR1 surface levels were quantified using Quantum Simply Cellular anti-mouse IgG kit. The numbers indicate the number of binding sites calculated on the cell surface.

FIG. 7 shows the cytotoxicity and CD8 T cell activation mediated by ROR1-targeted scMATCH3 molecules. (A) PBMCs were co-cultured with either MDA-MB-231 cells (top) or MCF7 cells (bottom) at an effector to target ratio of 10:1 for 40 hours. Controls include target cells alone (spontaneous lysis), as well as target cells treated with 1% Triton at the start of the experiment or at the end of the experiment (B) CD8 T cell activation and (C) CD4 T cell activation as shown by frequency of CD69+ cells, from the data set depicted in (A). One representative experiment of three is shown.

FIG. 8 shows the cytotoxicity and CD8 T cell activation mediated by ROR1-targeted scMATCH3 molecules. (A) PBMCs were co-cultured with either MDA-MB-231 cells (top) or MCF7 cells (bottom) at an effector to target ratio of 10:1 for 40 hours. Controls include target cells alone (spontaneous lysis), as well as target cells treated with 1% Triton at the start of the experiment or at the end of the experiment (B) CD8 T cell activation and (C) CD4 T cell activation as shown by frequency of CD69+ cells, from the data set depicted in (A). One experiment of two is shown.

FIG. 9 shows the schematic representation of the MATCH4 format.

FIG. 10 shows concentration-response curves of bivalent anti-ROR1 MATCH4 molecules PR02589, PR02590, PRO2591 and PR02592 to human ROR1 expressed on MDA-MB-231 cells (A, B), to human ROR1 expressed on JIMT-1 cells (C, D), and to ROR1-negative MCF-7 cells (E) by flow cytometry. All MATCH4 molecules demonstrated binding to ROR1-positive MDA-MB-231 cells (A, B) and ROR1-positive JIMT-1 cells (C, D) while no binding was found to ROR1-negative MCF-7 cells (E). An optimized variant of PRO2590, PRO2670, demonstrated binding to MDA-MB-231 (F) cells while no binding was found to ROR1-negative MCF-7 cells (G). The Fab fragment PRO2213 was included as reference anti-ROR1 antibody. Data points in the figure represent the mean of two technical replicates.

FIG. 11 shows ROR1 surface expression levels determined by ROR1 antibody staining of the tumor cells indicated, followed by flow cytometry. The blue histograms indicate ROR1 expression, while the negative control is indicated in red.

FIG. 12 shows the cytotoxicity mediated by scMATCH3 and MATCH4 molecules of interest across several ROR1-expressing cell lines. Pan-T cells were co-cultured with MDA-MB-231 cells (A, top left), JIMT-1 (A, bottom left), SKOV-3 (A, bottom right) or Jeko-1 cells (A, top right) at an effector to target ratio of 10:1 for 40 hours. Controls include target cells alone (spontaneous lysis), as well as target cells treated with 1% Triton at the start of the experiment or at the end of the experiment. The MATCH4 molecules are PRO2589, PRO2590, PRO2591, and PR02592, and the curves are color coded by domain; the scMATCH3 molecules are PR02507, PR02510, and PRO2557. One experiment of four is shown. MCF7 were used as reference cells (B), i.e. ROR1-negative cells, and treated as described for A. The error bars represent the variation between technical replicates.

FIG. 13 shows the T cell activation mediated by MATCH molecules of interest across several ROR1-expressing cell lines. Pan-T cells were co-cultured with MDA-MB-231 cells (A), JIMT-1 (B) or SKOV-3 (C) cells at an effector to target ratio of 10:1 for 40 hours. Supernatants were analyzed for LDH release and the cells were analyzed by flow cytometry for the presence of activated T cells, as shown by the frequency of CD69 expressing cells. The top graphs correspond to CD8 T cells, while the bottom correspond to CD4 T cells. The MATCH4 molecules are PRO2589, PR02590, PRO2591, and PR02592 and the curves are color coded by domain; the scMATCH3 molecules are PR02507, PR02510, and PR02557. One experiment of 3 (SKOV-3) or 4 (MDA-MB-231, JIMT-1) is shown. The error bars represent the variation between technical replicates.

FIG. 14 shows the absorption levels of pre-existing ADAs in 21 human serum samples for PRO2668 (A), PRO2669 (B), PR02510 (C) and PR02589 (D), determined by the ELISA-based pre-existing ADA binding assay described in example 10. The measurements were performed with spiked and unspiked serum samples (confirmation assay setup). Further shown are the corresponding reductions of absorbance levels (inhibition (%)) of spiked human serum samples of said molecules (E).

FIG. 15 shows the cytotoxicity and CD8 T cell activation mediated by ROR1 targeted scMATCH3 and scMATCH4 molecules. Pan-T cells were co-cultured with either MDA-MB-231 cells (A top) or JIMT-1 cells (A bottom) at an effector to target ratio of 10:1 for 40 hours. Controls include target cells alone (spontaneous lysis), as well as target cells treated with 1% Triton at the start of the experiment or at the end of the experiment. CD8 T cell activation as shown by frequency of CD8+CD69+ cells (B), from the data set depicted in A. One representative experiment of at least two independent experiments is shown.

FIG. 16 shows the PRO2668 mediated cytotoxicity by T cell-mediated depletion of exemplary solid tumor cell lines (A). Graph illustrating the EC₅₀ values from different solid tumor cell Ines in dependency of the ROR1 receptor density (B). Cytotoxicity by T cell-mediated depletion of hematological tumor cell lines (C). Tumor cell lines were co-cultured together with T cells for 40 h, and cytotoxicity was assessed using lactate dehydrogenase release relative to controls. The average number of ROR1 receptors on the cell surface, as well as average EC₅₀ values for cytotoxicity are shown in the graph insets.

FIG. 17 shows the PRO2668-mediated CD8 activation and proliferation at day 6 using MDA-MB-231 or JIMT-1 as solid tumor target cells (A). CD8 activation and proliferation at day 3 mediated by Z-138 as hematological tumor target cells (B). Targets and CellTrace violet-labeled healthy T cells were co-cultured at an E:T ratio of 5:1. The frequency of proliferating cells was determined by CellTrace violet dilution relative to controls. Activated cells were identified by CD25 labeling. One representative experiment of two is depicted.

FIG. 18 shows the histogram of CLL cells and actively dividing Jeko-1 cell (A). CLL cell samples do not proliferate. 5-Ethynyl-2′-deoxyuridine (EdU) incorporation was assessed on an actively dividing Jeko-1 tumor cell line (dark grey histogram) compared to CLL patient PBMCs (light grey histogram). The incorporation of EdU indicates cell division. The ROR1 surface expression and quantification is shown for CLL patient PBMCs (white histogram, relative to negative control in black) (B). Binding of PRO2668 gated on CLL patient tumor cells (C). PBMCs from a CLL patient were co-cultured with allogeneic healthy T cells at an E:T ratio of 5:1 for 40 h. Specific killing (D) was determined by the proportion of Annexin V and live/dead dye positive CLL cells upon treatment. T cells from D were stained for the activation marker CD69 (E). Supernatants from D were assessed for cytokine release using a cytometric bead array-based multiplexing system (F). Data depicted are one of four independent donors/experiments.

FIG. 19 shows the longitudinal tumor growth inhibition (median) in the presence of different doses of the scMATCH3 PRO2668 and the scMATCH4 PRO2670 molecules. Immunocompromised mice were subcutaneously implanted with Jeko-1 cells, followed by PBMC engraftment 3 days later. After tumor volumes reached 80-100 mm³, animals were randomized and dosing was initiated. Dosing occurred every five days. Longitudinal data were analyzed using a mixed effects model, followed by the Tukey's multiple comparisons test. ns: not significant; **p≤0.01; * p≤50.05; relative to control IgG (Palivizumab) at experiment end.

### DETAILED DESCRIPTION OF THE INVENTION

A vast number of hematological and solid cancers express ROR1, which makes ROR1-expressing cancers an attractive target for cancer therapy. However, existing bispecific anti-ROR1xCD3 therapeutic antibodies developed so far generally do not have the expected breadth of efficacy against these cancer types. In particular, these therapeutic bispecific anti-ROR1xCD3 antibodies often have a moderate potency against cancer cells expressing low levels of ROR1. Besides, these prior art antibodies typically exhibit alow percentage of maximum killing—often well below 50%—in cytotoxicity assays against many ROR1-expressing cancer cells, which indicates that their mode of action is not ideal. There is thus a need in the medical field for improved anti-ROR1xCD3 based therapeutics, which have higher efficacy to address a broad scope of ROR-expressing cancers, Including cancers that express ROR1 at moderate to low levels.

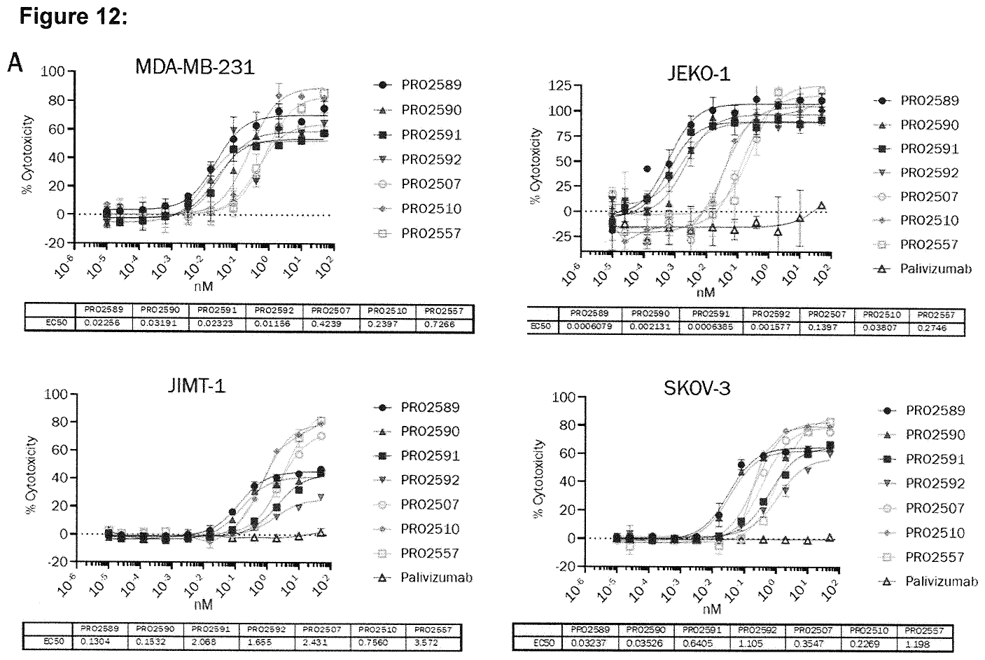

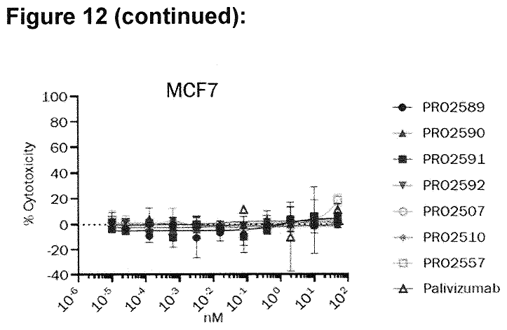

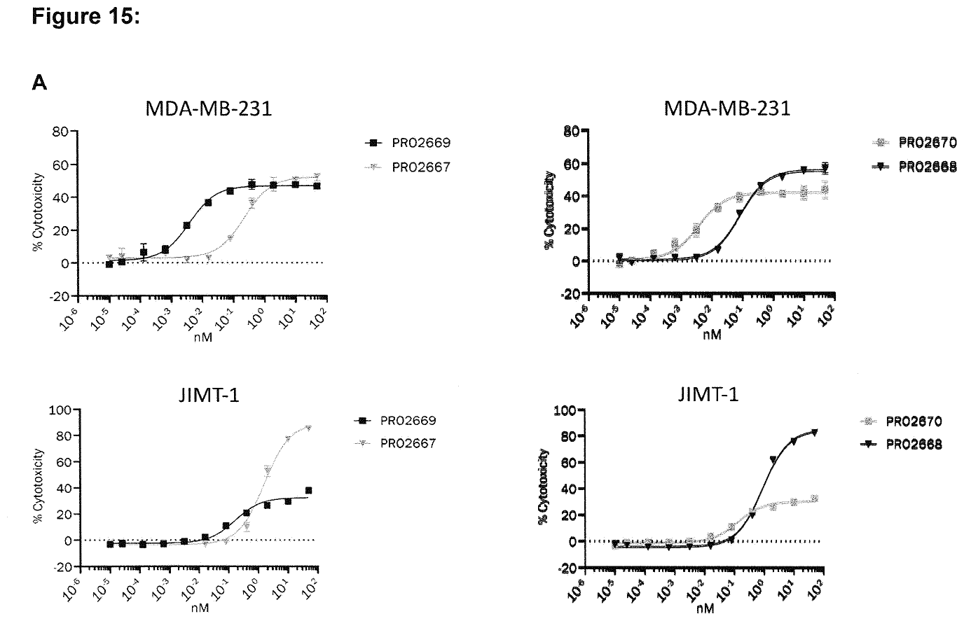

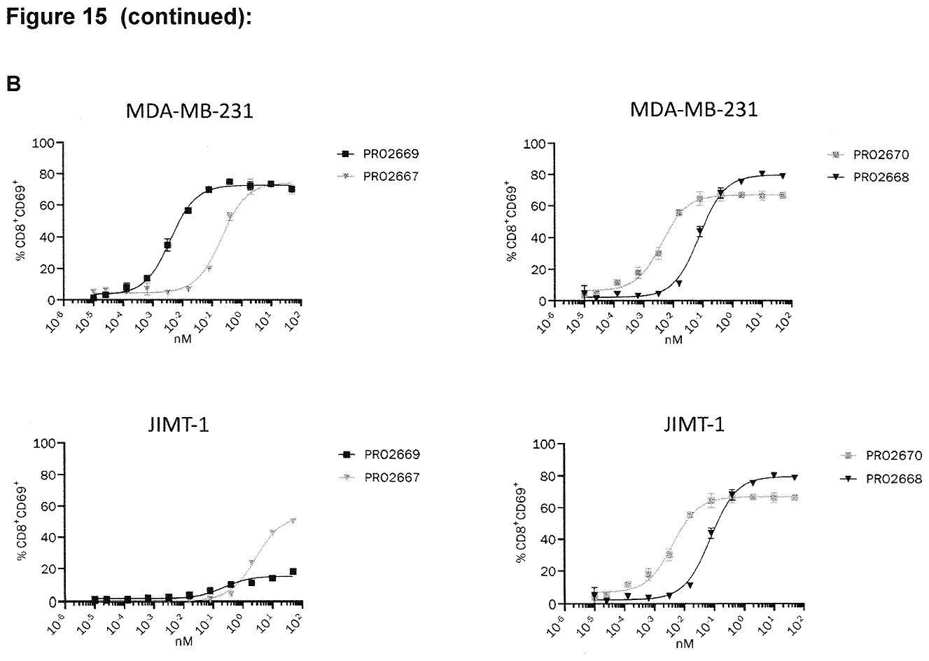

The present invention provides multispecific antibodies comprising one or two binding domains, which specifically bind to the extracellular domain of ROR1 (ROR1-BDs), and one binding domain, which specifically binds to CD3 (CD3-BD), wherein the CDR regions of the ROR1-BDs and CD3-BD have the above listed definitions. The multispecific antibodies of the present invention are capable to potently kill cancer cells that express high levels of ROR1 as well as cancer cells that express ROR1 only in low levels. Some of the multispecific antibodies of the present invention, e.g. PRO2589 and PR02591, exhibit a sub-pM EC₅₀ for killing high ROR1-expressing JEKO-1 cells, and some of these multispecific antibodies, e.g. PR02589 and PRO2590, exhibit an EC₅₀ for killing low ROR1-expressing SKOV-3 ovarian cancer cells of below 50 pM, as determined in T-cell driven cytotoxicity assays against said target cells (e.g. FIGS. 12 and 15).

Furthermore, the multispecific antibodies of the Invention, that comprise one ROR1-6D, as defined herein, exhibit a maximum killing of different ROR1-expressing cancer cells of 70% and above, as determined in a T cell driven cytotoxicity assay against said cancer cells. PR02510 for example exhibits a maximum killing of about 80% for MDA-MB-231 cancer cells, JEKO-1 cancer cells, JIMT-1 cancer cells and SKOV-3 cancer cells expressing high to low levels of ROR1 (e.g. FIGS. 12 and 15).

In addition, the anti-ROR1 x CD3 multispecific antibodies of the present invention exhibit very advantageous biophysical properties, in particular an excellent storage stability.

The multispecific antibodies of the present invention thus provide distinct therapeutic advantages over conventional therapies.

Unless defined otherwise, all technical and scientific terms used herein have the same meaning as commonly understood by those of ordinary skill in the art to which this invention pertains.

The terms “comprising” and “including” are used herein in their open-ended and non-limiting sense unless otherwise noted. With respect to such latter embodiments, the term “comprising” thus includes the narrower term “consisting of”.

The terms “a” and “an” and “the” and similar references in the context of describing the Invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, and the like, this is taken to mean also a single compound, salt, or the like.

In one aspect, the present invention relates to a multispecific antibody comprising:
a) one or two binding domains, which specifically bind to the extracellular domain of ROR1 (ROR1-BDs); and
b) one binding domain, which specifically binds to CD3 (CD3-BD);
wherein said ROR1-BDs and said CD3-BD comprise the CDR sequences as defined above.

In a further aspect, the present invention relates to a multispecific antibody comprising:
a) one or two binding domains, which specifically bind to the extracellular domain of ROR1 (ROR1-BDs); and
b) one binding domain, which specifically binds to CD3 (CD3-BD);
wherein said ROR1-BDs and said CD3-BD comprise the CDR sequences as defined above;
and wherein the multispecific antibody does not comprises an immunoglobulin Fc region.

The term “antibody” and the like, as used herein, includes whole antibodies or single chains thereof; and any antigen-binding fragment (i.e., “antigen-binding portion”) or single chains thereof; and molecules comprising antibody CDRs, VH regions or VL regions (including without limitation multispecific antibodies). A naturally occurring “whole antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), flanked by regions that are more conserved, termed framework regions (FRs). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “immunoglobulin Fc region” or “Fc region”, as used herein, is used to define a C-terminal region of an immunoglobulin heavy chain, i.e. the CH2 and CH3 domains of the heavy chain constant regions. The term “Fc region” includes native-sequence Fc regions and variant Fc regions, i.e. Fc regions that are engineered to exhibit certain desired properties, such as for example altered Fc receptor binding function and/or reduced or suppressed Fab arm exchange. An example of such an engineered Fc region is the knob-into-hole (KiH) technology (see for example Ridgway et al., Protein Eng. 9:617-21 (1996) and Spiess et al., J Biol Chem. 288(37):26583-93 (2013)). Native-sequence Fc regions include human IgG1, IgG2 (IgG2A, IgG2B), IgG3 and IgG4. “Fc receptor” or “FcR” describes a receptor that binds to the Fc region of an antibody. Particularly, the FcR is a native sequence human FcR, which binds an IgG antibody (a gamma receptor) and includes receptors of the FcγRI, FcγRII, and FcγRIII subclasses, including allelic variants and alternatively spliced forms of these receptors, FcγRil receptors including FcγRIIA (an “activating receptor”) and FcγRI IB (an “inhibiting receptor”), which have similar amino acid sequences that differ primarily in the cytoplasmic domains thereof. Activating receptor FcγRIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain, (see M. Daeron, Annu. Rev. Immunol. 5:203-234 (1997). FcRs are reviewed in Ravetch and Kinet, Annu. Rev. Immunol. 9:457-92 (1991); Capet et al., Immunomethods 4: 25-34 (1994); and de Haas et al., J. Lab. COin. Med. 126: 330-41 (1995). Other FcRs, including those to be identilied in the future, are encompassed by the term “FcR” herein. The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetle et al., Nature Biotechnology 15 (7): 637-40 (1997); Hinton et al., J. Biol. Chem. TJI (8): 6213-6 (2004); WO 2004/92219 (Hinton et al). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

The terms “binding domain”, “antigen-binding fragment thereof”, “antigen-binding portion” of an antibody, and the like, as used herein, refer to one or more fragments of an intact antibody that retain the ability to specifically bind to a given antigen (e.g., ROR1, CD3, hSA). Antigen-binding functions of an antibody can be performed by fragments of an intact antibody. Specifically, in case of the multispecific antibodies of the present Invention, the terms “binding domain”, “antigen-binding fragment thereof”, “antigen-binding portion”, and the like, as used herein, refer to a Fab fragment, i.e. a monovalent fragment consisting of the VL, VH, CL and CH1 domains; a F(ab)₂ fragment, i.e. a bivalent fragment comprising two Fab fragments linked by a disulfide bridge at the hinge region; an Fv fragment consisting of the VL and VH domains of a single arm of an antibody; a disulfide stabilized Fv fragment (dsFv); a single chain Fv fragment (scFv) and an scAb, i.e. a single chain consisting of the VL, VH and CL domains of a single arm of an antibody. Preferably, the binding domains of the antibodies of the present Invention are independently of each other selected from a Fv fragment, a scFv fragment and a single-chain Fv fragment (scFv). In particular embodiments, the binding domains of the antibodies of the present Invention are independently of each other selected from a single-chain Fv fragment (scFv). In other particular embodiments, the VL and VH domains of the scFv fragment are stabilized by an interdomain disulfide bond, in particular said VH sequence comprises a single cystelne residue in position 51 (AHo numbering) and said VL sequence comprises a single cysteine residue in position 141 (AHo numbering).

In one embodiment, the multispecific antibody of the invention does not comprise CH1 and/or CL regions. In particular embodiments, the multispecific antibody of the invention does neither comprise immunoglobulin Fc regions nor CH1 and CL regions.

The term “Complementarity Determining Regions” (“CDRs”) refers to amino acid sequences with boundaries determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); ImMunoGenTics (IMGT) numbering (Lefranc, M.-P., The Immunologist, 7,132-136 (1999); Lefranc, M.-P. et al., Dev. Comp. Immunol., 27, 55-77 (2003)) (“IMGT” numbering scheme); and the numbering scheme described in Honegger & Plückthun, J. Mol. Blol. 309 (2001) 657-670 (“AHo” numbering). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 24-34 (LCDR1), 50-56 (LCDR2), and 89-97 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to “Kabat”). Under IMGT, the CDRs of an antibody can be determined using the program IMGT/DomainGap Align.

In the context of the present invention, the numbering system suggested by Honegger & Plückthun (“AHo”) is used (Honegger & Plückthun, J. Mol. Biol. 309 (2001) 657-670), unless specifically mentioned otherwise. In particular, the following residues are defined as CDRs according to AHo numbering scheme: LCDR1 (also referred to as CDR-L1): L24-L42; LCDR2 (also referred to as CDR-L2): L58-L72; LCDR3 (also referred to as CDR-L3): L107-L138; HCDR1 (also referred to as CDR-H1): H27-H42; HCDR2 (also referred to as CDR-H2): H57-H76; HCDR3 (also referred to as CDR-H3): H108-H138. For the sake of clarity, the numbering system according to Honegger & Plückthun takes the length diversity into account that is found in naturally occurring antibodies, both in the different VH and VL subfamilies and, in particular, in the CDRs, and provides for gaps in the sequences. Thus, in a given antibody variable domain usually not all positions 1 to 149 will be occupied by an amino acid residue.

The term “binding specificity” as used herein refers to the ability of an individual antibody to react with one antigenic determinant and not with a different antigenic determinant. As used herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) Is an antibody that binds this target with greater affinity, avidity, more readily, and/or with greater duration than it binds to other targets. In its most general form (and when no defined reference is mentioned), “specific binding” is referring to the ability of the antibody to discriminate between the target of interest and an unrelated molecule, as determined, for example, in accordance with specificity assay methods known in the art. Such methods comprise, but are not limited to Western blots, ELISA, RIA, ECL, IRMA, SPR (Surface plasmon resonance) tests and peptide scans. For example, a standard ELISA assay can be carried out. The scoring may be carried out by standard color development (e.g. secondary antibody with horseradish peroxide and tetramethyl benzidine with hydrogen peroxide). The reaction in certain wells is scored by the optical density, for example, at 450 nm. Typical background (=negative reaction) may be about 0.1 OD; typical positive reaction may be about 1 OD. This means the ratio between a positive and a negative score can be 10-fold or higher. In a further example, an SPR assay can be carried out, wherein at least 10-fold, particularly at least 100-fold difference between a background and signal indicates on specific binding. Typically, determination of binding specificity is performed by using not a single reference molecule, but a set of about three to five unrelated molecules, such as milk powder, transferrin or the like.

Suitably, the antibodies of the invention are isolated antibodies. The term “isolated antibody”, as used herein, refers to an antibody that is substantially free of other antibodies having different antigenic specificities (e.g., an isolated antibody that specifically binds ROR1 and CD3 is substantially free of antibodies that specifically bind antigens other than ROR1 and CD3, or an isolated antibody that specifically binds ROR1, CD3 and hSA is substantially free of antibodies that specifically bind antigens other than ROR1, CD3 and hSA). Moreover, an isolated antibody may be substantially free of other cellular material and/or chemicals.

Suitably, the antibodies of the Invention are monoclonal antibodies. The term “monoclonal antibody” as used herein refers to antibodies that have substantially identical amino acid sequences or are derived from the same genetic source. A monoclonal antibody displays a binding specificity and affinity for a particular epitope, or binding specificities and affinities for specific epitopes.

Antibodies of the Invention Include, but are not limited to, chimeric, human and humanized antibodies.

The term “chimeric antibody” (or antigen-binding fragment thereof), as used herein, refers to an antibody molecule (or antigen-binding fragment thereof) in which (a) the constant region, or a portion thereof, is altered, replaced or exchanged so that the antigen-binding site (variable region) is linked to a constant region of a different or altered class, effector function and/or species; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “human antibody” (or antigen-binding fragment thereof), as used herein, is intended to include antibodies (and antigen-binding fragments thereof) having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies and antigen-binding fragments thereof of the Invention may include amino acid residues not encoded by human sequences (e.g., mutations introduced by random or site-specific mutagenesis in viro or by somatic mutation in vivo). This definition of a human antibody specifically excludes a humanized antibody comprising non-human antigen-binding residues. Human antibodies can be produced using various techniques known in the art, including phage-display libraries (Hoogenboom and Winter, J. Mol. Bid, 227:381 (1992); Marks et al., J. Mal. Biol, 222:581 (1991)). Also available for the preparation of human monoclonal antibodies are methods described in Cole et al, Monocional Antibodies and Cancer Therapy, Alan R. Uss, p. 77 (1985); Boemer et al., J. Immunol, 147(1):86-95 (1991). See also van Dijk and van de Winkel, Curr. Opin. Pharmacol, 5: 368-74 (2001). Human antibodies can be prepared by administering the antigen to a transgenic animal that has been modified to produce such antibodies in response to antigenic challenge, but whose endogenous loci have been disabled, e.g., Immunized xenomice (see, e.g., U.S. Pat. Nos. 6,075,181 and 6,150,584 regarding XENOMOUSE™ technology). See also, for example, Li et al, Proc. Na. Acad. Sci. USA, 103:3557-3562 (2006) regarding human antibodies generated via a human B-cel hybridoma technology.

The term “humanized” antibody (or antigen-binding fragment thereof), as used herein, refers to an antibody (or antigen-binding fragment thereof) that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). Additional framework region modifications may be made within the human framework sequences as well as within the CDR sequences derived from the germline of another mammalian species. The humanized antibodies of the invention may include amino acid residues not encoded by human sequences (e.g., mutations introduced by random or site-specific mutagenesis in vitro or by somatic mutation in vivo, or a conservative substitution to promote stability or manufacturing). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239: 1534-1536, 1988; Padlan, Molec. Immun., 28:489-498, 1991; and Padlan, Molec. Immun., 31:169-217, 1994. Other examples of human engineering technology include but are not limited to the Xoma technology disclosed in U.S. Pat. No. 5,766,886.

The term “recombinant humanized antibody” as used herein, includes all human antibodies that are prepared, expressed, created or isolated by recombinant means, such as antibodies Isolated from a host cell transformed to express the humanized antibody, e.g., from a transfectoma, and antibodies prepared, expressed, created or Isolated by any other means that involve splicing of all or a portion of a human immunoglobulin gene, sequences to other DNA sequences.

Preferably, the multispecific antibodies of the Invention are humanized. More preferably, the multispecific antibodies of the invention are humanized and comprise rabbit derived CDRs.

The term “multispecific antibody” as used herein, refers to an antibody that binds to two or more different epitopes on at least two or more different targets (e.g., ROR1 and CD3). The term “multispecific antibody” includes bispecific, trispecific, tetraspecific, pentaspecific and hexaspecific. Preferably, the multispecific antibodies of the invention are bispecific antibodies or trispecific antibodies, in particular trispecific antibodies. The term “bispecific antibody” as used herein, refers to an antibody that binds to at least two different epitopes on two different targets (e.g., ROR1 and CD3). The term “trispecific antibody” as used herein, refers to an antibody that binds to at least three different epitopes on three different targets (e.g., ROR1, CD3 and hSA).

The term “epitope” means a protein determinant capable of specific binding to an antibody. Epitopes usually consist of chemically active surface groupings of molecules such as amino acids or sugar side chains and usually have specific three-dimensional structural characteristics, as well as specific charge characteristics. “Conformational“and linear” epitopes are distinguished in that the binding to the former but not the latter is lost in the presence of denaturing solvents.

The term “conformational epitope” as used herein refers to amino acid residues of an antigen that come together on the surface when the polypeptide chain folds to form the native protein.

The term “linear epitope” refers to an epitope, wherein all points of interaction between the protein and the interacting molecule (such as an antibody) occurring linearly along the primary amino acid sequence of the protein (continuous).

The term “recognize” as used herein refers to an antibody antigen-binding fragment thereof that finds and interacts (e.g., binds) with its conformational epitope.

The term “avidity” as used herein refers to an informative measure of the overall stability or strength of the antibody-antigen complex. It is controlled by three major factors: antibody epitope affinity; the valency of both the antigen and antibody; and the structural arrangement of the interacting parts. Ultimately these factors define the specificity of the antibody, that is, the likelihood that the particular antibody is binding to a precise antigen epitope.

The multispecific antibodies of the invention comprise one or two ROR1-BDs.

The term “ROR1” refers in particular to human ROR1 with UniProt ID number 001973. Suitably, the ROR1-BD of the present invention targets human ROR1, in particular the extracellular domain (ECD) of ROR1, which sequence is shown in Table 6 (SEQ ID NO: 117). In particular embodiments, the ROR1-BD binds to the Ig-like domain of ROR1. The Ig-like domain of ROR1 is defined by residues 42 to 147 of SEQ ID NO: 117, in agreement with UniProt ID number 001973. In other particular embodiments the ROR1-BD does not block the binding of Wnt5a to ROR1.

Suitably, the ROR1-BDs, when being in scFv format, are characterized by one or more of the following parameters:
a. bind to the extracellular domain of human ROR1 with a monovalent dissociation constant (K_{D}) of 1 pM to 2 nM, particularly with a K_{D} of 1 pM to 1 nM, particularly of 1 to 500 pM, as measured by surface plasmon resonance (SPR);
b. bind to human ROR1-expressing MDA-MB-231 cells with an EC₅₀ of 5 pM to 10 nM, particularly with an EC₅₀ of 5 pM to 5 nM, particularly with an EC₅₀ of 5 pM to 4 nM, particularly with an EC₅₀ of 5 pM to 3 nM.

The term “MDA-MB-231 cells”, as used herein, refers toa breast cancer cell line that expresses ROR1. MDA-MB-231 cells are commercially available and serve as a model cell line for breast cancer (adenocarcinoma).

As used herein, the term “affinity” refers to the strength of interaction between the antibody and the antigen at single antigenic sites. Within each antigenic site, the variable region of the antibody “arm” interacts through weak non-covalent forces with antigen at numerous sites; the more interactions, the stronger the affinity.

“Binding affinity” generally refers to the strength of the total sum of non-covalent interactions between a single binding site of a molecule (e.g., of an antibody) and its binding partner (e.g., an antigen or, more specifically, an epitope on an antigen). Unless indicated otherwise, as used herein, “binding affinity”, “bind to”, “binds to” or “binding to” refers to intrinsic binding affinity that reflects a 1:1 interaction between members of a binding pair (e.g., an antibody fragment and an antigen). The affinity of a molecule X for its partner Y can generally be represented by the dissociation constant (K_{D}). Affinity can be measured by common methods known in the art, including those described herein. Low-affinity antibodies generally bind antigens slowly and tend to dissociate readily, whereas high-affinity antibodies generally bind antigens faster and tend to remain bound longer. A variety of methods of measuring binding affinity are known in the art, any of which can be used for purposes of the present invention. Specific illustrative and exemplary embodiments for measuring binding affinity, i.e. binding strength are described in the following.

The term “K_{assoc}”, “Kₐ” or “Kₒₙ”, as used herein, are intended to refer to the association rate of a particular antibody-antigen interaction, whereas the term “K_{dis}”, “K_{d}” or “K_{off}”, as used herein, is intended to refer to the dissociation rate of a particular antibody-antigen interaction. In one embodiment, the term “K_{D}”, as used herein, is intended to refer to the dissociation constant, which is obtained from the ratio of K_{D} to Kₐ (i.e. K_{d}/Kₐ) and is expressed as a molar concentration (M). The “K_{D}” or “K_{D} value” or “K_{D}” or “K_{D} value” according to this invention is in one embodiment measured by using surface plasmon resonance assays.

Affinity to recombinant human ROR1 was determined by surface plasmon resonance (SPR) measurements, as described in paragraphs [0176] and [0192] (scFvs); and [0219] and [0242] to [0244] (multispecific molecules). Affinities to recombinant human CD3, recombinant Cynomolgus CD3 and recombinant Marmoset CD3 were determined by surface plasmon resonance (SPR) measurements, as described in paragraphs [0208] (scFvs); and [0221] and [0247] (multispecific molecules). Affinities to recombinant human Serum Albumin (hSA), cynomolgus monkey serum albumin (cSA) and mouse serum albumin (mSA) were determined by surface plasmon resonance (SPR) measurements, as described in the paragraphs [0213] (scFvs); and [0223] and [0249] (multispecific molecules).

In some embodiments, the ROR1-BDs of the present invention or the ROR1-BDs present in the multispecific antibodies of the present Invention do not block the binding of Wnt5a to ROR1.

Suitably, the ROR1-BDs of the multispecific antibody of the present Invention, when being in scFv format, are further characterized by one or more of the following parameters:
c. have a melting temperature (Tm), determined by differential scanning fluorimetry (DSF), of at least 58° C., particularly of at least 59° C., particularly of at least 60° C., particularly of at least 61° C., in particular wherein said scFvs are formulated in 50 mM phosphate citrate buffer with 150 mM NaCl at pH 6.4;
d. have a loss in monomer content, after storage for four weeks at 4° C. of less than 3%, particularly less than 2%, particularly less than 1%, when said scFvs are at a starting concentration of 10 mg/mi, and in particular wherein said scFvs are formulated in 50 mM phosphate citrate buffer with 150 mM NaCl at pH 6.4; and/or
e. have a loss in monomer content, after storage for four weeks at 40° C. of less than 10%, when said scFvs are at a starting concentration of 10 mg/ml, and in particular wherein said scFvs are formulated in 50 mM phosphate citrate buffer with 150 mM NaCl at pH 6.4; and/or
f. have a loss in protein content, after storage for four weeks at 4° C. or 40° C. of less than 1%, when said scFvs are at a starting concentration of 10 mg/ml, and in particular wherein said scFvs are formulated in 50 mM phosphate citrate buffer with 150 mM NaCl at pH 6.4.

For most of the measurements, nano DSF (Prometheus NT.48, NaonTemper) method was used for determining the thermodynamic properties of the scFvs and the multispecific antibodies, as described for example in paragraphs [0197] and [0206] (scFv) and in paragraphs [0234] and [261] (multispecific formats). In case of the CD3-BD (scFvs) a DSF method was used as described in Egan, et al., MAbs, 9(1) (2017), 68-84; Niesen, et al., Nature Protocols, 2(9) (2007) 2212-2221. In this method, the midpoint of transition for the thermal unfolding of the scFv constructs is determined by Differential Scanning Fluorimetry using the fluorescence dye SYPRO® Orange (see Wong & Raleigh, Protein Science 25 (2016) 1834-1840). Samples in phosphate-citrate buffer at pH 6.4 are prepared at a final protein concentration of 50 μg/ml and containing a final concentration of 5× SYPRO® Orange in a total volume of 100 μl. Twenty-five microliters of prepared samples are added in triplicate to white-walled AB gene PCR plates. The assay is performed in a qPCR machine used as a thermal cycler, and the fluorescence emission is detected using the software's custom dye calibration routine. The PCR plate containing the test samples is subjected to a temperature ramp from 25° C. to 96° C. in increments of 1° C. with 30 s pauses after each temperature increment. The total assay time is about 2 h. The Tm is calculated by the software GraphPad Prism using a mathematical second derivative method to calculate the inflection point of the curve. The reported Tm is an average of three measurements.

The loss in monomer content is determined by area under the curve calculation of SE-HPLC chromatograms. SE-HPLC is a separation technique based on a solid stationary phase and a liquid mobile phase as outlined by the US Pharmacopeia (USP), chapter 621. This method separates molecules based on their size and shape utilizing a hydrophobic stationary phase and aqueous mobile phase. The separation of molecules is occurring between the void volume (V₀) and the total permeation volume (V_{T}) of a specific column. Measurements by SE-HPLC are performed on a Chromaster HPLC system (Hitachi High-Technologies Corporation) equipped with automated sample injection and a UV detector set to the detection wavelength of 280 nm. The equipment is controlled by the software EZChrom Elite (Agilent Technologies, Version 3.3.2 SP2) which also supports analysis of resulting chromatograms. Protein samples are cleared by centrifugation and kept at a temperature of 4-6° C. in the autosampler prior to injection. For the analysis of scFv samples the column Shodex KW403-4F (Showa Denko Inc., #F6989202) is employed with a standardized buffered saline mobile phase (50 mM sodium-phosphate pH 6.5, 300 mM sodium chloride) at the recommended flow rate of 0.35 ml/min. The target sample load per injection was 5 pg. Samples are detected by an UV detector at a wavelength of 280 nm and the data recorded by a suitable software suite. The resulting chromatograms are analyzed in the range of V₀ to V_{T} thereby excluding matrix associated peaks with >10 min elution time.

Suitably, the ROR1-BDs of the multispecific antibodies of the invention are binding domains provided in the present disclosure. The ROR1-BDs of the multispecific antibodies of the invention Include, but are not limited to, the humanized ROR1-BDs whose sequences are listed in Table 1.

Additionally, the multispecific antibodies of the invention comprise one CD3-BD. In particular embodiments, said CD3-BD is binding to CD3ε. Particularly, the multispecific antibodies of the invention comprise one CD3BD that targets human and cynomogus (Macaca fascicularis) CD3ε.

Suitably, the CD3-BD used in the present Invention, when being in scFv format, is characterized by one or more of the following parameters:
a) binds to human CD3ε with a dissociation constant (K_{D}) of less than 50 nM, particularly with a K_{D} of 0.1 to 50 nM, particularly of 0.1 to 30 nM, particularly of 0.1 to 15 nM, particularly of 0.1 to 10 nM, as measured by SPR;
b) binds to Macaca fasciculads (Cynomolgus) CD3 with a K_{D} of less than 50 nM, particularly with a K_{D} of 0.1 to 50 nM, particularly of 0.1 to 30 nM, particularly of 0.1 to 15 nM, particularly of 0.1 to 10 nM, as measured by SPR; and
c) has a melting temperature (Tm), determined by differential scanning fluorimetry (DSF), of at least 60° C., particularly of at least 65° C., more particularly of at least 67° C.

Suitably, the CD3-BD of the multispecific antibodies of the invention are binding domains provided in the present disclosure. The CD3-BD of the multispecific antibodies of the invention include, but are not limited to, the humanized CD3-BDs whose sequences are listed in Table 2.

In order to increase the number of specificities/functionalities at the same or lower molecular weight, it is advantageous to use antibodies comprising antibody fragments, such as Fv and Fab fragments and other antibody fragments. These smaller molecules retain the antigen-binding activity of the whole antibody and can also exhibit improved tissue penetration and pharmacokinetic properties in comparison to the whole immunoglobulin molecules. Whilst such fragments appear to exhibit a number of advantages over whole immunoglobulins, they also suffer from an increased rate of clearance from serum since they lack the Fc domain that Imparts a long half-life in vivo (Medasan et al., 1997, J. Immunol. 158:2211-2217). Molecules with lower molecular weights penetrate more efficiently into target tissues (e.g. solid cancers) and thus hold the promise for improved efficacy at the same or lower dose.

The Inventors have now found that the addition of a human serum albumin binding domain (hSA-BD) to the multispecific antibody of the invention does not interfere with the ability of the other binding domains to bind to their respective targets. This finding is insofar surprising as it cannot a prior be expected that all three or four binding domains remain functional without sterically or otherwise inhibiting each other in a complex multi-target, multi-cell in vivo situation.

The term “hSA” refers in particular to human serum albumin with UniProt ID number P02768. Human serum albumin (hSA) is 66.4 kDa abundant protein in human serum (50% of total protein) composed of 585 amino acids (Suglo, Protein Eng, Vol. 12, 1999, 439-446). Multifunctional hSA protein is associated with its structure that allowed binding and transporting a number of metabolizes such as fatty acids, met all ions, bilirubin and some drugs (Fanali, Molecular Aspects of Medicine, Vol. 33, 2012, 209-290). HSA concentration in serum is around 3.5-5 g/dl. Albumin-binding antibodies and fragments thereof may be used for example, for extending the in vivo serum half-life of drugs or proteins conjugated thereto.

In some embodiments, the hSA-BD is derived from a monoclonal antibody or antibody fragment.

Suitable hSA-BDs comprised in the multispecific antibodies of the invention are binding domains provided in the present disclosure. The hSA-BDs comprised in the multispecific antibodies of the invention include, but are not limited to, the humanized hSA-binding domains whose sequences are listed in Table 3.

In particular embodiments, the hSA-BD comprised in the multispecific antibodies of the Invention specifically bind to hSA and cynomolgus monkey serum albumin (cSA).

In other particular embodiments, the hSA-BD comprised in the multispecific antibodies of the Invention specifically bind to hSA, cSA and mouse serum albumin (mSA). In these particular embodiments, the hSA-BD comprised in the multlspecific antibodies of the Invention, when being in scFv format, are characterized by one or more of the following parameters:
a) bind to human serum albumin (hSA) with a dissociation constant (K_{D}) of 0.1 to 10 nM, particularly of 0.1 to 5 nM, as measured by SPR at pH 5.5 and pH 7.4;
b) bind to Macaca fascicularis (Cynomolgus) serum albumin (cSA) with a dissociation constant (K_{D}) of 0.1 to 10 nM, particularly of 0.1 to 5 nM, as measured by SPR at pH 5.5 and pH 7.4; and
c) bind to mouse serum albumin (mSA) with a dissociation constant (K_{D}) of 0.1 to 20 nM, particularly of 0.1 to 10 nM, as measured by SPR at pH 5.5 and pH 7.4.

Other suitable hSA-BD for use in the multispecific antibody of the invention comprises or is derived from an antibody selected from the group consisting of: (I) polypeptides that bind serum albumin (see, for example, Smith et al., 2001, Bioconjugate Chem. 12:750-756; EP0486525; U.S. Pat. No. 6,267,964; WO 2004/001064; WO 2002/076489; and WO 2001/45746); (ii) anti-serum albumin binding single variable domains described in Holt et al., Protein Engineering, Design & Selection, vol 21, 5, pp283-288, WO 2004/003019, WO 2008/096158, WO 2005/118642, WO 2006/0591056 and WO 2011/006915; (iii) anti-serum albumin antibodies described in WO 2009/040562, WO 2010/035012 and WO 2011/086091.

In particular embodiments, the multispecific antibody of the invention comprise: (I) one ROR1-BD; (ii) one CD3-BD; and (ii) one hSA-BD, i.e. the multispecific antibodies of these particular embodiments are monovalent for all three ROR1, CD3 and hSA specificities.

In further particular embodiments, the multispecific antibody of the invention comprise: (i) two ROR1-BD; (ii) one CD3-BD; and (ii) one hSA-BD, i.e. the multispecific antibodies of these particular embodiments are bivalent for ROR1 specificity and monovalent for both the CD3 and hSA specificities.

The term “multivalent antibody refers to a single binding molecule with more than one valency, where “valency” is described as the number of antigen-binding moieties that binds to epitopes on target molecules. As such, the single binding molecule can bind to more than one binding site on a target molecule and/or to more than one target molecule due to the presence of more than one copy of the corresponding antigen-binding moieties. Examples of multivalent antibodies include, but are not limited to bivalent antibodies, trivalent antibodies, tetravalent antibodies, pentavalent antibodies, hexavalent antibodies, and the like.

The term “monovalent antibody”, as used herein, refers to an antibody that binds to a single target molecule, and more specifically to a single epitope on a target molecule. Also, the term “binding domain‘ or’monovalent binding domain, as used herein, refers to a binding domain that binds to a single epitope on a target molecule.

In case the multispecific antibodies of the invention comprise two ROR1-BDs, said two ROR1-BDs either bind the same epitope or different epitopes on the extracellular domain of ROR1. Preferably, the two ROR1-BDs bind the same epitope on the extracellular domain of ROR1.

The term ‘same epitope’, as used herein, refers to an individual protein determinant on the protein capable of specific binding to more than one antibody, where that individual protein determinant is identical, i.e. consist of identical chemically active surface groupings of molecules such as amino acids or sugar side chains having identical three-dimensional structural characteristics, as well as identical charge characteristics for each of said antibodies. The term “different epitope”, as used herein in connection with a specific protein target, refers to individual protein determinants on the protein, each capable of specific binding to a different antibody, where these individual protein determinants are not identical for the different antibodies, i.e. consist of non-identical chemically active surface groupings of molecules such as amino acids or sugar side chains having different three-dimensional structural characteristics, as well as different charge characteristics. These different epitopes can be overlapping or non-overlapping.

In particular embodiments, the multispecific antibodies of the invention are bispecific and bivalent.

In further particular embodiments, the multispecific antibodies of the invention are bispecific and trivalent.

In further particular embodiments, the multispecific antibodies of the invention are trispecific and trivalent.

In further particular embodiments, the multispecific antibodies of the invention are trispecific and tetravalent.

Other variable domains used in the invention include amino acid sequences that have been mutated, yet have at least 80, 85, 90, 91, 92, 93, 94, 95, 96, 97, 98 or 99 percent identity in the CDR regions with the CDR regions depicted in the sequences described in Tables 1, 2 and 3. Other variable domains used in the invention include mutant amino acid sequences wherein no more than 1, 2, 3, 4 or 5 amino acds have been mutated in the CDR regions when compared with the CDR regions depicted in the sequence described in Tables 1, 2and 3.

Suitably, the VH domains of the binding domains of the multispecific antibodies of the invention belong to or are selected from a human antibody VH family. In preferred embodiments, the VH domains of the one or two ROR1-BDs, the CD3-BD and, if present, the hSA-BD of the multispecific antibodies of the invention belong to VH framework subtypes VH1a, VH1b, VH3 or VH4. In one particular embodiment, said VH domains belong to the VH framework subtype VH3 or VH4, particularly to the VH framework subtype VH3.

In the context of the present invention, the term belong to a VHx framework subtype (or Vκ/Vλ framework subtype) or “selected from a VHx framework subtype (or Vκ/Vλ framework subtype)” means that the VHNL framework sequences FR1 to FR4 (herein also referred to as the VH/VL framework regions FR1 to FR4) show the highest degree of homology to said human antibody VHx or VKXA framework subtype.

Examples of VH1a, VH1b, VH3 and VH4 sequences, and examples of other VHx sequences, are given in Knappik et al., J. Mol. Biol. 296 (2000) 57-86, or in WO 2019/057787. A specific example of a VH domain belonging to VH3 family is represented by SEQ ID NO: 71, and a specific example of a VH domain belonging to the VH1a, VH1b or VH4 framework subtype are represented by SEQ ID NOs: 72, 73 and 74 (Table 4, framework regions are marked in non-bold). In particular, framework regions FR1 to FR4 are taken from SEQ ID NO: 71 belonging to the VH3 family (Table 4, regions marked in non-bold). Suitably, a VH belonging to VH3 family, as used herein, is a VH comprising FR1 to FR4 having at least 85%, particularly at least 90%, more particularly at least 95% sequence identity to FR1 to FR4 of SEQ ID NO: 71. Alternative examples of VH3 and VH4 sequences, and examples of other VHx sequences, may be found in Knappik et al., J. Mol. Biol. 296 (2000) 57-86 or in WO 2019/057787.

Suitably, the VL domains of the binding domains used in the Invention comprise: V_{K} frameworks FR1, FR2 and FR3, particularly V_{K}1 or V_{K}3 frameworks, particularly V_{K}1 frameworks FR1 to FR3, and a framework FR4, which is selected from a V_{K} FR4. In the case where said binding domains are in an scFv-format, said binding domains comprise: V_{K} frameworks FR1, FR2 and FR3, particularly V_{K}1 or V_{K}3 frameworks, particularly V_{K}1 frameworks FR1 to FR3, and a framework FR4, which is selected from a a VA FR4.

Suitable V_{K}1 frameworks FR1 to FR3 as wel as an exemplary VA FR4 are set forth in SEQ ID NO: 75 (Table 4, framework regions are marked in non-bold). Alternative examples of V_{K}1 sequences, and examples of V_{K}2, V_{K}3 or V_{K}4 sequences, may be found in Knappik et al., J. Mol. Biol. 296 (2000) 57-86. Suitable V_{K}1 frameworks FR1 to FR3 comprise the amino acid sequences having at least 85, 90, 95 percent identity to amino acid sequences corresponding to FR1 to FR3 and taken from SEQ ID NO: 75 (Table 4, framework regions are marked in non-bold). Suitable VA FR4 are as set forth in SEQ ID NO: 76 to SEQ ID NO: 82 and in SEQ ID NO: 83 comprising a single cysteine residue, particular in a case where a second single cysteine is present in the corresponding VH chain, particulaly in position 51 (AHo numbering) of VH, for the formation of an inter-domain disulfide bond. In one embodiment, the VL domains of the binding domains of the multispecific antibodies of the invention, when being in scFv-format, comprises VA FR4 comprising the amino acid sequence having at least 85, 90, 95 percent identity to an amino acid sequence selected from any of SEQ ID NO: 76 to SEQ ID NO: 83, particularly to SEQ ID NO: 76 or 83.

In particular, the binding domains of the multispecific antibodies of the Invention comprise VH domains listed in Tables 1, 2 and 3. Suitably, the binding domains used the Invention comprise a VH amino acid sequence listed in one of Tables 1, 2 and 3, wherein no more than 10 amino acids in the framework sequences (i.e., the sequences which are not CDR sequences) have been mutated (wherein a mutation is, as various non-limiting examples, an addition, substitution or deletion). Suitably, the binding domains of the multispecific antibodies of the invention comprise a VH amino acid sequence listed in one of Tables 1, 2 and 3, wherein no more than 7 amino acids, particularly no more than 5, 4, 3 or 2 amino acids, particularly no more than 1 amino acid in the framework sequences (i.e., the sequences which are not CDR sequences) have been mutated (wherein a mutation is, as various non-limiting examples, an addition, substitution or deletion). Other binding domains used in the invention include amino acids that have been mutated, yet have at least 90, 91, 92, 93, 94, 95, 96, 97, 98 or 99 percent identity in the VH regions with the VH regions depicted in the corresponding sequences described in one of Tables 1, 2 and 3, including VH domains comprising at least positions 5 to 140 (AHo numbering), particularly at least positions 3 to 145, more particularly at least positions 2 to 147 of one of the sequences shown in Tables 1, 2 and 3, provided that such VH domains exhibit the functional features defined above in items 11 and 19.

In particular embodiments, the one or two ROR1-BDs, the CD3-BD and, if present, the hSA-BD of the multispecific antibodies of the Invention comprise VH domains as defined above, wherein the framework regions FR1, FR2, FR3 and FR4 of said VH domains have the following substitutions (AHo numbering): an arginine (R) at amino acid position 12; a threonine (T) at amino acid position 103 and a glutamine (Q) at amino acid position 144. Said VH domains may herein be referred to as “variant VH domains” or “modified VH domains” or “improved VH domains”. Likewise, binding domains comprising said VH domains may herein be referred to as “variant binding domains” or “modified binding domains” or “improved binding domains”. Specific examples of such variant VH domains are the VH domains of SEQ ID NOs: 8, 11, 21, 24, 37, 66 and 69.

It has been found by the Inventors that said modified ROR1-, CD3- and hSA-BDs, when being in scFv format, exhibit a significantly reduced immunogenicity, when compared to versions of said binding domains that do not comprise the above indicated substitutions in the VH framework regions. More specifically, said modified ROR1-, CD3- and hSA-BDs, when being in scFv format, exhibit a reduced binding to pre-existing anti-drug antibodies (ADA) present in human sera, in particular reduced binding to pre-existing ADAs, when compared to versions of said binding domains that do not comprise the above indicated substitutions in the VH framework regions, as determined in a pre-existing ADA binding assay, in particular as determined in a pre-existing ADA binding assay as defined in Example 10.

In particular, it has been found by the inventors that multispecific antibodies of the present invention, which comprise said modified ROR1-, CD3- and hSA-BDs, exhibit a significantly reduced immunogenicity, when compared to versions of multispecific antibodies of the present Invention that do not comprise said modified binding domains. More specifically, multispecific antibodies of the present invention, which comprise said modified ROR1-, CD3- and hSA-BDs, exhibit a reduced binding to pre-existing anti-drug antibodies (ADA) present in human sera, in particular reduced binding to pre-existing ADAs, when compared to versions of multispecific antibodies of the present invention that do not comprise said modified binding domains, as determined in a pre-existing ADA binding assay, in particular as determined in a pre-existing ADA binding assay as defined in Example 10.

Immunogenicity, i.e. the tendency of a therapeutic protein to induce an antibody response within the patient's body, can e.g. be predicted by its capacity to be recognized by anti-drug antibodies (ADAs) that are already present in human sera of healthy and untreated individuals, herein referred to as “pre-existing ADAs”.

Thus, for the purpose of the present invention, the term “immunogenicity”, as used herein, refers to the capacity of a therapeutic protein, e.g. an antibody, an antibody fragment or an antibody binding domain, to be recognized by pre-existing ADAs in human serum samples. Without intending to be bound by theory, it is believed that pre-existing ADA binding as well as the induction of the formation of ADAs during therapeutic treatment is linked with the occurrence of B cell and/or T cell epitopes on a therapeutic protein. The extent of such immunogenicity can be determined by an ELISA assay and can be expressed by the percentage of human serum samples, which contain measurable amounts of pre-existing ADAs and/or ADAs formed during therapeutic treatment, that recognize, i.e. bind to, the therapeutic protein in question, relative to the total number of tested human sera (percentage of positive serum samples). A reduction of immunogenicity between a therapeutic protein and a corresponding therapeutic protein being modified with the goal to reduce its immunogenicity can be measured by comparing the percentage of positive serum samples against the modified therapeutic protein, with the percentage of positive serum samples against the original therapeutic protein. A lower number or percentage of positive serum samples for the modified therapeutic protein indicates a reduction of immunogenicity relative to the original therapeutic protein.

A serum sample is deemed to contain measurable amounts of pre-existing ADAs, when the ELISA signal surpasses a certain threshold. This threshold is herein also referred to as the screening cut-point (SCP). The SCP can be calculated as defined below or set to an arbitrary value relative to the maximum ELISA signal obtained for the tested sera (e.g. 30%, 25%, 20%, 15%, 10% or 5% of the maximum ELISA signal obtained for the tested sera). Preferably, the SCP is calculated as defined below.

In particular, the binding domains of the multispecific antibodies of the Invention comprise a VL domain listed in one of Tables 1, 2 and 3. Suitably, the binding domains of the multispecific antibodies of the invention comprise a VL amino acid sequence listed in one of Tables 1, 2 and 3, wherein no more than about 10 amino acids in the framework sequences (i.e., the sequences which are not CDR sequences) have been mutated (wherein a mutation is, as various non-limiting examples, an addition, substitution or deletion). Suitably, the binding domains of the multispecific antibodies of the Invention comprise a VL amino acid sequence listed in one of Tables 1, 2 and 3, wherein no more than about 10 amino acids, particularly no more than 7 amino acids, particularly no more than 5, 4, 3, 2 amino adds, particularly no more than 1 amino acid in the framework sequences (i.e., the sequences which are not CDR sequences) have been mutated (wherein a mutation is, as various non-limiting examples, an addition, substitution or deletion). Other binding domains used in the invention include amino acids that have been mutated, yet have at least 90, 91, 92, 93, 94, 95, 96, 97, 98 or 99 percent identity in the VL regions with a VL region depicted in the sequences described in Tables 1, 2 and 3, including VL domains comprising at least positions 5 to 140 (AHo numbering), particularly at least positions 3 to 145, more particularly at least positions 2 to 147 of one of the sequences shown in Tables 1, 2 and 3, provided that such VL domains exhibit the functional features defined above in items 11 and 19.

In the context of the present invention, the term “binding domain used in the present Invention” relates to a binding domain as such, i.e. independent of a multispecific context, and, in particular, to a binding domain comprised in a multispecific construct, e.g. one of the binding domains comprised in a bispecific, trispecific or tetraspecific construct.

Suitably, the binding domains of the multispecific antibodies of the invention are independently from each other selected from the group consisting of: a Fab, a F(ab)₂, an Fv, an scFv, a dsFv, and an scAb; in particular from the group consisting of: an Fv, an scFv, and a dsFv. Particularly, the binding domains of the multispecific antibodies of the Invention are scFvs.

Suitably, the binding domains of the multispecific antibodies of the invention are operably linked. The binding domains of the multispecific antibodies of the invention are capable of binding to their respective antigens or receptors simultaneously. The term “simultaneously”, as used in this connection refers to the simultaneous binding of at least one of the ROR1-BDs and the CD3-BD to their respective antigens, or, in case where two ROR1-BDs are present in the multispecific antibody, the term “simultaneously” refers to the simultaneous binding of both ROR1-BDs and the CD3-BD to their respective antigens.

The multispecific antibodies of the present Invention comprising one or two ROR1-BDs, one CD3-BD and optionally one hSA-BD wherein said one or two ROR1-BDs, said CD3-BD and said optional hSA-BD are operably linked to each other.

The term “operably linked”, as used herein, indicates that two molecules (e.g., polypeptides, domains, binding domains) are attached in a way that each molecule retains functional activity. Two molecules can be “operably linked” whether they are attached directly or indirectly (e.g., via a linker, via a moiety, via a linker to a moiety). The term “linker” refers to a peptide or other moiety that is optionally located between binding domains or antibody fragments used in the invention. A number of strategies may be used to covalently link molecules together. These include, but are not limited to, polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. Choosing a suitable linker for a specific case where two polypeptide chains are to be connected depends on various parameters, including but not limited to the nature of the two polypeptide chains (e.g., whether they naturally oligomerize), the distance between the N- and the C-termini to be connected if known, and/or the stability of the linker towards proteolysis and oxidation. Furthermore, the linker may contain amino acid residues that provide flexibiity.

In the context of the present invention, the term “polypeptide linker” refers to a linker consisting of a chain of amino acid residues linked by peptide bonds that is connecting two domains, each being attached to one end of the linker. The polypeptide linker should have a length that is adequate to link two molecules in such a way that they assume the correct conformation relative to one another so that they retain the desired activity. In particular embodiments, the polypeptide linker has a continuous chain of between 2 and 30 amino acid residues (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues). In addition, the amino acid residues selected for inclusion in the polypeptide linker should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. In particular embodiments, the polypeptide linker is non-structured polypeptide. Useful linkers include glycine-serine, or GS inkers. By Gly-er or “GS” linkers is meant a polymer of glycines and serines in series (including, for example, (Gly-Ser)ₙ, (GSGGS)ₙ [SEQ ID NO: 118], (GGGGS)ₙ[SEQ ID NO: 119], and (GGGS)ₙ[SEQ ID NO: 120], where n is an integer of at least one), glycine-alanine polymers, alanine-serine polymers, and other flexible linkers such as the tether for the shaker potassium channel, and a large variety of other flexible linkers, as win be appreciated by those in the art Glycine-serine polymers are preferred since oligopeptides comprising these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single-chain antibodies.

In a group of embodiments, the multispecific antibody is in a format selected from any suitable format known in the art that is at least trispecific and does not comprise immunoglobulin Fc region(s) and CH1 and/or CL regions. This includes, by way of non-limiting example, formats based on a tandem tri-scFv (triplebody), a triabody, an scDb-scFv; a tandem tri-scFv or an scDb-scFv fused to the N- and/or the C-terminus of a heterodimerization domain other than heterodimeric Fc domains, and a MATCH (described in WO 2016/0202457; Egan T. et al., MABS 9 (2017) 68-84). Particularly, the format of the multispecific antibody is selected from an scDb-scFv, an scMATCH3, a MATCH3 and a MATCH4.

The term “scDB“or “single chain diabodies” refers to antibody fragments with two antigen-binding sites, which fragments comprise a VH connected to VL in the same polypeptide chain (VH-VL). By using a inker that is too short to allow pairing between the two domains on the same chain, the domains are forced to pair with the complementary domains of another chain to create two antigen-binding sites. Single chain diabodies may be bivalent or bispecific, preferably bispecific. Single chain diabodies are described more fully in, for example, EP 404 097, WO 93/01161, Hudson et al., Nat. Med. 9:129-134 (2003), and Hollinger et al., Proc. Natl. Acad. Sd. USA 90: 6444-6448 (1993). Triabodies are described in Hudson et al., Nat. Med. 9:129-134 (2003).

The bispecific scDb, in particular the bispecificmonomericscDb, particularly comprises two variable heavy chain domains (VH) or fragments thereof and two variable light chain domains (VL) or fragments thereof connected by linkers L1, L2 and L3 in the order VHA-L1-VLB-L2-VHB-L3-VLA, VHA-L1-VHB-L2-VLB-L3-VLA, VLA-L1-VLB-L2-VHB-L3-VHA, VLA-L1-VHB-L2-VLB-L3-VHA, VHB-L1-VLA-L2-VHA-L3-VLB, VHB-L1-VHA-L2-VLA-L3-VLB, VLB-L1-VLA-L2-VHA-L3-VHB or VLB-L1-VHA-L2-VLA-L3-VHB, wherein the VLA and VHA domains jointly form the antigen-binding site for the first antigen, and VLB and VHB jointly form the antigen-binding site for the second antigen.

The linker L1 particularly is a peptide of 2-10 amino acids, more particularly 3-7 amino acids, and most particularly 5 amino acids, and linker L3 particularly is a peptide of 1-10 amino acids, more particularly 2-7 amino acids, and most particularly 5 amino acids. In particular embodiments, the linker L1 and/or L3 comprises one or two units of four (4) glycine amino acid residues and one (1) serine amino acid residue (GGGGS)ₙ [SEQ ID NO: 121], wherein n=1 [SEQ ID NO: 85] or 2 [SEQ ID NO: 86], particularly n=1.

The middle linker L2 particularly is a peptide of 10-40 amino acids, more particularly 15-30 amino acids, and most particularly 20-25 amino acids. In particular embodiments, said linker L2 comprises one or more units of four (4) glycine amino acid residues and one (1) serine amino acid residue (GGGGS)ₙ[SEQ ID NO: 122], wherein n=1, 2, 3, 4, 5, 6, 7 or 8, particularly n-4 [SEQ ID NO: 84].

In one embodiment, the multispecific antibody of the invention comprises one ROR1-BD, one CD3-BD and one hSA-BD and is a single-chain protein, which has an scDb-scFv or an scMATCH3 format. The term “scDb-scFv” refers to an antibody format, wherein a single-chain Fv (scFv) fragment is fused by a flexible Gly-Ser linker to a single-chain diabody (scDb). In one embodiment, said flexible Gly-Ser linker is a peptide of 2-40 amino acids, e.g., 2-35, 2-30, 2-25, 2-20, 2-15, 2-10 amino acids, particularly 10 amino acids. In particular embodiments, said linker comprises one or more units of four (4) glycine amino acid residues and one (1) serine amino acid residue (GGGGS)ₙ [SEQ ID NO: 122], wherein n=1, 2, 3, 4, 5, 6, 7 or 8, particularly n=2 [SEQ ID NO: 86]. Particularly, the single-chain multispecific antibodies of this embodiment are in a scMATCH3 format.

Specific but non-limiting examples of scMATCH3 multispecific antibodies of the invention are PRO2286, PRO2287, PRO22507, PRO2508, PRO2509, PR02510, PR02557, PRO2596, PRO2667 and PRO2668, whose sequences are listed in Table 5.

In another embodiment, the multispecific antibody of the invention comprises two ROR1-BD, one CD3-BD and one hSA-BD and is a heterodimeric protein in a MATCH4 format described in WO 2016/0202457; Egan T., et al., MABS 9 (2017) 68-84.

Specific but non-limiting examples of heterodimeric MATCH4 multispecific antibodies of the invention are PR02589, PRO2590, PRO2591, PRO2592, PRO2658, PRO2659, PRO2669 and PRO2670, whose sequences are listed in Table 5.

The multispecific antibodies of the invention or fragments thereof or binding domains thereof, such as the ROR1-BDs can be produced using any convenient antibody-manufacturing method known in the art (see, e.g., Fischer, N. & Leger, O., Pathobiology 74 (2007) 3-14 with regard to the production of bispecific constructs; Hornig, N. & Färber-Schwarz, A., Methods Mol. Biol. 907 (2012)713-727, and WO 99/57150 with regard to bispecific diabodies and tandem scFvs). Specific examples of suitable methods for the preparation of the bispecific construct further include, inter alia, the Genmab (see Labrijn et al., Proc. Natl. Acad. Sci. USA 110 (2013) 5145-5150) and Merus (see de Kruif et al., Biotechnol. Bioeng. 106 (2010) 741-750) technologies.

These methods typically involve the generation of monoclonal antibodies, and the combination of the antigen-binding domains or fragments or parts thereof of two or more different monoclonal antibodies to give a bispecific or multispecific construct using known molecular cloning techniques.

The multispecific antibodies of the invention can be prepared by conjugating the constituent binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodimide, N-succinimidyl-5-acetyl-thioacetate (SATA), 5,5′-dithiobis (2-nitrobenzoic acid) (DTNB), 0-phenylenedimaleimide (oPDM), N-succinimidyl-3-(2-pyridyldithio)propionate (SPDP), and sulfosuccinimdyl 4-(N-maleimidomethyl)-cyclohexane-1-carboxylate (sulfo-SMCC) (see e.g., Karpovsky et al., 1984 J. Exp. Med. 160: 1686; Liu, M A et al., 1985 Proc. Natl. Acad. Sci. USA 82:8648). Other methods include those described in Paulus, 1985 Behring Ins. Mitt No. 78, 118-132; Brennan et al., 1985 Science 229:81-83, and Glennie et al., 1987 J. Immunol. 139: 2367-2375. Conjugating agents are SATA and sulfo-SMCC, both available from Pierce Chemical Co. (Rockford, Ill., USA).

When the binding specificities are antibodies, they can be conjugated by sulfhydryl bonding of the C-terminus hinge regions of the two heavy chains. In a particular embodiment, the hinge region is modified to contain an odd number of sulfhydryl residues, for example one, prior to conjugation.

Alternatively, two or more binding specificities can be encoded in the same vector and expressed and assembled in the same host cell. This method is particularly useful where the bispecific molecule is a mAb×Fab, a mAb×scFv, a mAb×dsFv or a mAb×Fv fusion protein. Methods for preparing multispecific antibodies and molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the multispecific antibodies to their specific targets can be confirmed by, for example, enzyme-linked immunosorbent assay (ELISA), radioimmunoassay (REA), FACS analysis, bioassay (e.g., growth Inhibition), or Western Blot assay. Each of these assays generally detects the presence of protein-antibody complexes of particular interest by employing a labeled reagent (e.g., an antibody) specific for the complex of Interest.

In a further aspect, the invention provides a nucleic acid or two nucleic acids encoding the multispecific antibody of the invention or fragments thereof or binding domains thereof, such as the ROR1-BDs. Such nucleic acids can be optimized for expression in mammalian cells.

The term “nucleic acid” is used herein interchangeably with the term “polynucleotide(s)” and refers to one or more deoxyribonucleotides or ribonudeotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphorates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The invention provides substantially purified nucleic acid molecules which encode polypeptides comprising segments or domains of the multispecific antibody, as described above, such as the ROR1-BDs. When expressed from appropriate expression vectors, polypeptides encoded by these nucleic acid molecules are capable of exhibiting antigen-binding capacities of the multispecific antibody or the ROR1-BD of the present invention.

The polynucleotide sequences can be produced by de novo solid-phase DNA synthesis or by PCR mutagenesis of an existing sequence (e.g., sequences as described in the Examples below) encoding the multispecific antibody of the invention or fragments thereof or binding domains thereof, such as the ROR1-BDs of the invention. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68: 109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., Nucleic Acids Res. 19:967, 1991; and Eckert et al., PCR Methods and Applications 1:17, 1991.

Also provided in the invention are expression vectors and host cells for producing the multispecific antibody of the invention or fragments thereof or binding domains thereof, such as the ROR1-BDs.

The term “vector” is intended to refer to a polynucleotide molecule capable of transporting another polynucleotide to which it has been linked. One type of vector is a “plasmid, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome.

Moreover, certain vectors are capable of directing the expression of genes to which they are operatively linked. Such vectors are referred to herein as recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. In this particular context, the term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

Various expression vectors can be employed to express the polynucleotides encoding the multispecific antibody chain(s) or fragments thereof or binding domains thereof, such as the ROR1-BDs. Both viral-based and non-viral expression vectors can be used to produce the antibodies in a mammalian host cell. Non-viral vectors and systems include plasmids, episomal vectors, typically with an expression cassette for expressing a protein or RNA, and human artificial chromosomes (see, e.g., Harrington et al., Nat Genet. 15:345, 1997). For example, non-viral vectors useful for expression of the IL-4R-binding polynucleotides and polypeptides in mammalian (e.g., human) ceils include pThioHis A, B and C, pcDNA3.1/His, pEBVHis A. B and C, (Invitrogen, San Diego, USA), MPS V vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68: 143, 1992.

The choice of expression vector depends on the intended host cells in which the vector is to be expressed. Typically, the expression vectors contain a promoter and other regulatory sequences (e.g., enhancers) that are operably linked to the polynucleotides encoding a multispecific antibody chain or a fragment. In one embodiment, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of a multispecific antibody chain or a fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20: 125, 1994; and Bitter et al., Meth. Enzymol., 153:516, 1987). For example, the SV40 enhancer or CMV enhancer may be used to increase expression in mammalian host cells.

Vectors to be used typically encode the multispecific antibody light and heavy chain variable domains. In certain cases they also encode constant regions or parts thereof. Such vectors allow expression of the variable regions as fusion proteins with the constant regions thereby leading to production of intact antibodies and antigen-binding fragments thereof. Typically, such constant regions are human.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be Identical to the parent cell, but are still included within the scope of the term “host cell” as used herein.

The host cells for harboring and expressing the multispecific antibody of the invention or fragments thereof or binding domains thereof, such as the ROR1-BDs, can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for Initiating and completing transcription and translation. Other microbes, such as yeast, can also be employed to express the multispecific antibodies of the invention. Insect cells in combination with baculovirus vectors can also be used.

In one embodiment, mammalian host cells are used to express and produce the multispecific antibody of the invention or fragments thereof or binding domains thereof, such as the ROR1-BDs. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various COS cell lines, Hela cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP pol III promoter, the constitutive MPS V promoter, the tetracycline-inducible CMV promoter (such as the human immediate-early CMV promoter), the constitutive CMV promoter, and promoter-enhancer combinations known in the art.

Methods for introducing expression vectors containing the polynucleotides of interest vary depending on the type of cellular host. For example, calcium chloride transfection is commonly utilized for prokaryotic cells, whereas calcium phosphate treatment or electroporation may be used for other cellular hosts. (See generally Green, M. R., and Sambrook, J., Molecular Cloning: A Laboratory Manual (Fourth Edition), Cold Spring Harbor Laboratory Press (2012)). Other methods include, e.g., electroporation, calcium phosphate treatment, liposome-mediated transformation, injection and microinjection, ballistic methods, virosomes, immunoliposomes, polycation-nucleic acid conjugates, naked DNA, artificial virions, fusion to the herpes virus structural protein VP22 (Elliot and O'Hare, Cell 88:223, 1997), agent-enhanced uptake of DNA, and ex vio transduction. For long-term, high-yield production of recombinant proteins, stable expression will often be desired. For example, cell lines which stably express the multispecific antibody of the invention or fragments thereof or binding domains thereof, such as the ROR1-BD, can be prepared using expression vectors of the Invention which contain viral origins of replication or endogenous expression elements and a selectable marker gene. Following the introduction of the vector, cells may be allowed to grow for 1 to 2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. The present invention thus provides a method of producing the multispecific antibody of the Invention or antigen-binding fragments thereof, such as the ROR1-BD, wherein said method comprises the step of culturing a host cell comprising a nucleic acid or a vector encoding the multispecific antibody of the invention or antigen-binding fragment thereof, such as the ROR1-BDs, whereby said multispecific antibody of the disclosure or an antigen-binding fragment thereof, such as the ROR1-BD, is expressed.

In one aspect the present invention relates to a method for producing the multispecific antibody or the ROR1-BD of the Invention, the method comprising the step of culturing a host cell expressing a nucleic acid encoding the multispecific antibody or the ROR1-BD of the invention. In particular, the present Invention relates to a method of producing the multispecific antibody or the ROR1-BD of the invention, the method comprising (1) providing a nucleic acid or two nucleic acids encoding the multispecific antibody or the ROR1-BD of the invention or one vector or two vectors encoding the multispecific antibody or the ROR1-BD of the invention, expressing said nucleic add or nucleic acids, or said vector or vectors, and collecting said multispecific antibody or said ROR1-BD from the expression system, or (ii) providing a host cell or host cells expressing a nucleic acid or nucleic acids encoding the multispecific antibody or the ROR1-BD of the invention, culturing said host cell or said host cells; and collecting said multispecific antibody or said ROR1-BD from the cell culture.

In a further aspect, the present invention relates to a pharmaceutical composition comprising the multispecific antibody of the invention, and a pharmaceutically acceptable carrier. “Pharmaceutically acceptable carrier” means a medium or diluent that does not interfere with the structure of the antibodies. Pharmaceutically acceptable carriers enhance or stabilize the composition, or facilitate preparation of the composition. Pharmaceutically acceptable carriers include solvents, dispersion media, coatings, antibacterial and antifungal agents, isotonic and absorption delaying agents, and the ike that are physiologically compatible.

Certain, of such carriers enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection, infusion or topical administration. For example, a pharmaceutically acceptable carrier can be a sterile aqueous solution.

The pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion), particularly for Intramuscular or subcutaneous administration. Depending on the route of administration, the active compound, i.e., the multispecific antibody of the invention, may be coated in a material to protect the compound from the action of acids and other natural conditions that may inactivate the compound.

The pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the multispecific antibody of the invention is employed in the pharmaceutical compositions of the Invention. The multispecific antibodies of the invention are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or Increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association wth the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

The multispecific antibody of the invention is usually administered on multiple occasions. Intervals between single dosages can be weekly, monthly or yearly. Intervals can also be irregular as indicated by measuring blood levels of the multispecific antibody of the invention in the patient. Alternatively, the multispecific antibody of the invention can be administered as a sustained release formulation, in which case less frequent administration is required. Dosage and frequency vary depending on the half-life of the antibody in the patient. In general, humanized antibodies show longer ha-life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

In one aspect, the present invention relates to the multispecific antibody of the invention or the pharmaceutical composition of the invention for use as a medicament. In a suitable embodiment, the present invention provides the multispecific antibody or the pharmaceutical composition for use in the treatment of a proliferative disease, in particular a cancer, more particularly a ROR1-expressing cancer, in a subject in need thereof.

In another aspect, the present Invention provides the pharmaceutical composition for use in the manufacture of a medicament for the treatment of a proliferative disease, in particular a cancer, more particularly a ROR1-expressing cancer.

In another aspect, the present invention relates to the use of the multispecific antibody or the pharmaceutical composition for treating a proliferative disease, in particular a cancer, more particularly a ROR1-expressing cancer, in a subject in need thereof.

In another aspect, the present invention relates to a method of treating a subject comprising administering to the subject a therapeutically effective amount of the multispecific antibody of the present invention. In a suitable embodiment, the present invention relates to a method for the treatment of a proliferative disease, in particular a cancer, more particularly a ROR1-expressing cancer, in a subject comprising administering to the subject a therapeutically effective amount of the multispecific antibody of the present invention.

The term “subject” includes human and non-human animals.

The term “animals” Include all vertebrates, e.g., non-human mammals and non-mammals, such as non-human primates, sheep, dog, cow, chickens, amphibians, and reptiles. Except when noted, the terms “patient” or “subject” are used herein interchangeably.

The terms “treatment”, “treating”, “treat”, “treated”, and the like, as used herein, refer to obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e.g., in a human, and includes: (a) inhibiting the disease, i.e., arresting its development; and (b) relieving the disease, i.e., causing regression of the disease.

The term “therapeutically effective amount” or “efficacious amount” refers to the amount of an agent that, when administered to a mammal or other subject for treating a disease, is sufficient to affect such treatment for the disease. The “therapeutically effective amount” will vary depending on the agent, the disease and its severity and the age, weight, etc., of the subject to be treated.

In one embodiment, the proliferative disease is a cancer. The term “cancer” refers to a disease characterized by the rapid and uncontrolled growth of aberrant cells. Cancer cells can spread locally or through the bloodstream and lymphatic system to other parts of the body. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. The term “cancer” is used herein to mean a broad spectrum of tumors, including all solid and hematological malignancies. In particular the cancer is a ROR1-expressing cancer.

Non limiting examples of ROR1-expressing cancer are: lymphocytic leukemia/small lymphocytic lymphoma (CLL/SLL), acute myeloid leukemia (AML), acute lymphoblastic leukemia (ALL), mantle cell lymphoma (MCL), hairy cell leukemia, follicular lymphoma (FL), marginal zone lymphoma (MZL), diffuse large B cell lymphoma (DLBCL), Richter's syndrome (RS), lung cancer, pancreatic cancer, prostate cancer, colon cancer, bladder cancer, breast cancer, ovarian cancer, glioblastoma, testicular cancer, uterine cancer, adrenal cancer, melanoma, neuroblastoma, sarcoma and renal cancer.

The multispecific antibody or the pharmaceutical composition of the invention, inhibits the growth of solid tumors, but also liquid tumors. The multispecific antibody or the pharmaceutical composition of the Invention, Is also suited to prevent the metastatic spread of tumors and the growth or development of micro metastases in a subject having a cancer.

Sequence listing (mutations designated according to AHo numbering scheme; the CDRs defined according to Numab CDR definition, unless specified otherwise)

TABLE 1
Examples of ROR1 binding domains of the present invention (modifications, if present, are shown in bold; CDR residues are
shown in bold and italic letters).

| SEQ ID | | |
|---|---|---|
| NUMBER | Ab region | Sequence |
| 55-38-D07 | | |
| SEQ ID NO: 1 | HCDR1 | GFDLSSYAVS |
| | (H27-H42; AHo numbering) | |
| SEQ ID NO: 2 | HCDR2 | IIYPRANTYYASWAKG |
| | (H57-H76; AHo numbering) | |
| SEQ ID NO: 3 | HCDR3 | RDRYDSGAYLYTTYFNL |
| | (H108-H138; AHo numbering) | |
| SEQ ID NO: 4 | LCDR1 | RASENIYSGLA |
| | (L24-L42; AHo numbering) | |
| SEQ ID NO: 5 | LCDR2 | RASTLAS |
| | (L58-L72; AHo numbering) | |
| SEQ ID NO: 6 | LCDR3 | QGGYYSSSSTYIA |
| | (L107-L138; AHo numbering) | |
| SEQ ID NO: 7 | VH | QSQVVESGGGLVQPGGSLRLSCAVSGFDLSSYAVSWVRQAPGKGLEWIGIIYPRANTY |
| | (55-38-D07-sc02) | YASWAKGRFTISKDNSKNTVYLQMNSLRAEDTAVYFCARDRYDSGAYLYTTYFNLWG |
| | (PRO2060) | QGTLVTVSS |
| SEQ ID NO: 8 | VH | QSQVVESGGGRVQPGGSLRLSCAVSGFDLSSYAVSWVRQAPGKGLEWIGIIYPRANTY |
| | (55-38-D07-sc02) mutations | YASWAKGRFTISKDNSKNTVYLQMNSLRAEDTATYFCARDRYDSGAYLYTTYFNLWG |
| | L12R, V103T, L144Q | QGTQVTVSS |
| SEQ ID NO. 9 | VL | DVQMTQSPSSLSASVGDRVTITCRASENIYSGLAWYQQKPGKPPKLLIYRASTLASGVS |
| | (55-38-D07-sc02) | SRFSGSGSGTDFTLTISSLQPEDFATYYCQGGYYSSSSTYIAFGTGTKVTVLG |
| | (PRO2060) | |
| SEQ ID NO: 10 | VH | QSQVVESGGGLVQPGGSLRLSCAVSGFDLSSYAVSWVRQAPGKCLEWIGIIYPRANTY |
| | (55-38-D07-sc06) mutation | YASWAKGRFTISKDNSKNTVYLQMNSLRAEDTAVYFCARDRYDSGAYLYTTYFNLWG |
| | G51C | QGTLVTVSS |
| | (PRO2291) | |
| SEQ ID NO: 11 | VH | QSQVVESGGGRVQPGGSLRLSCAVSGFDLSSYAVSWVRQAPGKCLEWIGIIYPRANTY |
| | (55-38-D07-sc06) mutations | YASWAKGRFTISKDNSKNTVYLQMNSLRAEDTATYFCARDRYDSGAYLYTTYFNLWG |
| | L12R, G51C, V103T, L144Q | QGTQVTVSS |
| SEQ ID NO: 12 | VL | DVQMTQSPSSLSASVGDRVTITCRASENIYSGLAWYQQKPGKPPKLLIYRASTLASGVS |
| | (55-38-D07-sc06) mutation | SRFSGSGSGTDFTLTISSLQPEDFATYYCQGGYYSSSSTYIAFGCGTKVTVLG |
| | T141C | |
| | (PRO2291) | |
| 55-39-G02 | | |
| SEQ ID NO: 13 | HCDR1 | GLSLSRNAMS |
| | (H27-H42; AHo numbering) | |
| | (present in 55-39-G02-sc02 and | |
| | 55-39-G02-sc05) | |
| SEQ ID NO: 14 | HCDR1 | GIDLSRNAMS |
| | (H27-H42; AHo numbering) | |
| | (present in 55-39-G02-sc03 and | |
| | 55-39-G02-sc06) | |
| SEQ ID NO: 15 | HCDR2 | IILTSGSTYYASWAKG |
| | (H57-H76; AHo numbering) | |
| SEQ ID NO: 16 | HCDR3 | RGIASSSLKSF |
| | (H108-H138; AHo numbering) | |
| SEQ ID NO: 17 | LCDR1 | QASQNVWNNNYLS |
| | (L24-L42; AHo numbering) | |
| SEQ ID NO: 18 | LCDR2 | TASTLAS |
| | (L58-L72; AHo numbering) | |
| SEQ ID NO: 19 | LCDR3 | AGGFSGEIRA |
| | (L107-L138; AHo numbering) | |
| SEQ ID NO: 20 | VH | QSQLVESGGGLVQPGGSLRLSCAVSGLSLSRNAMSWVRQAPGKGLEWIGIILTSGSTY |
| | (55-39-G02-sc02) | YASWAKGRFTISKDNSKNTVYLQMNSLRAEDTAVYFCVRGIASSSLKSFWGQGTLVTV |
| | (PRO2062) | SS |
| SEQ ID NO: | VH | QSQLVESGGGRVQPGGSLRLSCAVSGLSLSRNAMSWVRQAPGKGLEWIGIILTSGSTY |
| 21 | (55-39-G02-sc02) mutations | YASWAKGRFTISKDNSKNTVYLQMNSLRAEDTATYFCVRGIASSSLKSFWGQGTQVTV |
| | L12R, V103T, L144Q | SS |
| SEQ ID NO: 22 | VL | AQQLTQSPSSLSASVGDRVTITCQASQNVWNNNYLSWFQQKPGKPPKLLIVTASTLAS |
| | (55-39-G02-sc02) | GVSSRFSGSGSGTDFTLTISSLQPEDFATYYCAGGFSGEIRAFGTGTKVTVLG |
| | (PRO2062) | |
| SEQ ID NO: 23 | VH | QSQLVESGGGLVQPGGSLRLSCAVSGLSLSRNAMSWVRQAPGKCLEWIGIILTSGSTY |
| | (55-39-G02-sc05) mutation | YASWAKGRFTISKDNSKNTVYLQMNSLRAEDTAVYFCVRGIASSSLKSFWGQGTLVTV |
| | G51C | SS |
| | (PRO2292) | |
| SEQ ID NO: 24 | VH | QSQLVESGGGRVQPGGSLRLSCAVSGLSLSRNAMSWVRQAPGKCLEWIGIILTSGSTY |
| | (55-39-G02-sc05) mutations | YASWAKGRFTISKDNSKNTVYLQMNSLRAEDTATYFCVRGIASSSLKSFWGQGTQVTV |
| | L12R, G51C, V103T, L144Q | SS |
| SEQ ID NO: 25 | VL | AQQLTQSPSSLSASVGDRVTITCQASQNVWNNNYLSWFQQKPGKPPKLLIVTASTLAS |
| | (55-39-G02-sc05) mutation | GVSSRFSGSGSGTDFTLTISSLQPEDFATYYCAGGFSGEIRAFGCGTKVTVLG |
| | T141C | |
| | PRO2292) | |
| SEQ ID NO: 26 | VH | QSQLVESGGGLVQPGGSLRLSCAVSGIDLSRNAMSWVRQAPGKGLEWIGIILTSGSTY |
| | (55-39-G02-sc03) | YASWAKGRFTISKTSTTLDLQMNSLRAEDTAVYFCVRGIASSSLKSFWGQGTLVTVSS |
| | (PRO2271) | |
| SEQ ID NO: 27 | VL | AQQLTQSPSSLSASVGDRVTITCQASQNVWNNNYLSWFQQKPGKPPKLLIVTASTLAS |
| | (55-39-G02-sc03) | GVSSRFSGSGSGTDFTLTISSLQPEDFATYYCAGGFSGEIRAFGTGTKVTVLG |
| | (PRO2271) | |
| SEQ ID NO: 28 | VH | QSQLVESGGGLVQPGGSLRLSCAVSGIDLSRNAMSWVRQAPGKCLEWIGIILTSGSTY |
| | (55-39-G02-sc06) mutation | YASWAKGRFTISKTSTTLDLQMNSLRAEDTAVYFCVRGIASSSLKSFWGQGTLVTVSS |
| | G51C | |
| SEQ ID NO: 29 | VL | AQQLTQSPSSLSASVGDRVTITCQASQNVWNNNYLSWFQQKPGKPPKLLIVTASTLAS |
| | (55-39-G02-sc06) mutation | GVSSRFSGSGSGTDFTLTISSLQPEDFATYYCAGGFSGEIRAFGCGTKVTVLG |
| | T141C | |

TABLE 2
Examples of CD3 binding domains as used in the present invention (modifications, if present, are shown in bold; CDR
residues are shown in bold and italic letters).

| SEQ ID NUMBER | Ab region | Sequence |
|---|---|---|
| 28-21-D09 | | |
| SEQ ID NO: 30 | HCDR1 | GFSLSSYDMS |
| | (H27-H42; AHo numbering) | |
| SEQ ID NO: 31 | HCDR2 | ASYASGPTYYASWAKG |
| | (H57-H76; AHo numbering) | |
| SEQ ID NO: 32 | HCDR3 | RGGWTGTSHSNI |
| | (H108-H138; AHo numbering) | |
| SEQ ID NO: 33 | LCDR1 | QSSQSVFSNNYLA |
| | (L24-L42; AHo numbering) | |
| SEQ ID NO: 34 | LCDR2 | SASTLAS |
| | (L58-L72; AHo numbering) | |
| SEQ ID NO: 35 | LCDR3 | LGSYACSSADCYV |
| | (L107-L138; AHo numbering) | |
| SEQ ID NO: 36 | VH | EVqLVESGGGLVQPGGSLRLSCAASGFSLSSYDMSWVRQAPGKGLAWIGASYASGPT |
| | (28-21-D09-sc04) | YYASWAKGRFTISRDNSKNTVYLQMNSLRAEDTATYFCARGGWTGTSHSNIWGQGTL |
| | PRO726 | VTVSS |
| SEQ ID NO: 37 | VH | EVQLVESGGGRVQPGGSLRLSCAASGFSLSSYDMSWVRQAPGKGLAWIGASYASGPT |
| | (28-21-D09-sc04) mutations | YYASWAKGRFTISRDNSKNTVYLQMNSLRAEDTATYFCARGGWTGTSHSNIWGQGTQ |
| | L12R, L144Q | VTVSS: |
| SEQ ID NO: 38 | VL | DIQMTQSPSSLSASVGDRVTITCQSSQSVFSNNYLAWFQQKPGQSPKRLIYSASTLAS |
| | (28-21-D09-sc04) | GVPSRFSGSGSGTDFTLTISSLQPEDFATYYCLGSYACSSADCYVFGTGTKVTVLG |
| | PRO726 | |

TABLE 3
Examples of human serum albumin (hSA) binding domains as used in the present invention (modifications, if present, are
shown in bold; CDR residues are shown in bold italic letters).

| SEQ ID | | |
|---|---|---|
| NUMBER | Ab region | Sequence |
| 19-01-H04 | | |
| SEQ ID NO: 39 | HCDR1 | GFSLSSNAMG |
| | (H27-H42; AHo numbering) | |
| SEQ ID NO: 40 | HCDR2 | IISVGGFTYYASWAKG |
| | (H57-H76; AHo numbering) | |
| SEQ ID NO: 41 | HCDR3 | RDRHGGDSSGAFYL |
| | (H108-H138; AHo numbering) | |
| SEQ ID NO: 42 | LCDR1 | QSSESVYSNNQLS |
| | (L24-L42; AHo numbering) | |
| SEQ ID NO: 43 | LCDR2 | DASDLAS |
| | (L58-L72; AHo numbering) | |
| SEQ ID NO: 44 | LCDR3 | AGGFSSSSDTA |
| | (L107-L138; AHo numbering) | |
| SEQ ID NO: 45 | VH | EVQLVESGGGLVQPGGSLRLSCAASGFSLSSNAMGWVRQAPGKGLEYIGIISVGGFTY |
| | (19-01-H04-sc03) | YASWAKGRFTISRDNSKNTVYLQMNSLRAEDTATYFCARDRHGGDSSGAFYLWGQGT |
| | (PRO325) | LVTVSS |
| SEQ ID NO: 46 | VL | DIQMTQSPSSLSASVGDRVTITCQSSESVYSNNQLSWYQQKPGQPPKLLIYDASDLAS |
| | (19-01-H04-sc03) | GVPSRFSGSGSGTDFTLTISSLQPEDFATYYCAGGFSSSSDTAFGGGTKLTVLG |
| | (PRO325) | |
| SEQ ID NO: 47 | VH | EVQLVESGGGLVQPGGSLRLSCAASGFSLSSNAMGWVRQAPGKCLEYIGIISVGGFTY |
| | (19-01-H04-sc03) mutation | YASWAKGRFTISRDNSKNTVYLQMNSLRAEDTATYFCARDRHGGDSSGAFYLWGQGT |
| | G51C | LVTVSS |
| | (PRO325 Cys) | |
| SEQ ID NO: 48 | VL | DIQMTQSPSSLSASVGDRVTITCQSSESVYSNNQLSWYQQKPGQPPKLLIYDASDLAS |
| | (19-01-H04-sc03) mutation | GVPSRFSGSGSGTDFTLTISSLQPEDFATYYCAGGFSSSSDTAFGCGTKLTVLG |
| | G141C | |
| | (PRO325 Cys) | |
| 23-13-A01 | | |
| SEQ ID NO: 49 | HCDR1 | GFSFSSSYWIC |
| | (H27-H42; AHo numbering) | |
| SEQ ID NO: 50 | HCDR2 | CVFTGDGTTYYASWAKG |
| | (H57-H76; AHo numbering) | |
| SEQ ID NO: 51 | HCDR3 | RPVSVYYYGMDL |
| | (H108-H138; AHo numbering) | |
| SEQ ID NO: 52 | LCDR1 | QASQIISSRSA |
| | (L24-L42; AHo numbering) | |
| SEQ ID NO: 53 | LCDR2 | QASKLAS |
| | (L58-L72; AHo numbering) | |
| SEQ ID NO: 54 | LCDR3 | QCTYIDSNFGA |
| | (L107-L138; AHo numbering) | |
| SEQ ID NO: 55 | VH | EVQLVESGGGLVQPGGSLRLSCAASGFSFSSSYWICWVRQAPGKGLEWVGCVFTGDG |
| | (23-13-A01-sc03) | TTYYASWAKGRFTISRDNSKNTVYLQMNSLRAEDTATYFCARPVSVYYYGMDLWGQG |
| | (PRO459) | TLVTVSS |
| SEQ ID NO: 56 | VL | DWMTQSPSSLSASVGDRVTITCQASQIISSRSAWYQQKPGQPPKLLIYQASKLASGVP |
| | (23-13-A01-sc03) | SRFSGSGSGTDFTLTISSLQPEDFATYYCQCTYIDSNFGAFGGGTKLTVLG |
| | (PRO459) | |
| SEQ ID NO: 57 | VH | EVQLVESGGGLVQPGGSLRLSCAASGFSFSSSYWICWVRQAPGKCLEWVGCVFTGDG |
| | (23-13-A01-sc03) mutation | TTYYASWAKGRFTISRDNSKNTVYLQMNSLRAEDTATYFCARPVSVYYYGMDLWGQG |
| | G51C | TLVTVSS |
| | (PRO459 Cys) | |
| SEQ ID NO: 58 | VL | DWMTQSPSSLSASVGDRVTITCQASQIISSRSAWYQQKPGQPPKLLIYQASKLASGVP |
| | (23-13-A01-sc03) mutation | SRFSGSGSGTDFTLTISSLQPEDFATYYCQCTYIDSNFGAFGCGTKLTVLG |
| | G141C | |
| | (PRO459 Cys) | |
| 19-04-A10 | | |
| SEQ ID NO: 59 | HCDR1 | GFSLSSYAMN |
| | (H27-H42; AHo numbering) | |
| SEQ ID NO: 60 | HCDR2 | HINAGDIAYYATWAKG |
| | (H57-H76; AHo numbering) | |
| SEQ ID NO: 61 | HCDR3 | RGAGGFSTGPFKL |
| | (H108-H138; AHo numbering) | |
| SEQ ID NO: 62 | LCDR1 | QASESINSRLA |
| | (L24-L42; AHo numbering) | |
| SEQ ID NO: 63 | LCDR2 | DASDLTS |
| | (L58-L72; AHo numbering) | |
| SEQ ID NO: 64 | LCDR3 | QGYGGSSTTT |
| | (L 107-L138; AHo numbering) | |
| SEQ ID NO: 65 | VH | EVQLVESGGGLVQPGGSLRLSCAASGFSLSSYAMNWVRQAPGKGLEWIGHINAGDIAY |
| | (19-04-A10-sc02) | YATWAKGRFTISRDNSKNTVYLQMNSLRAEDTAVYFCARGAGGFSTGPFKLWGQGTL |
| | (PRO2155) | VTVSS |
| SEQ ID NO: 66 | VH | EVQLVESGGGRVQPGGSLRLSCAASGFSLSSYAMNWVRQAPGKGLEWIGHINAGDIA |
| | (19-04-A10-sc02) mutations | YYATWAKGRFTISRDNSKNTVYLQMNSLRAEDTATYYCARGAGGFSTGPFKLWGQGT |
| | L12R, V103T, L144Q | QVTVSS |
| SEQ ID NO: 67 | VL | AFELTQSPSSLSASVGDRVTITCQASESINSRLAWYQQKPGQPPKLLIYDASDLTSGVP |
| | (19-04-A10-sc02) | SRFSGSGSGTDFTLTISSLQPEDFATYYCQGYGGSSTTTFGGGTKLTVLG |
| | (PRO2155) | |
| SEQ ID NO: 68 | VH | EVQLVESGGGLVQPGGSLRLSCAASGFSLSSYAMNWVRQAPGKCLEWIGHINAGDIAY |
| | (19-04-A10-sc06) mutation | YATWAKGRFTISRDNSKNTVYLQMNSLRAEDTAVYFCARGAGGFSTGPFKLWGQGTL |
| | G51C | VTVSS |
| | (PRO2317) | |
| SEQ ID NO: 69 | VH | EVQLVESGGGRVQPGGSLRLSCAASGFSLSSYAMNWVRQAPGKCLEWIGHINAGDIA |
| | (19-04-A10-sc06) mutations | YYATWAKGRFTISRDNSKNTVYLQMNSLRAEDTATYFCARGAGGFSTGPFKLWGQGT |
| | L12R, G51C, V103T, L144Q | QVTVSS |
| SEQ ID NO: 70 | VL | AFELTQSPSSLSASVGDRVTITCQASESINSRLAWYQQKPGQPPKLLIYDASDLTSGVP |
| | (19-04-A10-sc06) mutation | SRFSGSGSGTDFTLTISSLQPEDFATYYCQGYGGSSTTTFGCGTKLTVLG |
| | G141C | |
| | (PRO2317) | |

TABLE 4
Other sequences related to the present invention.

| SEQ ID NUMBER | Ab region | Sequence |
|---|---|---|
| SEQ ID NO: 71 | VH3 | EVQLVESGGGLVQPGGSLRLSCAASGFSFSANYYPCWVRQAPGKGLEWIGCIYGGSSDI |
| | | TYDANWTKGRFTISRDNSKNTVYLQMNSLRAEDTAVYYCARSAWYSGWGGDLWGQGT |
| | | LVTVSS |
| SEQ ID NO: 72 | VH1a | QVQLVQSGAEVKKPGSSVKVSCKASGIDFNSNYYMCWVRQAPGQGLEWMGCIYVGSH |
| | | VNTYYANWAKGRVTITADESTSTAYMELSSLRSEDTAVYYCATSGSSVLYFKFWGQGTL |
| | | VTVSS |
| SEQ ID NO: 73 | VH1b | QVQLVQSGAEVKKPGASVKVSCKASGIDFNSNYYMCWVRQAPGQGLEWMGCIYVGSH |
| | | VNTYYANWAKGRVTMTRDTSISTAYMELSSLRSEDTAVYYCATSGSSVLYFKFWGQGTL |
| | | VTVSS |
| SEQ ID NO: 74 | VH4 | QVQLQESGPGLVKPSETLSLTCKVSGFSFSNSYWICWIRQPPGKGLEWIGCTFVGSSDS |
| | | TYYANWAKGRVTISVDSSKNQFSLKLSSVTAADTAVYYCARHPSDAVYGYANNLWGQG |
| | | TLVTVSS |
| SEQ ID NO: 75 | Vkappa1 | DIQMTQSPSSLSASVGDRVTITCQASQSINNVLAWYQQKPGKAPKLLIYRASTLASGVPS |
| | | RFSGSGSGTDFTLTISSLOPEDFATYYCQSSYGNYGDFGTGTKVTVLG |
| SEQ ID NO: 76 | VA germline- | FGTGTKVTVLG |
| | based FR4 (Sk17) | |
| SEQ ID NO: 77 | VA germline- | FGGGTKLTVLG |
| | based FR4 (Sk12) | |
| SEQ ID NO: 78 | VA germline- | FGGGTQLIILG |
| | based FR4 | |
| SEQ ID NO: 79 | VA germline- | FGEGTELTVLG |
| | based FR4 | |
| SEQ ID NO: 80 | VA germline- | FGSGTKVTVLG |
| | based FR4 | |
| SEQ ID NO: 81 | VA germline- | FGGGTQLTVLG |
| | based FR4 | |
| SEQ ID NO: 82 | VA germline- | FGGGTQLTALG |
| | based FR4 | |
| SEQ ID NO: 83 | VA germline- | FGCGTKVTVLG |
| | based FR4 G141C | |
| SEQ ID NO: 84 | Linker | GGGGSGGGGSGGGGSGGGGS |
| SEQ ID NO: 85 | Linker | GGGGS |
| SEQ ID NO: 86 | Linker | GGGGSGGGGS |

TABLE 5
Examples of multispecific antibodies of the present invention (modifications and linkers are
shown in bold).

| SEQ ID NUMBER | Ab Format | Sequence |
|---|---|---|
| PRO2286 | | |
| SEQ ID NO: 87 | scMATCH3 | DVVMTQSPSSLSASVGDRVTITCQASQIISSRSAWYQQKPGQPPKLLIYQASKLASGVPSRFSGSG |
| | | SGTDFTLTISSLQPEDFATYYCQCTYIDSNFGAFGCGTKLTVLGGGGGSEVQLVESGGGLVQPGG |
| | | SLRLSCAASGFSLSSYDMSWVRQAPGKGLAWIGASYASGPTYYASWAKGRFTISRDNSKNTVYL |
| | | QMNSLRAEDTATYFCARGGWTGTSHSNIWGQGTLVTVSSGGGGSGGGGSGGGGSGGGGSDIQ |
| | | MTQSPSSLSASVGDRVTITCQSSQSVFSNNYLAWFQQKPGQSPKRLIYSASTLASGVPSRFSGSG |
| | | SGTDFTLTISSLQPEDFATYYCLGSYACSSADCYVFGTGTKVTVLGGGGGSEVQLVESGGGLVQP |
| | | GGSLRLSCAASGFSFSSSYWICWVRQAPGKCLEWVGCVFTGDGTTYYASWAKGRFTISRDNSKN |
| | | TVYLQMNSLRAEDTATYFCARPVSVYYYGMDLWGQGTLVTVSSGGGGSGGGGSAQQLTQSPSS |
| | | LSASVGDRVTITCQASQNVWNNNYLSWFQQKPGKPPKLLIVTASTLASGVSSRFSGSGSGTDFTL |
| | | TISSLQPEDFATYYCAGGFSGEIRAFGTGTKVTVLGGGGGSGGGGSGGGGSGGGGSQSQLVES |
| | | GGGLVQPGGSLRLSCAVSGLSLSRNAMSWVRQAPGKGLEWIGIILTSGSTYYASWAKGRFTISKD |
| | | NSKNTVYLQMNSLRAEDTAVYFCVRGIASSSLKSFWGQGTLVTVSS |
| PRO2287 | | |
| SEQ ID NO: 88 | ScMATCH3 | DIQMTQSPSSLSASVGDRVTITCQSSESVYSNNQLSWYQQKPGQPPKLLIYDASDLASGVPSRFS |
| | | GSGSGTDFTLTISSLQPEDFATYYCAGGFSSSSDTAFGCGTKLTVLGGGGGSEVQLVESGGGLVQ |
| | | PGGSLRLSCAASGFSLSSYDMSWVRQAPGKGLAWIGASYASGPTYYASWAKGRFTISRDNSKNT |
| | | VYLQMNSLRAEDTATYFCARGGWTGTSHSNIWGQGTLVTVSSGGGGSGGGGSGGGGSGGGGS |
| | | DIQMTQSPSSLSASVGDRVTITCQSSQSVFSNNYLAWFQQKPGQSPKRLIYSASTLASGVPSRFS |
| | | GSGSGTDFTLTISSLQPEDFATYYCLGSYACSSADCYVFGTGTKVTVLGGGGGSEVQLVESGGGL |
| | | VQPGGSLRLSCAASGFSLSSNAMGWVRQAPGKCLEYIGIISVGGFTYYASWAKGRFTISRDNSKN |
| | | TVYLQMNSLRAEDTATYFCARDRHGGDSSGAFYLWGQGTLVTVSSGGGGSGGGGSAQQLTQSP |
| | | SSLSASVGDRVTITCQASQNVWNNNYLSWFQQKPGKPPKLLIVTASTLASGVSSRFSGSGSGTDF |
| | | TLTISSLQPEDFATYYCAGGFSGEIRAFGTGTKVTVLGGGGGSGGGGSGGGGSGGGGSQSQLVE |
| | | SGGGLVQPGGSLRLSCAVSGLSLSRNAMSWVRQAPGKGLEWIGIILTSGSTYYASWAKGRFTISK |
| | | DNSKNTVYLQMNSLRAEDTAVYFCVRGIASSSLKSFWGQGTLVTVSS |
| PRO2507 | | |
| SEQ ID NO: 89 | scMATCH3 | AFELTQSPSSLSASVGDRVTITCQASESINSRLAWYQQKPGQPPKLLIYDASDLTSGVPSRFSGSG |
| | | SGTDFTLTISSLQPEDFATYYCQGYGGSSTTTFGCGTKLTVLGGGGGSEVQLVESGGGLVQPGGS |
| | | LRLSCAASGFSLSSYDMSWVRQAPGKGLAWIGASYASGPTYYASWAKGRFTISRDNSKNTVYLQ |
| | | MNSLRAEDTATYFCARGGWTGTSHSNIWGQGTLVTVSSGGGGSGGGGSGGGGSGGGGSDIQM |
| | | TQSPSSLSASVGDRVTITCQSSQSVFSNNYLAWFQQKPGQSPKRLIYSASTLASGVPSRFSGSGS |
| | | GTDFTLTISSLQPEDFATYYCLGSYACSSADCYVFGTGTKVTVLGGGGGSEVQLVESGGGLVQPG |
| | | GSLRLSCAASGFSLSSYAMNWVRQAPGKCLEWIGHINAGDIAYYATWAKGRFTISRDNSKNTVYL |
| | | QMNSLRAEDTAVYFCARGAGGFSTGPFKLWGQGTLVTVSSGGGGSGGGGSDVQMTQSPSSLSA |
| | | SVGDRVTITCRASENIYSGLAWYQQKPGKPPKLLIYRASTLASGVSSRFSGSGSGTDFTLTISSLQP |
| | | EDFATYYCQGGYYSSSSTYIAFGTGTKVTVLGGGGGSGGGGSGGGGSGGGGSQSQVVESGGG |
| | | LVQPGGSLRLSCAVSGFDLSSYAVSWVRQAPGKGLEWIGIIYPRANTYYASWAKGRFTISKDNSKN |
| | | TVYLQMNSLRAEDTAVYFCARDRYDSGAYLYTTYFNLWGQGTLVTVSS |
| PRO2508 | | |
| SEQ ID NO: 90 | ScMATCH3 | AFELTQSPSSLSASVGDRVTITCQASESINSRLAWYQQKPGQPPKLLIYDASDLTSGVPSRFSGSG |
| | | SGTDFTLTISSLQPEDFATYYCQGYGGSSTTTFGCGTKLTVLGGGGGSEVQLVESGGGLVQPGGS |
| | | LRLSCAASGFSLSSYDMSWVRQAPGKGLAWIGASYASGPTYYASWAKGRFTISRDNSKNTVYLQ |
| | | MNSLRAEDTATYFCARGGWTGTSHSNIWGQGTLVTVSSGGGGSGGGGSGGGGSGGGGSDIQM |
| | | TQSPSSLSASVGDRVTITCQSSQSVFSNNYLAWFQQKPGQSPKRLIYSASTLASGVPSRFSGSGS |
| | | GTDFTLTISSLQPEDFATYYCLGSYACSSADCYVFGTGTKVTVLGGGGGSEVQLVESGGGLVQPG |
| | | GSLRLSCAASGFSLSSYAMNWVRQAPGKCLEWIGHINAGDIAYYATWAKGRFTISRDNSKNTVYL |
| | | QMNSLRAEDTAVYFCARGAGGFSTGPFKLWGQGTLVTVSSGGGGSGGGGSDVQMTQSPSSLSA |
| | | SVGDRVTITCRASENIYSGLAWYQQKPGKPPKLLIYRASTLASGVSSRFSGSGSGTDFTLTISSLQP |
| | | EDFATYYCQGGYYSSSSTYIAFGCGTKVTVLGGGGGSGGGGSGGGGSGGGGSQSQVVESGGG |
| | | LVQPGGSLRLSCAVSGFDLSSYAVSWVRQAPGKCLEWIGIIYPRANTYYASWAKGRFTISKDNSKN |
| | | TVYLQMNSLRAEDTAVYFCARDRYDSGAYLYTTYFNLWGQGTLVTVSS |
| PRO2509 | | |
| SEQ ID NO: 91 | scMATCH3 | AFELTQSPSSLSASVGDRVTITCQASESINSRLAWYQQKPGQPPKLLIYDASDLTSGVPSRFSGSG |
| | | SGTDFTLTISSLQPEDFATYYCQGYGGSSTTTFGCGTKLTVLGGGGGSEVQLVESGGGLVQPGGS |
| | | LRLSCAASGFSLSSYDMSWVRQAPGKGLAWIGASYASGPTYYASWAKGRFTISRDNSKNTVYLQ |
| | | MNSLRAEDTATYFCARGGWTGTSHSNIWGQGTLVTVSSGGGGSGGGGGGGGSGGGGSDIQM |
| | | TQSPSSLSASVGDRVTITCQSSQSVFSNNYLAWFQQKPGQSPKRLIYSASTLASGVPSRFSGSGS |
| | | GTDFTLTISSLQPEDFATYYCLGSYACSSADCYVFGTGTKVTVLGGGGGSEVQLVESGGGLVQPG |
| | | GSLRLSCAASGFSLSSYAMNWVRQAPGKCLEWIGHINAGDIAYYATWAKGRFTISRDNSKNTVYL |
| | | QMNSLRAEDTAVYFCARGAGGFSTGPFKLWGQGTLVTVSSGGGGSGGGGSAQQLTQSPSSLSA |
| | | SVGDRVTITCQASQNVWNNNYLSWFQQKPGKPPKLLIVTASTLASGVSSRFSGSGSGTDFTLTISS |
| | | LQPEDFATYYCAGGFSGEIRAFGTGTKVTVLGGGGGSGGGGSGGGGSGGGGSQSQLVESGGGL |
| | | VQPGGSLRLSCAVSGLSLSRNAMSWVRQAPGKGLEWIGIILTSGSTYYASWAKGRFTISKDNSKN |
| | | TVYLQMNSLRAEDTAVYFCVRGIASSSLKSFWGQGTLVTVSS |
| PRO2510 | | |
| SEQ ID NO: 92 | scMATCH3 | AFELTQSPSSLSASVGDRVTITCQASESINSRLAWYQQKPGQPPKLLIYDASDLTSGVPSRFSGSG |
| | | SGTDFTLTISSLQPEDFATYYCQGYGGSSTTTFGCGTKLTVLGGGGGSEVQLVESGGGLVQPGGS |
| | | LRLSCAASGFSLSSYDMSWVRQAPGKGLAWIGASYASGPTYYASWAKGRFTISRDNSKNTVYLQ |
| | | MNSLRAEDTATYFCARGGWTGTSHSNIWGQGTLVTVSSGGGGSGGGGSGGGGSGGGGSDIQM |
| | | TQSPSSLSASVGDRVTITCQSSQSVFSNNYLAWFQQKPGQSPKRLIYSASTLASGVPSRFSGSGS |
| | | GTDFTLTISSLQPEDFATYYCLGSYACSSADCYVFGTGTKVTVLGGGGGSEVQLVESGGGLVQPG |
| | | GSLRLSCAASGFSLSSYAMNWVRQAPGKCLEWIGHINAGDIAYYATWAKGRFTISRDNSKNTVYL |
| | | QMNSLRAEDTAVYFCARGAGGFSTGPFKLWGQGTLVTVSSGGGGSGGGGSAQQLTQSPSSLSA |
| | | SVGDRVTITCQASQNVWNNNYLSWFQQKPGKPPKLLIVTASTLASGVSSRFSGSGSGTDFTLTISS |
| | | LQPEDFATYYCAGGFSGEIRAFGCGTKVTVLGGGGGSGGGGSGGGGSGGGGSQSQLVESGGG |
| | | LVQPGGSLRLSCAVSGLSLSRNAMSWVRQAPGKCLEWIGIILTSGSTYYASWAKGRFTISKDNSKN |
| | | TVYLQMNSLRAEDTAVYFCVRGIASSSLKSFWGQGTLVTVSS |
| PRO2557 | | |
| SEQ ID NO: 93 | scMATCH3 | AFELTQSPSSLSASVGDRVTITCQASESINSRLAWYQQKPGQPPKLLIYDASDLTSGVPSRFSGSG |
| | | SGTDFTLTISSLQPEDFATYYCQGYGGSSTTTFGCGTKLTVLGGGGGSEVQLVESGGGLVQPGGS |
| | | LRLSCAASGFSLSSYDMSWVRQAPGKGLAWIGASYASGPTYYASWAKGRFTISRDNSKNTVYLQ |
| | | MNSLRAEDTATYFCARGGWTGTSHSNIWGQGTLVTVSSGGGGSGGGGSGGGGSGGGGSDIQM |
| | | TQSPSSLSASVGDRVTITCQSSQSVFSNNYLAWFQQKPGQSPKRLIYSASTLASGVPSRFSGSGS |
| | | GTDFTLTISSLQPEDFATYYCLGSYACSSADCYVFGTGTKVTVLGGGGGSEVQLVESGGGLVQPG |
| | | GSLRLSCAASGFSLSSYAMNWVRQAPGKCLEWIGHINAGDIAYYATWAKGRFTISRDNSKNTVYL |
| | | QMNSLRAEDTAVYFCARGAGGFSTGPFKLWGQGTLVTVSSGGGGSGGGGSAQQLTQSPSSLSA |
| | | SVGDRVTITCQASQNVWNNNYLSWFQQKPGKPPKLLIVTASTLASGVSSRFSGSGSGTDFTLTISS |
| | | LQPEDFATYYCAGGFSGEIRAFGCGTKVTVLGGGGGSGGGGSGGGGSGGGGSQSQLVESGGG |
| | | LVQPGGSLRLSCAVSGIDLSRNAMSWVRQAPGKCLEWIGIILTSGSTYYASWAKGRFTISKTSTTLD |
| | | LQMNSLRAEDTAVYFCVRGIASSSLKSFWGQGTLVTVSS |
| PRO2596 | | |
| SEQ ID NO: 94 | scMATCH3 | AFELTQSPSSLSASVGDRVTITCQASESINSRLAWYQQKPGQPPKLLIYDASDLTSGVPSRFSGSG |
| | | SGTDFTLTISSLQPEDFATYYCQGYGGSSTTTFGCGTKLTVLGGGGGSEVQLVESGGGLVQPGGS |
| | | LRLSCAASGFSLSSYDMSWVRQAPGKGLAWIGASYASGPTYYASWAKGRFTISRDNSKNTVYLQ |
| | | MNSLRAEDTATYFCARGGWTGTSHSNIWGQGTLVTVSSGGGGSGGGGGGGGSGGGGSDIQM |
| | | TQSPSSLSASVGDRVTITCQSSQSVFSNNYLAWFQQKPGQSPKRLIYSASTLASGVPSRFSGSGS |
| | | GTDFTLTISSLQPEDFATYYCLGSYACSSADCYVFGTGTKVTVLGGGGGSEVQLVESGGGLVQPG |
| | | GSLRLSCAASGFSLSSYAMNWVRQAPGKCLEWIGHINAGDIAYYATWAKGRFTISRDNSKNTVYL |
| | | QMNSLRAEDTAVYFCARGAGGFSTGPFKLWGQGTLVTVSSGGGGSGGGGSAQQLTQSPSSLSA |
| | | SVGDRVTITCQASQNVWNNNYLSWFQQKPGKPPKLLIVTASTLASGVSSRFSGSGSGTDFTLTISS |
| | | LQPEDFATYYCAGGFSGEIRAFGTGTKVTVLGGGGGSGGGGSGGGGSGGGGSQSQLVESGGGL |
| | | VQPGGSLRLSCAVSGIDLSRNAMSWVRQAPGKGLEWIGIILTSGSTYYASWAKGRFTISKTSTTLD |
| | | LQMNSLRAEDTAVYFCVRGIASSSLKSFWGQGTLVTVSS |
| PRO2589 | | |
| SEQ ID NO: 95 | MATCH4 | DVQMTQSPSSLSASVGDRVTITCRASENIYSGLAWYQQKPGKPPKLLIYRASTLASGVSSRFSGSG |
| | (chain_1) | SGTDFTLTISSLQPEDFATYYCQGGYYSSSSTYIAFGTGTKVTVLGGGGGSGGGGSGGGGSGGG |
| | | GSQSQVVESGGGLVQPGGSLRLSCAVSGFDLSSYAVSWVRQAPGKGLEWIGIIYPRANTYYASW |
| | | AKGRFTISKDNSKNTVYLQMNSLRAEDTAVYFCARDRYDSGAYLYTTYFNLWGQGTLVTVSSGGG |
| | | GSGGGGSAFELTQSPSSLSASVGDRVTITCQASESINSRLAWYQQKPGQPPKLLIYDASDLTSGVP |
| | | SRFSGSGSGTDFTLTISSLQPEDFATYYCQGYGGSSTTTFGCGTKLTVLGGGSGGSDIQMTQSPS |
| | | SLSASVGDRVTITCQSSQSVFSNNYLAWFQQKPGQSPKRLIYSASTLASGVPSRFSGSGSGTDFT |
| | | LTISSLQPEDFATYYCLGSYACSSADCYVFGTGTKVTVLG |
| SEQ ID NO: 96 | MATCH4 | DVQMTQSPSSLSASVGDRVTITCRASENIYSGLAWYQQKPGKPPKLLIYRASTLASGVSSRFSGSG |
| | (chain_2) | SGTDFTLTISSLQPEDFATYYCQGGYYSSSSTYIAFGTGTKVTVLGGGGGSGGGGSGGGGSGGG |
| | | GSQSQVVESGGGLVQPGGSLRLSCAVSGFDLSSYAVSWVRQAPGKGLEWIGIIYPRANTYYASW |
| | | AKGRFTISKDNSKNTVYLQMNSLRAEDTAVYFCARDRYDSGAYLYTTYFNLWGQGTLVTVSSGGG |
| | | GSGGGGSEVQLVESGGGLVQPGGSLRLSCAASGFSLSSYDMSWVRQAPGKGLAWIGASYASGP |
| | | TYYASWAKGRFTISRDNSKNTVYLQMNSLRAEDTATYFCARGGWTGTSHSNIWGQGTLVTVSSG |
| | | GGSGGGSGGGSGEVQLVESGGGLVQPGGSLRLSCAASGFSLSSYAMNWVRQAPGKCLEWIGHI |
| | | NAGDIAYYATWAKGRFTISRDNSKNTVYLQMNSLRAEDTAVYFCARGAGGFSTGPFKLWGQGTLV |
| | | TVSS |
| PRO2590 | | |
| SEQ ID NO: 97 | MATCH4 | DVQMTQSPSSLSASVGDRVTITCRASENIYSGLAWYQQKPGKPPKLLIYRASTLASGVSSRFSGSG |
| | (chain_1) | SGTDFTLTISSLQPEDFATYYCQGGYYSSSSTYIAFGCGTKVTVLGGGGGSGGGGSGGGGSGGG |
| | | GSQSQVVESGGGLVQPGGSLRLSCAVSGFDLSSYAVSWVRQAPGKCLEWIGIIYPRANTYYASW |
| | | AKGRFTISKDNSKNTVYLQMNSLRAEDTAVYFCARDRYDSGAYLYTTYFNLWGQGTLVTVSSGGG |
| | | GSGGGGSAFELTQSPSSLSASVGDRVTITCQASESINSRLAWYQQKPGQPPKLLIYDASDLTSGVP |
| | | SRFSGSGSGTDFTLTISSLQPEDFATYYCQGYGGSSTTTFGCGTKLTVLGGGSGGSDIQMTQSPS |
| | | SLSASVGDRVTITCQSSQSVFSNNYLAWFQQKPGQSPKRLIYSASTLASGVPSRFSGSGSGTDFT |
| | | LTISSLQPEDFATYYCLGSYACSSADCYVFGTGTKVTVLG |
| SEQ ID NO: 98 | MATCH4 | DVQMTQSPSSLSASVGDRVTITCRASENIYSGLAWYQQKPGKPPKLLIYRASTLASGVSSRFSGSG |
| | (chain_2) | SGTDFTLTISSLQPEDFATYYCQGGYYSSSSTYIAFGCGTKVTVLGGGGGSGGGGSGGGGSGGG |
| | | GSQSQVVESGGGLVQPGGSLRLSCAVSGFDLSSYAVSWVRQAPGKCLEWIGIIYPRANTYYASW |
| | | AKGRFTISKDNSKNTVYLQMNSLRAEDTAVYFCARDRYDSGAYLYTTYFNLWGQGTLVTVSSGGG |
| | | GSGGGGSEVQLVESGGGLVQPGGSLRLSCAASGFSLSSYDMSWVRQAPGKGLAWIGASYASGP |
| | | TYYASWAKGRFTISRDNSKNTVYLQMNSLRAEDTATYFCARGGWTGTSHSNIWGQGTLVTVSSG |
| | | GGSGGGSGGGSGEVQLVESGGGLVQPGGSLRLSCAASGFSLSSYAMNWVRQAPGKCLEWIGHI |
| | | NAGDIAYYATWAKGRFTISRDNSKNTVYLQMNSLRAEDTAVYFCARGAGGFSTGPFKLWGQGTLV |
| | | TVSS |
| PRO2591 | | |
| SEQ ID NO: 99 | MATCH4 | AQQLTQSPSSLSASVGDRVTITCQASQNVWNNNYLSWFQQKPGKPPKLLIVTASTLASGVSSRFS |
| | (chain_1) | GSGSGTDFTLTISSLQPEDFATYYCAGGFSGEIRAFGTGTKVTVLGGGGGSGGGGSGGGGSGGG |
| | | GSQSQLVESGGGLVQPGGSLRLSCAVSGIDLSRNAMSWVRQAPGKGLEWIGIILTSGSTYYASWA |
| | | KGRFTISKTSTTLDLQMNSLRAEDTAVYFCVRGIASSSLKSFWGQGTLVTVSSGGGGSGGGGSAF |
| | | ELTQSPSSLSASVGDRVTITCQASESINSRLAWYQQKPGQPPKLLIYDASDLTSGVPSRFSGSGSG |
| | | TDFTLTISSLQPEDFATYYCQGYGGSSTTTFGCGTKLTVLGGGSGGSDIQMTQSPSSLSASVGDR |
| | | VTITCQSSQSVFSNNYLAWFQQKPGQSPKRLIYSASTLASGVPSRFSGSGSGTDFTLTISSLQPED |
| | | FATYYCLGSYACSSADCYVFGTGTKVTVLG |
| SEQ ID NO: 100 | MATCH4 | AQQLTQSPSSLSASVGDRVTITCQASQNVWNNNYLSWFQQKPGKPPKLLIVTASTLASGVSSRFS |
| | (chain_2) | GSGSGTDFTLTISSLQPEDFATYYCAGGFSGEIRAFGTGTKVTVLGGGGGSGGGGSGGGGSGGG |
| | | GSQSQLVESGGGLVQPGGSLRLSCAVSGIDLSRNAMSWVRQAPGKGLEWIGIILTSGSTYYASWA |
| | | KGRFTISKTSTTLDLQMNSLRAEDTAVYFCVRGIASSSLKSFWGQGTLVTVSSGGGGSGGGGSEV |
| | | QLVESGGGLVQPGGSLRLSCAASGFSLSSYDMSWVRQAPGKGLAWIGASYASGPTYYASWAKG |
| | | RFTISRDNSKNTVYLQMNSLRAEDTATYFCARGGWTGTSHSNIWGQGTLVTVSSGGGSGGGSGG |
| | | GSGEVQLVESGGGLVQPGGSLRLSCAASGFSLSSYAMNWVRQAPGKCLEWIGHINAGDIAYYAT |
| | | WAKGRFTISRDNSKNTVYLQMNSLRAEDTAVYFCARGAGGFSTGPFKLWGQGTLVTVSS |
| PRO2592 | | |
| SEQ ID NO: 101 | MATCH4 | AQQLTQSPSSLSASVGDRVTITCQASQNVWNNNYLSWFQQKPGKPPKLLIVTASTLASGVSSRFS |
| | (chain_1) | GSGSGTDFTLTISSLQPEDFATYYCAGGFSGEIRAFGCGTKVTVLGGGGGSGGGGSGGGGSGGG |
| | | GSQSQLVESGGGLVQPGGSLRLSCAVSGIDLSRNAMSWVRQAPGKCLEWIGIILTSGSTYYASWA |
| | | KGRFTISKTSTTLDLQMNSLRAEDTAVYFCVRGIASSSLKSFWGQGTLVTVSSGGGGSGGGGSAF |
| | | ELTQSPSSLSASVGDRVTITCQASESINSRLAWYQQKPGQPPKLLIYDASDLTSGVPSRFSGSGSG |
| | | TDFTLTISSLQPEDFATYYCQGYGGSSTTTFGCGTKLTVLGGGSGGSDIQMTQSPSSLSASVGDR |
| | | VTITCQSSQSVFSNNYLAWFQQKPGQSPKRLIYSASTLASGVPSRFSGSGSGTDFTLTISSLQPED |
| | | FATYYCLGSYACSSADCYVFGTGTKVTVLG |
| SEQ ID NO: 102 | MATCH4 | AQQLTQSPSSLSASVGDRVTITCQASQNVWNNNYLSWFQQKPGKPPKLLIVTASTLASGVSSRFS |
| | (chain_2) | GSGSGTDFTLTISSLQPEDFATYYCAGGFSGEIRAFGCGTKVTVLGGGGGSGGGGSGGGGSGGG |
| | | GSQSQLVESGGGLVQPGGSLRLSCAVSGIDLSRNAMSWVRQAPGKCLEWIGIILTSGSTYYASWA |
| | | KGRFTISKTSTTLDLQMNSLRAEDTAVYFCVRGIASSSLKSFWGQGTLVTVSSGGGGSGGGGSEV |
| | | QLVESGGGLVQPGGSLRLSCAASGFSLSSYDMSWVRQAPGKGLAWIGASYASGPTYYASWAKG |
| | | RFTISRDNSKNTVYLQMNSLRAEDTATYFCARGGWTGTSHSNIWGQGTLVTVSSGGGSGGGSGG |
| | | GSGEVQLVESGGGLVQPGGSLRLSCAASGFSLSSYAMNWVRQAPGKCLEWIGHINAGDIAYYAT |
| | | WAKGRFTISRDNSKNTVYLQMNSLRAEDTAVYFCARGAGGFSTGPFKLWGQGTLVTVSS |
| PRO2658 | | |
| SEQ ID NO: 103 | MATCH4 | AQQLTQSPSSLSASVGDRVTITCQASQNVWNNNYLSWFQQKPGKPPKLLIVTASTLASGVSSRFS |
| | (chain_1) | GSGSGTDFTLTISSLQPEDFATYYCAGGFSGEIRAFGTGTKVTVLGGGGGSGGGGSGGGGSGGG |
| | | GSQSQLVESGGGLVQPGGSLRLSCAVSGLSLSRNAMSWVRQAPGKGLEWIGIILTSGSTYYASW |
| | | AKGRFTISKDNSKNTVYLQMNSLRAEDTAVYFCVRGIASSSLKSFWGQGTLVTVSSGGGGSGGG |
| | | GSAFELTQSPSSLSASVGDRVTITCQASESINSRLAWYQQKPGQPPKLLIYDASDLTSGVPSRFSG |
| | | SGSGTDFTLTISSLQPEDFATYYCQGYGGSSTTTFGCGTKLTVLGGGSGGSDIQMTQSPSSLSAS |
| | | VGDRVTITCQSSQSVFSNNYLAWFQQKPGQSPKRLIYSASTLASGVPSRFSGSGSGTDFTLTISSL |
| | | QPEDFATYYCLGSYACSSADCYVFGTGTKVTVLG |
| SEQ ID NO: 104 | MATCH4 | AQQLTQSPSSLSASVGDRVTITCQASQNVWNNNYLSWFQQKPGKPPKLLIVTASTLASGVSSRFS |
| | (chain_2) | GSGSGTDFTLTISSLQPEDFATYYCAGGFSGEIRAFGTGTKVTVLGGGGGSGGGGSGGGGSGGG |
| | | GSQSQLVESGGGLVQPGGSLRLSCAVSGLSLSRNAMSWVRQAPGKGLEWIGIILTSGSTYYASW |
| | | AKGRFTISKDNSKNTVYLQMNSLRAEDTAVYFCVRGIASSSLKSFWGQGTLVTVSSGGGGSGGG |
| | | GSEVQLVESGGGLVQPGGSLRLSCAASGFSLSSYDMSWVRQAPGKGLAWIGASYASGPTYYAS |
| | | WAKGRFTISRDNSKNTVYLQMNSLRAEDTATYFCARGGWTGTSHSNIWGQGTLVTVSSGGGSGG |
| | | GSGGGSGEVQLVESGGGLVQPGGSLRLSCAASGFSLSSYAMNWVRQAPGKCLEWIGHINAGDIA |
| | | YYATWAKGRFTISRDNSKNTVYLQMNSLRAEDTAVYFCARGAGGFSTGPFKLWGQGTLVTVSS |
| PRO2659 | | |
| SEQ ID NO: 105 | MATCH4 | AQQLTQSPSSLSASVGDRVTITCQASQNVWNNNYLSWFQQKPGKPPKLLIVTASTLASGVSSRFS |
| | (chain_1) | GSGSGTDFTLTISSLQPEDFATYYCAGGFSGEIRAFGCGTKVTVLGGGGGSGGGGSGGGGSGGG |
| | | GSQSQLVESGGGLVQPGGSLRLSCAVSGLSLSRNAMSWVRQAPGKCLEWIGIILTSGSTYYASWA |
| | | KGRFTISKDNSKNTVYLQMNSLRAEDTAVYFCVRGIASSSLKSFWGQGTLVTVSSGGGGSGGGG |
| | | SAFELTQSPSSLSASVGDRVTITCQASESINSRLAWYQQKPGQPPKLLIYDASDLTSGVPSRFSGS |
| | | GSGTDFTLTISSLQPEDFATYYCQGYGGSSTTTFGCGTKLTVLGGGSGGSDIQMTQSPSSLSASV |
| | | GDRVTITCQSSQSVFSNNYLAWFQQKPGQSPKRLIYSASTLASGVPSRFSGSGSGTDFTLTISSLQ |
| | | PEDFATYYCLGSYACSSADCYVFGTGTKVTVLG |
| SEQ ID NO: 106 | MATCH4 | AQQLTQSPSSLSASVGDRVTITCQASQNVWNNNYLSWFQQKPGKPPKLLIVTASTLASGVSSRFS |
| | (chain_2) | GSGSGTDFTLTISSLQPEDFATYYCAGGFSGEIRAFGCGTKVTVLGGGGGSGGGGSGGGGSGGG |
| | | GSQSQLVESGGGLVQPGGSLRLSCAVSGLSLSRNAMSWVRQAPGKCLEWIGIILTSGSTYYASWA |
| | | KGRFTISKDNSKNTVYLQMNSLRAEDTAVYFCVRGIASSSLKSFWGQGTLVTVSSGGGGSGGGG |
| | | SEVQLVESGGGLVQPGGSLRLSCAASGFSLSSYDMSWVRQAPGKGLAWIGASYASGPTYYASW |
| | | AKGRFTISRDNSKNTVYLQMNSLRAEDTATYFCARGGWTGTSHSNIWGQGTLVTVSSGGGSGGG |
| | | SGGGSGEVQLVESGGGLVQPGGSLRLSCAASGFSLSSYAMNWVRQAPGKCLEWIGHINAGDIAY |
| | | YATWAKGRFTISRDNSKNTVYLQMNSLRAEDTAVYFCARGAGGFSTGPFKLWGQGTLVTVSS |
| PRO2667 | | |
| SEQ ID NO: 107 | ScMATCH3 | AFELTQSPSSLSASVGDRVTITCQASESINSRLAWYQQKPGQPPKLLIYDASDLTSGVPSRFSGSG |
| | | SGTDFTLTISSLQPEDFATYYCQGYGGSSTTTFGCGTKLTVLGGGGGSEVQLVESGGGRVQPGG |
| | | SLRLSCAASGFSLSSYDMSWVRQAPGKGLAWIGASYASGPTYYASWAKGRFTISRDNSKNTVYL |
| | | QMNSLRAEDTATYFCARGGWTGTSHSNIWGQGTQVTVSSGGGGSGGGGSGGGGSGGGGSDIQ |
| | | MTQSPSSLSASVGDRVTITCQSSQSVFSNNYLAWFQQKPGQSPKRLIYSASTLASGVPSRFSGSG |
| | | SGTDFTLTISSLQPEDFATYYCLGSYACSSADCYVFGTGTKVTVLGGGGGSEVQLVESGGGRVQP |
| | | GGSLRLSCAASGFSLSSYAMNWVRQAPGKCLEWIGHINAGDIAYYATWAKGRFTISRDNSKNTVY |
| | | LQMNSLRAEDTATYFCARGAGGFSTGPFKLWGQGTQVTVSSGGGGSGGGGSDVQMTQSPSSLS |
| | | ASVGDRVTITCRASENIYSGLAWYQQKPGKPPKLLIYRASTLASGVSSRFSGSGSGTDFTLTISSLQ |
| | | PEDFATYYCQGGYYSSSSTYIAFGTGTKVTVLGGGGGSGGGGSGGGGSGGGGSQSQWESGG |
| | | GRVQPGGSLRLSCAVSGFDLSSYAVSWVRQAPGKGLEWIGIIYPRANTYYASWAKGRFTISKDNS |
| | | KNTVYLQMNSLRAEDTATYFCARDRYDSGAYLYTTYFNLWGQGTQVTVSS |
| PRO2668 | | |
| SEQ ID NO: 108 | ScMATCH3 | AFELTQSPSSLSASVGDRVTITCQASESINSRLAWYQQKPGQPPKLLIYDASDLTSGVPSRFSGSG |
| | | SGTDFTLTISSLQPEDFATYYCQGYGGSSTTTFGCGTKLTVLGGGGGSEVQLVESGGGRVQPGG |
| | | SLRLSCAASGFSLSSYDMSWVRQAPGKGLAWIGASYASGPTYYASWAKGRFTISRDNSKNTVYL |
| | | QMNSLRAEDTATYFCARGGWTGTSHSNIWGQGTQVTVSSGGGGSGGGGSGGGGSGGGGSDIQ |
| | | MTQSPSSLSASVGDRVTITCQSSQSVFSNNYLAWFQQKPGQSPKRLIYSASTLASGVPSRFSGSG |
| | | SGTDFTLTISSLQPEDFATYYCLGSYACSSADCYVFGTGTKVTVLGGGGGSEVQLVESGGGRVQP |
| | | GGSLRLSCAASGFSLSSYAMNWVRQAPGKCLEWIGHINAGDIAYYATWAKGRFTISRDNSKNTVY |
| | | LQMNSLRAEDTATYFCARGAGGFSTGPFKLWGQGTQVTVSSGGGGSGGGGSAQQLTQSPSSLS |
| | | ASVGDRVTITCQASQNVWNNNYLSWFQQKPGKPPKLLIVTASTLASGVSSRFSGSGSGTDFTLTIS |
| | | SLQPEDFATYYCAGGFSGEIRAFGCGTKVTVLGGGGGSGGGGSGGGGSGGGGSQSQLVESGG |
| | | GRVQPGGSLRLSCAVSGLSLSRNAMSWVRQAPGKCLEWIGIILTSGSTYYASWAKGRFTISKDNS |
| | | KNTVYLQMNSLRAEDTATYFCVRGIASSSLKSFWGQGTQVTVSS |
| PRO2669 | | |
| SEQ ID NO: 109 | MATCH4 | DVQMTQSPSSLSASVGDRVTITCRASENIYSGLAWYQQKPGKPPKLLIYRASTLASGVSSRFSGSG |
| | (chain_1) | SGTDFTLTISSLQPEDFATYYCQGGYYSSSSTYIAFGTGTKVTVLGGGGGSGGGGSGGGGSGGG |
| | | GSQSQVVESGGGRVQPGGSLRLSCAVSGFDLSSYAVSWVRQAPGKGLEWIGIIYPRANTYYASW |
| | | AKGRFTISKDNSKNTVYLQMNSLRAEDTATYFCARDRYDSGAYLYTTYFNLWGQGTQVTVSSGG |
| | | GGSGGGGSAFELTQSPSSLSASVGDRVTITCQASESINSRLAWYQQKPGQPPKLLIYDASDLTSG |
| | | VPSRFSGSGSGTDFTLTISSLQPEDFATYYCQGYGGSSTTTFGCGTKLTVLGGGSGGSDIQMTQS |
| | | PSSLSASVGDRVTITCQSSQSVFSNNYLAWFQQKPGQSPKRLIYSASTLASGVPSRFSGSGSGTD |
| | | FTLTISSLQPEDFATYYCLGSYACSSADCYVFGTGTKVTVLG |
| SEQ ID NO: 110 | MATCH4 | DVQMTQSPSSLSASVGDRVTITCRASENIYSGLAWYQQKPGKPPKLLIYRASTLASGVSSRFSGSG |
| | (chain_2) | SGTDFTLTISSLQPEDFATYYCQGGYYSSSSTYIAFGTGTKVTVLGGGGGSGGGGSGGGGSGGG |
| | | GSQSQVVESGGGRVQPGGSLRLSCAVSGFDLSSYAVSWVRQAPGKGLEWIGIIYPRANTYYASW |
| | | AKGRFTISKDNSKNTVYLQMNSLRAEDTATYFCARDRYDSGAYLYTTYFNLWGQGTQVTVSSGG |
| | | GGSGGGGSEVQLVESGGGRVQPGGSLRLSCAASGFSLSSYDMSWVRQAPGKGLAWIGASYAS |
| | | GPTYYASWAKGRFTISRDNSKNTVYLQMNSLRAEDTATYFCARGGWTGTSHSNIWGQGTQVTVS |
| | | SGGGSGGGSGGGSGEVQLVESGGGRVQPGGSLRLSCAASGFSLSSYAMNWVRQAPGKCLEWI |
| | | GHINAGDIAYYATWAKGRFTISRDNSKNTVYLQMNSLRAEDTATYFCARGAGGFSTGPFKLWGQG |
| | | TQVTVSS |
| PRO2670 | | |
| SEQ ID NO: 111 | MATCH4 | DVQMTQSPSSLSASVGDRVTITCRASENIYSGLAWYQQKPGKPPKLLIYRASTLASGVSSRFSGSG |
| | (chain_1) | SGTDFTLTISSLQPEDFATYYCQGGYYSSSSTYIAFGCGTKVTVLGGGGGGGGGGGGGSGGG |
| | | GSQSQVVESGGGRVQPGGSLRLSCAVSGFDLSSYAVSWVRQAPGKCLEWIGIIYPRANTYYASW |
| | | AKGRFTISKDNSKNTVYLQMNSLRAEDTATYFCARDRYDSGAYLYTTYFNLWGQGTQVTVSSGG |
| | | GGSGGGGSAFELTQSPSSLSASVGDRVTITCQASESINSRLAWYQQKPGQPPKLLIYDASDLTSG |
| | | VPSRFSGSGSGTDFTLTISSLQPEDFATYYCQGYGGSSTTTFGCGTKLTVLGGGSGGSDIQMTQS |
| | | PSSLSASVGDRVTITCQSSQSVFSNNYLAWFQQKPGQSPKRLIYSASTLASGVPSRFSGSGSGTD |
| | | FTLTISSLQPEDFATYYCLGSYACSSADCYVFGTGTKVTVLG |
| SEQ ID NO: 112 | MATCH4 | DVQMTQSPSSLSASVGDRVTITCRASENIYSGLAWYQQKPGKPPKLLIYRASTLASGVSSRFSGSG |
| | (chain_2) | SGTDFTLTISSLQPEDFATYYCQGGYYSSSSTYIAFGCGTKVTVLGGGGGSGGGGSGGGGSGGG |
| | | GSQSQVVESGGGRVQPGGSLRLSCAVSGFDLSSYAVSWVRQAPGKCLEWIGIIYPRANTYYASW |
| | | AKGRFTISKDNSKNTVYLQMNSLRAEDTATYFCARDRYDSGAYLYTTYFNLWGQGTQVTVSSGG |
| | | GGSGGGGSEVQLVESGGGRVQPGGSLRLSCAASGFSLSSYDMSWVRQAPGKGLAWIGASYAS |
| | | GPTYYASWAKGRFTISRDNSKNTVYLQMNSLRAEDTATYFCARGGWTGTSHSNIWGQGTQVTVS |
| | | SGGGSGGGSGGGSGEVQLVESGGGRVQPGGSLRLSCAASGFSLSSYAMNWVRQAPGKCLEWI |
| | | GHINAGDIAYYATWAKGRFTISRDNSKNTVYLQMNSLRAEDTATYFCARGAGGFSTGPFKLWGQG |
| | | TQVTVSS |
| PRO2677 | | |
| SEQ ID NO: 113 | MATCH4 | DVQMTQSPSSLSASVGDRVTITCRASENIYSGLAWYQQKPGKPPKLLIYRASTLASGVSSRFSGSG |
| | (chain_1) | SGTDFTLTISSLQPEDFATYYCQGGYYSSSSTYIAFGTGTKVTVLGGGGGSGGGGSGGGGSGGG |
| | | GSQSQVVESGGGRVQPGGSLRLSCAVSGFDLSSYAVSWVRQAPGKGLEWIGIIYPRANTYYASW |
| | | AKGRFTISKDNSKNTVYLQMNSLRAEDTATYFCARDRYDSGAYLYTTYFNLWGQGTQVTVSSGG |
| | | GGSGGGGSAFELTQSPSSLSASVGDRVTITCQASESINSRLAWYQQKPGQPPKLLIYDASDLTSG |
| | | VPSRFSGSGSGTDFTLTISSLQPEDFATYYCQGYGGSSTTTFGCGTKLTVLGGGSGGSDIQMTQS |
| | | PSSLSASVGDRVTITCQSSQSVFSNNYLAWFQQKPGQSPKRLIYSASTLASGVPSRFSGSGSGTD |
| | | FTLTISSLQPEDFATYYCLGSYACSSADCYVFGTGTKVTVLG |
| SEQ ID NO: 114 | MATCH4 | AQQLTQSPSSLSASVGDRVTITCQASQNVWNNNYLSWFQQKPGKPPKLLIVTASTLASGVSSRFS |
| | (chain_2) | GSGSGTDFTLTISSLQPEDFATYYCAGGFSGEIRAFGTGTKVTVLGGGGGSGGGGSGGGGSGGG |
| | | GSQSQLVESGGGRVQPGGSLRLSCAVSGLSLSRNAMSWVRQAPGKGLEWIGIILTSGSTYYASW |
| | | AKGRFTISKDNSKNTVYLQMNSLRAEDTATYFCVRGIASSSLKSFWGQGQLVTVSSGGGGSGGG |
| | | GSEVQLVESGGGRVQPGGSLRLSCAASGFSLSSYDMSWVRQAPGKGLAWIGASYASGPTYYAS |
| | | WAKGRFTISRDNSKNTVYLQMNSLRAEDTATYFCARGGWTGTSHSNIWGQGTQVTVSSGGGSG |
| | | GGSGGGSGEVQLVESGGGRVQPGGSLRLSCAASGFSLSSYAMNWVRQAPGKCLEWIGHINAGD |
| | | TAYYATWAKGRFTISRDNSKNTVYLQMNSLRAEDTATYFCARGAGGFSTGPFKLWGQGTQVTVSS |
| PRO2678 | | |
| SEQ ID NO: 115 | MATCH4 | DVQMTQSPSSLSASVGDRVTITCRASENIYSGLAWYQQKPGKPPKLLIYRASTLASGVSSRFSGSG |
| | (chain_1) | SGTDFTLTISSLQPEDFATYYCQGGYYSSSSTYIAFGTGTKVTVLGGGGGSGGGGSGGGGSGGG |
| | | GSQSQVVESGGGRVQPGGSLRLSCAVSGFDLSSYAVSWVRQAPGKGLEWIGIIYPRANTYYASW |
| | | AKGRFTISKDNSKNTVYLQMNSLRAEDTATYFCARDRYDSGAYLYTTYFNLWGQGTQVTVSSGG |
| | | GGSGGGGSAFELTQSPSSLSASVGDRVTITCQASESINSRLAWYQQKPGQPPKLLIYDASDLTSG |
| | | VPSRFSGSGSGTDFTLTISSLQPEDFATYYCQGYGGSSTTTFGCGTKLTVLGGGSGGSDIQMTQS |
| | | PSSLSASVGDRVTITCQSSQSVFSNNYLAWFQQKPGQSPKRLIYSASTLASGVPSRFSGSGSGTD |
| | | FTLTISSLQPEDFATYYCLGSYACSSADCYVFGTGTKVTVLG |
| SEQ ID NO: 116 | MATCH4 | AQQLTQSPSSLSASVGDRVTITCQASQNVWNNNYLSWFQQKPGKPPKLLIVTASTLASGVSSRFS |
| | (chain_2) | GSGSGTDFTLTISSLQPEDFATYYCAGGFSGEIRAFGTGTKVTVLGGGGGSGGGGSGGGGSGGG |
| | | GSQSQLVESGGGRVQPGGSLRLSCAVSGIDLSRNAMSWVRQAPGKGLEWIGIILTSGSTYYASWA |
| | | KGRFTISKTSTTLDLQMNSLRAEDTATYFCVRGIASSSLKSFWGQGTQVTVSSGGGGSGGGGSEV |
| | | QLVESGGGRVQPGGSLRLSCAASGFSLSSYDMSWVRQAPGKGLAWIGASYASGPTYYASWAKG |
| | | RFTISRDNSKNTVYLQMNSLRAEDTATYFCARGGWTGTSHSNIWGQGTQVTVSSGGGSGGGSG |
| | | GGSGEVQLVESGGGRVQPGGSLRLSCAASGFSLSSYAMNWVRQAPGKCLEWIGHINAGDIAYYA |
| | | TWAKGRFTISRDNSKNTVYLQMNSLRAEDTATYFCARGAGGFSTGPFKLWGQGTQVTVSS |

TABLE 6
Sequence of the extracellular domain of human ROR1 (residues 30-407) including the N-terminal signal peptide (residues 1-
29), according to UniProtKB-Q01973 (the extracellular part is shown in italic letters; the residues of the Ig-like domain, i. e. residues
42-147, are shown in bold and italic letters).

| SEQ ID NUMBER | Sequence |
|---|---|
| SEQ ID NO: 117 | MHRPRRRGTRPPLLALLAALLLAARGAAAQETELSVSAELVPTSSWNISSELNKDSYLTLDEPMNNITTSLGQTAELHCKV |
| | SGNPPPTIRWFKNDAPVVQEPRRLSFRSTIYGSRLRIRNLDTTDTGYFQCVATNGKEVVSSTGVLFVKFGPPPTASPGYS |
| | DEYEEDGFCQPYRGIACARFIGNRTVYMESLHMQGEIENQITAAFTMIGTSSHLSDKCSQFAIPSLCHYAFPYCDETSSVP |
| | KPRDLCRDECEILENVLCQTEYIFARSNPMILMRLKLPNCEDLPQPESPEAANCIRIGIPMADPINKNHKCYNSTGVDYRGT |
| | VSVTKSGRQCQPWNSQYPHTHTFTALRFPELNGGHSYCRNPGNQKEAPWCFTLDENFKSDLCDIPACDSKDSKEKNKM |
| | EILY |

Throughout the text of this application, should there be a discrepancy between the text of the specification (e.g., Tables 1 to 6) and the sequence listing, the text of the specification shall prevail.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

To the extent possible under the respective patent law, all patents, applications, publications, test methods, literature, and other materials cited herein are hereby incorporated by reference.

The following Examples illustrates the invention described above, but is not, however, intended to limit the scope of the invention in any way. Other test models known as such to the person skilled in the pertinent art can also determine the beneficial effects of the claimed invention.

### EXAMPLES

### Example 1: Generation and testing of anti-ROR1 molecules

### Aim of the Project

The goal of the project was to generate humanized monoclonal antibody fragments that specifically bind to the extracellular domain of human ROR1. A further goal was to identify anti-ROR1 antibody fragments that are potent and stable enough for incorporation into multispecific antibody formats.

### 1.1. Immunization

Nine rabbits were immunized with human ROR1 extracellular domain (ECD). Six rabbits were immunized following Numab's standard protocol consisting of four antigen injections over a period of 70 days, and three additional rabbits were immunized following an extended protocol, which consisted of the same initial schedule as the standard protocol with a final antigen injection four days before the end of the protocol at day 112.

1.2. Sorting and hit identification

3,740 single B cells derived from the six rabbits immunized following Numab's standard immunization protocol were sorted by FACS using the ROR1 extracellular domain. Single B cells were cultivated and B cell supernatant containing secreted antibodies of interest were collected over a period of four weeks. Out of those 3,740 sorted B cells, 993 clones showed binding to recombinant human ROR1 by ELISA. Positive clones were then tested for their ability to bind ROR1 expressing cells, and 627 clones showed binding to cancer cells expressing high ROR1 levels (MDA-MB-231), of which 99 clones showed an affinity to human ROR1 ECD lower than 800 pM. Sequences of heavy and light chains from those clones were retrieved, and the 15 ROR1“binding clones with the best binding affinities measured by SPR were selected to be produced as recombinant rabbit IgGs.

### 1.3. Manufacture of Recombinant Rabbit IgG

Following the selection of clones for hit confirmation, the rabbit antibodies were cloned, expressed, and purified for further characterization. The cloning of the corresponding light and heavy chain variable domains entailed the in vitro ligation of the DNA fragments into a suitable mammalian expression vector (pFUSE, Invivogen). The expression vectors for the rabbit antibody heavy and light chains were transfected into a mammalian suspension cell line (CHO—S) for transient heterologous expression (50 ml scale, Mirus CHOgro expression kit). Subsequently, secreted rabbit IgGs were affinity purified (Protein A affinity purification), buffer exchanged to PBS pH 7.4, and the final products were analyzed by sodium dodecyl sulfate polyacrylamide gel electrophoresis (SDS-PAGE, data not shown), UV absorbance at 280 nm, and size-exclusion high performance liquid chromatography (SE-HPLC) to verify identity, content, and purity. Out of the 15 selected rabbit IgGs, 13 were successfully produced.

1A. Hit Confirmation of Recombinant Rabbit IgG (rIgG)

The 13 successfully produced rIgGs were assessed for their binding affinity to ROR1-expressing cells by CELISA and to recombinant ROR1 by SPR. In addition, the binding region of the rIgGs on ROR1 was defined by assessing the binding of the rabbit IgGs to three chimeric ROR1/ROR2 variants.

### Binding to ROR1-Expressing Cells by CELISA

Binding to ROR1-expressing cells was assessed using MDA-MB-231 cells (ATCC, cat. HTB-26, human breast adenocarcinoma cells that express high levels of human ROR1) and MCF-7 (ATCC, cat. HTB-22, human breast cancer cells that do not express human ROR1). 40,000 cells were distributed to flat bottom tissue culture treated 96 well plates. The next day, plates were washed three times in overflow mode with 450 μl wash buffer (PBS, 0.2% BSA) per well, 50 μl of each point of the serial dilutions of each rabbit IgG tested as well as of the reference anti-ROR1 antibody PRO1842 (human IgG antibody comprising the VH-NL-sequences of SEQ-ID NOs: 129 and 130 as disclosed in the patent application US 2017/0306018 A1) were added and plates were incubated 1.5 h at room temperature (RT) under gentle agitation. After three washes with 450 μl wash buffer, 50 μl of HRP coupled goat anti-rabbit IgG or rabbit anti-human IgG antibodies were added to the respective wells. After a 1 h incubation at RT on a nutating mixer, plates were washed three times with 450 pI of washing buffer per well prior to the addition of 50 μl TMB (3,3′,5,5′-tetramethylbenzidine, KPL, Cat. No. 53-00-00). After 10 minutes development the enzymatic reaction was stopped by addition of 50 μl of 1M HCl per well and plate was read at 450 nm using 690 nm as a reference wavelength. For the recombinant production of PRO1842, mammalian expression plasmids (pcDNA3.1, host: CHO—S) were ordered from Gene Universal (Newark DE, United States). Expression and purification of PRO1842 was performed in a similar manner as described above in section 1.3 and yielded sufficient material and purity (>98% monomeric content by SE-HPLC) for the intended use.

Eleven clones showed binding to ROR1 expressing cells, and eight of these clones bound to ROR1 with an EC₅₀ similar to or better than the reference antibody PRO1842 (see Table 6).

### Binding Affinities by SPR

Binding kinetics (including affinity) of the rabbit IgGs to recombinant human ROR1 protein (His Tag, Acro Biosystems) were determined by SPR analysis on a Mass2 device (Sierra sensors, Bruker). Rabbit IgG molecules were captured on specific spots via an anti-rabbit IgG antibody (goat anti-rabbit IgG-Fc, Bethyl) covalently immobilized to a carboxylmethylated dextran surface (HCA sensorchip; Sierra sensors, Bruker) and a titration series of recombinant human ROR1 protein was injected as analyte. After each analyte injection cycle, every spot on the sensor chip was regenerated (Glycine-HCl, pH 1.5), and new rabbit IgG was subsequently re-captured. The binding kinetics to human ROR1 was measured using a mum-cycle kinetic assay, with ten analyte concentrations ranging from 0.176 to 90 nM, diluted in running buffer (HEPES buffered saline, 0.05% Tween-20, pH 7.5; Bloconcept). The apparent dissociation (k_{d}) and association (kₐ) rate constants and the apparent dissociation equilibrium constant (K_{D}) were calculated with the Sierra sensors analysis software (Sierra analyzer, Sierra sensors, Bruker) using one-to-one Langmuir binding model and quality of the fits was monitored based on relative Chi². The binding level was calculated as the maximum stability binding achieved normalized to the theoretical Rₘₐₓ.

The affinity to recombinant ROR1 was determined for eleven rabbit IgGs. No binding was observed for one clone and the best binding affinity measured was 1.06 nM. The results are summarized in Table 7. Reference anti-ROR1 antibody PRO1776 (human IgG4 antibody comprising the VH-NL-sequences of SEQ-ID NOs: 5 and 7 as disclosed in the patent application WO 2014/031174) was also measured with a similar setup. For the recombinant production of PRO1776, mammalian expression plasmids (pcDNA3.1, host: CHO—S) were ordered from Gene Universal (Newark DE, United States). Expression and purification of PRO1776 was performed in a similar manner as described above in section 1.3 and yielded sufficient material and purity (>98% monomeric content by SE-HPLC) for the intended use.

Identification of Rabbit IgG (rIgG) Binding Region on ROR1

In order to define the binding region of the rIgGs on ROR1, the extent of binding to HEK293T cells transiently transfected with three chimeric ROR1/ROR2 variants was assessed by CELISA. ROR1/ROR2 variants were generated by exchanging each of the three domains of ROR1, i.e. the Ig-like domain of ROR1 (region 1), the Frizzled domain of ROR1 (region 2) and the Kringle domain of ROR1 (region 3), with the corresponding region in ROR2. To exclude potential ROR2 binders, binding to ROR2 was assessed by ELISA first; no ROR2 binders were identified using this assay.

Plates were then coated with 25,000 cells per well on flat bottom poly-D lysine treated 96 well plates. The next day, cells were transfected with the corresponding constructs and incubated at 37° C., 5% CO₂. Twenty-four hours later, cells were washed with 450 μl wash buffer (PBS, 0.2% BSA) and samples were added (10 pg/ml rIgG) and incubated for 1.5 h at RT under gentle agitation. After 3 washes with 450 μl wash buffer, 50 μl of an HRP-coupled goat anti-rabbit IgG antibody were added to each well. After 1 h of incubation at RT on a nutating mixer, plates were washed three times with 450 μl of washing buffer per well prior to the addition of 50 μl TMB (3,3′,5,5′-tetramethylbenzidine, KPL, Cat. No. 53-00-00). After 10 minutes, the development of the enzymatic reaction was stopped by the addition of 50 μl of 1M HCl per well, and the plate was read at 450 nm using 690 nm as a reference wavelength. The binding levels of the rIgGs were calculated relative to binding of the rabbit IgG to the wt ROR1. A clear reduction of the binding level of the rIgG to a specific variant in comparison to the wt ROR1 protein would indicate localization of the rIgG epitope within the segment of human ROR1 replaced by the respective ROR2 sequence.

A clearly defined binding region was identified for 12 rabbit IgGs. Nine rIgGs bound to region 1 (Ig-like domain of ROR1), and three antibodies bound to region 2 (Frizzled domain of ROR1). No rIgG bound to region 3 (Kringle domain of ROR1). The results are summarized in Table 7.

TABLE 7
Characterization of binding of 15 rabbits IgGs by SPR and CELISA

| | | | | Binding to ROR1 expressing | | |
|---|---|---|---|---|---|---|
| | | | | cells (MDA-MB-231) | | |
| | | | | | rel. EC₅₀ | |
| | | | | | (EC₅₀, | |
| | Affinity to hROR1 by SPR | | | | reference/ | Binding |
| Clone ID | kₐ (1/Ms) | k_{d} (1/s) | k_{D} (M) | EC₅₀ (nM) | EC₅₀, sample) | region |
| 55-06-E06 | 2.68E+05 | 3.50E−03 | 1.31E−08 | 0.04 | 1.92 | 1 |
| 55-18-H06 | | Not producible | | | | |
| 55-19-D06 | 1.37E+05 | 3.61E−04 | 2.63E−09 | 0.05 | 1.72 | 1 |
| 55-21-E10 | 2.45E+05 | 2.87E−04 | 1.17E−09 | 0.09 | 1.06 | 1 |
| 55-29-G11 | | Not measured | | 2.17 | 0.04 | 2 |
| 55-32-E10 | | Not measured | | NB | NA | 2 |
| 55-33-A06 | 2.05E+05 | 4.12E−04 | 2.01E−09 | 0.05 | 2.49 | 1 |
| 55-35-D08 | 6.82E+04 | 7.34E−04 | 1.08E−08 | 1.70 | 0.07 | 2 |
| 55-38-D07 | 2.21E+05 | 2.97E−04 | 1.34E−09 | 0.06 | 1.94 | 1 |
| 55-38-F04 | NB | NB | NB | NB | NA | NB |
| 55-39-A08 | 3.55E+04 | 3.75E−05 | 1.06E−09 | 0.31 | 0.29 | 1 |
| 55-39-G02 | 2:29E+05 | 3.37E−04 | 1.47E−09 | 0.06 | 1.42 | 1 |
| 55-40-E06 | | Not producible | | | | |
| 55-42-D02 | 6.70E+04 | 4.32E−04 | 6.44E−09 | 0.08 | 1.01 | 1 |
| 55-42-E05 | 1.82E+05 | 5.51E−04 | 3.03E−09 | 0.03 | 2.48 | 1 |
| PRO1776 | 3.06E+05 | 1.73E−02 | 5.65E−08 | NA | NA | 1 |
| PRO1842 | | Not measured | | 12.38 | 1 | Not assessed |

NA: not applicable
NB: no binding
PRO1776: reference antibody (human IgG4 antibody comprising the VH-/VL-sequences of SEQ-ID:
NOs: 5 and 7 as disclosed in patent application WO 2014/031174)

PRO1842: reference antibody (human IgG antibody comprising the VH-NL-sequences of SEQ-ID NOs: 129 and 130 as disclosed in patent application US 2017/0306018 A1)

Eight clones showed a binding affinity to ROR1-expressing MDA-MB-231 cells better or comparable to reference anti-ROR1 antibody PRO1842. From these eight clones, the seven best clones were selected to be expressed as two version of humanized single chain Fys on Numab framework.

### 1.5. ScFv Generation

### ScFv Generation

The purpose of sequence humanization is to reduce the risk of immunogenicity to our molecules of interest and to stabilize the antibody variable domain (Fv fragment) to enable its use as a building block for the assembly of multispecific formats. Therefore, humanization involves the transfer of important residues from the donor (rabbit) complementarity-determining region (CDR) sequences onto a human acceptor framework to increase humanness and improve stability, without significantly impacting functionality.

For the lead ROR1 scFv domain generation, seven rabbit monoclonal antibody clones were selected as shown in Table 7. The humanization of these clones comprised the transfer of the rabbit CDRs onto one of Numab's proprietary human variable domain acceptor scaffolds. In this process, the amino acid sequences of the six CDR regions were characterized using Numab's CDR definitions (Table 8) and grafted onto Numab's proprietary and highly stable, fully human VH3/Vk1-lambda-capped acceptor framework; these constructs are known as “CDR grafts”.

TABLE 8
Numab CDR definition relative to AHo (Honegger) and Kabat numbering

| Region | AHo numbering | Kabat numbering |
|---|---|---|
| CDR1-VL | 24-42 | 24-34 |
| CDR2-VL | 58-72 | 50-56 |
| CDR3-VL | 107-138 | 89-97 |
| CDR1-VH | 27-42 | 26-35A/35B/. . . |
| CDR2-VH | 57-76 | 50-65 |
| CDR3-VH | 108-138 | 94-102 |

Exclusive engraftment of rabbit CDRs onto a human acceptor framework is the most basic grafting strategy, herein referred to as “CDR graf”. Besides the CDR graft, an additional grafting variant was designed that contains defined patterns of rabbit framework residues, herein referred to as “Full graft”. Details of these grafting variants are as follows:

CDR graft: Engraftment of rabbit CDRs on Numab's acceptor framework using Numab's CDR definition. No back-mutation of donor framework residues.
Full graft Maximal graft containing donor Fv core residues, donor VL/VH interface residues, as well as donor framework residues that potentially interact with the antigen.

Table 9 summarizes the CDR and Full grafts of ROR1 humanized domains. Rabbit framework residues which were engrafted onto the human framework are listed for VL and VH domains separately (using AHo numbering).

Mammalian expression plasmids (pcDNA3.1, host: CHO—S) of humanized scFv constructs were ordered at 1 mg scale from Gene Universal. Plasmids were used for transient transfection of CHO—S cells as described below.

### ScFv Manufacturing

Expression of mammalian scFv constructs was performed in CHO—S cells using CHOgro transient transfection kit (Mirus). Cultures were harvested by centrifugation followed by filtration after a maximum of 7 days of expression at 37° C. (or when cell viability <70% was reached). Proteins were purified from clarified culture supernatants by Protein L affinity chromatography. With the exception of clone 55-06-E06-derived scFvs, all molecules exhibited fractions with a monomeric content >95% post capture as assessed by SE-HPLC analysis. All molecules were directly re-buffered to 50 mM phosphate-citrate buffer with 150 mM NaCl at pH 6.4 by dialysis. The CDR-graft of clone 55-42-E05 (PRO2065) exhibited a low expression titer and therefore could not be taken forward. For quality control of the manufactured material, standard analytical methods such as SE-HPLC, UV₂₈₀ and SDS-PAGE were applied. The manufacture of humanized anti-ROR1 scFv molecules is summarized in Table 10.

As one scFv could not be expressed (55-42-E05-sc01/PRO2065), only 13 out of 14 scFvs were subjected to subsequent pharmacodynamic characterization.

TABLE 9
Overview of CDR and Full grafts of ROR1 humanized domains; donor framework back-mutations are indicated separately for VL
and VH domains.

| | | | Grafting | Engrafted rabbit framework | Engrafted rabbit framework |
|---|---|---|---|---|---|
| PRO ID | Description | Framework | Strategy | residues, VL | residues, VH |
| PRO2055 | 55-06-E06-sc01 | Vk1/VH3 | CDR | — | — |
| PRO2056 | 55-06-E06-sc02 | Vk1/VH3 | Full | D1A, I2Q, M4L, A51P | E1Q, V2S, L4V, A25V, R82K, Y105F |
| PRO2057 | 55-33-A06-sc01 | Vk1/VH3 | CDR | — | — |
| PRO2058 | 55-33-A06-sc02 | Vk1/VH3 | Full | D1A, A51P, F89Y | E1Q, V2S, L4V, A25V, R82K, Y105F |
| PRO2059 | 55-38-D07-sc01 | Vk1/VH3 | CDR | — | — |
| PRO2060 | 55-38-D07-sc02 | Vk1/VH3 | Full | I2V, A51P | E1Q, V2S, L4V, A25V, R82K, Y105F |
| PRO2061 | 55-39-G02-sc01 | Vk1/VH3 | CDR | — | — |
| PRO2062 | 55-39-G02-sc02 | Vk1/VH3 | Full | D1A, I2Q, M4L, Y44F, A51P, | E1Q, V2S, A25V, R82K, Y105F |
| | | | | Y57V | A107V |
| PRO2063 | 55-19-D06-sc01 | Vk1/VH3 | CDR | — | — |
| PRO2064 | 55-19-D06-sc02 | Vk1/VH3 | Full | D1A, I2Q, M4L, Y44F, A51P, | E1Q, V2S, L4V, A25V, R82K, Y105F |
| | | | | P52L, L54V | |
| PRO2065 | 55-42-E05-sc01 | Vk1/VH3 | CDR | — | — |
| PRO2066 | 55-42-E05-sc02 | Vk1/VH3 | Full | D1A, I2Q, M4L, A51P | E1Q, V2S, L4V, A25V, W54Y, R82K, |
| | | | | | Y105F |
| PRO2067 | 55-21-E10-sc01 | Vk1/VH3 | CDR | — | — |
| PRO2068 | 55-21-E10-sc02 | Vk1/VH3 | Full | A51P, L54Q, G82R, | E1Q, V2S, G56A, R82K, Y105F |

TABLE 10
ScFv manufacture of humanized CDR and full grafts
from 7 selected rabbit clones

| | | Titer post | Final titer | Purity by |
|---|---|---|---|---|
| | | capture | [mg/l | SE-HPLC |
| Protein ID | Description | [mg/L] | Expression] | [% monomer] |
| PRO2055 | 55-06-E06-sc01 | 33.5 | 18.8 | 90.6 |
| PRO2056 | 55-06-E06-sc02 | 9.5 | 7.1 | 92.4 |
| PRO2057 | 55-33-A06-sc01 | 33.5 | 32.0 | 99.2 |
| PRO2058 | 55-33-A06-sc02 | 37.0 | 34.9 | 99.4 |
| PRO2059 | 55-38-D07-sc01 | 44.0 | 43.6 | 94.2 |
| PRO2060 | 55-38-D07-sc02 | 34.0 | 30.6 | 94.3 |
| PRO2061 | 55-39-G02-sc01 | 3.5 | 3.1 | 95.5 |
| PRO2062 | 55-39-G02-sc02 | 13.0 | 11.0 | 99.8 |
| PRO2063 | 55-19-D06-sc01 | 13.5 | 11.3 | 99.5 |
| PRO2064 | 55-21-E10-sc01 | 50.0 | 14.3 | 99.8 |
| PRO2066 | 55-42-E05-sc02 | 8.5 | 8.0 | 98.5 |
| PRO2067 | 55-19-D06-sc02 | 13.0 | 53.6 | 99.1 |
| PRO2068 | 55-21-E10-sc02 | 18.0 | 17.5 | 98.2 |

### 1.6. ScFv Characterization

### CoM Binding to ROR1 Expressing MDA-MB-231 Cells by Flow Cytometry

MDA-MB-231 cells (ATCC, cat. HTB-26, human breast adenocarcinoma cells that express high levels of human ROR1) and MCF-7 (ATCC, cat. HTB-22, human breast cancer cells that do not express human ROR1) were harvested and cell number was determined. Cell suspensions were centrifuged for 5 min at 400×g and 100 μl of cell suspensions (50,000 cells) diluted in PBS-EB (1×DPBS, 2% FCS H.I., 2 mM EDTA) were added to designated wells in a non-binding 96-wel plate. After three washing steps with PBS-EB, cells were centrifuged and washing buffer was aspirated. 3-fold serial dilutions of scFvs and the reference anti-ROR1 Fab fragment PRO2213 (human Fab fragment comprising the VH-NL-sequences of SEQ-ID NOs: 129 and 130 as disclosed in the patent application US 2017/0306018 A1) starting at a concentration of 39 nM (scFvs) or 63 nM (PRO2213) were prepared and then added to the plates with cells. After incubation at 4° C. for 1 h, plates were washed three times using 100 μl of PBS-EB. Cells incubated with scFvs were re-suspended with 100 μl of Numab's framework specific detection antibody (PRO2268, rabbit 1 gG) and incubated at 4° C. for 30 min. Following a washing step, PRO2268 was subsequentially detected by the addition of anti-rabbit IgG antibody labeled with APC at a concentration of 2 μg/ml and incubated for 1 h at 4° C. Cells incubated with the reference Fab fragment PRO2213 were re-suspended with 100 μl of goat anti-human F(ab′)2 Alexa Fluor 647-conjugated antibody (Jackson Immuno Research, cat. 109-606-097) at a concentration of 2.8 pg/ml and incubated for 1 h at 4° C. Next, cells were washed again three times using 100 μl of PBS-EB per well. The cell pellets were re-suspended with 50 μl PBS-EB and analyzed with the NovoCyte 2060 flow cytometer device. The fluorescence intensity of the APC channel was recorded for each sample and the geometric mean of the fluorescence intensity (MFI) was calculated. The data were single-referenced (subtracted for fluorescence intensity found on cells incubated with detection reagents only), and subsequent concentration-response curves were fitted using a 4-PL fit (GraphPad Prism software). PRO2213 was produced from a papain digest of the human IgG PR01842. PRO2213 could be produced in sufficient quantity and purity (>98% monomeric content by SE-HPLC) for the intended use.

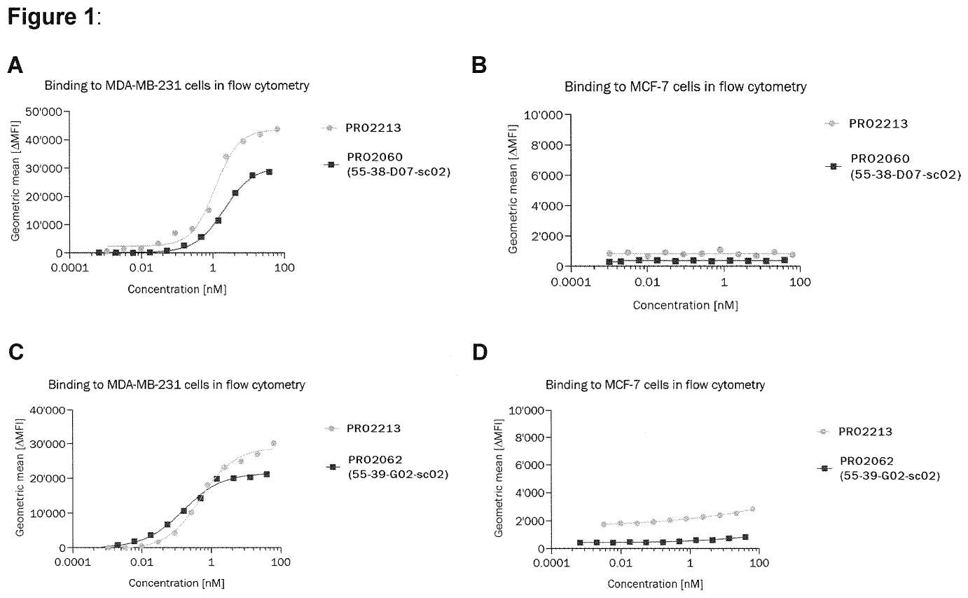

The apparent binding affinity to cell surface human ROR1 was assessed, and four scFvs showed an EC₅₀ for binding to human ROR1 expressing cells no more than three times lower than the reference Fab fragment PRO2213. Two scFvs showed no binding to cells expressing human ROR1 (Table 11). To test for unspecific binding, all scFvs were tested for binding to human ROR1 negative MCF-7 cells in flow cytometry and none of the tested scFvs demonstrated binding to MCF-7 cells. For anti-ROR1 scFvs PR02060 and PR02062, the apparent binding affinity to plasma membrane-based human ROR1 was assessed in three and two independent flow cytometry experiments, respectively. The mean EC_{w} and mean relative EC₅₀ values obtained in these experiments for PRO2060 and PR02062 are also shown in Table 11. Both scFvs demonstrated specific binding to human ROR1-positive MDA-MB-231 cells while no binding was found to ROR1-negative MCF-7 cells (see FIG. 1). Compared to the reference Fab fragment PRO2213, PRO2060 showed an approximately three times lower apparent binding affinity while PRO2062 showed an approximately three times higher apparent binding affinity.

TABLE 11
Apparent binding affinity of anti-ROR1 scFvs to plasma membrane-based
human ROR1 expressed on MDA-MB-231 cells by flow cytometry

| | | | | Rel. maximum |
|---|---|---|---|---|
| | | | rel. EC₅₀ | binding |
| Protein | Clone | EC₅₀ | (EC_{50, PRO2213}/ | (MFI_{scFv}/ |
| ID | description | [nM] | EC_{50, scFv}) | MFI_{PRO2213}) |
| PRO2055 | 55-06-E06-sc01 | 2.26 | 0.29 | 0.49 |
| PRO2056 | 55-06-E06-sc02 | 3.13 | 0.21 | 0.31 |
| PRO2057 | 55-33-A06-sc01 | no binding | | |
| PRO2058 | 55-33-A06-sc02 | not calculable | | 0.26 |
| PRO2059 | 55-38-D07-sc01 | 3.5 | 0.33 | 0.54 |
| PRO2061 | 55-39-G02-sc01 | 25.63 | 0.02 | 0.13 |
| PRO2063 | 55-19-D06-sc01 | 30.8 | 0.02 | 0.13 |
| PRO2064 | 55-21-E10-sc01 | 16.4 | 0.04 | 0.41 |
| PRO2066 | 55-42-E05-sc02 | 0.59 | 1.21 | 0.89 |
| PRO2067 | 55-19-D06-sc02 | | no binding | |
| PRO2068 | 55-21-E10-sc02 | 19.75 | 0.05 | 0.08 |
| PRO2060 | 55-38-D07-sc02 | 2.93 | 0.33 | 1.00 |
| PRO2062 | 55-39-G02-sc02 | 0.26 | 2.77 | 0.85 |

PRO2060 and PR02062 were measured in three and two independent experiments, respectively, and mean EC₅₀, mean rel. EC₅₀, mean rel. maximum binding values were calculated. PRO2213 was used as reference anti-ROR1 antibody.

### Binding Affinity by SPR

Binding kinetics (including affinity) of the scFvs to recombinant human ROR1 protein (Fc-Tag, Acro Biosystems) were determined by SPR analysis on a T200 device (Biacore, Cytiva). Recombinant human ROR1 molecules were covalently immobilized to a carboxylmethylated dextran surface (CM5 sensorchip; Biacore, Cytiva) and a titration series of each scFv was injected as analyte. After each analyte injection-cycle, every flow channel on the sensor chip was regenerated (Glycine, pH 2.0), and a new concentration of scFv was injected. The binding kinetics to human ROR1 was measured using a multi-cycle kinetic assay, with nine analyte concentrations ranging from 0.005 to 30 nM, diluted in running buffer (HEPES buffered saline, 0.05% Tween-20, pH 7.5; Bloconcept). The apparent dissociation (k_{d}) and association (kₐ) rate constants and the apparent dissociation equilibrium constant (K_{D}) were calculated with the Biacore analysis software (Biacore Evaluation software version 3.2, Cytiva) using a one-to-one Langmuir binding model and quality of the fits was monitored based on Chi2 and U-value, which are measures for the quality of the curve fitting. The binding level was calculated as the maximum stability binding achieved normalized to the theoretical Rₘₐₓ.

All 13 scFvs were assessed for their binding affinity to ROR1 expressing cells and recombinant hROR1. The data were correlated and are displayed in Table 12.

The affinity to hROR1 was determined for 8 scFvs. The affinities ranged from 160 nM to 0.098 nM. Three scFv showed a better affinity than the Fab fragment reference PRO2213. For three scFvs the affinity could not be determined due to technical reasons, and no binding was observed for 2 scFvs (Table 12).

TABLE 12
Summary of affinity measurements to hROR1 for selected scFvs.

| Protein ID | Description | ka (1/Ms) | kd (1/s) | KD (M) |
|---|---|---|---|---|
| PRO2055 | 55-06-E06-sc01 | NOT CALCULABLE | | |
| PRO2056 | 55-06-E06-sc02 | NOT CALCULABLE | | |
| PRO2057 | 55-33-A06-sc01 | NO BINDER | | |
| PRO2058 | 55-33-A06-sc02 | 4.28E+05 | 9.37E−03 | 2.19E−08 |
| PRO2059 | 55-38-D07-sc01 | 2.18E+05 | 4.09E−04 | 1.88E−09 |
| PRO2060 | 55-38-D07-sc02 | 2.50E+05 | 9.79E−05 | 4.16E−10 |
| PRO2061 | 55-39-G02-sc01 | 1.24E+06 | 2.00E−01 | 1.61E−07 |
| PRO2062 | 55-39-G02-sc02 | 4.45E+06 | 4.55E−04 | 1.03E−10 |
| PRO2063 | 55-19-D06-sc01 | 1.83E+07 | 1.66E−01 | 9.07E−09 |
| PRO2064 | 55-21-E10-sc01 | NOT CALCULABLE | | |
| PRO2066 | 55-42-E05-sc02 | 3.39E+06 | 7.99E−04 | 2.36E−10 |
| PRO2067 | 55-19-D06-sc02 | NOT CALCULABLE | | |
| PRO2068 | 55-21-E10-sc02 | 7.16E+05 | 8.68E−02 | 1.21E−07 |
| PRO2213 | n/a | 6.61E+05 | 3.95E−04 | 5.98E−10 |

Those domains demonstrating non-binding (SPR and cell binding) or indeterminate data were excluded from further analysis. Four scFvs derived from three different clones showing the best binding to human ROR1-expressing cells as well as the highest affinities to recombinant human ROR1 ECD by SPR were selected for further biophysical characterization. These domains include PR02060, PRO2062, PRO2059 and PRO2066.

As cynomolgus monkey ROR1 is 100% identical to human ROR1, no further binding assessment was required to show cross-species binding of these domains.

1.7. Biophysical Characterization of scFv Domains (Storage Stability and DSF)

### Storage Stability

Humanized scFvs were subjected to a four-week stability study, in which the scFvs were formulated in aqueous buffer (50 mM NaCiP, 150 mM NaCl, pH 6.4) at 10 mg/ml and stored at temperatures of <80° C., 4° C. and 40° C. for 28 days. The fractions of monomers and oligomers in the formulation were evaluated by integration of SE-HPLC peak areas at different time points over the course of the study. Table 13 summarizes the percentage of monomeric content and the percentage of monomer loss relative to day 0. Changes in protein concentration were monitored by UV-Vis measurement at 280 nm over the course of the study. As there were however no notable changes in protein concentration observed for any of the samples relative to day 0, data is not shown. Thermal stability was analyzed by nDSF (NanoTemper) to determine the onset of unfolding (Tₒₙₛₑₜ) and midpoint of unfolding (Tₘ). The results are shown in Table 13.

PRO2059 and PRO2060 (derivatives of clone 55-38-007) exhibit a good stability profile with only minor monomer loss at all tested temperatures and the overall best thermal stability of the four molecules. PRO2062 (derivative of clone 55-39-G02) is inferior to PR02059 and PRO2060 with regard to the extent of monomer loss at 40° C. and thermal stability. PRO2066 (derivative of clone 55-42-E05) exhibits considerable monomer loss at 4° C. and 40° C. and therefore can be considered as the least stable molecule in this study (despite its acceptable thermal stability with a Tm of 70.6° C.). [0213] in summary, PRO2060 and PRO2062 were taken forward as lead domains. These domains were selected over the other domains based on a combination of pharmacodynamic and biophysical characteristics. Both molecules bind to different epitopes of ROR1 ECD region 1, allowing additional candidate diversity. These lead domains were taken forward for optimization by protein engineering to improve the T cell score and the stability, as outlined in the next section.

### 1.8. Generation of Optimized Single Chain Domains

PRO2062 has a comparably high, in sMco calculated, T cell score (1,662), which indicates a higher likelihood of stimulating immune responses when introduced into humans. In order to reduce the T cell score, one of the CDRs (CDR1H) was modified. Within rabbits, the first half of CDR1H is more conserved whereas the second half is more variable. Contrary to all other common CDR definitions (IMGT, Chothia and Honegger), the kabat CDR definition includes only the variable second half of CDR1H. To minimize the risk for disrupting binding, two mutations were introduced in the conserved CDR1H part since it is likely that the variable CDR1H part is more important for the interaction with the antigen. By analyzing sequences from the same project, a CDR1H sequence from another clone could be identified that lowers the T cell score and that only differs by two amino acids in the conserved CDR1H part. In addition, this CDR1H sequence is similar to the PRO2062 variable CDR1H sequence, indicating that the two mutations (L271 and S28D) in the conserved CDR1H part could fit well into the protein structure. Furthermore, in silico mutagenesis indicate that the two mutations are not destabilizing. In addition to T cell reducing mutations, the so caged outer loop of the heavy chain of the donor was grafted since this is important for correct positioning of the CDR3H and could serve as compensation for the modifications in CDR1H. Briefly, the segment (AHo residues 82-87) in FW3H was replaced by KTST (R82K, D83T, N84S, N87T (S85 and K86 in the framework were deleted)), AHo89 was changed from Val to Leu (VH3 consensus), and AHo90 was changed from Tyr to Asp (donor). The resulting T cell score of PRO2271 with T cell score reducing mutations in CDR1H and outer loop is 1,160. One additional variant (PRO2272, 55-39-G02-sc04) having the same T cell score reducing mutations and outer loop as PRO2271 but with fewer donor residues than PRO2271 was created. PRO2292, i.e. PRO2062 with a disulfide between AHo 141VL-51VH, has a relatively high T cell score value (1,662).

TABLE 13
28 days storage stability study at 10 mg/mL and temperatures of −80° C., 4° C. and 40° C. and thermal stability by DSF of CDR and
Full grafts.

| | | Temp. | Conc. | monomeric content [%] | | | | | % monomeric content loss | | | | DSF | |
|---|---|---|---|---|---|---|---|---|---|---|---|---|---|---|
| Protein ID | Description | [° C.] | [mg/ml] | d0 | d1 | d7 | d14 | d28 | d1 | d7 | d14 | d28 | Tm [° C.] | Tonset [° C.] |
| PRO2059 | 55-38-D07-sc01 | −80 | 9.47 | 94.3 | 94.3 | 94.3 | 94.3 | 94.2 | 0.0 | 0.0 | −0.1 | 0.0 | 85.6 | 69.7 |
| | | 4 | | 94.3 | 94.5 | 94.9 | 96 | 94.2 | −0.3 | −0.6 | −1.9 | 0.1 | | |
| | | 40 | | 94.3 | 94.5 | 94.1 | 93.7 | 92.9 | −0.3 | 0.2 | 0.6 | 1.4 | | |
| PRO2060 | 55-38-D07-sc02 | −80 | 10.2 | 98.1 | 98.1 | 98.2 | 98.3 | 98.3 | 0.0 | −0.1 | −0.3 | −0.2 | 74.4 | 58.8 |
| | | 4 | | 98.1 | 98.1 | 98.2 | 98.2 | 98.2 | 0.0 | −0.1 | −0.2 | −0.1 | | |
| | | 40 | | 98.1 | 97.9 | 97.5 | 96.8 | 95.2 | 0.2 | 0.6 | 1.3 | 3 | | |
| PRO2062 | 55-39-G02-sc02 | −80 | 9.4 | 99.3 | 99.3 | 99.2 | 99.3 | 99.2 | 0.0 | 0.1 | 0 | 0.1 | 61.7 | 56.0 |
| | | 4 | | 99.3 | 99.3 | 99.2 | 99.2 | 99.1 | 0.0 | 0.1 | 0.1 | 0.2 | | |
| | | 40 | | 99.3 | 94.3 | 90.5 | 90.7 | 90.3 | 5.0 | 8.8 | 8.7 | 9.1 | | |
| PRO2066 | 55-42-E05-sc02 | −80 | 9.54 | 96.1 | 95.8 | 95.2 | 95.4 | 94.8 | 0.4 | 1.0 | 0.8 | 1.4 | 70.6 | 61.0 |
| | | 4 | | 96.1 | 94.9 | 90.6 | 88.1 | 85.8 | 1.3 | 5.8 | 8.3 | 10.8 | | |
| | | 40 | | 96.1 | 88.4 | 87.9 | 87.9 | 87.4 | 8.1 | 8.6 | 8.6 | 9.1 | | |

NA: not measured,
ND: non-determinable

PRO2060 has a T cell score of 949 and therefore no T cell score optimization was needed. PRO2291, i.e. PRO2060 with a disulfide between AHo 141VL-51VH, has the same T cell score value of 949 as PRO2060. The optimized variants are summarized in Table 14.

TABLE 14
Overview of PRO2062 and PRO2060 variants

| | | T cell | CDR | | |
|---|---|---|---|---|---|
| PRO ID | Domain ID | score | mutations | VL FW mutations | VH FW mutations |
| PRO2062 | 55-39-G02 | 1,662 | — | see table above | see table above |
| | sc02- | | | | |
| PRO2271 | 55-39-G02 | 1,160 | L27I, | PRO2062 mutations | PRO2062 mutations + |
| | sc03 | | S28D | | R82K, D83T, N84S |
| | | | | | N87T, Y90D (S85 and |
| | | | | | K86 in the framework are |
| | | | | | deleted); V89L (back to |
| | | | | | VH3 consensus |
| PRO2292 | 55-39-G02- | 1,662 | — | PRO2062 mutations + | PRO2062 mutations + |
| | sc05 | | | T141C | G51C |
| PRO2060 | 55-38-D07- | 949 | — | see table above | see table above |
| | sc02 | | | | |
| PRO2291 | 55-38-D07- | 949 | — | PRO2060 mutations + | PRO2060 mutations |
| | sc06 | | | T141C | G51C |

### 1.9. Characterization of Optimized Single Chain Domains

Cell Binding of Opimized scFvs to ROR1 Expressing MDA-MB-231 Cells by Flow Cytometry

Plasma membrane binding of optimized scFvs to human ROR1-expressing MDA-MB-231 cells by flow cytometry was performed as described above. In brief, 5-fold serial dilutions of optimized scFvs of anti-ROR1 clone 55-39-G02 (PRO2271, PRO2292) and of clone 55-38-D07 (PRO2291) as wel as of reference Fab fragment PRO2213 starting at a concentration of 40 nM were prepared and then added to the plates with cells. After incubation at 4° C. for 1 h, plates were washed and incubated with specific detection antibodies. Fluorescence intensity of APC channel was recorded for each sample using NovoCyte 2060 flow cytometer and the geometric mean of fluorescence intensity MFI was calculated.

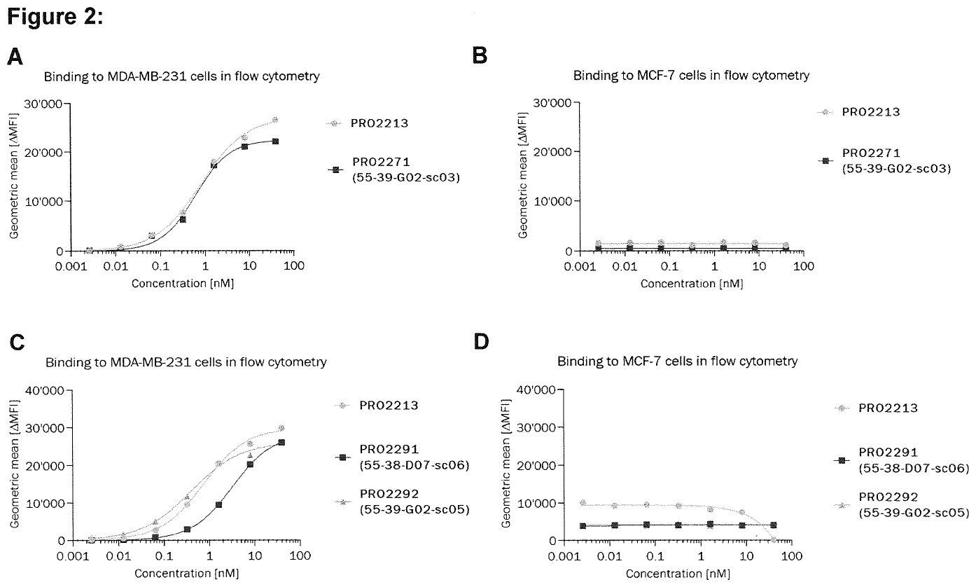

The apparent binding affinity of optimized scFvs PRO2271, PRO2291 and PRO2292 to plasma membrane human ROR1 was assessed in flow cytometry experiments. The calculated EC₅₀, rel. EC₅₀ as well as rel. maximum binding values obtained in these experiments are shown Table 15. As shown in FIG. 2, all scFvs demonstrated binding to ROR1 positive MDA-MB-231 cells while no binding was found to ROR1 negative MCF-7 cells. PRO2271, variant of PRO2062 with T cell score optimization and outer loop grafting, showed an around two times lower apparent binding affinity when compared to the original scFv PRO2062. PRO2291 and PR02292, which are variants of PRO2060 and PRO2062 including VL-VH interdomain disulfide bond for stabilization, demonstrated apparent binding affinities comparable to the apparent binding affinities of the parental scFvs PR02060 and PR02062.

TABLE 15
Apparent binding affinity of optimized anti-ROR1 scFvs
PRO2271, PRO2291 and PRO2292 to human ROR1
expressed on MDA-MB-231 cells by flow cytometry.
PRO2213 was used as reference anti-ROR1 antibody.

| | | | rel. EC₅₀ | Rel. maximum |
|---|---|---|---|---|
| | | EC₅₀ | EC_{50, PRO2213}/ | binding (MFI_{scFv}/ |
| Protein ID | Description | [nM] | EC_{50, scFV}) | MFI_{PRO2213}) |
| PRO2271 | 55-39-G02-sc03 | 0.63 | 1.34 | 0.87 |
| PRO2291 | 55-38-D07-sc06 | 2.74 | 0.27 | 1.18 |
| PRO2292 | 55-39-G02-sc05 | 0.27 | 2.61 | 1.18 |

### Binding Affinity by SPR

Binding kinetics (including affinity) of the optimized scFv PR02071, PRO2291 and PR02292 to recombinant human ROR1 protein (Fc-Tag, Acro Biosystems) were determined by SPR analysis on a T200 device (BWacore, Cytiva), as described in section 1.6.

Affinity to human ROR1 was determined together with the non-optimized versions as comparison. The results of the SPR analysis are summarized in Table 16.

TABLE 16
Summary of affinity measurement to hROR1 for optimized scFv, compared to
the non-optimized (in the same experiment).

| | | | | | Binding level normalized |
|---|---|---|---|---|---|
| Protein # | Protein description | kₐ (1/Ms) | k_{d} (1/s) | K_{D} (M) | to theoretical Rₘₐₓ (%) |
| PRO2271 | 55-39-G02-sc03 | 1.59E+06 | 5.78E−04 | 3.64E−10 | 52.7 |
| PRO2291 | 55-38-D07-sc06 | 2.69E+05 | 3.02E−04 | 1.12E−09 | 26.9 |
| PRO2292 | 55-39-G02-sc05 | 6.80E+06 | 6.16E−04 | 9.06E−11 | 49.6 |
| PRO2060 | 55-38-D07-sc02 | 3.35E+05 | 9.56E−05 | 2.85E−10 | 17.3 |
| PRO2062 | 55-39-G02-sc02 | 5.29E+06 | 4.90E−04 | 9.26E−11 | 57.8 |

1.10. Biophysical Characterization of scFv Domains (Storage Stability and DSF)

Optimized variants of humanized scFvs were subjected to a four-week stability study, in which the scFvs were formulated in aqueous buffer (50 mM NaCiP, 150 mM NaCl, pH 6.4) at 10 mg/ml and stored at temperatures of <−80° C., 4° C. and 40° C. for four weeks. The fraction of monomers and oligomers in the formulation were evaluated by integration of SE-HPLC peak areas at different time points over the course of the study. Table 17 summarizes monomeric content in % and % monomer loss relative to d0. Changes in protein concentration were monitored by UV-Vis measurement at 280 nm over the course of the study. As there were no notable differences observed for any of the samples relative to d0, data is not shown. Thermal stability was analyzed by nDSF (NanoTemper) determining the onset of unfolding (Tₒₙₛₑₜ) and midpoint of unfolding (Tₘ). Results are shown in Table 17.

The optimized derivative of clone 55-39-G02 (PRO2271) exhibits similar stability as PRO2062. Derivatives of clone 55-38-D07 and 55-39-G02 with VL-VH disulfide (PRO2291 and PRO2292) both exhibit improved stability as compared to their counterparts without disulfide (PRO2060 and PR02062) and can be considered as the overall most stable scFv domains.

### Example 2: Generation and Testing of Anti-CD3 Molecules

The identification, selection and humanization of the anti-CD3 rabbit IgG clone 28-21-D09 as well as the production and characterization of the corresponding humanized anti-CD3 scFv 28-21-D09-sc04 (PR0726) were performed as described in patent application WO 2018/224441, which is herewith incorporated by reference. The characterization of the anti-CD3 scFv PRO726 is briefly outlined in the following.

2.1. Characterization of the Anti-CD3 scFv 28-21-D09-Sc04 (PR0726)

### Binding Affinity and Species Cross-Reactivity (FS)

Binding kinetics (including affinity) of the selected binding domain 28-21-D09-sc04 (PR0726) to recombinant human CD3 epsilon extra-cellular domain protein (hCD3ε; His-Tag, SinoBiological) was determined by SPR analysis on a T200 device (Biacore, Cytiva). Recombinant hCD3ε molecules were covalently immobilized to a carboxymethylated dextran surface (CM5 sensorchip; Biacore, Cytiva) and a titration series of PR0726 was injected as analyte. After each analyte injection-cycle, every flow channel on the sensor chip was regenerated (Glycine, pH 2.0), and a new concentration of PR0726 was injected.

TABLE 17
28 d storage stability study at 10 mg/mL and temperatures of −80° C., 4° C. and 40° C. and thermal
stability by DSF of optimized scFv domains.

| | | | | | | | | | % monomeric | | | | DSF | |
|---|---|---|---|---|---|---|---|---|---|---|---|---|---|---|
| | | Temp. | Conc. | monomeric content [%] | | | | | content loss | | | | Tm | Tonset |
| PRO ID | Description | [° C.] | [mg/ml] | d0 | d1 | d7 | d14 | d28 | d1 | d7 | d14 | d28 | [° C.] | [° C.] |
| PRO2271 | 55-39-G02-sc03 | −80 | 10.0 | 99.5 | 99.5 | 99.6 | 99.5 | 99.3 | 0.1 | 0.0 | 0.0 | 0.3 | ND | ND |
| | | 4 | | 99.5 | 99.5 | 99.3 | 99.2 | 99.0 | 0.0 | 0.2 | 0.3 | 0.6 | | |
| | | 40 | | 99.5 | 90.5 | 90.6 | 90.5 | 90.7 | 9.1 | 9.0 | 9.1 | 8.9 | | |
| PRO2291 | 55-38-D07-sc06 | −80 | 9.3 | 99.6 | NA | NA | NA | 99.6 | NA | NA | NA | 0.0 | 72.0 | 57.7 |
| | | 4 | | 99.6 | 99.6 | NA | 99.6 | 99.6 | 0.0 | NA | 0.0 | 0.0 | | |
| | | 40 | | 99.6 | 99.6 | NA | NA | 98.5 | 0.0 | NA | NA | 1.1 | | |
| PRO2292 | 55-39-G02-sc05 | −80 | 9.5 | 96.6 | NA | NA | NA | 96.6 | NA | NA | NA | 0.0 | ND | 59.7 |
| | | 4 | | 96.6 | 96.5 | 96.6 | 96.7 | 96.6 | 0.1 | −0.1 | −0.1 | 0.0 | | |
| | | 40 | | 96.6 | 96.6 | 96.5. | 96.5 | 96:3 | 0.0 | 0.1 | 0.1 | 0.3 | | |

NA: not measured,
ND: non-determinable

TABLE 18
Summary of affinity measurement to hCD3ε and cCD3ε for the selected scFv PRO726 (domain 28-21D09-sc04).

| Protein ID | Ligand | Kₐ₁ (1/Ms) | K_{d1} (1/s) | K_{D1} (M) | Kₐ₂ (1/Ms) | K_{d2} (1/s) | K_{D2} (M) | K_{D} (M) |
|---|---|---|---|---|---|---|---|---|
| PRO726 | hCD3ε | 6.5E+05 | 5.5E−03 | 8.3E−09 | 1.4E−03 | 2.4E−03 | 1.8E+00 | 5.3E−09 |
| PRO726 | cCD3ε | 8.2E+05 | 6.2E−03 | 7.5E−09 | 1.5E−03 | 2.6E−03 | 1.8E+00 | 4.8E−09 |

The binding kinetics to hCD3ε was measured using a multi-cycle kinetic assay, with eight analyte concentrations ranging from 0.7 to 90 nM, 1:2 diluted in running buffer (HEPES buffered saline, 0.05% Tween-20, pH 7.5; Bioconcept). The apparent dissociation (k_{d1}) and association (kₐ₁) rate constants, the second reaction constants (kₐ₂ and k_{d2}) and the apparent dissociation equilibrium constant (K_{D}) were calculated with the Biacore analysis software (Biacore Evaluation software Version 3.2, Cytiva) using a two-state binding model and quality of the fits was monitored based on relative Chi2. The binding level was calculated as the maximum stability binding achieved normalized to the theoretical Rmax.

Binding kinetics to recombinant Cynomolgus monkey CD3ε (cCD3ε; His-Tag; SinoBiological) were measured as mentioned above for hCD3ε, with the exception that cCD3εwas used instead of hCD3ε. Results are summarized in Table 18.

Biophysical characterization

Humanized CD3“binding domain was subjected to a four-week stability study, in which the scFv was formulated in aqueous buffer (50 mM NaCiP, 150 mM NaCl, pH 6.4) at 10 mg/ml and stored at temperatures of <−80° C., 4° C. and 40° C. for four weeks. The fraction of monomers and oligomers in the formulation were evaluated by integration of SE-HPLC peak areas at different time points over the course of the study. Table 19 summarizes monomeric content in % and % monomer loss relative to d0. Changes in protein concentration were monitored by UV-Vis measurement at 280 nm over the course of the study. There was no notable loss of protein content observed for any of the samples relative to d0, data is not shown.

Thermal unfolding data obtained from Differential Scanning Fluorimetry (DSF) measurements is shown in Table 19. Resulting midpoint of thermal unfolding (Tm) and onset temperature (Tonset) of unfolding have been determined by fitting of data to a Boltzmann equation.

TABLE 19
Four-week stability study of 28-21-D09-sc04 domain.

| Protein | | Temp. | Conc. | [% monomer content] | | | | | % monomer loss | | | | Tm |
|---|---|---|---|---|---|---|---|---|---|---|---|---|---|
| ID | Description | [° C.] | [mg/ml] | d0 | d1 | d7 | d14 | d28 | d1 | d7 | d14 | d28 | [° C.] |
| PRO726 | 28-21-D09-sc04 | −80.0 | 10.0 | 93.9 | 93.1 | 93.3 | NA | 93.1 | 0.8 | 0.7 | NA | 0.9 | 68.1 |
| | | 4.0 | | 93.9 | 88.0 | 86.8 | 85.8 | 86.0 | 6.3 | 7.6 | 8.7 | 8.4 | |
| | | 40.0 | | 93.9 | 86.4 | 87.1 | 86.7 | 85.8 | 8.1 | 7.3 | 7.7 | 8.7 | |

NA: not measured

### Example 3: Generation and Testing of Anti-hSA Molecules

The identification, selection, humanization as well as the production and characterization of the humanized anti-hSA binding domain 19-01-H04-sc03 (PRO0325) and 23-13-A01-sc03 (PR00459) were performed as described in the patent application EP 19 206 959.9, which is herewith incorporated by reference. The identification, selection, humanization as well as the production and characterization of the humanized anti-hSA binding domain19-04-A10-sc02 (PRO2155) was performed analogous to the procedures described in the patent application EP 19 206 959.9. The characterization of the anti-CD3 scFv PR02155 is briefly outlined in the following.

3.1. Characterization of the Anti-hSA scFvs 19-04-A10-Sc02 (PRO2155) Binding Affinity and Species Cross-Reactivity

Binding kinetics (including affinity) of the selected domain 19-04-A10-sc02 to human serum albumin (hSA, Sigma-Aldrich A3782) were determined by SPR analysis on a T200 device (Biacore, Cytiva) both at pH 7.4 and pH 5.5. hSA molecules were covalently immobilized to a carboxymethylated dextran surface (CM5 sensorchip, Biacore, Cytiva) and a titration series of each scFv molecule was injected as analyte. After each analyte injection-cycle, every flow channel on the sensor chip was regenerated (Glycine, pH 2.0), and a new concentration of scFv molecule was injected. The binding kinetics to hSA were measured using a multi-cycle kinetic assay, with eleven concentrations from 0.044 to 45 nM (1:2) diluted in a relevant running buffer (PBS 0.05% Tween-20, or PBS 0.05% Tween-20, pH 5.5). The apparent dissociation (k_{d}) and association (kₐ) rate constants, and the apparent dissociation equilibrium constant (K_{D}) were calculated with the Biacore analysis software (Biacore Evaluation software Version 3.2, Cytiva) using a one-to-one Langmuir binding model and quality of the fits was monitored based on relative Chi2. The binding level was calculated as the maximum stability binding achieved normalized to the theoretical Rmax.

Binding kinetics of the selected scFv were also determined for the cynomolgus monkey serum albumin (cSA, Molecular Innovations CYSA) and for the mouse serum albumin (mSA, Sigma-Aldrich A3559) as described above, with the difference that cSA or mSA were used instead of hSA. Binding kinetics to hSA, mSA and cSA at pH 5.5 and pH 7.4 are summarized in Table 20.

TABLE 20
Summary of affinity measurement to hSA, cSA and mSA for the serum albumin binding
scFv molecule PRO2155 (domain 19-04-A10-sc02).

| Protein | | Tested | | Kₐ | k_{d} | K_{D} |
|---|---|---|---|---|---|---|
| #ID | Protein description | pH | Antigen | (1/Ms) | (1/s) | (M) |
| PRO2155 | 19-04-A10-sc02 | pH5.5 | CSA | 9.1E+05 | 2.1E−03 | 2.3E−09 |
| PRO2155 | 19-04-A10-sc02 | pH5.5 | HSA | 9.0E+05 | 3.0E−03 | 3.3E−09 |
| PRO2155 | 19-04-A10-sc02 | pH5.5 | MSA | 7.8E+04 | 2.0E−04 | 2.5E−09* |
| PRO2155 | 19-04-A10-sc02 | pH7.4 | CSA | 5.8E+05 | 9.1E−04 | 1.6E−09 |
| PRO2155 | 19-04-A10-sc02 | pH7.4 | HSA | 5.5E+05 | 1.2E−03 | 2.2E−09 |
| PRO2155 | 19-04-A10-sc02 | pH7.4 | MSA | 3.2E+04 | 2.9E−04 | 9.0E−09* |

*biphasic binding was corrected with bulk correction in 1:1 fit, resulting in lower max responses

Biophysical characterization

HSA-domains 19-04-A10-sc02 (PR02155) and 19-04-A10-sc06 (sc02 domain with VL-VH disulfide, VL-T141CNH-G51C, AHo numbering; PR02317) were subjected to a four-week stability study, in which the scFvs were formulated in aqueous buffer (50 mM NaCiP, 150 mM NaCl, pH 6.4) at 10 mg/ml and stored at temperatures of <−80° C., 4° C. and 40° C. for four weeks. The fractions of monomers and oligomers in the formulation were evaluated by integration of SE-HPLC peak areas at different time points over the course of the study. Table 21 summarizes monomeric content in % and % monomer loss relative to d0. Changes in protein concentration were monitored by UV-Vis measurement at 280 nm over the course of the study. As there was no notable protein content loss observed for any of the samples relative to d0, data is not shown. Thermal stability was analyzed by nDSF (NanoTemper) determining the onset of unfolding (Tonset) and midpoint of unfolding (Tm). DSF results are shown in Table 21.

TABLE 21
Four-week stability study of 19-04-A10-sc02 and 19-04-A10-sc06 anti-hSA domains.

| | | Temp. | [mg/ml] | monomer content [%] | | | | | % monomer loss | | | | Tm |
|---|---|---|---|---|---|---|---|---|---|---|---|---|---|
| Protein ID | Description | [° C.] | Conc. | d0 | d1 | d7 | d14 | d28 | d1 | d7 | d14 | d28 | [° C.] |
| PRO2155 | 19-04-A10-sc02 | −80 | 10.4 | 99.4 | NA. | NA | NA | 99.5 | NA | NA | NA | −0.1 | 75.3 |
| | | 4.0 | | 99.4 | 99.4 | NA | NA | 99.1 | 0.1 | NA | NA | 0.3 | |
| | | 40.0 | | 99.4 | 98.6 | NA | NA | 89.1 | −0.8 | NA | NA | 10.4 | |
| PRO2317 | 19-04-A10-sc06 | 40.0 | 10.5 | 99.2 | 98.8 | 98.7 | 98.6 | 98.4 | 0.4 | 0.6 | 0.7 | 0.9 | 78.4 |

NA: not measured

### Example 4: Generation and Production of scMATCH3 Anti-ROR1xCD3xhSA Multispecific Antibodies

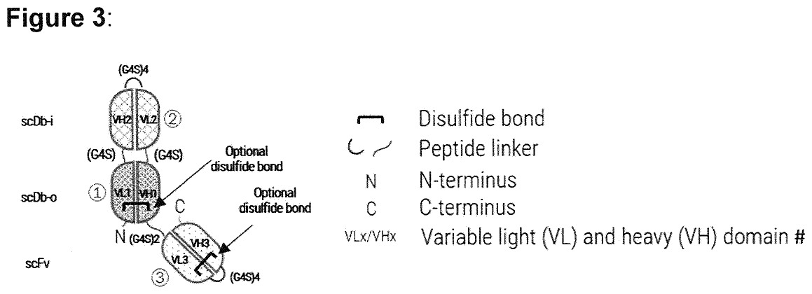

HSA-, CD3- and ROR1“binding domains were combined in the trispecific scMATCH3 format. The scMATCH3 format consists solely of variable domains connected by GS-linkers of different lengths as depicted in FIG. 3. In this format, split variable domains are located on a single peptide chain (sc) which assemble into fully functional trispecific molecules. Similar as to scFv domains, VL/VH disulfide bonds may be incorporated for stabilization of individual domains. ScMATCH3 molecules can be expressed recombinantly in mammalian cells and a conventional affinity chromatography step can be used for their purification.

Expression of mammalian scMATCH3 constructs was performed in CHO—S cells using CHOgro transient transfection kit (Mirus). Cultures were harvested after 5-7 days (when cell viability <70% was reached) expression at 37° C. by centrifugation followed by filtration. Proteins were purified from clarified culture supernatants by Protein L affinity chromatography followed by size exclusion chromatography (SEC) in 50 mM phosphate-citrate buffer with 300 mM sucrose at pH 6.5. Monomeric content of SEC fractions was assessed by SE-HPLC analysis and fractions with a monomeric content >95% were pooled. For quality control of the manufactured material, standard analytical methods such as SE-HPLC, UV₂₈₀ and SDS-PAGE were applied. Molecule domain composition and a manufacture summary of scMATCH3 molecules are shown in Table 22.

The scMATCH3 molecules PRO2667 and RPO2668, which comprise modified or improved ROR1-, CD3- and hSA-binding domains, were produced at 1 l scale at Evitria AG (Schlieren, Switzerland) using their proprietary mammalian expression system. Proteins were purified from clarified culture supernatants by Protein L (CaptoL, Cytiva) affinity chromatography followed by SEC in 50 mM phosphate-citrate buffer with 300 mM sucrose at pH 6.5. Monomeric content of SEC fractions was assessed by SE-HPLC analysis and fractions with a monomeric content >95% were pooled. For quality control of the manufactured material, standard analytical methods such as SE-HPLC, UV₂₈₀ and SDS-PAGE were applied. A manufacture summary of these scMATCH3 molecules is shown in Table 23. Thermal stability of molecules has been assessed by nDSF using Prometheus NT.48 device (NanoTemper).

TABLE 22
ScMATCH3 molecule architecture, final titer and monomeric purity by SE−HPLC.

| | | Domain 1, | Domain 2, | Domain 3, | | Monomeric |
|---|---|---|---|---|---|---|
| | | scDb-o position | scDb-i position | scFv position | Final Titer | content |
| PRO ID | Format | (hSA) | (CD3) | (ROR1) | [mg/l culture] | [%] |
| PRO2507 | scMATCH3 | 19-04-A10-sc06 | 28-21-D09-sc04 | 55-38-D07-sc02 | 1.9 | 99.2 |
| PRO2508 | scMATCH3 | 19-04-A10-sc06 | 28-21-D09-sc04 | 55-38-D07-sc06 | 2.3 | 99.1 |
| PRO2509 | scMATCH3 | 19-04-A10-sc06 | 28-21-D09-sc04 | 55-39-G02-sc02 | 2.2 | 98.7 |
| PRO2510 | scMATCH3 | 19-04-A10-sc06 | 28-21-D09-sc04 | 55-39-G02-sc05 | 2.7 | 99.0 |
| PRO2557 | scMATCH3 | 19-04-A10-sc06 | 28-21-D09-sc04 | 55-39-G02-sc06 | 2.0 | 98.4 |

TABLE 23
Manufacture of scMATCH3 molecules PRO2667 and RPO2668

| | | Capture | | Purity by | nDSF | nDSF |
|---|---|---|---|---|---|---|
| | | titer | Titer | SE-HPLC | T_{M1} | T_{M2} |
| Protein ID | Format | [mg/l] | [mg/l] | [% monomer] | [° C.] | [° C.] |
| PRO2667 | scMATCH3 | 66.3 | 25.7 | 98.7 | 62.5 | 71.2 |
| PRO2668 | scMATCH3 | 47.1 | 23.9 | 99.7 | 64.6 | 74.0 |

### Example 5: Pharmacodynamic Characterization of scMATCH3 Molecules

### 5.1. Binding Affinity by SPR

The binding kinetics for nine multispecific scMATCH3 molecules to the three targets human ROR1 (hROR1), human CD3ε (h CD3ε) and hSA were tested by SPR.

Binding Affinity to hROR1 by SPR

Binding kinetics (including affinity) of the nine scMATCH3 molecules to recombinant human ROR1 protein (Fc-Tag, Acro Biosystems) were determined by SPR analysis on a T200 device (Biacore, Cytiva), as described in section 1.6. Recombinant human ROR1 molecules were covalently immobilized to a carboxymethylated dextran surface (CM5 sensorchip; Biacore, Cytiva) and a titration series of each MATCH3 molecule was injected as analyte. After each analyte injection-cycle, every flow channel on the sensor chip was regenerated (Glycine, pH 2.0 and 3 M MgC₂), and a new concentration of MATCH3 molecule was injected. The binding kinetics to human ROR1 were measured using a multi-cycle kinetic assay, with nine analyte concentrations ranging from 0.088 to 45 nM, 1:2 diluted in running buffer (HEPES buffered saline, 0.05% Tween-20, pH 7.5; Bloconcept). The apparent dissociation (k_{d}) and association (kₐ) rate constants and the apparent dissociation equilibrium constant (K_{D}) were calculated with the Biacore analysis software (Biacore Evaluation software Version 3.2, Cytiva) using a one-to-one Langmuir binding model, and quality of the fits was monitored based on Chi2 and U-value, which are measures for the quality of the curve fitting. The binding level was calculated as the maximum stability binding achieved normalized to the theoretical Rmax.

Binding kinetics to hROR1 were determined for nine scMATCH3 with affinities ranging from 1.6 nM to 0.12 nM (Table 24). hROR1 affinities for all tested scMATCH3 molecules were similar to the respective parental scFv.

TABLE 24
Summary of affinity measurement to hROR1 for scMATCH3 molecules.

| Protein ID | Protein Description | ka (1/Ms) | kd (1/s) | KD (M) |
|---|---|---|---|---|
| PRO2287 | 19-01-H04-sc02/3 diS scDb-o/28-21-D09-sc04 | 1.3E+06 | 4.0E−04 | 3.2E−10 |
| | scDb-i/55-39-G02-sc02 scFv | | | |
| PRO2507 | 19-04-A10-sc06 scDb-o/28-21-D09-sc04 | 8.2E+04 | <1.00E−05 | 1.2E−10 |
| | scDb-i/55-38-D07-sc02-scFv | | | |
| PRO2508 | 19-04-A10-sc06 scDb-o/28-21-D09-sc04 | 7.6E+04 | <1.00E−05 | 1.3E−10 |
| | scDb-i/55-38-D07-sc06-scFv | | | |
| PRO2509 | 19-04-A10-sc06 scDb-o/28-21-D09-sc04 | 1.3E+06 | 4.7E−04 | 3.6E−10 |
| | scDb-i/55-39-G02-sc02-scFv | | | |
| PRO2510 | 19-04-A10-sc06 scDb-o/28-21-D09-sc04 | 1.3E+06 | 6.0E−04 | 4.6E−10 |
| | scDb-i/55-39-G02-sc05-scFv | | | |
| PRO2286 | 23-13-A01-sc02 diS scDb-o/28-21-D09-sc04 | 1.4E+06 | 4.5E−04 | 3.3E−10 |
| | scDb-i/55-39-G02-sc02-scFv | | | |
| PRO2557 | 19-04-A10-sc06 scDb-o/28-21-D09-sc04 | 4.2E+05 | 6.7E−04 | 1.6E−09 |
| | scDb-i/55-39-G02-sc06-scFv | | | |
| PRO2667 | 19-04-A10-sc06(RTQ) scDb-o/28-21-D09-sc04 | 9.5E+04 | 9.2E−05 | 9.7E−10 |
| | (RTQ) scDb-i/55-38-D07-sc02(RTQ)-scFv | | | |
| PRO2668 | 19-04-A10-sc06(RTQ) scDb-o/28-21-D09-sc04 | 2.0E+06 ± | 5.6E−04 ± | 2.8E−10 ± |
| | (RTQ) scDb-i/55-39-G02-sc05(RTQ)-scFv | 7.6E+04 | 6.7E−05 | 2.2E−11 |

Binding Affinity to hCD3ε by SPR

Binding kinetics (including affinity) of the nine selected scMATCH3 molecules to recombinant human CD3 epsilon extra-cellular domain protein (hCD3ε; His-Tag, SinoBiological) were determined by SPR analysis on a T200 device (Biacore, Cytiva). Recombinant hCD3ε molecules were covalently immobilized to a carboxymethylated dextran surface (CM5 sensorchip; Biacore, Cytiva) and a titration series of each MATCH3 molecule was injected as analyte. After each analyte injection-cycle, every flow channel on the sensor chip was regenerated (Glycine, pH 2.0 and 3 M MgCl₂), and a new concentration of MATCH3 molecule was injected. The binding kinetics to hCD3ε were measured using a multi-cycle kinetic assay, with nine analyte concentrations ranging from 0.088 to 45 nM, 1:2 diluted in running buffer (HEPES buffered saline, 0.05% Tween-20, pH 7.5; Bioconcept). The apparent dissociation (kₐ₁) and association (kₐ₁) rate constants, the second reaction constants (kₐ₂ and k_{d}) and the apparent dissociation equilibrium constant (K_{D}) were calculated with the Biacore analysis software (Biacore Evaluation software Version 3.2, Cytiva) using a two-state binding model, and quality of the fits was monitored based on relative Chi2. The binding level was calculated as the maximum stability binding achieved normalized to the theoretical Rmax.

Binding kinetics to hCD3ε were determined for 9 MATCH3 with affinities ranging from 9.4 nM to 1.5 nM (Table 25), hCD3ε affinities for all tested MATCH3 molecules were similar to the parental scFv 28-21-D09-sc04.

TABLE 25
Summary of affinity measurement to hCD3ε for selected MATCH3 molecules.

| Protein | | kₐ₁ | k_{d1} | kₐ₂ | k_{d2} | K_{D} |
|---|---|---|---|---|---|---|
| #ID | Protein Description | (1/Ms) | (1/s) | (1/s) | (1/s) | (M) |
| PRO2287 | 19-01-H04-sc02/3 diS scDb-o/ | 8.7E+05 | 3.4E−03 | 1.7E−03 | 2.0E−03 | 2.1E−09 |
| | 28-21-D09-sc04 scDb-i/55- | | | | | |
| | 39-G02-sc02 scFV | | | | | |
| PRO2507 | 19-04-A10-sc06 scDb-o/28-21- | 3.4E+05 | 6.0E−03 | 6.9E−03 | 7.8E−03 | 9.4E−09 |
| | D09-sc04 scDb-1/55-38-D07- | | | | | |
| | sc02 scFv | | | | | |
| PRO2508 | 19-04-A10-sc06 scDb-o/28-21 | 3.8E+05 | 6.2E−03 | 6.8E−03 | 7.4E−03 | 8.5E−09 |
| | D09-sc04 scDb-i/55-38-D07- | | | | | |
| | sc06 scFv | | | | | |
| PRO2509 | 19-04-A10-sc06 scDb-o/28-21- | 1.0E+06 | 3.4E−03 | 9.6E−04 | 1.6E−03 | 2.1E−09 |
| | D09-sc04 scDb-i/55-39-G02- | | | | | |
| | sc02 scFv | | | | | |
| PRO2510 | 19-04-A10-sc06 scDb-o/28-21- | 1.0E+06 | 3.6E−03 | 1.3E−03 | 2.0E−03 | 2.1E−09 |
| | D09-sc04 scDb-i/55-39-G02- | | | | | |
| | Sc05 scFV | | | | | |
| PRO2286 | 23-13-A01-sc02 diS scDb- | 1.1E+06 | 2.7E−03 | 6.9E−04 | 1.1E−03 | 1.5E−09 |
| | o/28-21-D09-sc04 scDb-i/55- | | | | | |
| | 39-G02-sc02 scFv | | | | | |
| PRO2557 | 19-04-A10-sc06 scDb-o/28-21- | 4.3E+05 | 3.1E−03 | 4.2E−04 | 1.4E−03 | 5.5E−09 |
| | D09-sc04 scDb-1/55-39-G02- | | | | | |
| | sc06 scFv | | | | | |
| PRO2667 | 19-04-A10-sc06(RTQ) scDb-o/ | 5.4E+05 | 4.1E−03 | 4.5E−03 | 6.1E−03 | 4.4E−09 |
| | 28-21-D09-sc04 (RTQ) scDb-i/ | | | | | |
| | 55-38-D07-sc02(RTQ)-scFv | | | | | |
| PRO2668 | 28-21-D09-sc04 (RTQ) scDb-i/ | 2.5E+06 ± | 4.7E−03 ± | 4.9E−03 ± | 2.3E−03 ± | 1.8E−09 ± |
| | 19-04-A10-sc06(RTQ) scDb-o/ | 1.4E+06 | 8.5E−04 | 1.6E−03 | 7.2E−04 | 9.2E−10 |
| | 55-39-G02-sc05(RTQ)-scFv | | | | | |

### Binding Affinity to hSA by SPR

Binding kinetics (including affinity) of the seven selected scMATCH3 molecules to human serum albumin (hSA, Sigma-Aldrich) were determined by SPR analysis on a T200 device (Biacore, Cytiva), hSA molecules were covalently immobilized to a carboxymethylated dextran surface (CM5 sensorchip; Biacore, Cytiva) and a titration series of each MATCH3 molecule was injected as analyte (Exp. ND032-0033). After each analyte injection-cycle, every flow channel on the sensor chip was regenerated (Glycine, pH 2.0), and a new concentration of MATCH3 molecule was injected. The binding kinetics to hSA were measured using a multi-cycle kinetic assay, with ten analyte concentrations ranging from 0.352 to 180 nM, 1:2 diluted in a relevant running buffer (PBS, 0.05% Tween-20, pH 5.5). The apparent dissociation (k_{d}) and association (kₐ) rate constants, and the apparent dissociation equilibrium constant (K_{D}) were calculated with the Biacore analysis software (Biacore Evaluation software Version 3.2, Cytiva) using a one-to-one Langmuir binding model, and quality of the fits was monitored based on relative Chi2 and U-value, which are measures for the quality of the curve fitting. The binding level was calculated as the maximum stability binding achieved normalized to the theoretical Rmax.

Binding kinetics to hSA was determined for seven MATCH3 molecules (Table 26), hSA affinity at pH 5.5 for all tested MATCH3 molecules is deemed sufficient to extend the systemic half-life of the MATCH3 molecules.

TABLE 26
Summary of affinity measurement to hSA for selected MATCH3 molecules.

| Protein | | | | |
|---|---|---|---|---|
| #ID | Protein description | kₐ (1/Ms) | k_{d} (1/s) | K_{D} (M) |
| PRO2287 | 19-01-H04-sc02/3 diS scDb-o/28-21-D09-sc04 | 1.7E+06 | 3.0E−04 | 1.7E−10 |
| | scDb-i/55-39-G02-sc02 scFv | | | |
| PRO2507 | 19-04-A10-sc06 scDb-o/28-21-D09-sc04 | 4.0E+05 | 1.7E−03 | 4.3E−09 |
| | scDb-i/55-38-D07-sc02 scFv | | | |
| PRO2508 | 19-04-A10-sc06 scDb-o/28-21-D09-sc04 | 4.2E+05 | 1.8E−03 | 4.3E−09 |
| | scDb-i/55-38-D07-sc06 scFv | | | |
| PRO2509 | 19-04-A10-sc06 scDb-o/28-21-D09-sc04 | 9.4E+05 | 2.0E−03 | 2.1E−09 |
| | scDb-i/55-39-G02-sc02 scFv | | | |
| PRO2510 | 19-04-A10-sc06 scDb-o/28-21-D09-sc04 | 1.0E+06 | 2.1E−03 | 2.0E−09 |
| | scDb-i/55-39-G02-sc05 scFv | | | |
| PRO2286 | 23-13-A01-sc02 diS scDb-o/28-21-D09-sc04 | 1.1E+06 | 6.7E−05 | 6.2E−11 |
| | scDb-i/55-39-G02-sc02 scFv | | | |
| PRO2557 | 19-04-A10-sc06 scDb-o/28-21-D09-sc04 | 4.5E+05 | 2.0E−03 | 4.4E−09 |
| | scDb-i/55-39-G02-sc06 scFv | | | |
| PRO2558 | 19-04-A10-sc06 scDb-o/28-21-D09-sc04 | 4.6E+05 | 2.0E−03 | 4.4E−09 |
| | scDb-i/56-22-A05-sc02 scFv | | | |
| PRO2667 | 19-04-A10-sc06(RTQ) scDb-o/28-21-D09-sc04 | 3.8E+05 | 1.2E−03 | 3.2E−09 |
| | (RTQ) scDb-i/55-38-D07-sc02(RTQ)-scFv | | | |
| PRO2668 | 19-04-A10-sc06(RTQ) scDb-o/28-21-D09-sc04 | 1.1E+06 ± | 1.5E−03 ± | 1.4E−09 ± |
| | (RTQ) scDb-i/55-39-G02-sc05(RTQ)-scFv | 3.9E+05 | 2.0E−04 | 3.6E−10 |

5.2. Cell Binding of scMATCH3 Molecules to Human ROR1 by Flow Cytometry

Cell binding of anti-ROR1 scMATCH3 molecules to ROR1-expressing MDA-MB-231 cells by flow cytometry was performed as described in section 1.6. In brief, 5-fold serial dilutions of scMATCH3 molecules starting at 40 nM (PRO2286, PRO2287) or 120 nM (PRO2507, PR02508, PR02509, PRO2510, PR02557) as well as of reference Fab fragment PRO2213 starting at a concentration of 40 nM or 120 nM were prepared and then added to the plates with cells. After incubation at 4° C. for 1 h in presence of 1 mg/mi of human serum albumin (hSA), plates were washed and incubated with specific detection antibodies. Fluorescence intensity of APC channel was recorded for each sample using NovoCyte 2060 flow cytometer and the geometric mean of fluorescence intensity MFI was calculated.

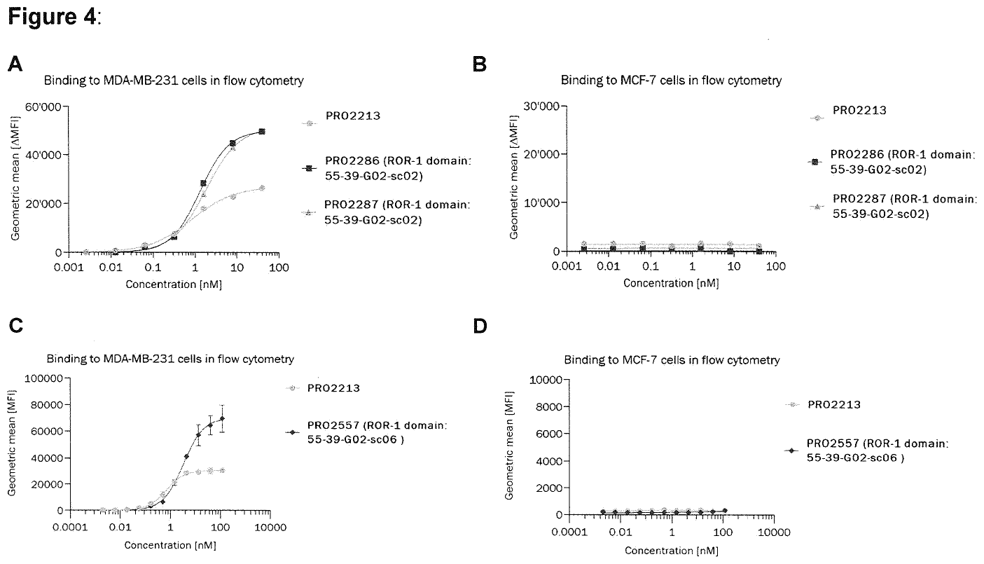

The binding of anti-ROR1xhSAxCD3 scMATCH3 molecules to plasma membrane human ROR1 was assessed by flow cytometry. The calculated EC₅₀, rel. EC₅₀ as well as rel, maximum binding values obtained in these experiments are shown Table 27. As shown in FIG. 4, scMATCH3 molecules PRO2286 and PRO2287, both molecules harboring the same anti-ROR1-BD 55-39-G02-sc02 but different anti-hSA-BD, demonstrated binding to ROR1-positive MDA-MB-231 cells while no binding was found to ROR1-negative MCF-7 cells. Compared to the rel. EC₅₀ values of the parental scFv PRO2062 (anti-ROR1 domain 55-39-G02-sc02), both scMATCH3 molecules showed an around five times lower EC₅₀“binding (rel. EC₅₀ of PRO2286=0.63, rel. EC₅₀ of PRO2287=0.47; mean rel. EC₅₀ of PR02062=2.77). It is assumed that this reduced EC₅₀ binding of scMATCH3 molecules is due to the steric hindrance caused by hSA bound to the scMATCH3 molecules. Along that line, other scMATCH3 molecules harboring either the anti-ROR1 domain 55-39-G02-scO2 (PR02509) or optimized variants of it (PRO2510: 55-39-G02-sc05, PRO2557: 55-39-G02-sc06) showed EC₅₀ binding reduced by five- to ten-fold when compared to their respective scFv. In detail, the rel. EC₅₀ values of PRO2509, PRO2510 and PR02557 were 0.53 (2.77 for corresponding scFv, PR02062), 0.3 (2.61 for PRO2292), and 0.21, respectively.

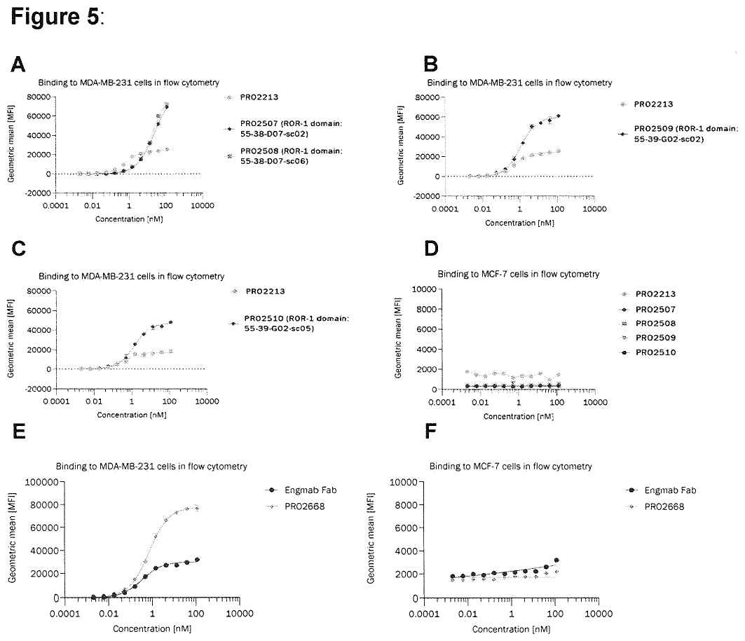

An optimized variant of PR02510, PR02668, was measured in flow cytometry using the same experimental setup. In this experiment, PRO2668 showed a binding to MDA-MB-231 cells comparable to PRO2510 as indicated by a similar binding potency as well as rel, maximum binding value. Dose-response curves of these anti-ROR1xhSAxCD3 scMATCH3 molecules can be found in FIG. 4 and FIG. 5. Regarding the scMATCH3 molecules built with the anti-ROR1 domain 55-38-D07-sc02 (PRO2507) and 55-38-D07-sc06 (PR02508), their EC₅₀ binding were found to be reduced as well when compared to their respective scFv. In detail, the rel. EC₅₀ values of PRO2507 and PRO2508 were 0.03 (0.33 for corresponding scFv, PRO2060) and 0.04 (0.27 for PRO2291), respectively. Concentration-response curves of these anti-ROR1xhSAxCD3 scMATCH3 molecules can be found in FIG. 5.

In summary, all scMATCH3 molecules demonstrated specific binding to human ROR1 expressing cells and a higher EC₅₀ binding was found for the molecules harboring the anti-ROR1 clone 55-39-G02 when compared to the series of molecules built with the anti-ROR1 clone 55-38-D07. Probably due to steric hindrance caused by hSA bound to the scMATCH3 molecules, a five to ten times lower binding was found for scMATCH3 molecules when compared to their corresponding scFv. The results of the cell binding data are summarized in Table 27. Calculated EC₅₀, rel. EC₅₀ as well as rel. maximum binding values are shown. The Fab fragment PRO2213 was used as reference anti-ROR1 antibody.

TABLE 27
Binding of anti-ROR1xhSAxCD3 scMATCH3 molecules PRO2286, PRO2287, PRO2507, PRO2508, PRO2509,
PRO2510, PRO2557 and PRO2668 to human ROR1 expressed on MDA-MB-231 cells by flow cytometry.

| | | Domain 1, | Domain 2, | Domain 3, | | | Rel. maximum |
|---|---|---|---|---|---|---|---|
| | | scDb-o | scDb-i | scFv | | rel. EC₅₀ | binding |
| | | position | position | position | EC₅₀ | (EC_{50, PRO2213}/ | (MFI_{scMATCH3}/ |
| Protein ID | Format | (hSA) | (CD3) | (ROR1) | [nM] | EC_{50, scMATCH3}) | MFIP_{RO2213}) |
| PRO2286 | scMATCH3 | 23-13-A01-sc02 diS | 28-21-D09-sc04 | 55-39-G02-sc02 | 1.33 | 0.63 | 1.87 |
| PRO2287 | scMATCH3 | 19-01-H04-sc02 diS | 28-21-D09-sc04 | 55-39-G02-sc02 | 1.83 | 0.46 | 1.98 |
| PRO2507 | scMATCH3 | 19-04-A10-sc06 | 28-21-D09-sc04 | 55-38-D07-sc02 | 26.86 | 0.03 | 2.88 |
| PRO2508 | scMATCH3 | 19-04-A10-sc06 | 28-21-D09-sc04 | 55-38-D07-sc06 | 18.74 | 0.04 | 3.00 |
| PRO2509 | scMATCH3 | 19-04-A10-sc06 | 28-21-D09-sc04 | 55-39-G02-sc02 | 1.12 | 0.53 | 2.57 |
| PRO2510 | scMATCH3 | 19-04-A10-sc06 | 28-21-D09-sc04 | 55-39-G02-sc05 | 1.36 | 0.30 | 2.61 |
| PRO2557 | scMATCH3 | 19-04-A10-sc06 | 28-21-D09-sc04 | 55-39-G02-sc06 | 3.27 | 0.21 | 2.43 |
| PRO2668 | scMATCH3 | 19-04-A10-sc06 | 28-21-D09-sc04 | 55-39-G02-sc05 | 0.74 | 0.52 | 2.89 |

### Example 6: Potency Assessment: Target Cell Cytotoxicity and T Cell Activation Mediated by scMATCH3 Molecules

In order to understand the capacity of these scMATCH3 molecules for promoting tumor cell killing and CD8 T cell activation, we performed a series of cytotoxicity assays to compare the potencies and T cell activation profiles between our lead candidates.

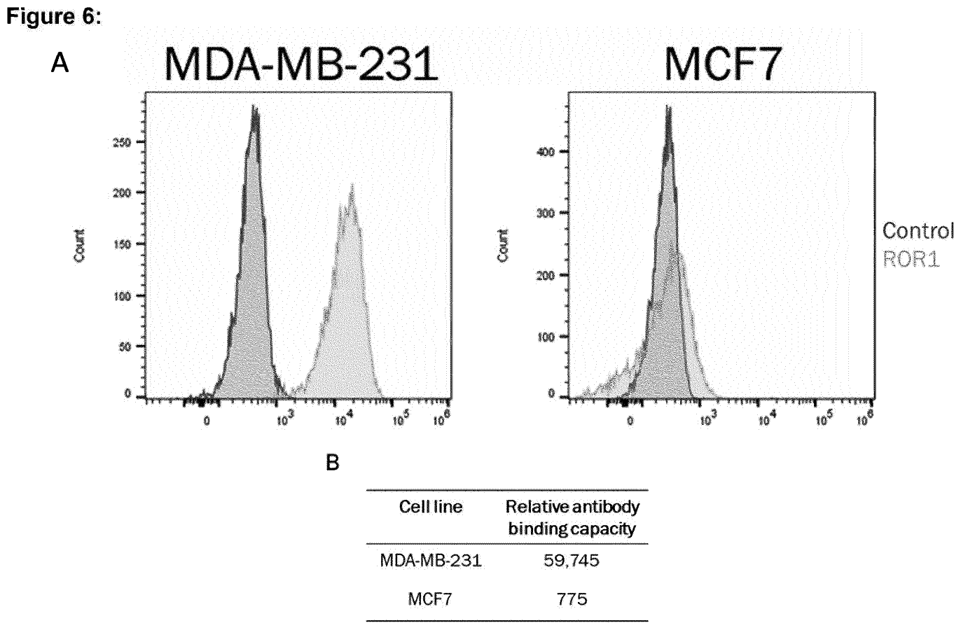

First, the expression levels of ROR1 on several cell lines were tested, together with a quantification of the relative receptor levels on the surface (FIG. 6). Cells were incubated sequentially with Zombie NIR live/dead APC-Cy7 (Biolegend, 1:500) at 4° C. for 20 minutes, followed by a washing step, and then the anti-ROR1 PE (Biolegend, 1:25) at 4° C. for 20 minutes, followed by washing and acquisition on a Novocyte flow cytometer (ACEA). FIG. 6A demonstrates the surface ROR1 expression on MDA-MB-231 and MCF7 cells. Receptor levels were quantified using the Quantum Simply Cellular anti-mouse IgG kit (Bangs Laboratories), following manufacturer's instructions. The beads and cells were acquired on an Attune NxT flow cytometer (ThermoFisher), and data analyzed using FlowJo software (BD). Relative surface levels of ROR1 were higher on MDA-MB-231 cells compared to MCF7 cells. The surface levels of ROR1 on MCF7 cells were considered to be very low, comparable to healthy adult tissue in humans. MCF7 cells were thus used as negative control (FIG. 6B).

These assays were performed using peripheral blood mononuclear cells (PBMCs) isolated from buffy coats using SepMate tubes with Lymphoprep (StemCell Technologies) to create a density gradient following manufacturer's instructions.

Isolated PBMCs were co-cultured for 40 hours with ROR1-positive and negative tumor cells of interest at an effector-to-target ratio of 10:1. Following the co-culture, 100 μl of supernatant was aspirated to test for lactate dehydrogenase (LDH) release from dying cells using a cytotoxicity detection kit from Roche. The remaining supernatant was frozen at −80° C. for downstream multiplexed cytokine analysis. The cells were labeled with the following reagents or antibodies (all from Biolegend) for flow cytometry analysis of specific T cell activation: Fixable live/dead Aqua, anti-human CD4 APC-Cy7 (done OKT4), anti-human CD11c PE-Cy7 (clone Bu15), anti-human CD8 PerCP-Cy5.5 (clone SKI), and anti-human CD69 PE (done FN50). The cells were acquired on a flow cytometer (Attune, ThermoFisher Scientific), and analyzed using FlowJo software (BD) for the upregulation of CD69 as a measure of both CD4 and CD8 T cell activation.

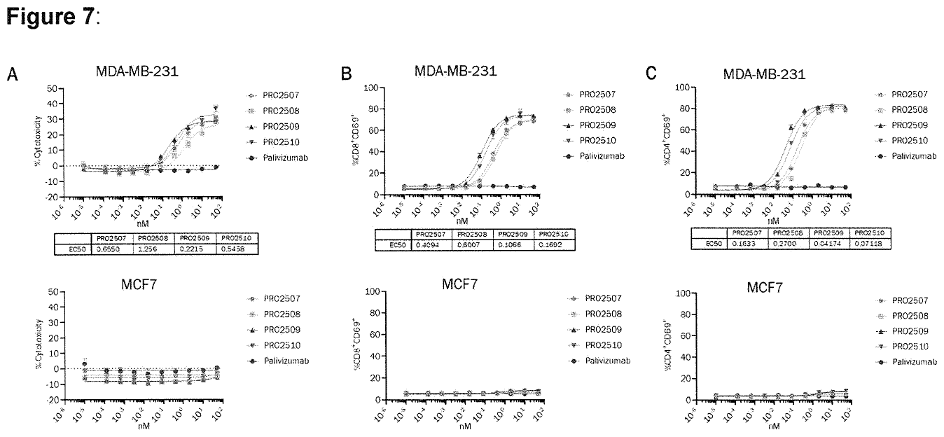

All scMATCH3 molecules tested resulted in the specific killing of ROR1-positive cells such as MDA-MB-231, and minimal to no activity was observed on the ROR1-negative cell line MCF7 (FIG. 7A). PRO2060 derived scMATCH3 molecules PRO2507 and PRO2508 exhibited slightly inferior potencies as compared to the PRO2062-derived molecules PR02509 and PR02510, which was in line with the higher observed affinity for PR02062. CD8 and CD4 T cell activation tracked very closely with the cytotoxicity data (FIGS. 7B and 7C).

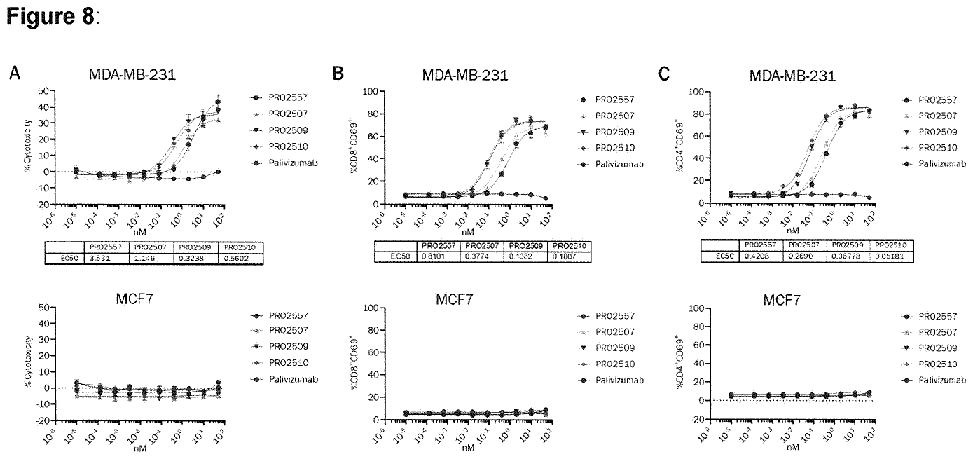

The scMATCH3 molecule PR02557, whose domain was derived from PRO2062 and T cell score optimized in order to reduce immunogenicity, also mediated specific killing of ROR1-positive cells (FIG. 8A). Much like the other scMATCH3 molecules, the CD8 and CD4 T cell activation correlated with the observed cytotoxicity (FIG. 8B and FIG. 8C, respectively). Table 28 summarizes the average EC₅₀ values obtained for specific killing of MDA-MB-231 cells for scMATCH3 molecules across experiments.

TABLE 28
Summary of key cytotoxicity data from scMATCH3 studies
performed with PBMCs

| | | | # exp. | EC₅₀ (nM) Cytotoxicity |
|---|---|---|---|---|
| PRO ID | Format | ROR1 domain | (n) | (MDA-MB-231) |
| PRO2507 | scMATCH3 | PRO2060 | 4 | 1.0 |
| PRO2508 | scMATCH3 | PRO2060 + diS | 2 | 1.7 |
| PRO2509 | scMATCH3 | PRO2062 | 4 | 0.31 |
| PRO2510 | scMATCH3 | PRO2062 + diS | 4 | 0.43 |
| PRO2557 | scMATCH3 | PRO2271 | 2 | 3.1 |
| | | (PRO2062 with | | |
| | | T cell score | | |
| | | modification) | | |

### Example 7: Biophysical Characterization scMATCH3 Molecules

ScMATCH3 molecules were subjected to a four-week stability study, in which the molecules were formulated in aqueous buffer (50 mM NaCiP, 300 mM sucrose, pH 6.5) at 1 mg/ml and stored at <−80° C., 4° C. and 40° C. for four weeks. The fraction of monomers and oligomers in the formulation were evaluated by integration of SE-HPLC peak areas at different time points over the course of the study. Table 29 summarizes monomeric content in % and % monomer loss relative to d0. Changes in protein concentration were monitored by UV-Vis measurement at 280 nm over the course of the study and are shown in Table 30. Thermal stability was analyzed by nDSF (NanoTemper) determining the onset of unfolding (Tₒₙₛₑₜ) and midpoint of unfolding (Tₘ). Nano DSF midpoint of unfolding (Tₘ) results are shown in the last columns of Table 29.

TABLE 29
ScMATCH3 stability assessment at a concentration of 1 mg/ml, change of
monomeric content over time.

| | | | | | | | % monomeric | | | |
|---|---|---|---|---|---|---|---|---|---|---|
| Protein | Temp. | Conc. | monomeric content [%] | | | | content loss | | | Tm |
| ID | [° C.] | [mg/ml] | do | d7 | d14 | d28 | d7 | d14 | d28 | [° C.] |
| PRO2507 | −80 | 1.05 | 98.5 | NA | NA | 98.8* | NA | NA | −0.4* | 63.7 |
| | 4.0 | | 98.5 | 98.6 | 98.8 | 98.8 | −0.1 | 0.4 | −0.4 | |
| | 40.0 | | 98.5 | 98.0 | 97.7 | 96 | 0.5 | 0.8 | 2.5 | |
| PRO2508 | 80 | 1.02 | 98.4 | NA | NA | 98.8* | NA | NA | −0.4* | 65.1 |
| | 4.0 | | 98.4 | 98.6 | 98.7 | 98.7 | −0.2 | −0.3 | −0.3 | |
| | 40.0 | | 98.4 | 98.0 | 97.7 | 96.2 | 0.5 | 0.7 | 2.3 | |
| PRO2509 | −80 | 1.03 | 98.4 | NA | NA | 98.7* | NA | NA | −0.3* | 61.4 |
| | 4.0 | | 98.4 | 98.5 | 98.7 | 98.5 | 0.0 | −0.3 | −0.1 | |
| | 40.0 | | 98.4 | 97.5 | 97.2 | 95.1 | 0.9 | 1.2 | 3.4 | |
| PRO2510 | −80 | 1.04 | 98.2 | NA | NA | 98.5* | NA | NA | −0.3* | 63.8 |
| | 4.0 | | 98.2 | 98.4 | 98.6 | 98.5 | −0.2 | −0.4 | −0.2 | |
| | 40.0 | | 98.2 | 97.8 | 97.7 | 96.1 | 0.5 | 0.5 | 2.2 | |

*sample was subjected to 5 repeated freeze/thaw cycles before measurement
NA: not measured

TABLE 30
ScMATCH3 stability assessment at a concentration of 1 mg/ml,
change of protein content over time.

| | | | Protein concentration | | | | | | |
|---|---|---|---|---|---|---|---|---|---|
| | Temp. | Conc. | [mg/ml] | | | | % content loss | | |
| Protein ID | [° C.] | [mg/mL] | do | d7 | d14 | d28 | d7 | d14 | d28 |
| PRO2507 | −80 | 1.1 | 1.05 | NA | NA | 1.02* | NA | NA | 3.2* |
| | 4.0 | | 1.05 | 1.12 | 1.07 | 1.01 | 6.4 | 1.7 | 3.8 |
| | 40.0 | | 1.05 | 1.11 | 1.07 | 1.02 | −5.3 | −1.7 | 3.6 |
| PRO2508 | −80 | 1.0 | 1.02 | NA | NA | 0.96* | NA | NA | 6.1* |
| | 4.0 | | 1.02 | 1.10 | 0.98 | 0.94 | 7.5 | 3.9 | 8.0 |
| | 40.0 | | 1.02 | 1.06 | 1.02 | 0.97 | 4.0 | 0.3 | 4.8 |
| PRO2509 | −80 | 1.0 | 1.03 | NA | NA | 0.94 | NA | NA | 8.5* |
| | 4.0 | | 1.03 | 1.05 | 0.98 | 0.92 | −2.3 | 4.5 | 10.3 |
| | 40.0 | | 1.03 | 1.03 | 1.00 | 1.09 | −0.6 | 3.0 | −6.5 |
| PRO2510 | −80 | 1.0 | 1.04 | NA | NA | 1.05* | NA | NA | −0.6* |
| | 4.0 | | 1.04 | 1.00 | 0.97 | 1.03 | 3.4 | 6.5 | 0.5 |
| | 40.0 | | 1.04 | 0.99 | 0.93 | 1.03 | 5.0 | 10.2 | 1.0 |

*sample was subjected to 5 repeated freeze/thaw cycles before measurement
NA: not measured

Molecules containing 55-38-D07-derived anti-ROR1 domains (PR02507 and PRO2508) and molecules containing 55-39-G02-derived anti-ROR1 domains (PR02509 and PR02510) exhibit very similar monomer stability over the course of 28d at a concentration of 1 mg/ml. There is no notable change in monomeric content at temperatures of −80° C. and 4° C. as well as upon repeated freeze-thawing (5×) as performed with the d28/−80° C. before SE-HPLC/UV measurement.

SDS-PAGE analysis of day 0 (d0) and day 28 (d28) samples revealed, beside a main band corresponding to the calculated molecular weight of the molecules, the existence of low molecular weight (LMW) species in all (reduced) samples at d0 of the study. The reason for their occurrence is not entirely dear, may however be related to the generic, non-optimized expression and purification conditions used for the manufacture of the molecules or unfavorable SDS-PAGE sample preparation conditions. When comparing d28 samples to d0 samples, generally no notable increase of LWM species in 4° C. and 40° C. samples could be detected over time. In all samples incubated for 28 days under accelerated conditions (40° C.) the fraction of LMW species is clearly increased, indicating time- and temperature-dependent fragmentation of scMATCH3 molecules. As all molecules exhibit similarly favorable stability profiles, deselection of molecules based on accelerated stability data is not envisaged.

PRO2509 and PR02510 seem to be slightly better than PRO2507 and PR02508 with respect to thermal stability as assessed by nDSF as their onset of unfolding (Tₒₙₛₑₜ) is considerably higher for the molecules containing the 55-39-G02-derived ROR1 domain.

### Example 8: Generation and Production of MATCH4 Anti-ROR1xCD3xhSA Multispecific Antibodies

The ROR1“binding domains from the scMATCH3 molecules were incorporated into a MATCH4 molecule format to further enhance potency through the use of two ROR1“binding domains. This would result in bivalent targeting similar to conventional full-length antibodies, but with the additional benefit of T cell targeting (CD3 domain) and half-life extension (hSA domain).

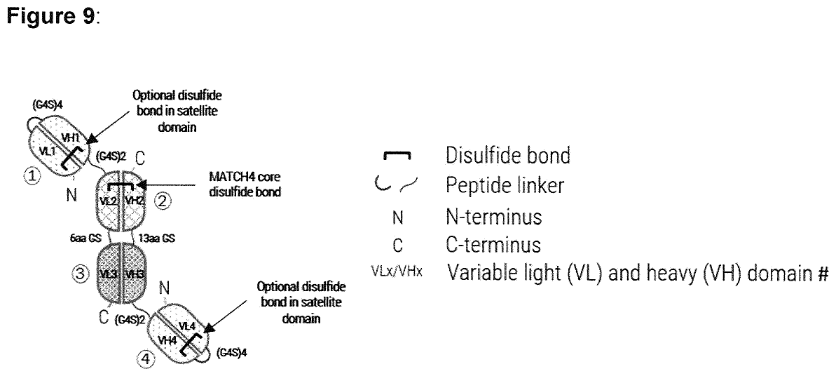

The MATCH4 is a format developed by Numab that consists solely of variable domains connected by GS-4inkers of different length that allow for the specific pairing of matching domain pairs only. The MATCH4 format can be expressed recombinantly from mammalian cells. For the purification, a conventional affinity chromatography step can be used. The architecture of ND032 MATCH4 molecules is depicted in FIG. 9. This format requires that the dimer subunits consist of a core of two split variable domain pairs, each respective subunit possessing either two VL domains or two VH domains positioned in tandem, thereby driving heterodimerization of the two protein chains. The dimer-forming tandem variable domains on the respective MATCH4 chains are organized in anti-parallel N-term-C-term orientation as their counterpart chain. Both chains are co-expressed in mammalian cells into fully functional tetra-specific molecules. Traditional Gly-Ser linkers between the variable domains were used to connect them. Further, the antiparallel MATCH4 format is amenable to the introduction of a disulfide bridge in one of the core domains as indicated in FIG. 9. Additional VL/VH disulfide bonds may be contained for stabilization of individual domains.

Expression of mammalian MATCH4 constructs has been performed at 1 l scale at Evitria AG (Schlieren, Switzerland) using their proprietary mammalian expression system. Proteins were purified from clarified culture supernatants by Protein L affinity chromatography followed by SEC in 50 mM phosphate-citrate buffer with 300 mM sucrose at pH 6.5. Monomeric content of SEC fractions was assessed by SE-HPLC analysis and fractions with a monomeric content >95% were pooled. For quality control of the manufactured material, standard analytical methods such as SE-HPLC, UV₂₈₀ and SDS-PAGE were applied. Molecule composition and a manufacture summary of MATCH4 molecules are shown in Table 31. Manufacture summary of the MATCH4 molecules PR02669 and RPO2670, which comprise modified or improved ROR1-, CD3- and hSA binding domains, are shown in Table 32.

TABLE 31
MATCH4 molecule architecture, final titer and monomeric purity by SE−HPLC.

| | | | | | | Final | Monomeric |
|---|---|---|---|---|---|---|---|
| | | | | | Titer | titer | content |
| | Domain 1 | Domain 2 | Domain 3 | Domain 4 | [mg/l | [mg/l | [%] |
| PRO ID | (ROR1) | (SA) | (CD3) | (ROR1) | culture] | culture] | (SEC1/SEC2) |
| PRO2589 | 55-38- | 19-04-A10-sc02 diS | 28-21-D09- | 55-38-D07- | 129.3 | 28.8 | 95.2/96.4* |
| | D07-sc02 | (19-04-A10-sc06) | sc04 | sc02 | | | |
| PRO2590 | 55-38- | 19-04-A10-sc02 diS | 28-21-D09- | 55-38-D07- | 53.4 | 23.0 | 98.3/96.6* |
| | D07-sc06 | (19-04-A10-sc06) | sc04 | sc06 | | | |
| PRO2591 | 55-39- | 19-04-A10-sc02 diS | 28-21-D09- | 55-39-G02- | 107.4 | 54.8 | 89.0/96.4* |
| | G02-sc03 | (19-04-A10-sc06) | sc04 | sc03 | | | |
| PRO2592 | 55-39- | 19-04-A10-sc02 diS | 28-21-D09- | 55-39-G02- | 125.8 | 41.7 | 95.1/NA |
| | G02-sc06 | (19-04-A10-sc06) | sc04 | sc06 | | | |

*Two size exclusion chromatography (SEC) polishing runs have been performed of which material has not been pooled, material with higher monomeric content was used for further characterization of molecules

TABLE 32
Manufacture of MATCH4 molecules PRO2669 and RPO2670

| | | | | Purity by | | |
|---|---|---|---|---|---|---|
| | | Capture | | SE-HPLC | nDSF. | nDSF |
| | | titer | Titer | [% | T_{M1} | T_{M2} |
| Protein ID | Format | [mg/l] | [mg/l] | monomer] | [° C.] | [° C.] |
| PRO2669 | MATCH4 | 99.1 | 15.4 | 98.5 | 63.1 | 71.2 |
| PRO2670 | MATCH4 | 50.2 | 21.0 | 98.7 | 61.5 | 76.2 |

### Example 9: Pharmacodynamic Characterization of MATCH4 Molecules

### 9.1. Binding Affinity by SPR

The binding kinetics for four multispecific MATCH4 molecules to the three targets hROR1, hCD3ε and hSA were tested by SPR.

Binding Affinity to hROR1 by SPR

Binding kinetics (including affinity) with and without avidity of the four sMATCH4 molecules to recombinant human ROR1 protein were determined by SPR analysis on a T200 device (Biacore, Cytiva) with two different assay designs.

Binding affinity without avidity was determined by using a proprietary anti-framework rabbit IgG (rlgG21-57-20-B11) immobilized to a carboxymethylated dextran surface (CM5 sensorchip; Biacore, Cytiva) to capture the MATCH4 molecules on the surface. A titration series of human ROR1 (Peprotech 160-054) molecule was injected as analyte. After each capture-analyte injection cycle, every flow channel on the sensor chip was regenerated (3 M MgCl₂), and a MATCH4 molecule was captured again followed by a different concentration of human ROR1. The binding kinetics to human ROR1 were measured using a multi-cycle kinetic assay, with ten analyte concentrations ranging from 0.088 to 90 nM, 1:2 diluted in running buffer (HEPES buffered saline, 0.05% Tween-20, pH 7.5; Bioconcept).

Binding with avidity was determined similarly to the procedures described in section 1.6. Recombinant human ROR1 molecules (Fc-Tag, Acro Biosystems RO1-H5250) were covalently immobilized to a carboxymethylated dextran surface (CM5 sensorchip; Biacore, Cytiva) and a titration series of each MATCH4 molecule was injected as analyte. After each analyte injection-cycle, every flow channel on the sensor chip was regenerated (Glycine, pH 2.0 and 3 M MgCl₂), and a new concentration of MATCH4 molecule was injected. The binding kinetics to human ROR1 were measured using a multi-cycle kinetic assay, with nine analyte concentrations ranging from 0.088 to 45 nM, 1:2 diluted in running buffer (HEPES buffered saline, 0.05% Tween-20, pH 7.5; Bioconcept).

For both assays, the apparent dissociation (k_{d}) and association (kₐ) rate constants and the apparent dissociation equilibrium constant (K_{D}) were calculated with the Biacore analysis software (Biacore Evaluation software Version 3.2, Cytiva) using a one-to-one Langmuir binding model and quality of the fits was monitored based on Chi2 and U-value, which are measures for the quality of the curve fitting. The binding level was calculated as the maximum stability binding achieved normalized to the theoretical Rmax.

Binding kinetics to hROR1 were determined for four MATCH4 with affinities ranging from 5 nM to 1.2 nM. hROR1 affinities with avidity were determined on the four MATCH4 molecules and ranged from 136 pM to 22 pM. The SPR results are summarized in Table 33.

Binding Affinity to hCD3ε by SPR

Binding kinetics (including affinity) of the four MATCH4 molecules to recombinant human CD3 epsilon extra-cellular domain protein (hCD3ε; His-Tag, SinoBiological) were determined by SPR analysis on a T200 device (Biacore, Cytiva). Recombinant hCD3ε molecules were covalently immobilized to a carboxymethylated dextran surface (CM5 sensorchip; Biacore, Cytiva) and a titration series of each MATCH4 molecule was injected as analyte. After each analyte injection-cycle, every flow channel on the sensor chip was regenerated (Glycine, pH 2.0 and 3 M MgCl₂), and a new concentration of MATCH4 molecule was injected. The binding kinetics to hCD3ε were measured using a multi-cycle kinetic assay, with nine analyte concentrations ranging from 0.088 to 45 nM, 1:2 diluted in running buffer (HEPES buffered saline, 0.05% Tween-20, pH 7.5; Bioconcept). The apparent dissociation (k_{d1}) and association (kₐ₁) rate constants, the second reaction constants (kₐ₂ and k_{d2}) and the apparent dissociation equilibrium constant (K_{D}) were calculated with the Biacore analysis software (Biacore Evaluation software Version 3.2, Cytiva) using a two-state binding model, and the quality of the fits was monitored based on relative Chi2. The binding level was calculated as the maximum stability binding achieved normalized to the theoretical Rmax.

Binding kinetics to hCD3ε were determined for four MATCH4 with affinities ranging from 6.3 nM to 4.7 nM (Table 34). hCD3ε affinity for all tested MATCH4 molecules was similar to the parental scFv 28-21-D09-sc04.

Binding affinity to hSA by SPR

Binding kinetics (including affinity) of the four MATCH4 molecules to human serum albumin (hSA; Sigma-Aldrich A3782) were determined by SPR analysis on a T200 device (Biacore, Cytiva). Human serum albumin molecules were covalently immobilized to a carboxymethylated dextran surface (CM5 sensorchip; Biacore, Cytiva) and a titration series of each MATCH4 molecule was injected as analyte. After each analyte injection-cycle, every flow channel on the sensor chip was regenerated (pulses of Glycine, pH 1.5 and 3 M MgCl₂), and a new concentration of MATCH4 molecule was injected. The binding kinetics to hSA were measured using a multi-cycle kinetic assay, with ten analyte concentrations ranging from 0.18 to 90 nM, 1:2 diluted in running buffer (PBS-P+pH 5.5, PBS to pH 5.5 with HCl, 0.05% Tween20). The apparent dissociation (k_{d}) and association (kₐ) rate constants and the apparent dissociation equilibrium constant (K_{D}) were calculated with the Biacore analysis software (Biacore Evaluation software Version 3.2, Cytiva) using a one-to-one Langmuir binding model, and quality of the fits was monitored based on Chi2 and U-value, which are measures for the quality of the curve fitting. The binding level was calculated as the maximum stability binding achieved normalized to the theoretical Rmax.

Binding kinetics to hSA were determined for four MATCH4 with affinities ranging from 6.7 nM to 5.5 nM (Table 35). hSA affinity for all tested MATCH4 molecules was similar to the parental scFv 19-04-A10-sc02.

### 9.2. Cell Binding of MATCH4 Molecules to Human ROR1 by Flow Cytometry

Cell binding of anti-ROR1 MATCH4 molecules (PR02589, PRO2590, PR02591, PR02592, and PRO2658) to ROR1-expressing MDA-MB-231 cells by flow cytometry was performed as described above. In brief, 5-fold serial dilutions of MATCH4 molecules starting at 120 nM as well as of reference Fab fragment PRO2213 starting at a concentration of 120 nM were prepared and then added to the plates with cells. After incubation at 4° C. for 1 h in presence of 1 mg/ml of human serum albumin (hSA), plates were washed and incubated with specific detection antibodies. Fluorescence intensity of the APC channel was recorded for each sample using NovoCyte 2060 flow cytometer and the geometric mean of fluorescence intensity MFI was calculated.

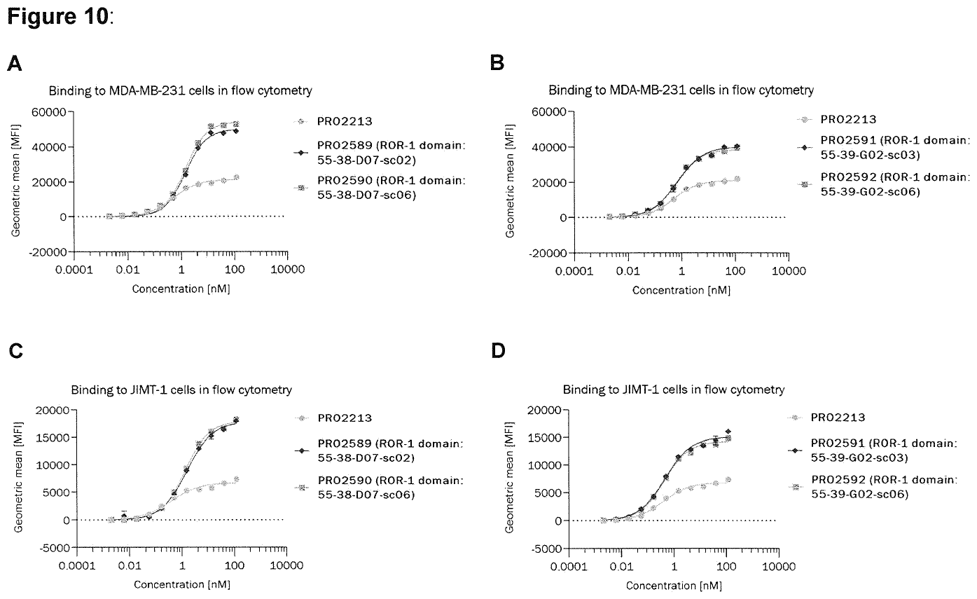

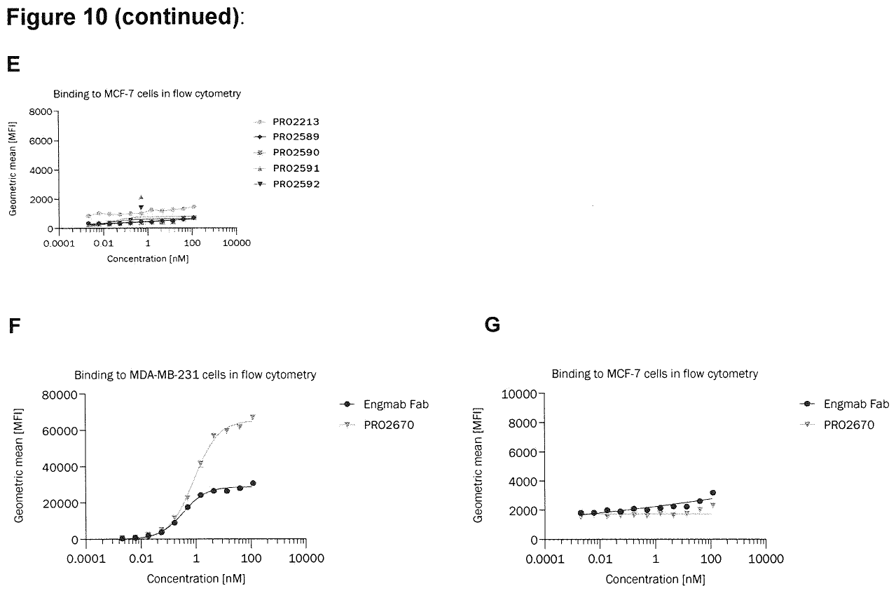

The binding of MATCH4 molecules to plasma membrane-based human ROR1 was assessed by flow cytometry. The calculated EC₅₀, rel. EC₅₀ as well as rel. maximum binding values obtained in these experiments are shown Table 36. The Fab fragment PRO2213 was used as reference anti-ROR1 antibody. All MATCH4 molecules demonstrated binding to ROR1 positive MDA-MB-231 cells while no binding was found to ROR1-negative MCF-7 cells (see FIG. 10). The EC₅₀ binding of the MATCH4 molecules harboring the ROR1 clone 55-38-D07 (PR02589 and PR02590) increased by a factor of 10 when compared to the EC₅₀ of the corresponding scMATCH3 molecules. For instance, the rel. EC₅₀ of PRO2589 is 0.45, which is roughly ten times better than the rel. EC₅₀ of the scMATCH3 PRO2507 (0.03). Similarly, the EC₅₀ binding of the MATCH4 molecules harboring ROR1 clone 55-39-G02 (PRO2591 and PR02592) increased by factor of 5 (compare rel. EC₅₀ of PRO2592 with the rel. EC₅₀ of PR02557). It is believed that the increase in binding is due to the bivalent nature of MATCH4 molecules which results in avidity effects. Among the MATCH4 molecules, PR02591 (ROR1 domain: 55-39-G02-sc03) showed the best EC₅₀ binding, which is roughly 2.5 times better than reference Fab fragment PRO2213. An optimized variant of PR02590, PRO2670, was measured in flow cytometry using the same experimental setup. In this experiment, we found that PR02670 binds to MDA-MB-231 cells with a potency comparable to PRO2590 indicated by similar rel. EC₅₀ value.

In summary, all MATCH4 molecules demonstrated specific binding to human ROR1-expressing cells and a higher binding was found for the molecules harboring the anti-ROR1 clone 55-39-G02 when compared to the series of molecules built with the anti-ROR1 clone 55-38-D07. Probably due to avidity effects, all bivalent MATCH4 molecules demonstrated an increased EC₅₀ binding when compared to corresponding monovalent scMATCH3 molecules.

TABLE 33
Summary of affinity measurement to hROR1 for MATCH4 molecules with and without avidity.

| Protein | | Affinity/ | | | |
|---|---|---|---|---|---|
| #ID | Protein description | Avidity | kₐ (1/Ms) | k_{d} (1/s) | K_{D} (M) |
| PRO2589 | 55-38-D07-sc02 scFv (G4S)2 19-04-A10-sc02 diS_(G2S)2_28-21-D09-sc04 | Affinity | 9.4E+04 | 1.1E−04 | 1.2E−09 |
| | VL/55-38-D07-sc02_(G4S)2_28-21-D09-sc04 _(G3S)3G_19-04-A10-sc02 diS VH | | | | |
| PRO2590 | 55-38-D07-sc02 diS scFv (G4S)2 19-04-A10-sc02 diS_(G2S)2_28-21-D09-sc04 | Affinity | 5.9E+04 | 1.7E−04 | 2.9E−09 |
| | VL/55-38-D07-sc06_(G4S)2_28-21-D09-sc04 _(G3S)3G_19-04-A10-sc02 diS VH | | | | |
| PRO2591 | 55-39-G02-sc03_(G4S)2_19-04-A10-sc02 diS_(G2S)2_28-21-D09-sc04 VL/55- | Affinity | 1.1E+05 | 3.4E−04 | 3.2E−09 |
| | 39-G02-sc03_(G4S)2_28-21-D09-sc04 _(G3S)3G_19-04-A10-sc02 diS VH | | | | |
| PRO2592 | 55-39-G02-sc06_(G4S)2_19-04-A10-sc02 diS_(G2S)2_28-21-D09-sc04 VL/55- | Affinity | 9.8E+04 | 4.9E−04 | 5.0E−09 |
| | 39-G02-sc06_(G4S)2_28-21-D09-sc04 _(G3S)3G_19-04-A10-sc02 diS VH | | | | |
| PRO2669 | 55-38-D07-sc02(RTQ) scFv (G4S)2 19-04-A10-sc02(RTQ) diS_(G2S)2_28-21- | Affinity | 5.66E+04 | <1.00E−05 | 1.77E−10 |
| | D09-sc04(RTQ) VL/55-38-D07-sc02(RTQ)_(G4S)2_28-21-D09-sc04(RTQ)_ | | | | |
| | (G3S)3G_19-04-A10-sc02(RTQ) diS VH | | | | |
| PRO2670 | 55-38-D07-sc02(RTQ) diS scFv (G4S)2 19-04-A10-sc02(RTQ) diS_(G2S)2_28- | Affinity | 4.60E+04 | 1.32E−04 | 2.87E−09 |
| | 21-D09-sc04(RTQ) VL/55-38-D07-sc06(RTQ)_(G4S)2_28-21-D09-sc04(RTQ)_ | | | | |
| | (G3S)3G_19-04-A10-sc02(RTQ) diS VH | | | | |
| PRO2589 | 55-38-D07-sc02 scFv (G4S)2 19-04-A10-sc02 diS (G2S)2_28-21-D09-sc04 | Avidity | 7.55E+05 | 7.02E−05 | 9.30E−11 |
| | VL/55-38-D07-sc02_(G4S)2_28-21-D09-sc04 _(G3S)3G_19-04-A10-sc02 diS VH | | | | |
| PRO2590 | 55-38-D07-sc02 diS scFv (G4S)2 19-04-A10-sc02 diS_(G2S)2_28-21-D09-sc04 | Avidity | 4.53E+05 | 6.15E−05 | 1.36E−10 |
| | VL/55-38-D07-sc06_(G4S)2_28-21-D09-sc04 _(G3S)3G_19-04-A10-sc02 diS VH | | | | |
| PRO2591 | 55-39-G02-sc03_(G4S)2_19-04-A10-sc02 diS_(G2S)2_28-21-D09-sc04 VL/55- | Avidity | 1.81E+06 | 4.10E−05 | 2.27E−11 |
| | 39-G02-sc03_(G4S)2_28-21-D09-sc04 _(G3S)3G_19-04-A10-sc02 diS VH | | | | |
| PRO2592 | 55-39-G02-sc06_(G4S)2_19-04-A10-sc02 diS_(G2S)2_28-21-D09-sc04 VL/55- | Avidity | 1.98E+06 | 5.69E−05 | 2.87E−11 |
| | 39-G02-sc06_(G4S)2_28-21-D09-sc04 _(G3S)3G_19-04-A10-sc02 diS VH | | | | |
| PRO2669 | 55-38-D07-sc02(RTQ) scFv (G4S)2 19-04-A10-sc02(RTQ) diS_(G2S)2_28-21- | Avidity | 1.0E+06 | 2.6E−05 | 2.6E−11 |
| | D09-sc04(RTQ) VL/55-38-D07-sc02(RTQ)_(G4S)2_28-21-D09-sc04(RTQ)_ | | | | |
| | (G3S)3G_19-04-A10-sc02(RTQ) diS VH | | | | |
| PRO2670 | 55-38-D07-sc02(RTQ) diS scFv (G4S)2 19-04-A10-sc02(RTQ) diS_(G2S)2_28- | Avidity | 7.6E+05 | 8.1E−05 | 1.1E−10 |
| | 21-D09-sc04(RTQ) VL/55-38-D07-sc06(RTQ)_(G4S)2_28-21-D09-sc04(RTQ)_ | | | | |
| | (G3S)3G_19-04-A10-sc02(RTQ) diS VH | | | | |

TABLE 34
Summary of affinity measurement to hCD3E for selected MATCH4 molecules.

| | | Kₐ₁ | k_{d1} | Kₐ₂ | K_{d2} | K_{D} |
|---|---|---|---|---|---|---|
| Protein # | Protein description | (1/Ms) | (1/s) | (1/s) | (1/s) | (M) |
| PRO2589 | 55-38-D07-sc02 scFV (G4S)2 19-04-A10-sc02 diS_(G2S)2_28- | 4.4E+05 | 6.5E−03 | 6.3E−03 | 4.2E−03 | 5.9E−09 |
| | 21-D09-sc04 VL/55-38-D07-sc02_(G4S)2_28-21-D09-sc04_ | | | | | |
| | (G3S)3G_19-04-A10-sc02 diS VH | | | | | |
| PRO2590 | 55-38-D07-sc02 diS scFv (G4S)2 19-04-A10-sc02 | 4.5E+05 | 5.8E−03 | 5.5E−03 | 4.3E−03 | 5.7E−09 |
| | diS_(G2S)2_28-21-D09-sc04 VL/55-38-D07-sc06_(G4S)2_28- | | | | | |
| | 21-D09-sc04_(G3S)3G_19-04-A10-sc02 diS VH | | | | | |
| PRO2591 | 55-39-G02-sc03_(G4S)2_19-04-A10-sc02 diS_(G2S)2_28-21- | 4.2E+05 | 4.23E−03 | 2.7E−03 | 2.4E−03 | 4.7E−09 |
| | D09-sc04 VL/55-39-G02-sc03_(G4S)2_28-21-D09-sc04_ | | | | | |
| | (G3S)3G_19-04-A10-sc02 diS VH | | | | | |
| PRO2592 | 55-39-G02-sc06_(G4S)2_19-04-A10-sc02 diS_(G2S)2_28-21- | 4.3E+05 | 5.5E−03 | 3.6E−03 | 3.6E−03 | 6.3E−09 |
| | D09-sc04 VL/55-39-G02-sc06_(G4S)2_28-21-D09-sc04_ | | | | | |
| | (G3S)3G_19-04-A10-sc02 diS VH | | | | | |
| PRO2669 | 55-38-D07-sc02(RTQ) scFv (G4S)2 19-04-A10-sc02(RTQ) | 8.1E+05 | 4.7E−03 | 5.6E−03 | 4.7E−03 | 2.7E−09 |
| | diS_(G2S)2_28-21-D09-sc04(RTQ) VL/55-38-D07- | | | | | |
| | Sc02(RTQ)_(G4S)2_28-21-D09-sc04(RTQ)_(G3S)3G_19-04- | | | | | |
| | A10-sc02(RTQ) diS VH | | | | | |
| PRO2670 | 55-38-D07-sc02(RTQ) diS scFv (G4S)2 19-04-A10-sc02(RTQ) | 8.2E+05 | 4.8E−03 | 5.4E−03 | 4.7E−03 | 2.7E−09 |
| | diS_(G2S)2_28-21-D09-sc04(RTQ) VL/55-38-D07- | | | | | |
| | Sc06(RTQ)_(G4S)2_28-21-D09-sc04(RTQ)_(G3S)3G_19-04- | | | | | |
| | A10-sc02(RTQ) diS VH | | | | | |

TABLE 35
Summary of affinity measurement to hSA for selected MATCH4 molecules.

| Protein #ID | Protein description | kₐ (1/Ms) | k_{d} (1/s) | K_{D} (M) |
|---|---|---|---|---|
| PRO2589 | 55-38-D07-sc02 scFv (G4S)2 19-04-A10-sc02 diS_(G2S)2_28-21-D09-sc04 VL/55- | 3.3E+05 | 1.8E−03 | 5.5E−09 |
| | 38-D07-sc02_(G4S)2_28-21-D09-sc04 _(G3S)3G_19-04-A10-sc02 diS VH | | | |
| PRO2590 | 55-38-D07-sc02 diS scFv (G4S)2 19-04-A10-sc02 diS_(G2S)2_28-21-D09-sc04 | 2.9E+05 | 2.0E−03 | 6.7E−09 |
| | VL/55-38-D07-sc06_(G4S)2_28-21-D09-sc04 _(G3S)3G_19-04-A10-sc02 diS VH | | | |
| PRO2591 | 55-39-G02-sc03_(G4S)2_19-04-A10-sc02 diS_(G2S)2_28-21-D09-sc04 VL/55-39- | 3.3E+05 | 1.9E−03 | 5.6E−09 |
| | G02-sc03_(G4S)2_28-21-D09-sc04 _(G3S)3G_19-04-A10-sc02 diS VH | | | |
| PRO2592 | 55-39-G02-sc06_(G4S)2_19-04-A10-sc02 diS_(G2S)2_28-21-D09-sc04 VL/55-39- | 3.6E+05 | 2.3E−03 | 6.3E−09 |
| | G02-sc06_(G4S)2_28-21-D09-sc04 _(G3S)3G_19-04-A10-sc02 diS VH | | | |
| PRO2669 | 55-38-D07-sc02(RTQ) scFv (G4S)2 19-04-A10-sc02(RTQ) diS_(G2S)2_28-21- | 4.1E+05 | 1.3E−03 | 3.0E−09 |
| | D09-sc04(RTQ) VL/55-38-D07-sc02(RTQ)_(G4S)2_28-21-D09-sc04(RTQ)_ | | | |
| | (G3S)3G_19-04-A10-sc02(RTQ) diS VH | | | |
| PRO2670 | 55-38-D07-sc02(RTQ) diS scFv (G4S)2 19-04-A10-sc02(RTQ) diS (G2S)2_28-21- | 3.7E+05 | 1.3E−03 | 3.5E−09 |
| | D09-sc04(RTQ) VL/55-38-D07-sc06(RTQ)_(G4S)2_28-21-D09-sc04(RTQ)_ | | | |
| | (G3S)3G_19-04-A10-sc02(RTQ) diS VH | | | |

TABLE 36
Binding of bivalent anti-ROR1 MATCH4 molecules PRO2589, PRO2590, PRO2591, PRO2592 and PRO2670 to human
ROR1 expressed on MDA-MB-231 cells by flow cytometry

| | | | | | | | Rel. |
|---|---|---|---|---|---|---|---|
| | | | | | | | maximum |
| | | | | | | rel. EC₅₀ | binding |
| | Domain 1 | Domain 2 | Domain 3 | Domain 4 | EC₅₀ | EC_{50, PRO2213}/ | (MFI_{MATCH4}/ |
| PRO ID | (ROR1) | (hSA) | (CD3) | (ROR1) | [nM] | EC_{50, MATCH4)} | MFIP_{RO2213}) |
| PRO2589 | 55-38-D07-sc02 | 19-04-A10-sc02 diS | 28-21-D09-sc04 | 55-38-D07-sc02 | 1.55 | 0.45 | 2.67 |
| | | (19-04-A10-sc06) | | | | | |
| PRO2590 | 55-38-D07-sc06 | 19-04-A10-sc02 diS | 28-21-D09-sc04 | 55-38-D07-sc06 | 1.23 | 0.56 | 2.76 |
| | | (19-04-A10-sc06) | | | | | |
| PRO2591 | 55-39-G02-sc03 | 19-04-A10-sc02 diS | 28-21-D09-sc04 | 55-39-G02-sc03 | 0.28 | 2.45 | 2.21 |
| | | (19-04-A10-sc06) | | | | | |
| PRO2592 | 55-39-G02-sc06 | 19-04-A10-sc02 diS | 28-21-D09-sc04 | 55-39-G02-sc06 | 0.72 | 0.94 | 2.41 |
| | | (19-04-A10-sc06) | | | | | |
| PRO2670 | 55-38-D07-sc06 | 19-04-A10-sc02(RTQ) | 28-21-D09-sc04 | 55-38-D07-sc06 | 0.85 | 0.39 | 2.68 |
| | | diS (19-04-A10- | (RTQ) | (RTQ) | | | |
| | | sc06(RTQ) | | | | | |

### 9.3. Target Cell Cytotoxicity and T Cell Activation Mediated by MATCH4 Molecules

A series of cytotoxicity assays was performed to compare the potencies and T cell activation profiles of our lead candidates, in order to understand the capacity of the MATCH4 molecules for promoting tumor cell killing and CD8 T cell activation.

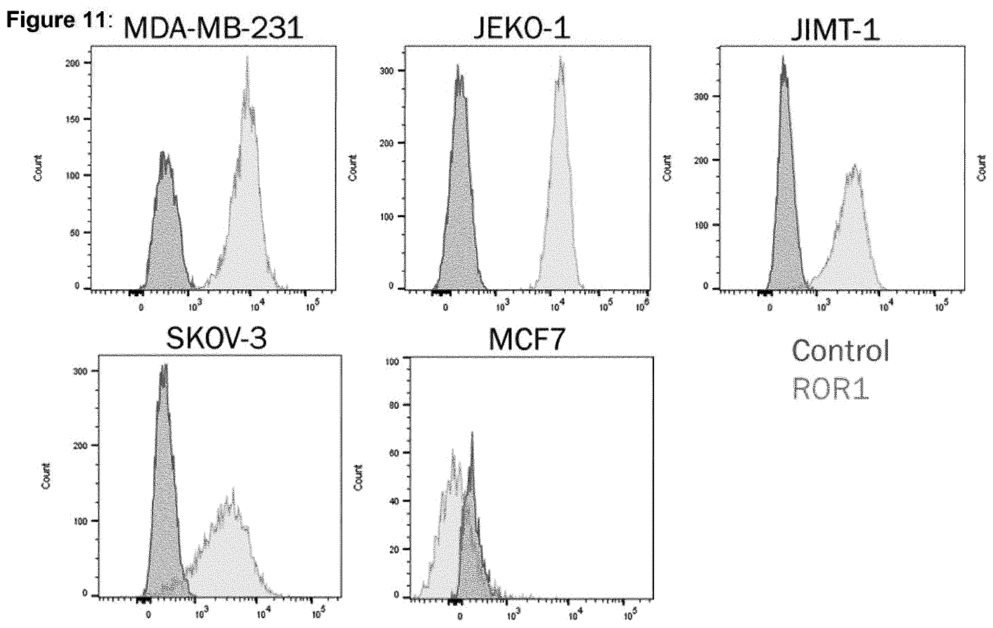

First, the expression levels of ROR1 on several cell lines were tested in parallel. Briefly, cells were incubated sequentially with Zombie NIR live/dead APC-Cy7 (Biolegend, 1:500) at 4° C. for 20 minutes, followed by a washing step, and then the anti-ROR1 PE (Biolegend, 1:25) at 4° C. for 20 minutes, followed by washing and acquisition on an Attune flow cytometer (Thermofisher Scientific). The measured surface ROR1 expression on MDA-MB-231, Jeko-1, JIMT-1, SKOV-3, and MCF7 cells are shown in FIG. 11. The highest ROR1 levels were found on MDA-MB-231 and Jeko-1 cells. JIMT-1 and SKOV-3 cells show low expression levels of ROR1, and MCF7 cells showed a very low ROR1 expression level. MCF7 cells were thus used as negative control for ROR1.

Cytotoxicity assays were performed using Pan-T cells, isolated from peripheral blood mononuclear cells (PBMCs) isolated from buffy coats. PBMCs were isolated using SepMate tubes with Lymphoprep (StemCell Technologies) to create a density gradient following manufacturer's instructions. After PBMC isolation, Pan-T cells were isolated using the Pan-T isolation kit from Miltenyi following manufacturer's instructions.

Isolated Pan-T cells were co-cultured for 40 hours with ROR1-positive and negative tumor cells of interest at an effector-to-target ratio of 10:1. Following the co-culture, 100 μl of supernatant were aspirated to test for lactate dehydrogenase (LDH) released from dying cells using the cytotoxicity detection kit from Roche. The remaining supernatant was frozen at −80° C. for downstream multiplexed cytokine analysis. The remaining cells were labeled with the following reagents or antibodies (all from Biolegend): Fixable live/dead Aqua, anti-human CD4 APC-Cy7 (clone OKT4), anti-human CD11c PE-Cy7 (clone Bu15), anti-human CD8 PerCP-Cy5.5 (clone SKI), and anti-human CD69 PE (clone FN50). The cells were acquired on a flow cytometer (Attune, ThermoFisher Scientific), and analyzed using FlowJo software (BD) for the upregulation of CD69 as a measure of both CD4 and CD8 T cell activation.

All MATCH4 molecules tested resulted in the specific killing of high ROR1 positive cells such as MDA-MB-231 and Jeko-1 (see FIG. 12A), and minimal to no activity was observed on ROR1 negative cells, MCF7 (see FIG. 12B). The EC₅₀ values of the MATCH4 molecules were superior to the corresponding scMATCH3 (PR02589 and PR02590 vs PRO2507, PRO2591 and PR02592 vs. PR02557/PRO2510). However, for low ROR1-expressing cells, i.e. JIMT-1 and SKOV-3 (FIG. 12A bottom), PR02589 and PR02590 (ROR1 domain derived from PRO2060) appear to have somewhat better EC₅₀ values compared to PR02591 and PRO2592 (ROR1 domain PRO2271). The PRO2060-derived MATCH4 molecules also appear to exhibit somewhat better EC₅₀ values compared to the scMATCH3 molecule PRO2507 on low ROR1-expressing cells, while this difference does not seem to be present when comparing the PRO2271-derived MATCH4 and scMATCH3 molecules. Interestingly, on cells expressing low levels of ROR1, the MATCH4 molecules seem to exhibit an overall lower maximal killing compared to the scMATCH3 molecules (FIG. 12A bottom). The cytotoxicity results are summarized in Table 37.

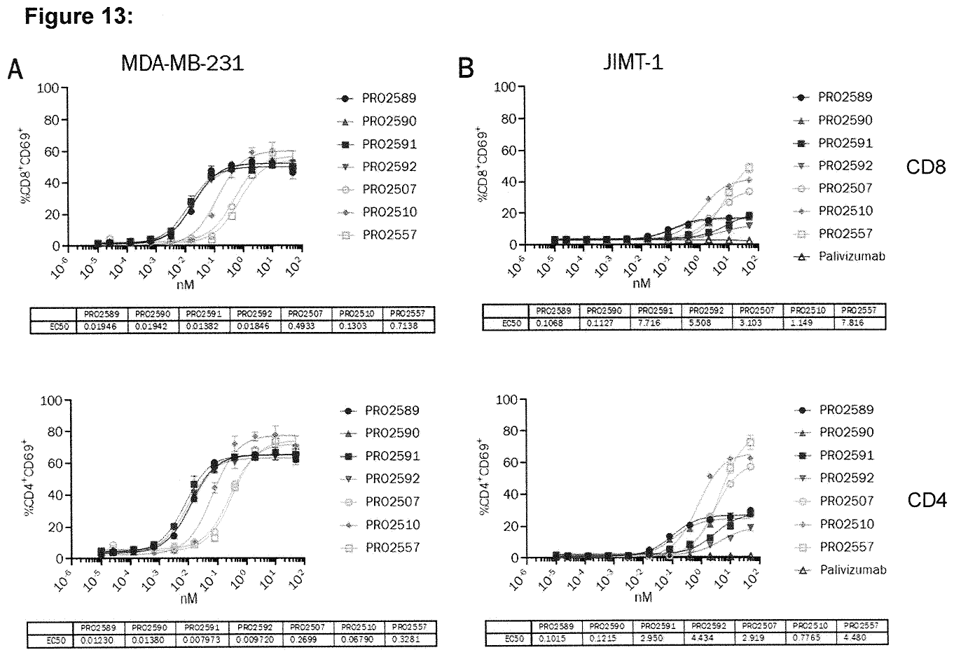

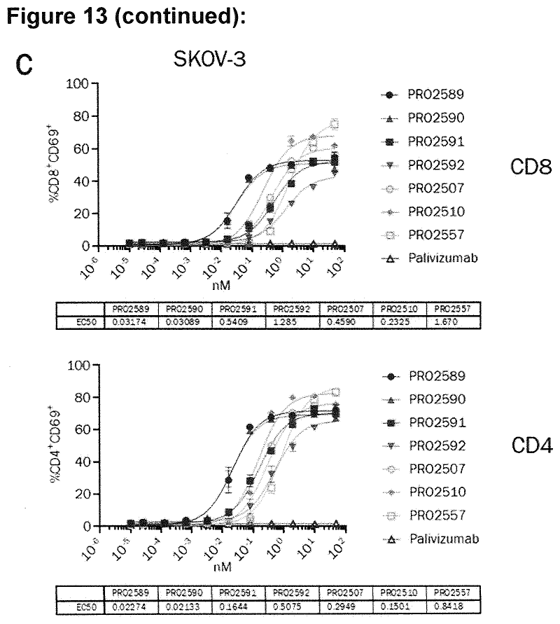

Furthermore, T cell activation was examined as measured through the increase in frequency of cells expressing CD69. CD8-based (FIG. 13 top) and CD4-based (FIG. 13 bottom) T cell activation tracked very closely with the cytotoxicity data. The highest frequencies for activated CD8 and CD4 T cells were obtained with the MDA-MB-231 cells, which also express the highest levels of ROR1 (FIG. 13A). The PR02060-based MATCH4 molecules have lower EC₅₀ values compared to the PRO2271-based MATCH4 molecules. However, these T cell populations had overall higher frequencies of activated cells when examining the scMATCH3 molecules, as compared to the MATCH4 molecules. The results are summarized in Tables 38 and 39.

The largest difference in the activity of the MATCH4 molecules seems to be present on the low ROR1 expressing cells, i.e. SKOV-3 and JIMT-1 cells. Due to the expected heterogeneity of ROR1 expression levels between patients and across tumor indications, it would be advantageous to effectively target tumors with lower ROR1 expression levels. Based on these data, it appears that the PR02592 has the lowest potencies (cytotoxicity and T cell activation) as well as lowest maximal values.

### 9.4. Biophysical Characterization of Lead MATCH4 Molecules

### Storage Stability and Melting Temperature

MATCH4 molecules were subjected to a 14 day stability study, in which the molecules were formulated in aqueous buffer (50 mM phosphate-citrate buffer with 300 mM sucrose at pH 6.5) at 1 mg/ml and stored at <−80° C., 4° C. and 40° C. for 14 days. The fraction of monomers and oligomers in the formulation were evaluated by integration of SE-HPLC peak areas at different time points over the course of the study. Table 40 summarizes monomeric content in % and % monomer loss relative to day 0. Changes in protein concentration were monitored by UV-Vis measurement at 280 nm over the course of the study and are shown in Table 41. Thermal stability was analyzed by nDSF (NanoTemper) determining the onset of unfolding (Tₒₙₛₑₜ) and midpoint of unfolding (Tₘ). Tₘ results are shown in Table 40.

All four MATCH4 molecules exhibit excellent stability profiles and do not show considerable monomeric content loss or protein content loss after 14 days incubation. There is no notable change in monomeric content at temperatures of −80° C. and 4° C. as well as upon repeated freeze-thawing (5×) as performed with the day 14/−80° C. sample before SE-HPLC/UV measurement.

### Example 10: Pre-Existing ADA Binding Assay

### 10.1. General Assay Procedure:

A method was developed at Numab to detect pre-existing anti-drug-antibodies in human serum, using a direct assay format.

96 well half-area plates were coated with 100 ng/ml of the test molecule (MATCH3 or scFv format) for 2 hours at room temperature. The plates were blocked for 1 hour with PBS containing 0.2% Tween and 1% BSA. Individual human sera were then added at a dilution of 1:20 (5% serum) or 1:100 (1% serum), either unspiked (screening assay) or spiked (confirmatory assay) with the same molecule as coated in the corresponding well. The spiking concentration ranged from 60 to 115 nM and spiked samples were pre-incubated for 1 hour. Antibodies bound to the molecules coated on the plate where then detected with 100 ng/ml rabbit anti-human IgG-HRP for 1 hour. TMB substrate was added as substrate and after a short incubation, the enzymatic reaction was stopped with 1 M HC. The optical density of each well was read at 450 nm. All steps were performed at room temperature. Between each step, plates were washed three times with 450 μl wash buffer. Except for the blocking and washing steps, all assay components were added in a volume of 25 μl/well and duplicates were used. For the incubation steps, the ELISA plates were placed on a rotating mixer (40 rpm). Generally, a first round of measurement was performed with unspiked human sera (screening assay). Then a screening cut-point (SCP) was calculated for each plate. Unspiked samples with signal below the SCP were termed “screening negative” and were not taken into account in the confirmatory assay. Unspiked samples with signal above the SCP were termed “screening positive”.

TABLE 37
Summary of key cytotoxicity data performed with Pan-T cells from MATCH4 and selected scMATCH3 molecules

| | | | Avg. EC₅₀ | Avg. % | Avg. EC₅₀ | | | | | |
|---|---|---|---|---|---|---|---|---|---|---|
| | | Domain 3, | (nM) | max. | (nM) CD8 | Avg. EC₅₀ | Avg. % | Avg. EC₅₀ | Avg. EC₅₀ | Avg. % |
| | | scFv | Cytotoxicity | lysis | T cell act. | (nM) | max. | (nM) CD8 | (nM) | max. |
| | | position | (MDA- | (MDA- | (MDA- | Cytotoxicity | lysis | T cell act. | Cytotoxicity | lysis |
| PRO ID | Format | (ROR1) | MB-231) | MB-231) | MB-231) | (SKOV3) | (SKOV3) | (SKOV3) | (JIMT-1) | (JIMT-1) |
| PRO2589 | MATCH4 | PRO2060 | 0.015 | 56.9 | 0.012 | 0.043 | 54.6 | 0.031 | 0.10 | 41 |
| PRO2590 | MATCH4 | PRO2291 | 0.016 | 52.2 | 0.013 | 0.049 | 51.8 | 0.034 | 0.13 | 38.6 |
| | | (PRO2060 + | | | | | | | | |
| | | diS) | | | | | | | | |
| PRO2591 | MATCH4 | PRO2271 | 0.011 | 47.2 | 0.010 | 3.5 | 58.7 | 0.68 | 2.4 | 34.5 |
| PRO2592 | MATCH4 | PRO2271 + | 0.008 | 47.1 | 0.011 | 5.37 | 44.7 | 1.6 | 3.3 | 24.9 |
| | | diS | | | | | | | | |
| PRO2507 | scMATCH3 | PRO2060 | 0.47 | 64.6 | 0.36 | 0.42 | 79.1 | 0.34 | 2. | 63.9 |
| PRO2510 | scMATCH3 | PRO2062 | 0.14 | 65.5 | 0.087 | 0.25 | 83.1 | 0.19 | 1.1 | 72.1 |
| PRO2557 | scMATCH3 | PRO2271 + | 0.74 | 78.2 | 0.52 | 1.3 | 88 | 1.2 | 3.4 | 83 |
| | | diS | | | | | | | | |

TABLE 38
Summary of CD8 T cell activation data from cytotoxicity assays with selected MATCH4 and selected scMATCH3
molecules

| | | | | | | Avg. % | Avg. EC₅₀ | Avg. % |
|---|---|---|---|---|---|---|---|---|
| | | | Avg. EC₅₀ (nM) | Avg. % max. | Avg. EC₅₀ (nM) | max. | (nM) CD8 T | max. |
| | | | CD8 T cell act. | CD8 T cell | CD8 T cell act. | CD8 T cell | cell act. | CD8 T cell |
| Protein | | | (MDA-MB-231) | act. (MDA- | (SKOV3) | act. | (JIMT-1) | act. |
| ID | Format | ROR1 domain | n = 3-5 | MB-231) | n = 2-3 | (SKOV3) | n = 3-5 | (JIMT-1) |
| PRO2589 | MATCH4 | 55-38-D07-sc02 | 0.012 | 68.5 | 0.032 | 66.7 | 0.08 | 23.8 |
| PRO2590 | MATCH4 | 55-38-D07-sc06 | 0.013 | 68.8 | 0.034 | 63.7 | 0.09 | 22.5 |
| PRO2591 | MATCH4 | 55-39-G02-sc03 | 0.010 | 67.5 | 0.68 | 62.7 | 4.6 | 20.3 |
| PRO2592 | MATCH4 | 55-39-G02-sc06 | 0.011 | 67 | 1.6 | 54.7 | 9.8 | 15.5 |
| PRO2507 | scMATCH3 | 55-38-D07-sc02 | 0.36 | 68 | 0.34 | 64.3 | 2.9 | 40.8 |
| PRO2510 | scMATCH3 | 55-39-G02-sc05 | 0.087 | 70.4 | 0.19 | 79.3 | 1.1 | 47.6 |
| PRO2557 | scMATCH3 | 55-39-G02-sc06 | 0.52 | 65.3 | 1.2 | 81.5 | 5.4 | 48.7 |

TABLE 39
Summary of CD4 T cell activation data from cytotoxicity assays with selected MATCH4 and selected scMATCH3
molecules

| | | | | | Avg. EC₅₀ | | Avg. EC₅₀ | |
|---|---|---|---|---|---|---|---|---|
| | | | Avg. EC₅₀ (nM) | Avg. % max. | (nM) CD4T | Avg. % | (nM) CD4 T | |
| | | | CD4 T cell act. | CD4 T cell | cell act. | max. CD4 | cell act. | Avg. % max. |
| Protein | | | (MDA-MB-231) | act. (MDA- | (SKOV3) | T cell act. | (JIMT-1) | CD4 T cell |
| ID | Format | ROR1 domain | n = 3-5 | MB-231) | n = 2-3 | (SKOV3) | n = 3-5 | act. (JIMT-1) |
| PRO2589 | MATCH4 | 55-38-D07-sc02 | 0.006 | 79 | 0.02 | 80.3 | 0.09 | 36.7 |
| PRO2590 | MATCH4 | 55-38-D07-sc06 | 0.007 | 79.3 | 0.02 | 78.3 | 0.11 | 35.3 |
| PRO2591 | MATCH4 | 55-39-G02-sc03 | 0.005 | 80.3 | 0.38 | 78.7 | 3.9 | 30.5 |
| PRO2592 | MATCH4 | 55-39-G02-sc06 | 0.005 | 79 | 0.76 | 75 | 9.1 | 23 |
| PRO2507 | scMATCH3 | 55-38-D07-sc02 | 0.14 | 81.8 | 0.22 | 83.3 | 2.4 | 63.2 |
| PRO2510 | scMATCH3 | 55-39-G02-sc05 | 0.036 | 82 | 0.13 | :87.3 | 0.8 | 68 |
| PRO2557 | scMATCH3 | 55-39-G02-sc06 | 0.20 | 77.7 | 0.55 | 87.5 | 3.3 | 69.3 |

TABLE 40
MATCH4 stability assessment at a concentration of 1 mg/ml, change of monomeric content over time.

| | | | | | | | | | | nDSF (Nano |
|---|---|---|---|---|---|---|---|---|---|---|
| | Temp. | Conc. | monomeric content [%]* | | | | % monomeric content loss | | | Temper) |
| Protein ID | [° C.] | [mg/ml] | d0 | d1 | d7 | d14 | d1 | d7 | d14 | Tm [° C.] |
| PRO2589 | −80 | 1.02 | 96.2 | NA | NA | 97.1* | NA | NA | −0.9* | 62.0 |
| | 4 | | 96.2 | 96.6 | 96.9 | 98 | −0.5 | −0.7 | −1.9 | |
| | 40 | | 96.2 | 96.4 | 96.9 | 97.1 | −0.2 | −0.7 | −0.9 | |
| PRO2590 | −80 | 1.06 | 98.9 | NA | NA | 99.2* | NA | NA | −0.2* | 62.9 |
| | 4 | | 98.9 | 98.9 | 99.0 | 99 | 0.0 | −0.1 | −0.1 | |
| | 40 | | 98.9 | 99.1 | 98.7 | 98.5 | −0.2 | 0.2 | 0.5 | |
| PRO2591 | −80 | 1.09 | 94.2 | NA | NA | 95.6* | NA | NA | −1.4* | 55.0 |
| | 4 | | 94.2 | 95.2\hat{ } | 96.4\hat{ } | 96.8\hat{ } | −1.1\hat{ } | −2.3\hat{ } | −2.7\hat{ } | |
| | 40 | | 94.2 | 97.4\hat{ } | 97.5\hat{ } | 95.1\hat{ } | −3.4\hat{ } | −3.5\hat{ } | −1* | |
| PRO2592 | −80 | 1.06 | 96.8 | NA | NA | NA | NA | NA | NA | 58.4 |
| | 4 | | 96.8 | 96.9 | 97.6 | 97.8 | −0.1 | −0.8 | −1 | |
| | 40 | | 96.8 | 97.1 | 96.8 | 97.1 | −0.3 | 0.0 | −0.3 | |

*sample was subjected to 5 repeated freeze/thaw cycles before measurement
\hat{ }considerable increase of monomeric content as compared to do of the study, may be due to dilution of a higher concentrated initial sample to the study concentration of 1 mg/ml
NA: not measured,
ND: non-determinable

TABLE 41
MATCH4 stability assessment at a concentration of 1 mg/ml, change of protein content over time.

| | Temp. | Conc. | protein concentration [mg/ml] | | | | % content loss | | |
|---|---|---|---|---|---|---|---|---|---|
| Protein ID | [° C.] | [mg/ml] | d0 | d1 | d7 | d14 | d1 | d7 | d14 |
| PRO2589 | −80 | 1.02 | 1.0 | NA | NA | 1.0* | NA | NA | 1.2* |
| | 4 | | 1.0 | 1.0 | 1.0 | 1.1 | −1.1 | 5.9 | −11.1 |
| | 40 | | 1.0 | 0.9 | 1.0 | 1.1 | 10.6 | 6.6 | −12.8 |
| PRO2590 | 80 | 1.06 | 1.1 | NA | NA | 1.0* | NA | NA | 3.9* |
| | 4 | | 1.1 | 1.0 | 1.0 | 1.1 | 1.4 | 8.7 | −4.3 |
| | 40 | | 1.1 | 1.0 | 1.1 | 1.1 | 1.9 | −6.5 | −2.9 |
| PRO2591 | −80 | 1.09 | 1.1 | NA | NA | 1.0* | NA | NA | 5.9* |
| | 4 | | 1.1 | 1.0 | 1.1 | 1.1 | 5.2 | −2.2 | 0.1 |
| | 40 | | 1.1 | 1.0 | 1.1 | 1.1 | 5.9 | 0.0 | 3.2 |
| PRO2592 | 80 | 1.06 | 1.1 | NA | NA | 1.0* | NA | NA | 6.6* |
| | 4 | | 1.1 | 1.0 | 1.0 | 1.0 | 6.2 | 2.6 | 7.3 |
| | 40 | | 1 1 | 1.0 | 1.0 | 1.0 | 6.9 | 5.3 | 6.1 |

*sample was subjected to 5 repeated freeze/thaw cycles before measurement

With most of these “screening positive” samples, a second round of measurement was performed with spiked human sera (confirmatory assay), to determine whether the initially detected binding of antibodies in the respective sera sample is specific to the test molecule. A decrease of the absorbance signal in the spiked wells indicates that the signal observed in the unspiked well of the initial screening assay is specific to the molecule coated on the plate. The resulting percent inhibition (% inhibition) required to confirm specificity was either set at 20 to 30% or a confirmatory cut-point was calculated in percent (% CCP). In the latter case, only samples where percent inhibition was above the CCP were confirmed positive. % inhibition was calculated as reduction of the initial signal obtained for unspiked serum (screening assay) as follows: % CCP=initial signal (1-(spiked serum/signal unspiked serum))*100.

In an alternative procedure, the initial screening assay was not performed. Instead, the test samples were directly analyzed using the confirmatory assay procedure. For data analysis however, the same calculations were performed, which at least involves the calculation of the SCP and the % CCP.

### 10.2. Determination/Calculation of Screening Cut Point (SCP), Normalization Factor (NF) and Floating Cut Point (FCP)

For each test compound, 40 individual human serum samples of healthy untreated subjects were analyzed. In other cases 20 individual human serum samples of healthy untreated subjects were analyzed.

### Screening Cut-Point (SCP):

The screening cut point (SCP) is the threshold at which a signal is considered positive (screening positive). It is calculated such that 5% false positive sera are included.

The screening cut point (SCP) is calculated as follows:

$$SCP = mean N + 1.645 \times SDN$$

### Wherein:

“Mean N” corresponds to mean signal from all unspiked individual sera measured for a specific test compound;
“SDN” corresponds to standard deviation calculated from all unspiked Individual sera measured for a specific test compound.

### Normalization Factor (NF):

The normalization factor (NF) is calculated as follow:

NF=SCP −Negative control mean

### Wherein:

“SCP” is as defined above; and
“negative control mean” corresponds to mean signal from the negative controls (pooled individual human sera, same on each plate) analyzed in duplicates (L. e. 2 wells) per plate.

At least two NC samples (L. e. 4 wells) must be taken into account for the calculation of the normalization factor.

### Calculation of the Floating Cut Point (FCP):

After the determination of the SCP and the NF, the Floating Cut Point (FCP) for each plate was used as the reference cut point. The FCP takes into account the analytical variability of each analytical run, by normalizing the SCP with the negative controls of the plate. The FCP is calculated for each analytical run as follows:

FCP=NF+Mean NC

### Wherein:

“Mean NC” refers to the mean signal of the negative control samples;
“NF” refers to the normalization factor, as defined above.

### 10.3. Pre-Existing ADA Binding Assay Results for Improved Multispecific Antibody Variants:

The variants PRO2668 and PRO2669 and, as comparison, the corresponding unmodified references PR02510 and PRO2589, have been measured for their immunogenic properties using the pre-existing ADA binding assay described above. The measurements were directly performed in the confirmatory assay setup using 20 human serum samples (19 in case of PR02589).

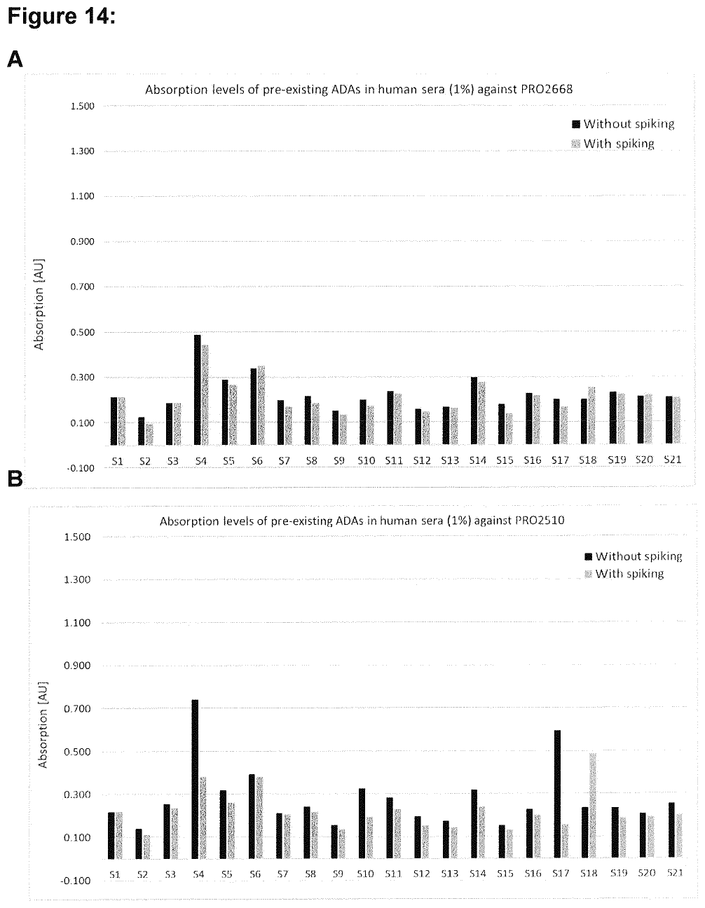

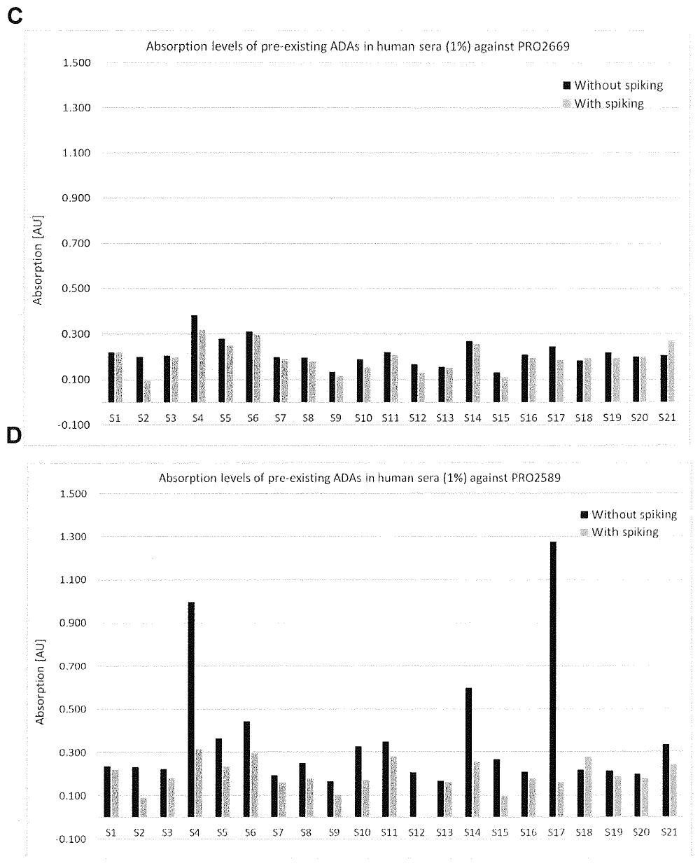

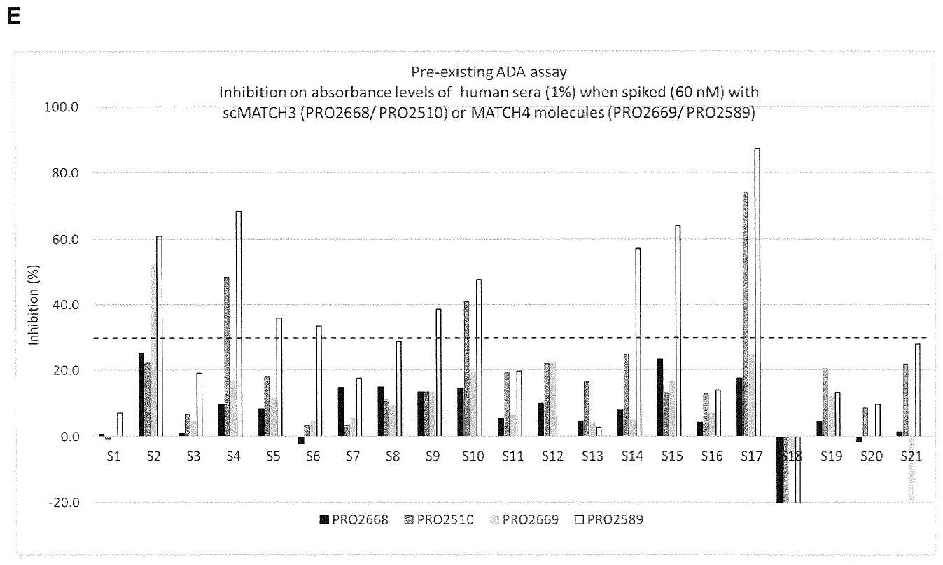

The screening positives sera were then further analyzed by taking into account a % CCP of 30%. The number of positive serum samples for each tested molecule are summarized in Table 42. Graphs of absorption levels of pre-existing ADAs in human serum as well as the reduction of absorbance level of spiked human serum (ADA binding inhibition) for PR02741 and the reference PRO2660 are shown in FIG. 14.

TABLE 42
Number of positive serum samples above 30% CCP of PRO2668 and
PRO2669 and the references PRO2510 and PRO2589:

| | No of positive serum | % positives of total |
|---|---|---|
| | samples above 30% | tested serum |
| PRO Number | CCP | samples |
| PRO2668 | 0 | 0 |
| PRO2510 | 3 | 15 |
| PRO2669 | 1 | 5 |
| PRO2589 | 10 | 52.6 |

### Example 11: Target Cell Cytotoxicity and T Cell Activation Mediated by scMATCH3 and MATCH4 Variants PRO2667. PR02668. PR02669 nd PRO2670

### Methods

These assays were performed using human peripheral blood mononuclear cells (PBMCs) isolated from buffy coats using SepMate tubes with Lymphoprep (Stemcell Technologies) to create a density gradient following manufacturer's instructions. Pan T cells were isolated from PBMCs using the Pan T cell isolation kit from Miltenyi Biotec, following manufacturer's instructions.

Isolated Pan-T were co-cultured for 40 hours with ROR1-positive and negative tumor cells of interest at an effector-to-target ratio of either 10:1 for adherent cells or 5:1 for cells in suspension. Following the co-culture, 100 μl of supernatant was aspirated to test for lactate dehydrogenase (LDH) release from dying cells using a cytotoxicity detection kit from Roche. The remaining supernatant was frozen at −80° C. for downstream multiplexed cytokine analysis. The cells were labeled with the following reagents or antibodies (all from Biolegend) for flow cytometry analysis of specific T cell activation: Fixable live/dead Aqua, anti-human CD4 APC-Cy7 (clone OKT4), anti-human CD11c PE-Cy7 (clone Bul5), anti-human CD8 PerCP-Cy5.5 (clone SKI), and anti-human CD69 PE (clone FN50). The cells were acquired on a flow cytometer (Attune, Thermofisher Scientific), and analyzed using FlowJo software (BD) for the upregulation of CD69 as a measure of both CD4 and CD8 T cell activation.

### Results

The use of all scMATCH3 (PR02667 and PRO2668) and MATCH4 (PRO2669 and PRO2670) molecules tested resulted in the specific killing of ROR1 positive MDA-MB-231 and JIMT-1 cos as shown in FIG. 15A. The potency of the MATCH4 molecules was superior to that of the scMATCH3 molecules, however the level of maximum lysis achieved on the lower ROR1 expressing JIMT-1 cells was higher when using the scMATCH3 molecules. Similar results were obtained when examining CD8 T cell activation, as gauged by the frequency of CD69 expressing CD8 T cells (FIG. 15B).

### Example 12: Potency of PR02668 to Induce Specific T Cell-Mediated Lysis of ROR1-Positive Solid and Hematological Cancer Cell Lines

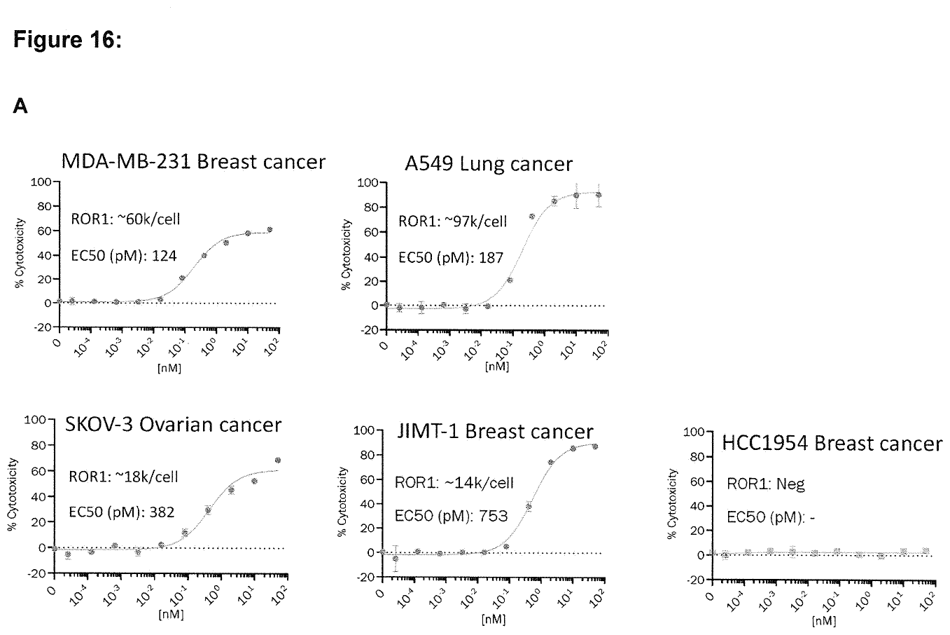

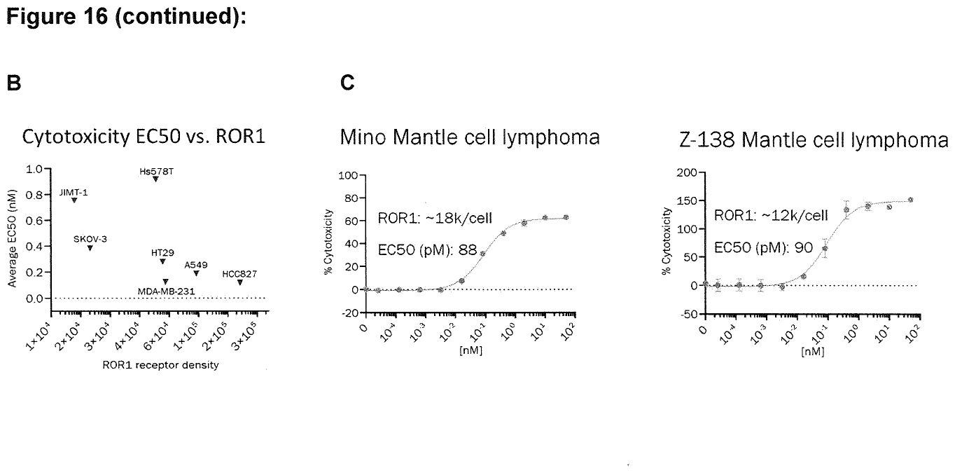

The cytotoxicity mediated by PRO2668 was assessed on cell lines representing solid (FIGS. 16A and 16B) and hematological malignancies (FIG. 16C). Cytotoxicity was observed using ROR1-positive cell lines as targets, while the ROR1-negative line HCC1954 was not lysed, indicating the ROR1 dependence of PRO2668 (FIG. 16A). The potency of specific lysis overall Improved with increasing ROR1 density (FIG. 16B). Specific killing of mantle cell lymphoma lines Mino and Z-138 was also observed (FIG. 16C). These data indicate that PRO2668 is able to mediate specific killing on a range of ROR1-expressing tumor cell lines.

### Example 13: Potency of PRO2668 to Mediate ROR1-Dependent CD8 T Cell Activation and Proliferation

### Methods

Isolated healthy Pan-T cells were labeled with CelTrace Violet following manufacturer's instructions and co-cultured for 3 days with ROR1-positive tumor cells at an effector-to-target ratio 5:1. After 3 and/or 6 days of co-culture, the cells were labeled with the following reagents or antibodies (al from Biolegend) for flow cytometry analysis of specinc T cell activation: Fixable live/dead Aqua, anti-human CD4 APC-Cy7 (clone OKT4), anti-human CD19 PE (clone HIB19), anti-human CD8 PerCP-Cy5.5 (clone SKI), anti-human CD3 Alexa Fluor 488 (clone OKT3), and anti-human CD25 BV650 (clone BC96). The cells were acquired on a flow cytometer (Attune, ThermoFisher Scientific), and analyzed using FlowJo software (BD). Dividing cells had a reduced level of fluorescence compared to non-dividing cells (dye dilution). The upregulation of CD25 frequency was used as a measure of CD8 T cell activation.

### Results

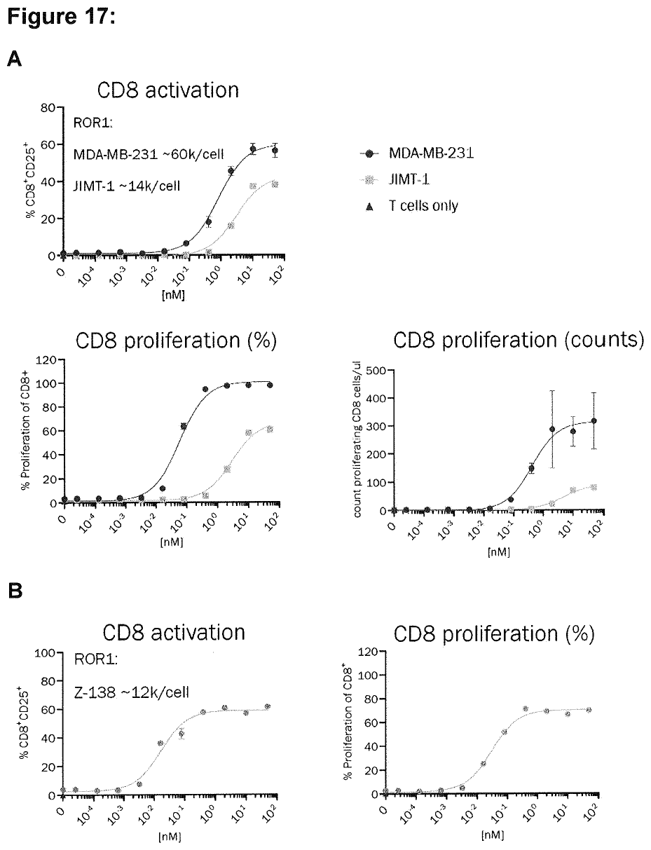

CD8 T cell activation was observed upon co-culture with ROR1-expressing breast cancer cell lines MDA-MB-231 and JIMT-1 (FIG. 17A). The extent of activation appeared to correlate with ROR1 expression, as higher frequencies of activated T cells were observed with higher ROR1 expression. CD8 T cell proliferation was observed in response to both ROR1-expressing cell Ones, when examining frequencies of dividing cells (cels with reduced CellTrace Violet fluorescence) or dividing cell counts per microliter. The extent of proliferation was higher when using targets with higher ROR1 expression. CD8 T cell activation and proliferation was also observed in response to the mantle cell lymphoma line Z-138 (FIG. 17B). These data indicate that engagement of ROR1-expressing targets and T cells with PRO2668 leads to T cell activation and proliferation.

### Example 14: Potency of PRO2668 to Mediate Specific Lysis of Non-Mitotic. Low ROR1-Expressing Primary Human CLL Patient Samples

### Methods

These assays were performed using CLL patient samples (IQBiosciences) and healthy human Pan-T cells isolated from peripheral blood mononuclear cells (PBMCs). PBMCs were Isolated from buffy coats using SepMate tubes with Lymphoprep (StemCod Technologies) to create a density gradient following manufacturer's,instructions. Pan T cells were Isolated from PBMCs using the Pan T cell Isolation kit from Miltenyi Biotec, following manufacturer's Instructions.

CLL patient samples were assessed for their ability to divide by examining 5-ethynyl-2′-deoxyuridine (EdU) incorporation using the Click-it Plus Flow Cytometry assay kit and following manufacturer's Instructions. Dividing cells were positively labeled compared to non-dividing cells. The receptor density was assessed using the protocol outilned above, e. g. as outlined in section 9.3. Cell binding of PRO2668 was assessed by titrating increasing concentrations of PRO2668 in the presence of human serum albumin, followed by extensive washing and detection with a fluorescently labeled secondary reagent designed to detect the framework. Samples were acquired on a flow cytometer and analyzed using FlowJo.

Isolated Pan-T were co-cultured for 40 hours with CLL patient samples at an effector:target ratio of 5:1. Following the co-culture, the remaining supernatant was frozen at −80° C. for downstream multiplexed cytokine analysis. The cells were labeled with the following reagents or antibodies (all from Biolegend) for flow cytometry analysis of cell death and specific T cell activation: Fixable live/dead Aqua, anti-human CD4 APC-Cy7 (done OKT4), anti-human CD3 Alexa Fluor 488 (clone OKT3), anti-human CD8 PerCP-Cy5.5 (clone SKI), and anti-human CD69 BV650 (clone FN50), anti-human CD19 BV421 (clone SJ25C1), Annexin V APC, anti-human ROR1 PE (clone 2A2). The cells were acquired on a flow cytometer (Attune, ThermoFisher Scientific), and analyzed using FlowJo software (BD) for the upregulation of CD69 as a measure of both CD4 and CD8 T cell activation. Dying CLL cells were gated as CD19+ Annexin V+L/D+. Cyokine release was assessed from frozen supernatants using the LEGENDplex™ Multi-Analyte Flow Assay Kit, Human Essential Immune Response Panel 13-plex as per manufacturer's instructions.

### Results

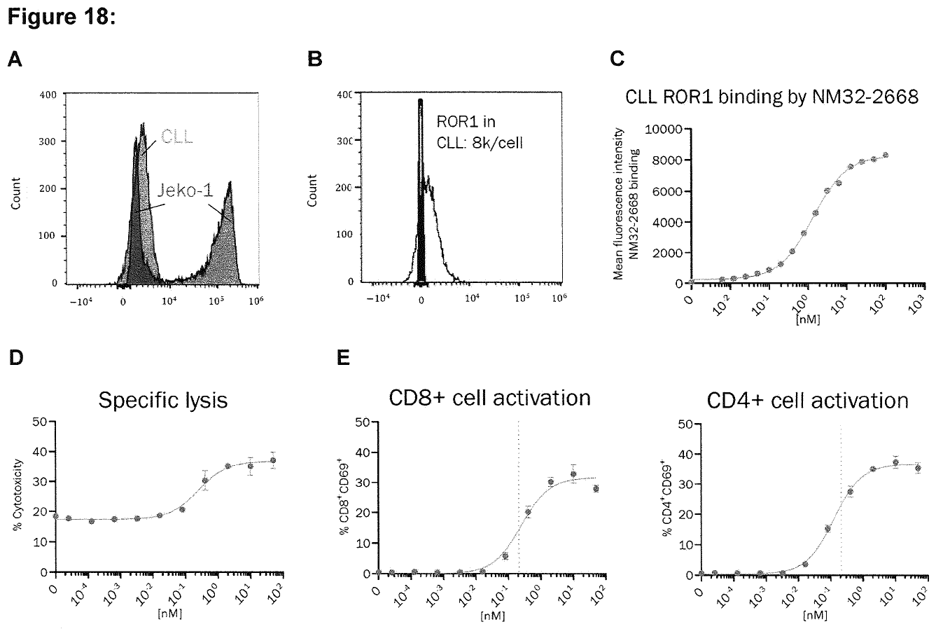

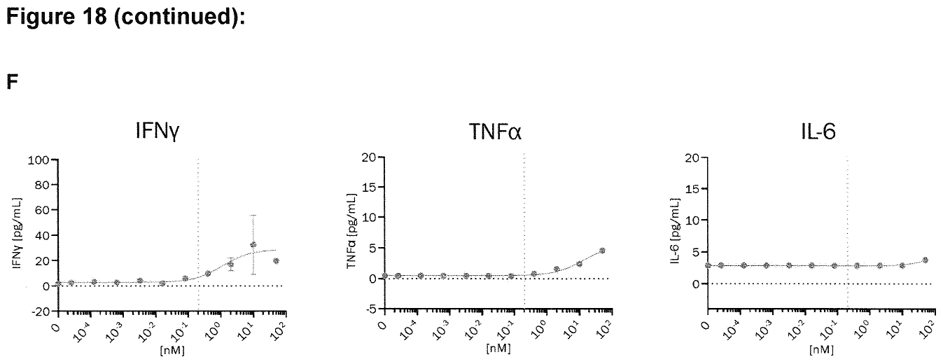

The lack of proliferation of CLL PBMCs in the periphery create a challenge for drugs that require active proliferation for their activity (Hu et al., Blood Adv. 5 (2021) 3152-3162). The lack of proliferation of CLL patient samples from peripheral blood was confirmed using EdU incorporation (FIG. 18A). In contrast to the actively dividing Jeko-1 cell line, the CLL patient sample did not divide. These samples expressed low levels of ROR1 (8,000 receptors per cells, FIG. 18B). Saturation of the ROR1 receptor was observed on these cells (FIG. 18C). Upon co-culture with healthy allogeneic Pan-T cells and CLL patient samples, specific lysis (FIG. 18D) and concomitant CD8 and CD4 activation were observed (FIG. 18E). Cytokine release was modest for some cytokines (IFNg and TNFa) and not observed for others like IL-6 (FIG. 18F). These data indicate that PRO2668 is able to bind and specifically lyse non-dividing CLL patient samples, resulting in T cell activation and modest levels of cytokine release.

### Example 15: In Vivo Efficacy of PRO2668 and PRO2670

### Methods

Frozen PBMCs were thawed in a 37° C. water bath, centrifuged, resuspended in PBS, and stored on ice for inoculation. The JeKo-1 tumor cells were maintained in vfro with RPMI1640 medium supplemented with 20% fet all bovine serum at 37C in an atmosphere of 5% CO₂. The cells were harvested during exponential growth phase and quantitated using a cell counter before tumor inoculation.

Each treatment group consisted of five animals. Each mouse was inoculated subcutaneously in the right upper flank region with 5×10⁶ JeKo-1 tumor cells in 0.1 ml of PBS mixed with Matrigel (1:1) for tumor development. 1×10⁷ PBMC were implanted intraperitoneally 3 days after tumor inoculation. Randomization was performed when the mean tumor size reached 80-100 mm³.

Dosing was initiated upon randomization (day 0) and performed every 5 days thereafter. After tumor Inoculation, the animals were checked daily for morbidity and mortality. During routine monitoring, the animals were checked for any effects of tumor growth and treatments on behavior such as mobility, food and water consumption, body weight gain/loss (body weights were measured twice a week after randomization), eye/hair matting and any other abnormalities. Mortality and observed clinical signs were recorded for individual animals in detail.

Tumor volumes were measured twice a week after randomization in two dimensions using a caliper, and the volume was expressed in mm³ using the formula: V=(L×W×W)2, where V is tumor volume, L is tumor length (the longest tumor dimension) and W is tumor width (the longest tumor dimension perpendicular to L). The animals were sacrificed if they lost over 20% of their body weight relative to the weight at the first day of treatment; If an individual mouse with tumor volume ≥3,000 mm³ (the individual mouse sacrificed); or the mean tumor volume of a group ≥2,000 mm³ (all mice in the same group sacrificed).

### Results

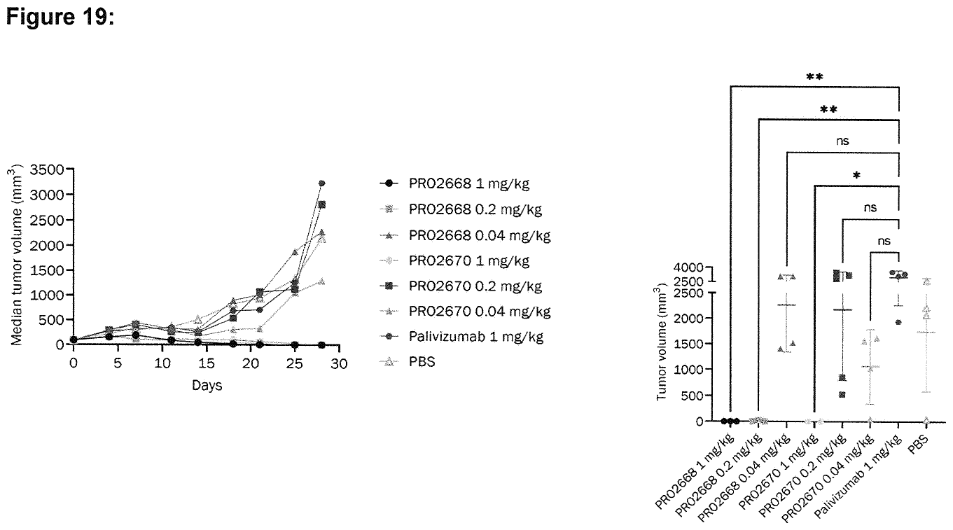

PRO2668 was tested in an in vivo efficacy study using a Jeko-1 human xenograft model. Jeko-1 cells were implanted subcutaneously, and human PBMCs were administered 3 days after tumor engraftment. As can be depicted from FIG. 19, dosing with 1 mg/kg and 0.2 mg/kg of PRO2668 resulted in significant tumor growth inhibition compared to the irrelevant protein control palivizumab. No different in efficacy relative to control was observed for 0.04 mg/kg of PRO2668. Anti-tumor efficacy was observed for PRO2670 at the 1 mg/kg dose, whereas the lower doses did not demonstrate significantly different tumor volumes relative to controls. While both molecules appear to be efficacious, PRO2668 demonstrates efficacy also at the lower dose of 0.2 mg/kg.

### Example 16: Biophysical characterization of scMATCH3 and MATCH4 variants PRO2667. PR02668. PRO2669 nd PRO2670

### Storage Stability and Melting Temperature

MATCH4 molecules were subjected to a 28 day stability study, in which the molecules were formulated in aqueous buffer (50 mM phosphate-citrate buffer with 300 mM sucrose at pH 6.5) at 1 mg/ml and stored at <−80° C., 4° C. and 40° C. for 14 days. The fraction of monomers and oligomers in the formulation were evaluated by integration of SE-HPLC peak areas at different time points over the course of the study. Table 43 summarizes monomeric content in % and % monomer loss relative to day 0. Changes in protein concentration were monitored by UV-Vis measurement at 280 nm over the course of the study and are shown in Table 44. Thermal stability was analyzed by nDSF (NanoTemper) determining the onset of unfolding (Tₒₙₛₑₜ) and midpoint of unfolding (Tₘ). Tₘ results are shown in Table 43.

All four MATCH4 molecules exhibited excellent stability profiles and did not show considerable monomeric content loss or protein content loss after 28 days incubation. There was no notable change in monomeric content at temperatures of −80° C. and 4° C. as well as upon repeated freeze-thawing (5×) as performed with the day 28/−80C sample before SE-HPLC/UV measurement and only minor monomeric content loss upon storage for 28 days at 40° C.

TABLE 43
MATCH4 stability assessment at a concentration of 1 mg/ml, change of monomeric
content over time.

| | | | | | | | | | | | | nDSF (Nano |
|---|---|---|---|---|---|---|---|---|---|---|---|---|
| Protein | Temp. | Conc. | monomeric content [%] | | | | | % monomeric content loss | | | | Temper) Tₘ |
| ID | [° C.] | [mg/ml] | d0 | d1 | d7 | d14 | d28 | d1 | d7 | d14 | d28 | [° C.] |
| PRO2667 | −80 | 1.07 | 98.7 | NA | NA | NA | 98.6* | NA | NA | NA | 0.1* | 62.5 |
| | 4 | | 98.7 | 98.5 | 98.6 | 98.8 | 98.6 | 0.2 | 0.1 | −0.1 | 0.2 | |
| | 40 | | 98.7 | 98.5 | 97.2 | 97.0 | 95.6 | 0.2 | 1.5 | 1.8 | 3.2 | |
| PRO2668 | −80 | 1.01 | 98.6 | NA | NA | NA | 99.8* | NA | NA | NA | −1.2* | 64.6 |
| | 4 | | 98.6 | 99.4 | 99.7 | 99.7 | 99.7 | −0.8 | −1.1 | −1.1 | −1.1 | |
| | 40 | | 98.6 | 99.4 | 98.8 | 97.2 | 96.7 | −0.8 | −0.2 | 1.5 | 2.0 | |
| PRO2669 | −80 | 1.06 | 98.5 | − NA | NA | NA | 98.9* | NA | NA | NA | −0.4* | 63.1 |
| | 4 | | 98.5 | 98.7 | 98.6 | 99.0 | 98.5 | −0.1 | −0.1 | −0.5 | 0.0 | |
| | 40 | | 98.5 | 98.9 | 97.5 | 97.0 | 92.7 | −0.4 | 1.1 | 1.6 | 5.9 | |
| PRO2670 | −80 | 1.06 | 98.7 | NA | NA | NA | 100* | NA | NA | NA | −1.4* | 61.5 |
| | 4 | | 98.7 | 98.9 | 98.9 | 98.8 | 100.0 | −0.3 | −0.3 | −0.1 | −1.4 | |
| | 40 | | 98.7 | 99.2 | 98.6 | 97.2 | 97.4 | −0.5. | 0.1 | 1.4 | 1.3 | |

*sample was subjected to 5 repeated freeze/thaw cycles before measurement
NA: not measured

TABLE 44
MATCH4 stability assessment at a concentration of 1 mg/ml, change of protein content over
time.

| Protein | Temp. | Conc. | protein concentration [mg/ml] | | | | | % content loss | | | |
|---|---|---|---|---|---|---|---|---|---|---|---|
| ID | [° C.] | [mg/ml] | d0 | d1 | d7 | d14 | d28 | d1 | d7 | d14 | d28 |
| PRO2667 | −80 | 1.07 | 1.07 | NA | NA | NA | 1.04* | NA | NA | NA | 3.0* |
| | 4 | | 1.07 | 1.03 | 1.06 | 1.04 | 1.08 | 3.9 | 0.5 | 2.6 | −1.2 |
| | 40 | | 1.07 | 1.16 | 1.01 | 1.06 | 1.08 | −8.1 | 5.2 | 0.9 | −1.3 |
| PRO2668 | −80 | 1.01 | 1.01 | NA | NA | NA | 1.11* | NA | NA | NA | −10.3* |
| | 4 | | 1.01 | 1.20 | 1.13 | 1.06 | 1.14 | −19.1 | −11.9 | −5.1 | −12.7 |
| | 40 | | 1.01 | 1.09 | 1.02 | 0.99 | 0.99 | −8.1 | −0.7 | 2.5 | 1.5 |
| PRO2669 | −80 | 1.06 | 1.06 | NA | NA | NA | 1.03 | NA | NA | NA | 3.0* |
| | 4 | | 1.06 | 1.10 | 1.02 | 1.03 | 1.13 | −3.6 | 3.6 | 2.2 | −7.2 |
| | 40 | | 1.06 | 1.06 | 0.99 | 1.04 | 1.04 | −0.2 | 6.1 | 2.0 | 1.5 |
| PRO2670 | −80 | 1.06 | 1.06: | NA | NA | NA | 1.16* | NA | NA | NA | 0.2* |
| | 4 | | 1.06 | 1.18 | 1.08 | 1.08 | 1.15 | −11.4 | −1.5 | −1.7 | −8.7 |
| | 40 | | 1.06 | 1.16 | 1.04 | 0.99 | 1.04 | −9.0 | 1.7 | 6.6 | 2.0 |

*sample was subjected to 5 repeated freeze/thaw cycles before measurement
NA: not measured

1. A multispecific antibody comprising:
a) one or two binding domains, which specifically bind to the extracellular domain of ROR1 (ROR1-BDs); and
b) one binding domain, which specifically binds to CD3 (CD3-BD);
wherein
the multispecific antibody does not comprises an immunoglobulin Fc region;
said ROR1-BDs comprise independently from each other a set of CDR sequences selected from the set consisting of
the HCDR1 sequence of SEQ ID NO: 1,
the HCDR2 sequence of SEQ ID NO: 2,
the HCDR3 sequence of SEQ ID NO: 3,
the LCDR1 sequence of SEQ ID NO: 4,
the LCDR2 sequence of SEQ ID NO: 5, and
the LCDR3 sequence of SEQ ID NO: 6;
and/or the set consisting of
the HCDR1 sequence of SEQ ID NO: 13 or 14,
the HCDR2 sequence of SEQ ID NO: 15,
the HCDR3 sequence of SEQ ID NO: 16,
the LCDR1 sequence of SEQ ID NO: 17,
the LCDR2 sequence of SEQ ID NO: 18, and
the LCDR3 sequence of SEQ ID NO: 19;
said CD3-BD comprises the set of CDR sequences consisting of
the HCDR1 sequence of SEQ ID NO: 30,
the HCDR2 sequence of SEQ ID NO: 31,
the HCDR3 sequence of SEQ ID NO: 32,
the LCDR1 sequence of SEQ ID NO: 33,
the LCDR2 sequence of SEQ ID NO: 34, and
the LCDR3 sequence of SEQ ID NO: 35.

2. The multispecific antibody of claim 1, where in case said multispecific antibody comprises two ROR1-BDs, said two ROR1-BDs comprise the same sets of CDRs, i.e. both ROR1-BDs either comprise CDRs of SEQ ID NOs: 1 to 6 or CDRs of SEQ ID NOs: 13/14 to 19.

3. The multispecific antibody of claim 1, wherein said antibody further comprises one binding domain, which specifically binds to human serum albumin (hSA-BD).

4. The multispecific antibody of claim 1, wherein the multispecific antibody has one or more of the following features 1) to 5):
1) said multispecific antibody does not comprise CH1 and/or CL regions;
2) said one or two ROR1-BDs, said CDR-BD and said hSA-BD, if present, comprise VH1a, VH1b, VH3 or VH4 domain framework sequences FR1 to FR4; particularly VH3 or VH4 domain framework sequences FR1 to FR4; particularly VH3 domain framework sequences FR1 to FR4; or said one or two ROR1-BDs, said CDR-BD and said hSA-BD, if present, comprise a VH domain comprising VH framework regions FR1, FR2, FR3 and FR4, which are selected from a VH framework subtype, particularly from the VH framework subtypes VH1a, VH1b, VH3 and VH4, particularly from the VH framework subtypes VH3 and VH4, particularly are of the VH3 subtype; wherein said VH framework regions FR1, FR2, FR3 and FR4 have the following substitutions (AHo numbering): an arginine (R) at amino acid position 12; a threonine (T) at amino acid position 103 and a glutamine (Q) at amino acid position 144;
3) said one or two ROR1-BDs, said CDR-BD and said hSA-BD, if present, comprise a VL domain comprising VL framework regions FR1, FR2 and FR3, which are selected from V_{K} subtypes, particularly from the V_{K}1 and V_{K}3 subtypes, particularly are of the V_{K}1 subtype, and a VL framework FR4, which is a VA FR4, particularly is a VA FR4 comprising an amino acid sequence having at least 70, 80, 90 percent identity to any of SEQ ID NO: 76 to SEQ ID NO: 83, more particularly a VA FR4 selected from any of SEQ ID NO: 76 to SEQ ID NO: 83, particularly a VA FR4 according to SEQ ID NO: 76 or 83;
4) said multispecific antibody is humanized, in particular said multispecific antibody is humanized and comprises rabbit derived CDRs;
5) said multispecific antibody is trispecific, in particular trispecific and trivalent or trispecific and tetravalent.

5. The multispecific antibody of claim 1, wherein the format of said multispecific antibody is selected from an scDb-scFv, an scMATCH3, a MATCH3 and a MATCH4.

6. The multispecific antibody of claim 1, wherein said one or two ROR1-BDs comprise
a) a VH sequence being at least 90, 91, 92, 93, 94, 95, 96, 97, 98 or 99 percent identical to any one of the amino acid sequences selected from SEQ ID NOs: 7 and 10; and
b) a VL sequence being at least 90, 91, 92, 93, 94, 95, 96, 97, 98 or 99 percent identical to any one of the amino acid sequences selected from SEQ ID NOs: 9 and 12;
or
a) a VH sequence being at least 90, 91, 92, 93, 94, 95, 96, 97, 98 or 99 percent identical to any one of the amino acid sequences selected from SEQ ID NOs: 20, 23, 26 and 28; and
b) a VL sequence being at least 90, 91, 92, 93, 94, 95, 96, 97, 98 or 99 percent identical to any one of the amino acid sequences selected from SEQ ID NOs: 22, 25, 27 and 29;
or
a) a VH sequence being at least 90, 91, 92, 93, 94, 95, 96, 97, 98 or 99 percent identical to any one of the amino acid sequences selected from SEQ ID NOs: 8 and 11; and
b) a VL sequence being at least 90, 91, 92, 93, 94, 95, 96, 97, 98 or 99 percent identical to any one of the amino acid sequences selected from SEQ ID NOs: 9 and 12;
or
a) a VH sequence being at least 90, 91, 92, 93, 94, 95, 96, 97, 98 or 99 percent identical to any one of the amino acid sequences selected from SEQ ID NOs: 21 and 24; and
b) a VL sequence being at least 90, 91, 92, 93, 94, 95, 96, 97, 98 or 99 percent identical to any one of the amino acid sequences selected from SEQ ID NOs: 22 and 25.

7. The multispecific antibody of claim 1, wherein said one or two ROR1-BDs comprise
a) a VH sequence of SEQ ID NO: 7 and a VL sequence of SEQ ID NO: 9; or
b) a VH sequence of SEQ ID NO: 10 and a VL sequence of SEQ ID NO: 12; or
c) a VH sequence of SEQ ID NO: 20 and a VL sequence of SEQ ID NO: 22; or
d) a VH sequence of SEQ ID NO: 23 and a VL sequence of SEQ ID NO: 25; or
e) a VH sequence of SEQ ID NO: 8 and a VL sequence of SEQ ID NO: 9; or
f) a VH sequence of SEQ ID NO: 11 and a VL sequence of SEQ ID NO: 12; or
g) a VH sequence of SEQ ID NO: 21 and a VL sequence of SEQ ID NO: 22; or
h) a VH sequence of SEQ ID NO: 24 and a VL sequence of SEQ ID NO: 25; or
i) a VH sequence of SEQ ID NO: 26 and a VL sequence of SEQ ID NO: 27; or
j) a VH sequence of SEQ ID NO: 28 and a VL sequence of SEQ ID NO: 29;
particularly wherein said one or two ROR1-BDs comprise
a) a VH sequence of SEQ ID NO: 7 and a VL sequence of SEQ ID NO: 9; or
b) a VH sequence of SEQ ID NO: 10 and a VL sequence of SEQ ID NO: 12; or
c) a VH sequence of SEQ ID NO: 23 and a VL sequence of SEQ ID NO: 25; or
d) a VH sequence of SEQ ID NO: 8 and a VL sequence of SEQ ID NO: 9; or
e) a VH sequence of SEQ ID NO: 11 and a VL sequence of SEQ ID NO: 12; or
f) a VH sequence of SEQ ID NO: 24 and a VL sequence of SEQ ID NO: 25.

8. The multispecific antibody of claim 1, wherein said CD3-BD comprises
a) a VH sequence being at least 90, 91, 92, 93, 94, 95, 96, 97, 98 or 99 percent identical to any one of the amino acid sequences selected from SEQ ID NO: 36 or 37; and
b) a VL sequence being at least 90, 91, 92, 93, 94, 95, 96, 97, 98 or 99 percent identical to any one of the amino acid sequences selected from SEQ ID NO: 38;
in particular
a) a VH domain comprising the amino acid sequence of SEQ ID NO: 36 or 37, and
b) a VL domain comprising the amino acid sequence of SEQ ID NO: 38.

9. The multispecific antibody of claim 4, wherein said hSA-BD comprises
(i) a VH domain comprising the amino acid sequence of SEQ ID NO: 47 and a VL domain comprising the amino acid sequence of SEQ ID NO: 48; or
(ii) a VH domain comprising the amino acid sequence of SEQ ID NO: 45 and a VL domain comprising the amino acid sequence of SEQ ID NO: 46; or
(iii) a VH domain comprising the amino acid sequence of SEQ ID NO: 55 and a VL domain comprising the amino acid sequence of SEQ ID NO: 56; or
(iv) a VH domain comprising the amino acid sequence of SEQ ID NO: 57 and a VL domain comprising the amino acid sequence of SEQ ID NO: 58; or
(v) a VH domain comprising the amino acid sequence of SEQ ID NO: 65 and a VL domain comprising the amino acid sequence of SEQ ID NO: 67; or
(vi) a VH domain comprising the amino acid sequence of SEQ ID NO: 66 and a VL domain comprising the amino acid sequence of SEQ ID NO: 67; or
(vii) a VH domain comprising the amino acid sequence of SEQ ID NO: 68 and a VL domain comprising the amino acid sequence of SEQ ID NO: 70;
(viii) a VH domain comprising the amino acid sequence of SEQ ID NO: 69 and
a VL domain comprising the amino acid sequence of SEQ ID NO: 70; or in particular
(v) a VH domain comprising the amino acid sequence of SEQ ID NO: 65 and a VL domain comprising the amino acid sequence of SEQ ID NO: 67; or
(vi) a VH domain comprising the amino acid sequence of SEQ ID NO: 66 and a VL domain comprising the amino acid sequence of SEQ ID NO: 67; or
(vii) a VH domain comprising the amino acid sequence of SEQ ID NO: 68 and a VL domain comprising the amino acid sequence of SEQ ID NO: 70
(viii) a VH domain comprising the amino acid sequence of SEQ ID NO: 69 and a VL domain comprising the amino acid sequence of SEQ ID NO: 70.

10. A ROR1-BD as defined of claim 1.

11. A nucleic acid or two nucleic acids encoding the multispecific antibody of claim 1.

12. A vector or two vectors comprising the nucleic acid or the two nucleic acids of claim 11.

13. A host cell or host cells comprising the vector or the two vectors of claim 12.

14. A pharmaceutical composition comprising the multispecific antibody of claim 1 and a pharmaceutically acceptable carrier.

15. The multispecific antibody of claim 1 for use in the treatment of a ROR1-expressing cancer, in particular a ROR1-expressing cancer selected from chronic lymphocytic leukemia/small lymphocytic lymphoma (CLL/SLL), acute myeloid leukemia (AML), acute lymphoblastic leukemia (ALL), mantle cell lymphoma (MCL), hairy cell leukemia, follicular lymphoma (FL), marginal zone lymphoma (MZL), Non-Hodgkin lymphoma (NHL), diffuse large B cell lymphoma (DLBCL), Richter's syndrome (RS), lung cancer, pancreatic cancer, prostate cancer, colon cancer, bladder cancer, breast cancer, ovarian cancer, glioblastoma, testicular cancer, uterine cancer, adrenal cancer, melanoma, neuroblastoma, sarcoma and renal cancer.


SEQUENCE LISTING (122 sequences)

SEQ ID NO: 1 - protein, 10 aa, Artificial sequence
  artificial antibody-based sequence
GFDLSSYAVS

SEQ ID NO: 2 - protein, 16 aa, Artificial sequence
  artificial antibody-based sequence
IIYPRANTYYASWAKG

SEQ ID NO: 3 - protein, 17 aa, Artificial sequence
  artificial antibody-based sequence
RDRYDSGAYLYTTYFNL

SEQ ID NO: 4 - protein, 11 aa, Artificial sequence
  artificial antibody-based sequence
RASENIYSGLA

SEQ ID NO: 5 - protein, 7 aa, Artificial sequence
  artificial antibody-based sequence
RASTLAS

SEQ ID NO: 6 - protein, 13 aa, Artificial sequence
  artificial antibody-based sequence
QGGYYSSSSTYIA

SEQ ID NO: 7 - protein, 124 aa, Artificial sequence
  artificial antibody-based sequence
QSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSS

SEQ ID NO: 8 - protein, 124 aa, Artificial sequence
  artificial antibody-based sequence
QSQVVESGGGRVQPGGSLRLSCAVSGFDLSSYAVSWVRQAPGKGLEWIGIIYPRANTYYASWAKGRFTISKDNSKNTVYLQMNSLRAEDTATYFCARDRYDSGAYLYTTYFNLWGQGTQVTVSS

SEQ ID NO: 9 - protein, 112 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLG

SEQ ID NO: 10 - protein, 124 aa, Artificial sequence
  artificial antibody-based sequence
QSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGQGTLVTVSS

SEQ ID NO: 11 - protein, 124 aa, Artificial sequence
  artificial antibody-based sequence
QSQVVESGGGRVQPGGSLRLSCAVSGFDLSSYAVSWVRQAPGKCLEWIGIIYPRANTYYASWAKGRFTISKDNSKNTVYLQMNSLRAEDTATYFCARDRYDSGAYLYTTYFNLWGQGTQVTVSS

SEQ ID NO: 12 - protein, 112 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGCGTKVTVLG

SEQ ID NO: 13 - protein, 10 aa, Artificial sequence
  artificial antibody-based sequence
GLSLSRNAMS

SEQ ID NO: 14 - protein, 10 aa, Artificial sequence
  artificial antibody-based sequence
GIDLSRNAMS

SEQ ID NO: 15 - protein, 16 aa, Artificial sequence
  artificial antibody-based sequence
IILTSGSTYYASWAKG

SEQ ID NO: 16 - protein, 11 aa, Artificial sequence
  artificial antibody-based sequence
RGIASSSLKSF

SEQ ID NO: 17 - protein, 13 aa, Artificial sequence
  artificial antibody-based sequence
QASQNVWNNNYLS

SEQ ID NO: 18 - protein, 7 aa, Artificial sequence
  artificial antibody-based sequence
TASTLAS

SEQ ID NO: 19 - protein, 10 aa, Artificial sequence
  artificial antibody-based sequence
AGGFSGEIRA

SEQ ID NO: 20 - protein, 118 aa, Artificial sequence
  artificial antibody-based sequence
QSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSS

SEQ ID NO: 21 - protein, 118 aa, Artificial sequence
  artificial antibody-based sequence
QSQLVESGGGRVQPGGSLRLSCAVSGLSLSRNAMSWVRQAPGKGLEWIGIILTSGSTYYASWAKGRFTISKDNSKNTVYLQMNSLRAEDTATYFCVRGIASSSLKSFWGQGTQVTVSS

SEQ ID NO: 22 - protein, 111 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLG

SEQ ID NO: 23 - protein, 118 aa, Artificial sequence
  artificial antibody-based sequence
QSQLVESGGGLVQPGGSLRLSCAVSGLSLSRNAMSWVRQAPGKCLEWIGIILTSGSTYYASWAKGRFTISKDNSKNTVYLQMNSLRAEDTAVYFCVRGIASSSLKSFWGQGTLVTVSS

SEQ ID NO: 24 - protein, 118 aa, Artificial sequence
  artificial antibody-based sequence
QSQLVESGGGRVQPGGSLRLSCAVSGLSLSRNAMSWVRQAPGKCLEWIGIILTSGSTYYASWAKGRFTISKDNSKNTVYLQMNSLRAEDTATYFCVRGIASSSLKSFWGQGTQVTVSS

SEQ ID NO: 25 - protein, 111 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLG

SEQ ID NO: 26 - protein, 116 aa, Artificial sequence
  artificial antibody-based sequence
QSQLVESGGGLVQPGGSLRLSCAVSGIDLSRNAMSWVRQAPGKGLEWIGIILTSGSTYYASWAKGRFTISKTSTTLDLQMNSLRAEDTAVYFCVRGIASSSLKSFWGQGTLVTVSS

SEQ ID NO: 27 - protein, 111 aa, Artificial sequence; identical to SEQ ID NO: 22
  artificial antibody-based sequence

SEQ ID NO: 28 - protein, 116 aa, Artificial sequence
  artificial antibody-based sequence
QSQLVESGGGLVQPGGSLRLSCAVSGIDLSRNAMSWVRQAPGKCLEWIGIILTSGSTYYASWAKGRFTISKTSTTLDLQMNSLRAEDTAVYFCVRGIASSSLKSFWGQGTLVTVSS

SEQ ID NO: 29 - protein, 111 aa, Artificial sequence; identical to SEQ ID NO: 25
  artificial antibody-based sequence

SEQ ID NO: 30 - protein, 10 aa, Artificial sequence
  artificial antibody-based sequence
GFSLSSYDMS

SEQ ID NO: 31 - protein, 16 aa, Artificial sequence
  artificial antibody-based sequence
ASYASGPTYYASWAKG

SEQ ID NO: 32 - protein, 12 aa, Artificial sequence
  artificial antibody-based sequence
RGGWTGTSHSNI

SEQ ID NO: 33 - protein, 13 aa, Artificial sequence
  artificial antibody-based sequence
QSSQSVFSNNYLA

SEQ ID NO: 34 - protein, 7 aa, Artificial sequence
  artificial antibody-based sequence
SASTLAS

SEQ ID NO: 35 - protein, 13 aa, Artificial sequence
  artificial antibody-based sequence
LGSYACSSADCYV

SEQ ID NO: 36 - protein, 119 aa, Artificial sequence
  artificial antibody-based sequence
EVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSS

SEQ ID NO: 37 - protein, 119 aa, Artificial sequence
  artificial antibody-based sequence
EVQLVESGGGRVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTQVTVSS

SEQ ID NO: 38 - protein, 114 aa, Artificial sequence
  artificial antibody-based sequence
DIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 39 - protein, 10 aa, Artificial sequence
  artificial antibody-based sequence
GFSLSSNAMG

SEQ ID NO: 40 - protein, 16 aa, Artificial sequence
  artificial antibody-based sequence
IISVGGFTYYASWAKG

SEQ ID NO: 41 - protein, 14 aa, Artificial sequence
  artificial antibody-based sequence
RDRHGGDSSGAFYL

SEQ ID NO: 42 - protein, 13 aa, Artificial sequence
  artificial antibody-based sequence
QSSESVYSNNQLS

SEQ ID NO: 43 - protein, 7 aa, Artificial sequence
  artificial antibody-based sequence
DASDLAS

SEQ ID NO: 44 - protein, 11 aa, Artificial sequence
  artificial antibody-based sequence
AGGFSSSSDTA

SEQ ID NO: 45 - protein, 121 aa, Artificial sequence
  artificial antibody-based sequence
EVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 46 - protein, 112 aa, Artificial sequence
  artificial antibody-based sequence
DIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLG

SEQ ID NO: 47 - protein, 121 aa, Artificial sequence
  artificial antibody-based sequence
EVQLVESGGGLVQPGGSLRLSCAASGFSLSSNAMGWVRQAPGKCLEYIGIISVGGFTYYASWAKGRFTISRDNSKNTVYLQMNSLRAEDTATYFCARDRHGGDSSGAFYLWGQGTLVTVSS

SEQ ID NO: 48 - protein, 112 aa, Artificial sequence
  artificial antibody-based sequence
DIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGCGTKLTVLG

SEQ ID NO: 49 - protein, 11 aa, Artificial sequence
  artificial antibody-based sequence
GFSFSSSYWIC

SEQ ID NO: 50 - protein, 17 aa, Artificial sequence
  artificial antibody-based sequence
CVFTGDGTTYYASWAKG

SEQ ID NO: 51 - protein, 12 aa, Artificial sequence
  artificial antibody-based sequence
RPVSVYYYGMDL

SEQ ID NO: 52 - protein, 11 aa, Artificial sequence
  artificial antibody-based sequence
QASQIISSRSA

SEQ ID NO: 53 - protein, 7 aa, Artificial sequence
  artificial antibody-based sequence
QASKLAS

SEQ ID NO: 54 - protein, 11 aa, Artificial sequence
  artificial antibody-based sequence
QCTYIDSNFGA

SEQ ID NO: 55 - protein, 121 aa, Artificial sequence
  artificial antibody-based sequence
EVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 56 - protein, 110 aa, Artificial sequence
  artificial antibody-based sequence
DVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGGGTKLTVLG

SEQ ID NO: 57 - protein, 121 aa, Artificial sequence
  artificial antibody-based sequence
EVQLVESGGGLVQPGGSLRLSCAASGFSFSSSYWICWVRQAPGKCLEWVGCVFTGDGTTYYASWAKGRFTISRDNSKNTVYLQMNSLRAEDTATYFCARPVSVYYYGMDLWGQGTLVTVSS

SEQ ID NO: 58 - protein, 110 aa, Artificial sequence
  artificial antibody-based sequence
DVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGCGTKLTVLG

SEQ ID NO: 59 - protein, 10 aa, Artificial sequence
  artificial antibody-based sequence
GFSLSSYAMN

SEQ ID NO: 60 - protein, 16 aa, Artificial sequence
  artificial antibody-based sequence
HINAGDIAYYATWAKG

SEQ ID NO: 61 - protein, 13 aa, Artificial sequence
  artificial antibody-based sequence
RGAGGFSTGPFKL

SEQ ID NO: 62 - protein, 11 aa, Artificial sequence
  artificial antibody-based sequence
QASESINSRLA

SEQ ID NO: 63 - protein, 7 aa, Artificial sequence
  artificial antibody-based sequence
DASDLTS

SEQ ID NO: 64 - protein, 10 aa, Artificial sequence
  artificial antibody-based sequence
QGYGGSSTTT

SEQ ID NO: 65 - protein, 120 aa, Artificial sequence
  artificial antibody-based sequence
EVQLVESGGGLVQPGGSLRLSCAASGFSLSSYAMNWVRQAPGKGLEWIGHINAGDIAYYATWAKGRFTISRDNSKNTVYLQMNSLRAEDTAVYFCARGAGGFSTGPFKLWGQGTLVTVSS

SEQ ID NO: 66 - protein, 120 aa, Artificial sequence
  artificial antibody-based sequence
EVQLVESGGGRVQPGGSLRLSCAASGFSLSSYAMNWVRQAPGKGLEWIGHINAGDIAYYATWAKGRFTISRDNSKNTVYLQMNSLRAEDTATYYCARGAGGFSTGPFKLWGQGTQVTVSS

SEQ ID NO: 67 - protein, 109 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGGGTKLTVLG

SEQ ID NO: 68 - protein, 120 aa, Artificial sequence
  artificial antibody-based sequence
EVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 69 - protein, 120 aa, Artificial sequence
  artificial antibody-based sequence
EVQLVESGGGRVQPGGSLRLSCAASGFSLSSYAMNWVRQAPGKCLEWIGHINAGDIAYYATWAKGRFTISRDNSKNTVYLQMNSLRAEDTATYFCARGAGGFSTGPFKLWGQGTQVTVSS

SEQ ID NO: 70 - protein, 109 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLG

SEQ ID NO: 71 - protein, 122 aa, Artificial sequence
  artificial antibody-based sequence
EVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSS

SEQ ID NO: 72 - protein, 121 aa, Artificial sequence
  artificial antibody-based sequence
QVQLVQSGAEVKKPGSSVKVSCKASGIDFNSNYYMCWVRQAPGQGLEWMGCIYVGSHVNTYYANWAKGRVTITADESTSTAYMELSSLRSEDTAVYYCATSGSSVLYFKFWGQGTLVTVSS

SEQ ID NO: 73 - protein, 121 aa, Artificial sequence
  artificial antibody-based sequence
QVQLVQSGAEVKKPGASVKVSCKASGIDFNSNYYMCWVRQAPGQGLEWMGCIYVGSHVNTYYANWAKGRVTMTRDTSISTAYMELSSLRSEDTAVYYCATSGSSVLYFKFWGQGTLVTVSS

SEQ ID NO: 74 - protein, 124 aa, Artificial sequence
  artificial antibody-based sequence
QVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSS

SEQ ID NO: 75 - protein, 108 aa, Artificial sequence
  artificial antibody-based sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGTGTKVTVLG

SEQ ID NO: 76 - protein, 11 aa, Artificial sequence
  artificial antibody-based sequence
FGTGTKVTVLG

SEQ ID NO: 77 - protein, 11 aa, Artificial sequence
  artificial antibody-based sequence
FGGGTKLTVLG

SEQ ID NO: 78 - protein, 11 aa, Artificial sequence
  artificial antibody-based sequence
FGGGTQLIILG

SEQ ID NO: 79 - protein, 11 aa, Artificial sequence
  artificial antibody-based sequence
FGEGTELTVLG

SEQ ID NO: 80 - protein, 11 aa, Artificial sequence
  artificial antibody-based sequence
FGSGTKVTVLG

SEQ ID NO: 81 - protein, 11 aa, Artificial sequence
  artificial antibody-based sequence
FGGGTQLTVLG

SEQ ID NO: 82 - protein, 11 aa, Artificial sequence
  artificial antibody-based sequence
FGGGTQLTALG

SEQ ID NO: 83 - protein, 11 aa, Artificial sequence
  artificial antibody-based sequence
FGCGTKVTVLG

SEQ ID NO: 84 - protein, 20 aa, Artificial sequence
  artificial antibody-based sequence
GGGGSGGGGSGGGGSGGGGS

SEQ ID NO: 85 - protein, 5 aa, Artificial sequence
  artificial antibody-based sequence
GGGGS

SEQ ID NO: 86 - protein, 10 aa, Artificial sequence
  artificial antibody-based sequence
GGGGSGGGGS

SEQ ID NO: 87 - protein, 753 aa, Artificial sequence
  artificial antibody-based sequence
DVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSFSSSYWICWVRQAPGKCLEWVGCVFTGDGTTYYASWAKGRFTISRDNSKNTVYLQMNSLRAEDTATYFCARPVSVYYYGMDLWGQGTLVTVSSGGGGSGGGGSAQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSS

SEQ ID NO: 88 - protein, 755 aa, Artificial sequence
  artificial antibody-based sequence
DIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSNAMGWVRQAPGKCLEYIGIISVGGFTYYASWAKGRFTISRDNSKNTVYLQMNSLRAEDTATYFCARDRHGGDSSGAFYLWGQGTLVTVSSGGGGSGGGGSAQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSS

SEQ ID NO: 89 - protein, 758 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSDVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSS

SEQ ID NO: 90 - protein, 758 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSDVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGQGTLVTVSS

SEQ ID NO: 91 - protein, 751 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSAQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSS

SEQ ID NO: 92 - protein, 751 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSAQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGLSLSRNAMSWVRQAPGKCLEWIGIILTSGSTYYASWAKGRFTISKDNSKNTVYLQMNSLRAEDTAVYFCVRGIASSSLKSFWGQGTLVTVSS

SEQ ID NO: 93 - protein, 749 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSAQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGIDLSRNAMSWVRQAPGKCLEWIGIILTSGSTYYASWAKGRFTISKTSTTLDLQMNSLRAEDTAVYFCVRGIASSSLKSFWGQGTLVTVSS

SEQ ID NO: 94 - protein, 749 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSAQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGIDLSRNAMSWVRQAPGKGLEWIGIILTSGSTYYASWAKGRFTISKTSTTLDLQMNSLRAEDTAVYFCVRGIASSSLKSFWGQGTLVTVSS

SEQ ID NO: 95 - protein, 495 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 96 - protein, 518 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 97 - protein, 495 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGQGTLVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 98 - protein, 518 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 99 - protein, 486 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGIDLSRNAMSWVRQAPGKGLEWIGIILTSGSTYYASWAKGRFTISKTSTTLDLQMNSLRAEDTAVYFCVRGIASSSLKSFWGQGTLVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 100 - protein, 509 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGIDLSRNAMSWVRQAPGKGLEWIGIILTSGSTYYASWAKGRFTISKTSTTLDLQMNSLRAEDTAVYFCVRGIASSSLKSFWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 101 - protein, 486 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGIDLSRNAMSWVRQAPGKCLEWIGIILTSGSTYYASWAKGRFTISKTSTTLDLQMNSLRAEDTAVYFCVRGIASSSLKSFWGQGTLVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 102 - protein, 509 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGIDLSRNAMSWVRQAPGKCLEWIGIILTSGSTYYASWAKGRFTISKTSTTLDLQMNSLRAEDTAVYFCVRGIASSSLKSFWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 103 - protein, 488 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 104 - protein, 511 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 105 - protein, 488 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGLSLSRNAMSWVRQAPGKCLEWIGIILTSGSTYYASWAKGRFTISKDNSKNTVYLQMNSLRAEDTAVYFCVRGIASSSLKSFWGQGTLVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 106 - protein, 511 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGLSLSRNAMSWVRQAPGKCLEWIGIILTSGSTYYASWAKGRFTISKDNSKNTVYLQMNSLRAEDTAVYFCVRGIASSSLKSFWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 107 - protein, 758 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGRVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTQVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGRVQPGGSLRLSCAASGFSLSSYAMNWVRQAPGKCLEWIGHINAGDIAYYATWAKGRFTISRDNSKNTVYLQMNSLRAEDTATYFCARGAGGFSTGPFKLWGQGTQVTVSSGGGGSGGGGSDVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGRVQPGGSLRLSCAVSGFDLSSYAVSWVRQAPGKGLEWIGIIYPRANTYYASWAKGRFTISKDNSKNTVYLQMNSLRAEDTATYFCARDRYDSGAYLYTTYFNLWGQGTQVTVSS

SEQ ID NO: 108 - protein, 751 aa, Artificial sequence
  artificial antibody-based sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGRVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTQVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGRVQPGGSLRLSCAASGFSLSSYAMNWVRQAPGKCLEWIGHINAGDIAYYATWAKGRFTISRDNSKNTVYLQMNSLRAEDTATYFCARGAGGFSTGPFKLWGQGTQVTVSSGGGGSGGGGSAQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLGGGGGSGGGGSGGGGSGGGGSQSQLVESGGGRVQPGGSLRLSCAVSGLSLSRNAMSWVRQAPGKCLEWIGIILTSGSTYYASWAKGRFTISKDNSKNTVYLQMNSLRAEDTATYFCVRGIASSSLKSFWGQGTQVTVSS

SEQ ID NO: 109 - protein, 495 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGRVQPGGSLRLSCAVSGFDLSSYAVSWVRQAPGKGLEWIGIIYPRANTYYASWAKGRFTISKDNSKNTVYLQMNSLRAEDTATYFCARDRYDSGAYLYTTYFNLWGQGTQVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 110 - protein, 518 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGRVQPGGSLRLSCAVSGFDLSSYAVSWVRQAPGKGLEWIGIIYPRANTYYASWAKGRFTISKDNSKNTVYLQMNSLRAEDTATYFCARDRYDSGAYLYTTYFNLWGQGTQVTVSSGGGGSGGGGSEVQLVESGGGRVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTQVTVSSGGGSGGGSGGGSGEVQLVESGGGRVQPGGSLRLSCAASGFSLSSYAMNWVRQAPGKCLEWIGHINAGDIAYYATWAKGRFTISRDNSKNTVYLQMNSLRAEDTATYFCARGAGGFSTGPFKLWGQGTQVTVSS

SEQ ID NO: 111 - protein, 495 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGCGTKVTVLGGGGGSGGGGSGGGGSGGGGSQSQVVESGGGRVQPGGSLRLSCAVSGFDLSSYAVSWVRQAPGKCLEWIGIIYPRANTYYASWAKGRFTISKDNSKNTVYLQMNSLRAEDTATYFCARDRYDSGAYLYTTYFNLWGQGTQVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 112 - protein, 518 aa, Artificial sequence
  artificial antibody-based sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGCGTKVTVLGGGGGSGGGGSGGGGSGGGGSQSQVVESGGGRVQPGGSLRLSCAVSGFDLSSYAVSWVRQAPGKCLEWIGIIYPRANTYYASWAKGRFTISKDNSKNTVYLQMNSLRAEDTATYFCARDRYDSGAYLYTTYFNLWGQGTQVTVSSGGGGSGGGGSEVQLVESGGGRVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTQVTVSSGGGSGGGSGGGSGEVQLVESGGGRVQPGGSLRLSCAASGFSLSSYAMNWVRQAPGKCLEWIGHINAGDIAYYATWAKGRFTISRDNSKNTVYLQMNSLRAEDTATYFCARGAGGFSTGPFKLWGQGTQVTVSS

SEQ ID NO: 113 - protein, 495 aa, Artificial sequence; identical to SEQ ID NO: 109
  artificial antibody-based sequence

SEQ ID NO: 114 - protein, 511 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGRVQPGGSLRLSCAVSGLSLSRNAMSWVRQAPGKGLEWIGIILTSGSTYYASWAKGRFTISKDNSKNTVYLQMNSLRAEDTATYFCVRGIASSSLKSFWGQGQLVTVSSGGGGSGGGGSEVQLVESGGGRVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTQVTVSSGGGSGGGSGGGSGEVQLVESGGGRVQPGGSLRLSCAASGFSLSSYAMNWVRQAPGKCLEWIGHINAGDIAYYATWAKGRFTISRDNSKNTVYLQMNSLRAEDTATYFCARGAGGFSTGPFKLWGQGTQVTVSS

SEQ ID NO: 115 - protein, 495 aa, Artificial sequence; identical to SEQ ID NO: 109
  artificial antibody-based sequence

SEQ ID NO: 116 - protein, 509 aa, Artificial sequence
  artificial antibody-based sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGRVQPGGSLRLSCAVSGIDLSRNAMSWVRQAPGKGLEWIGIILTSGSTYYASWAKGRFTISKTSTTLDLQMNSLRAEDTATYFCVRGIASSSLKSFWGQGTQVTVSSGGGGSGGGGSEVQLVESGGGRVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTQVTVSSGGGSGGGSGGGSGEVQLVESGGGRVQPGGSLRLSCAASGFSLSSYAMNWVRQAPGKCLEWIGHINAGDIAYYATWAKGRFTISRDNSKNTVYLQMNSLRAEDTATYFCARGAGGFSTGPFKLWGQGTQVTVSS

SEQ ID NO: 117 - protein, 406 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILY

SEQ ID NO: 118 - protein, 5 aa, Artificial sequence
  artificial antibody-based sequence
  Repeat (1)..(5): n repeats with n being an integer of at least one
GSGGS

SEQ ID NO: 119 - protein, 5 aa, Artificial sequence
  artificial antibody-based sequence
  Repeat (1)..(5): n repeats with n being an integer of at least one
GGGGS

SEQ ID NO: 120 - protein, 5 aa, Artificial sequence
  artificial antibody-based sequence
  Repeat (1)..(4): n repeats with n being an integer of at least one
GGGGS

SEQ ID NO: 121 - protein, 5 aa, Artificial sequence
  artificial antibody-based sequence
  Repeat (1)..(5): n repeats with n being 1 or 2
GGGGS

SEQ ID NO: 122 - protein, 5 aa, Artificial sequence
  artificial antibody-based sequence
  Repeat (1)..(5): n repeats with n being 1, 2, 3, 4, 5, 6, 7 or 8
GGGGS